(19) United States
(12) Patent Application Publication
(10) Pub. No.: US 2003/0143544 A1
(43) Pub. Date: Jul. 31, 2003

(54) Diagnosis and treatment of vascular disease

(75) Inventor: Jeanette McCarthy, San Diego, CA (US)

Correspondence Address: LAHIVE & COCKFIELD, 28 STATE STREET, BOSTON, MA, 02109, US

(73) Assignee: Vitivity, Inc., Cambridge, MA

(21) Appl. No.: 10/043,715

(22) Filed: Jan. 9, 2002

(51) Int. Cl. C12Q 1/68; G06F 19/00; G01N 33/48; G01N 33/50

(52) U.S. Cl. 435/6; 702/20

(57) ABSTRACT

The present invention is based at least in part on the discovery of polymorphisms within the endothelin-1 (EDN1) gene. Accordingly, the invention provides nucleic acid molecules having a nucleotide sequence of an allelic variant of an EDN1 gene. The invention also provides methods for identifying specific alleles of polymorphic regions of an EDN1 gene, methods for determining whether a subject has or is at risk of developing a disease which is associated with a specific allele of a polymorphic region of an EDN1 gene, e.g., a vascular disease, based on detection of one or more polymorphisms within the EDN1 gene, and kits for performing such methods. The invention further provides methods for identifying a subject who has, or is at risk for developing, a vascular disease or disorder as a candidate for a particular clinical course of therapy or a particular diagnostic evaluation. The invention further provides methods for selecting a clinical course of therapy or a diagnostic evaluation to treat a subject who is at risk for developing, a vascular disease or disorder.

### BACKGROUND OF THE INVENTION

Cardiovascular disease is a major health risk throughout the industrialized world. Coronary artery disease (CAD), or atherosclerosis, involves the progressional narrowing of the arteries due to a build-up of atherosclerotic plaque. Myocardial infarction (MI), e.g., heart attack, results when the heart is damaged due to reduced blood flow to the heart caused by the build-up of plaque in the coronary arteries.

Coronary artery disease, the most prevalent of cardiovascular diseases, is the principal cause of heart attack, stroke, and gangrene of the extremities, and thereby the principle cause of death in the United States. CAD is a complex disease involving many cell types and molecular factors (described in, for example, Ross, 1993, Nature 362: 801-809). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. Injury or dysfunction of the vascular endothelium is a common feature of may conditions that predispose a subject to accelerated development of atherosclerotic cardiovascular disease. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDLs are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

These lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque.

Such plaques occlude the blood vessel concerned and, thus, restrict the flow of blood, resulting in ischemia. Ischemia is a condition characterized by a lack of oxygen supply in tissues of organs due to inadequate perfusion. Such inadequate perfusion can have a number of natural causes, including atherosclerotic or restenotic lesions, anemia, or stroke. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease. Ischemia may occur in any organ, however, that is suffering a lack of oxygen supply.

One of the most important risk factors for coronary artery disease is a familial history. Although family history subsumes both genetic and shared environmental factors, studies suggest that CAD has a very strong genetic component (Marenberg, et al. (1994) NEJM 330:1041). Despite the importance of family history as a risk factor for CAD, it's incomplete genetic basis has not been elucidated. Therefore, the identification of genes which are involved in the development of CAD and MI would be beneficial.

A family of structurally and pharmacologically distinct peptides, the endothelins, has been identified and sequenced in humans (Inoue et al. (1989) Proc. Natl. Acad. Sci. USA 86(8):2863). Three isoforms of human endothelin have been identified: endothelins-1, -2, and -3. Endothelin-1 (EDN1) is a potent, 212-amino acid vasoconstrictor peptide produced by vascular endothelial cells. Inoue et al. ((1989) J. Biol. Chem. 264(25):14954-9) cloned the full length of the human preproendothelin-1 gene and the corresponding cDNA and determined the complete nucleotide sequence. The 2,026-nucleotide mRNA, excluding the poly(A) tail, is encoded in 5 exons distributed over 6,836 bp. Endothelin-1 was originally isolated from the supernatant of porcine aortic endothelial cell cultures and is the most potent vasoconstrictor known. Subsequent cloning and sequence analysis from a human placental cDNA library showed that human endothelin-1 is identical to porcine endothelin. In addition to its vasoconstrictor action, endothelin has effects on the central nervous system and on neuronal excitability. Benatti et al. ((1993) J Clin Invest. 91(3): 1149-56) demonstrated that at least 2 preproendothelin-1 mRNAs are produced from a single gene by use of different promoters; the 2 molecules share the same coding sequence but differ in the 5-prime untranslated region. Analysis of the tissue distribution of the 2 mRNAs showed a tissue-type specificity for one mRNA in brain and heart tissues. Maemura, et al. ((1996) Am J Clin Med. 24(2):165-8) found that the highest expression of EDN1 mRNA was detected in the lung in adult mice, whereas in the embryo the gene is predominantly expressed in the epithelium and mesenchyme of the pharyngeal arches and in the endothelium of the large arteries.

It would be beneficial to identify polymorphic regions within genes which are associated with a vascular disease or disorder, such as coronary artery disease or myocardial infarction. It would further be desirable to provide prognostic, diagnostic, pharmacogenomic, and therapeutic methods utilizing the identified polymorphic regions.

### SUMMARY OF THE INVENTION

The present invention is based, at least in part, on the identification of polymorphic regions within the endothelin-1 (EDN1) gene which are associated with specific diseases or disorders, including vascular diseases or disorders. In particular, single nucleotide polymorphisms (SNPs) in this gene which are associated with premature coronary artery disease (CAD) (or coronary heart disease) and myocardial infarction (MI) have been identified. Accordingly, SNPs in this gene, as identified herein, in combination with each other, or with other SNPs in the EDN1 gene or other genes, can be utilized to predict, in a subject, an increased risk for developing a vascular disease, e.g., CAD and/or MI.

One polymorphism identified in the EDN1 gene is a change from a thymidine (T) to a cytidine (C) at residue 157790 of the reference sequence GI 2791272 (polymorphism ID No. G456a4). This SNP is a non-coding variant and thus does not result in a change in the amino acid sequence of EDN1 (SEQ ID NO:2). Another polymorphism identified in the EDN1 gene is a change from a guanine (G) to a thymidine (T) at residue 159908 of the reference sequence GI 2791272 (polymorphism ID No. G456a3). This SNP is a missense variant, and thus results in a change from a lysine (K) to an asparagine (N) in the amino acid sequence of the EDN1 protein (SEQ ID NO:2). These two SNPs are in strong linkage disequilibrium with each other.

It has been found that in the population tested, individuals who carry at least one copy of the variant allele of the G456a4 SNP (C) or the variant allele of the G456a3 SNP (T), but not both variant alleles together, are at an increased risk of vascular disease, e.g., CAD or MI.

Thus, the invention relates to polymorphic regions and in particular, SNPs identified as described herein in combination with each other or with other polymorphisms in the EDN1 gene or in other genes. The invention also relates to the use of these SNPs, and other SNPs in the EDN1 gene or in other genes, particularly those in linkage disequilibrium with these SNPs, for diagnosis, prediction of clinical course of therapy and treatment response for vascular disease. The SNPs identified herein may further be used in the development of new treatments for vascular disease based upon comparison of the variant and normal versions of the gene or gene product (e.g., the reference sequence), and development of cell-culture based and animal models for research and treatment of vascular disease. The invention further relates to novel compounds and pharmaceutical compositions for use in the diagnosis and treatment of such disorders. In preferred embodiments, the vascular disease is CAD or MI.

In one embodiment, the polymorphic regions of the invention are associated with responsiveness to vascular disease or disorder therapies, e.g., clinical courses of therapy, including, but not limited to lifestyle changes, medications, medical devices, such as a defibrillator, a stent, a device used in coronary revascularization, a pacemaker, and any combination thereof, surgical or non-surgical intervention or procedures such as percutaneous transluminal coronary angioplasty, laser angioplasty, implantation of a stent, coronary bypass grafting, implantation of a defibrillator, implantation of a pacemaker, and any combination thereof. The medical devices described in the methods of the invention can also be used in combination with a modulator of EDN1 gene expression or EDN1 polypeptide activity.

Furthermore, the polymorphic regions of the invention are also useful in the determination of use of further diagnostic protocols, including, but not limited to, diagnostic vascular imaging, genetic analysis, familial health history analysis, lifestyle analysis, exercise stress tests, or any combination thereof.

The polymorphisms of the invention may thus be used, or in combination with each other or with polymorphisms in the EDN1 gene or in other genes, in prognostic, diagnostic, and therapeutic methods. For example, the polymorphisms of the invention can be used to determine whether a subject has, or is, or is not at risk of developing a disease or disorder associated with a specific allelic variant of an EDN1 polymorphic region, e.g., a disease or disorder associated with aberrant EDN1 activity, e.g., a vascular disease or disorder. The invention thus relates to isolated nucleic acid molecules and methods of using these molecules. The nucleic acid molecules of the invention include specific allelic variants which differ from the EDN1 reference sequence set forth in SEQ ID NO:1 (GI 2791272), or a portion thereof. The preferred nucleic acid molecules of the invention comprise an EDN1 polymorphic region or portion thereof, having the polymorphisms shown in Table 1, polymorphisms in linkage disequilibrium with the polymorphisms shown in Table 1, and combinations thereof. Nucleic acids of the invention can function as probes or primers, e.g., in methods for determining the allelic identity of an EDN1 polymorphic region in a nucleic acid of interest.

The nucleic acids of the invention can also be used, in combination with each other or with other polymorphisms in the EDN1 gene or in other genes, to determine whether a subject is at risk of developing a disease associated with a specific allelic variant of an EDN1 polymorphic region, e.g., a disease or disorder associated with aberrant EDN1 activity, e.g., a vascular disease or disorder such as CAD or MI. The nucleic acids of the invention can further be used to prepare EDN1 polypeptides encoded by specific alleles, such as mutant (variant) alleles. Such polypeptides can be used in therapy. Polypeptides encoded by specific EDN1 alleles, such as variant EDN1 polypeptides, can also be used as immunogens and selection agents for preparing, isolating or identifying antibodies that specifically bind EDN1 proteins encoded by these alleles. Accordingly, such antibodies can be used to detect variant EDN1 proteins. The nucleic acid molecules of the invention can be double- or single-stranded. Accordingly, in one embodiment of the invention, a complement of the nucleotide sequence is provided wherein the polymorphism has been identified; i.e., where there has been a single nucleotide change from a thymidine to a cytidine in a single strand, the complement of that strand will contain a change from a adenine to a guanine at the corresponding nucleotide residue. The invention further provides allele-specific oligonucleotides that hybridize to a gene comprising a polymorphism of the present invention or to its complement.

The polymorphisms of the present invention, in combination with each other, or with previously identified polymorphisms, are shown herein to be associated with specific disorders, e.g., vascular diseases or disorders. Examples of vascular diseases or disorders include, without limitation, atherosclerosis, coronary artery disease (CAD), myocardial infarction (MI), ischemia, stroke, peripheral vascular diseases, venous thromboembolism and pulmonary embolism.

The invention further provides vectors comprising the nucleic acid molecules of the present invention; host cells transfected with said vectors whether prokaryotic or eukaryotic; and transgenic non-human animals which contain a heterologous form of a functional or non-functional EDN1 allele described herein. Such a transgenic animal can serve as an animal model for studying the effect of specific EDN1 allelic variations, including mutations, as well as for use in drug screening and/or recombinant protein production.

The invention further provides methods for determining at least a portion of an EDN1 gene. In a preferred embodiment, the method comprises contacting a sample nucleic acid comprising an EDN1 gene sequence with a probe or primer having a sequence which is complementary to an EDN1 gene sequence, carrying out a reaction that would amplify and/or detect differences in a region of interest within the EDN1 gene sequence, and comparing the result of each reaction with that of a reaction with a control (known) EDN1 gene (e.g., an EDN1 gene from a human not afflicted with a vascular disease or disorder e.g., CAD, MI, or another disease associated with an aberrant EDN1 activity) so as to determine the molecular structure of the EDN1 gene sequence in the sample nucleic acid. The method of the invention can be used for example in determining the molecular structure of at least a portion of an exon, an intron, a 5′ upstream regulatory element, or the 3′ untranslated region. In a preferred embodiment, the method comprises determining the identity of nucleotides present at residues 157790 and 159908 of the reference sequence GI 2791272 (the EDN1 gene), or the complements thereof.

In another preferred embodiment, the method comprises determining the nucleotide content of at least a portion of an EDN1 gene, such as by sequence analysis. In yet another embodiment, determining the molecular structure of at least a portion of an EDN1 gene is carried out by single-stranded conformation polymorphism (SSCP). In yet another embodiment, the method is an oligonucleotide ligation assay (OLA). Other methods within the scope of the invention for determining the molecular structure of at least a portion of an EDN1 gene include hybridization of allele-specific oligonucleotides, sequence specific amplification, primer specific extension, and denaturing high performance liquid chromatography (DHPLC). In at least some of the methods of the invention, the probe or primer is allele specific. Preferred probes or primers are single stranded nucleic acids, which optionally are labeled.

The methods of the invention can be used for determining the identity of a nucleotide or amino acid residue within a polymorphic region of a human EDN1 gene present in a subject. For example, the methods of the invention can be useful for determining whether a subject has, or is or is not at risk of developing, a disease or condition associated with a specific allelic variant of a polymorphic region in the human EDN1 gene, e.g., a vascular disease or disorder.

In one embodiment, the disease or condition is characterized by an aberrant EDN1 activity, such as aberrant EDN1 protein level, which can result from aberrant expression of an EDN1 gene. The disease or condition can be CAD, MI, or another vascular disease. Accordingly, the invention provides methods for predicting vascular diseases associated with aberrant EDN1 activity.

The invention also provides a method of identifying subjects which are at increased risk of developing CAD and/or MI, wherein the method comprises the steps of i) identifying in DNA from a subject at least one sequence polymorphism, as compared with the reference EDN1 gene sequence which comprises SEQ ID NO:1, in an EDN1 gene sequence; and ii) identifying the subject based on the identified polymorphism.

In another embodiment, the invention also provides a method for identifying a subject as a candidate for a particular clinical course of therapy for a vascular disease or disorder, e.g., CAD or MI, for example, treatment with medications, lifestyle changes, use of medical devices such as a defibrillator, a stent, a device used in coronary revascularization, a pacemaker, and any combination thereof and/or surgical devices, such as, but not limited to, angioplasty devices, used in, for example, surgical procedures such as percutaneous transluminal coronary balloon angioplasty (PTCA) or laser angioplasty, implantation of a stent, or surgical intervention, such as coronary bypass grafting (CABG), or any combination thereof, wherein the method comprises the steps of obtaining a nucleic acid sample from the subject, determining the identity of the nucleotides present at nucleotide positions 157790 and 159908 of SEQ ID NO:1, or the complements thereof, and identifying the subject based on the identified nucleotides, as a subject who is a candidate for a particular clinical course of therapy for a vascular disease or disorder.

In yet another embodiment, the invention provides a method of identifying a subject as a candidate for further diagnostic evaluation for a vascular disease or disorder or for the risk of a vascular disease or disorder, such as, for example, cardiovascular imaging, such as angiography, cardiac ultrasound, coronary angiogram, magnetic resonance imagery, nuclear imaging, CT, myocardial perfusion imagery, or electrocardiogram, genetic analysis, e.g., identification of additional polymorphisms, familial health history analysis, lifestyle analysis, or exercise stress tests, alone or in combination, wherein the method comprises the steps of obtaining a nucleic acid sample from the subject, determining the identity of one or more of the nucleotides present at nucleotide positions 157790 and 159908 of SEQ ID NO: 1, or the complements thereof, and identifying the subject based on the identified nucleotides, as a subject who is or is not a candidate for further diagnostic evaluation, or who would or would not benefit from further diagnostic evaluation for a vascular disease or disorder.

In a further embodiment, the invention provides a method for treating a subject having a disease or condition associated with a specific allelic variant of a polymorphic region of an EDN1 gene. In one embodiment, the method comprises the steps of (a) determining the identity of the allelic variant; and (b) administering to the subject a clinical course of therapy that compensates for the effect of the specific allelic variant e.g., treatment with medications, lifestyle changes, surgical devices, such as, but not limited to, angioplasty devices, used in, for example, percutaneous transluminal coronary balloon angioplasty (PTCA) or laser angioplasty, implantation of a stent, or surgical procedures, such as percutaneous transluminal coronary angioplasty, laser angioplasty, implantation of a stent, coronary bypass grafting, implantation of a defibrillator, implantation of a pacemaker, and any combination thereof. In one embodiment, the clinical course of therapy is administration of an agent or modulator which modulates, e.g., agonizes or antagonizes, EDN1 nucleic acid expression or EDN1 protein levels. In a preferred embodiment, the modulator is selected from the group consisting of a nucleic acid, a ribozyme, an antisense EDN1 nucleic acid molecule, an EDN1 protein or polypeptide, an antibody, a peptidomimetic, or a small molecule.

In a preferred embodiment, the specific allelic variant is a mutation. The mutation can be located, e.g., in a 5′ upstream regulatory element, a 3′ regulatory element, an intron, or an exon of the gene. Thus, for example, in a subject having at least one copy of the variant allele (C) at nucleotide position 157790 of GI 279272, in combination with at least one copy of the reference allele (G) at nucleotide position 159908 of GI 279272, or the complements thereof, or at least one copy of the reference allele (T) at nucleotide position 157790 of GI 279272 in combination with at least one copy of the variant allele (T) at nucleotide position 159908 of GI 279272, or the complements thereof, vascular disorders such as CAD or MI, can be treated, prevented, or ameliorated by administering to the subject a particular clinical course of treatment sufficient to treat, prevent, or ameliorate the vascular disease or disorder.

Additionally, the invention provides a method of identifying a subject who is susceptible to a vascular disorder, which method comprises the steps of i) providing a nucleic acid sample from a subject; and ii) detecting in the nucleic acid sample one or more EDN1 gene polymorphisms, that correlate with the vascular disorder with a P value less than or equal to 0.05, the existence of the polymorphism being indicative of susceptibility to the vascular disorder.

The invention also provides a method of treating vascular disorders which method comprises the step of i) identifying in genetic material of a subject an EDN1 gene polymorphism that correlates with increased responsiveness to a clinical course of treatment as compared with responsiveness of a subject lacking the polymorphism; and ii) administering the clinical course of therapy to the subject.

The invention further provides forensic methods based on detection of polymorphisms within the EDN1 gene.

The invention also provides probes and primers comprising oligonucleotides, which correspond to a region of nucleotide sequence which hybridizes to at least 6 consecutive nucleotides of the sequence set forth as SEQ ID NOs:3 or 4, or to the complement of the sequences set forth as SEQ ID NOs:3 or 4, or naturally occurring mutants or variants thereof. In preferred embodiments, the probe/primer further includes a label attached thereto, which is capable of being detected.

In another embodiment, the invention provides a kit for amplifying and/or for determining the molecular structure of at least a portion of an EDN1 gene, comprising a probe or primer capable of hybridizing to an EDN1 gene and instructions for use. In a preferred embodiment, determining the molecular structure of a region of an EDN1 gene comprises determining the identity of the allelic variant of the polymorphic region. Determining the molecular structure of at least a portion of an EDN1 gene can comprise determining the identity of at least one nucleotide or determining the nucleotide composition, e.g., the nucleotide sequence an EDN1 gene.

A kit of the invention can be used, e.g., for determining whether a subject is or is not at risk of developing a disease associated with a specific allelic variant of a polymorphic region of an EDN1 gene, e.g., CAD or MI. In a preferred embodiment, the invention provides a kit for determining whether a subject is or is not at risk of developing a vascular disease such as, for example, atherosclerosis, CAD, MI, ischemia, stroke, peripheral vascular diseases, venous thromboembolism and pulmonary embolism. The kit of the invention can also be used in selecting the appropriate clinical course of treatment for a subject. Thus, determining the allelic variants of EDN1 polymorphic regions of a subject can be useful in predicting how a subject will respond to a specific drug, e.g., a drug for treating a disease or disorder associated with aberrant EDN1, e.g., a vascular disease or disorder.

Other features and advantages of the invention will be apparent from the following detailed description and claims.

### BRIEF DESCRIPTION OF THE FIGURES

FIG. 1 depicts the nucleotide sequence corresponding to reference sequence GI 2791272 (SEQ ID NO:1) for the EDN1 gene.

FIG. 2 depicts the amino acid sequence corresponding to reference sequence GI 4503461 (SEQ ID NO:2) for the EDN1 protein.

### DETAILED DESCRIPTION OF THE INVENTION

The present invention is based, at least in part, on the discovery that two SNPs in the EDN1 gene, identified herein as G456a4 and G456a3, have been identified which are associated with an increased risk of vascular disease, e.g., MI and CAD, in a subject. The G456a4 SNP is a change from a thymidine (T) to a cytidine (C) in the EDN1 gene at residue 157790 of the reference sequence GI 2791272 (polymorphism ID No. G456a4). This SNP is a non-coding variant and thus does not result in a change in the amino acid sequence of EDN1 (SEQ ID NO:2). The G456a3 SNP is a change from a guanine (G) to a thymidine (T) in the EDN1 gene at residue 159908 of the reference sequence GI 2791272 (polymorphism ID No. G456a3). This SNP is a missense variant, and thus results in a change in the amino acid sequence of the EDN1 protein (SEQ ID NO:2) from a lysine (K) to an asparagine (N).

In the population tested, individuals who carried at least one copy of either variant allele (allele C for the G456a4 SNP or allele T for the G456a3 SNP), but not both variant alleles, were found to be at increased risk of CAD and MI.

Comparing individuals who were carriers of one variant allele (e.g., carriers of either allele C for the G456a4 SNP in combination with allele G for the G456a3 SNP or carriers of allele T for the G456a4 SNP in combination with allele T for the G456a3 SNP), to those with both variants (e.g., carriers of allele C for the G456a4 SNP in combination with allele T for the G456a3 SNP) or neither variant (carriers of allele T for the G456a4 SNP in combination with allele G for the G456a3 SNP) gave an odds ratio of 2.53 for CAD (p=0.000002) and an odds ratio of 2.27 for MI (p=0.0004). Therefore, subjects having at least one copy of the variant allele (C) at nucleotide position 157790 of GI 2791272, in combination with at least one copy of the reference allele (G) at nucleotide position 159908 of GI 2791272, or the complements thereof, or at least one copy of the reference allele (T) at nucleotide position 157790 of GI 2791272, in combination with at least one copy of the variant allele (T) at nucleotide position 159908 of GI 2791272, or the complements thereof, have an increased risk of vascular disease than individuals with both variants (C at nucleotide position 157790 of GI 2791272 and T at nucleotide position 159908 of GI 2791272) or individuals with neither variant (T at nucleotide position 157790 of GI 2791272 and G at nucleotide position 159908 of GI 2791272).

These results suggest that two different haplotypes in the EDN1 gene are associated with vascular disease, e.g., CAD and MI. As used herein, the term “haplotype” refers to a set of polymorphisms which are in linkage disequilibrium with each other. That is, the polymorphisms comprising the haplotype segregate together. The first haplotype is comprised of allele C for the SNP G456a4 (the variant allele) and allele G for the SNP G456a3 (the reference allele). The second haplotype is comprised of allele T for the SNP G456a4 (the reference allele) and allele T for the SNP G456a3 (the variant allele). These two haplotypes each result in increased risk of vascular disease relative to all other haplotypes of these two SNPs (see Table 2, in the Examples section).

Without intending to be limited by theory, the true causative variant(s) which underlies this increased risk may be located in another position in the END1 gene and be in linkage disequilibrium with both of the risk haplotypes defined here. Alternatively, two or more causative variants may exist in the END1 gene, each being represented by a distinct haplotype (two of which are described here) with which it is in linkage disequilibrium.

The term “linkage” describes the tendency of genes, alleles, loci or genetic markers to be inherited together as a result of their location on the same chromosome. It can be measured by percent recombination between the two genes, alleles, loci, or genetic markers. The term “linkage disequilibrium,” also referred to herein as “LD,” refers to a greater than random association between specific alleles at two marker loci within a particular population. In general, linkage disequilibrium decreases with an increase in physical distance. If linkage disequilibrium exists between two markers, or SNPs, then the genotypic information at one marker, or SNP, can be used to make probabilistic predictions about the genotype of the second marker.

The polymorphisms of the present invention are single nucleotide polymorphisms (SNPs) at a specific nucleotide residues within the EDN1 gene. The EDN1 gene has at least two alleles, referred to herein as the reference allele and the variant allele. The reference allele (i.e., the consensus sequence, or wild type allele) has been designated based on it's frequency in a general U.S. Caucasian population sample. The reference allele is the more common of the two alleles; the variant is the more rare of the two alleles. Nucleotide sequences in GenBank may correspond to either allele and correspond to the nucleotide sequence of the nucleotide sequence which has been deposited in GenBank™ and given a specific Accession Number (e.g., GI 2791272, the reference sequence for the EDN1 gene). The reference sequence for the amino acid sequence of EDN1 protein is set forth as SEQ ID NO:2. The variant allele differs from the reference allele by at least one nucleotide at the site identified in Table 1, and those in linkage disequilibrium therewith. The present invention thus relates to nucleotides comprising variant alleles of the EDN1 reference sequence and/or complements of the variant allele to be used in combination with each other or in combination with other SNPs to predict the risk of vascular disease.

The invention further relates to nucleotides comprising portions of the variant alleles and/or portions of complements of the variant alleles which comprise the site of the polymorphism and are at least 5 nucleotides or basepairs in length. Portions can be, for example., 5-10, 5-15, 10-20, 2-25, 10-30, 10-50 or 10-100 bases or basepairs long. For example, a portion of a variant allele which is 17 nucleotides or basepairs in length includes the polymorphism (i.e., the nucleotide(s) which differ from the reference allele at that site) and twenty additional nucleotides or basepairs which flank the site in the variant allele. These additional nucleotides and basepairs can be on one or both sides of the polymorphism. The polymorphisms which are the subject of this invention are defined in Table 1 with respect to the reference sequence identified in Table 1, and those polymorphisms in linkage disequilibrium with the polymorphisms of the present invention.

It is understood that the invention is not limited by this exemplified reference sequence, as variants of this sequence which differ at locations other than the SNP site identified herein can also be utilized. The skilled artisan can readily determine the SNP sites in these other reference sequences which correspond to the SNP site identified herein by aligning the sequence of interest with the reference sequences specifically disclosed herein, and programs for performing such alignments are commercially available. For example, the ALIGN program in the GCG software package can be used, utilizing a PAM120 weight residue table, a gap length penalty of 12 and a gap penalty of 4, for example.

The polymorphic regions of the present invention are associated with specific diseases or disorders and have been identified in the human EDN1 gene by analyzing the DNA of cell lines derived from an ethnically diverse population by methods described in Cargill, et al. (1999) Nature Genetics 22:231-238.

Cases which were used to identify associations between vascular disease and SNPs were comprised of 352 U.S. Caucasian subject with premature coronary artery disease were identified in 15 participating medical centers, fulfilling the criteria of either myocardial infarction, surgical or percutaneous revascularization, or a significant coronary artery lesion diagnosed before age 45 in men or age 50 in women and having a living sibling who met the same criteria. These cases were compared with a random sample of 418 Caucasian controls drawn from the general U.S. population in Atlanta, Ga.

The allelic variants of the present invention were identified by performing denaturing high performance liquid chromatography (DHPLC) analysis, variant detector arrays (Affymetrix™), the polymerase chain reaction (PCR), and/or single stranded conformation polymorphism (SSCP) analysis of genomic DNA from independent individuals as described in the Examples, using PCR primers complementary to intronic sequences surrounding each of the exons, 3′ UTR, and 5′ upstream regulatory element sequences of the human EDN1 gene.

The presence of at least two polymorphisms in the human EDN1 gene in the population studied were identified. The preferred polymorphisms of the invention are listed in Table 1. Table 1 contains a “polymorphism ID No.” in column 2, which is used herein to identify the variants, e.g., G456a4 and G456a3. In Table 1, the nucleotide sequences flanking the polymorphisms are provided in column 8. There are 15 nucleotides flanking the polymorphic nucleotide residues (i.e., 15 nucleotides 5′ of the polymorphism and 15 nucleotides 3′ of the polymorphism). Column 9 indicates the SEQ ID NO. that is used to identify each polymorphism. SEQ ID NOs:3 and 4 comprise the sequence shown in column 8 where the variant nucleotide residues are indicated by a lower-case letter.

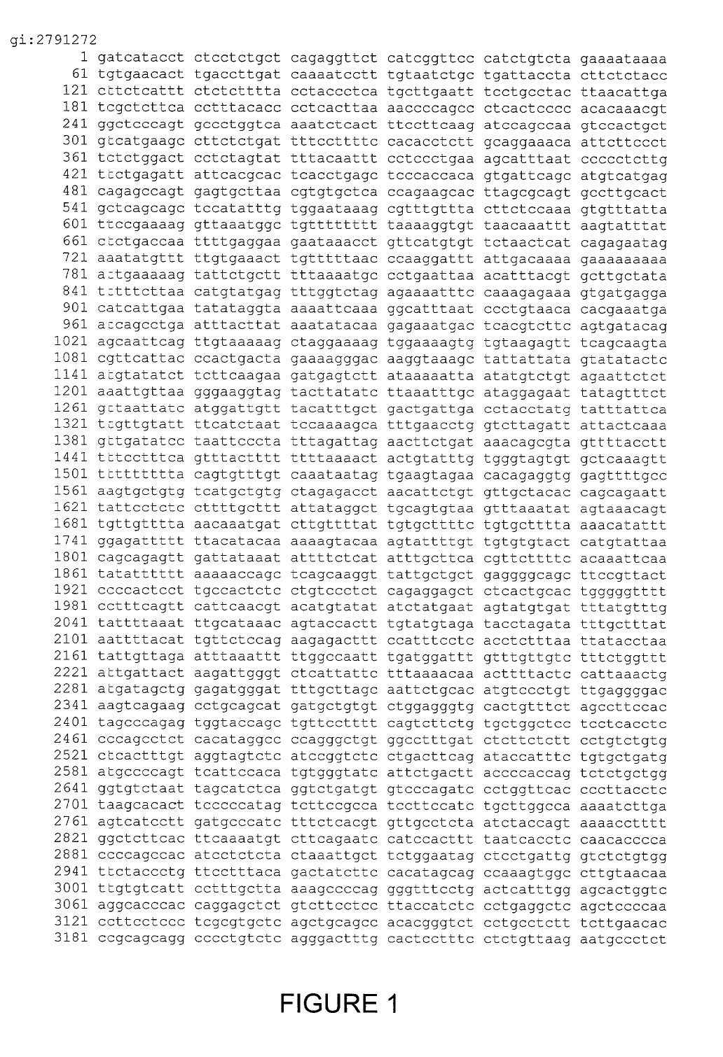

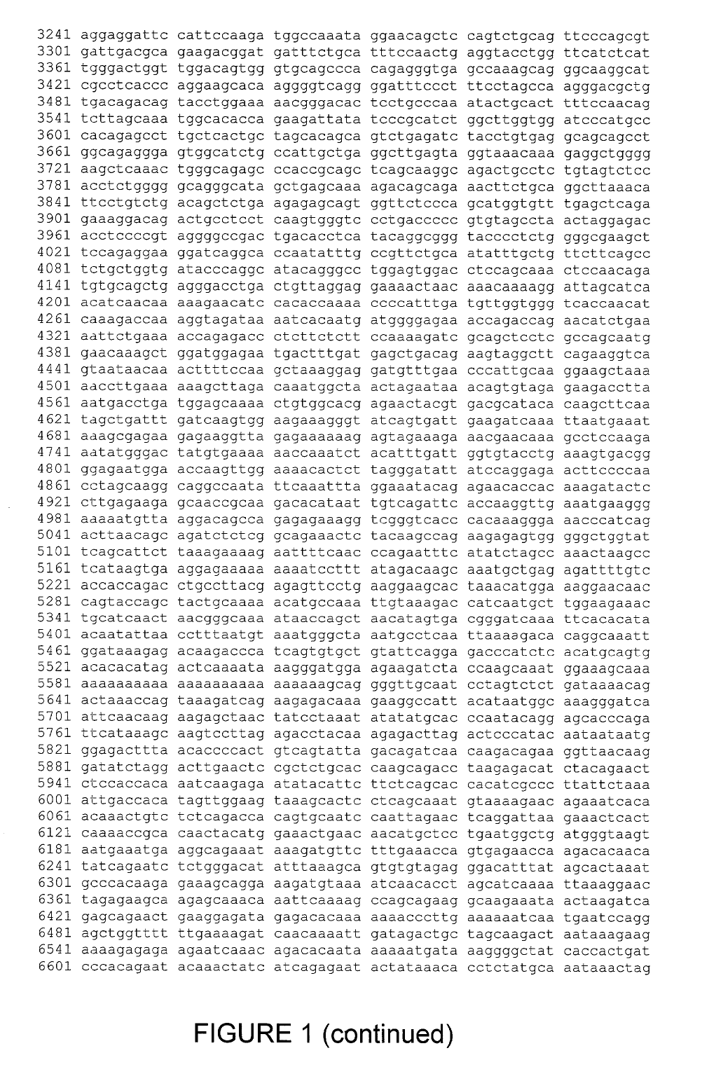

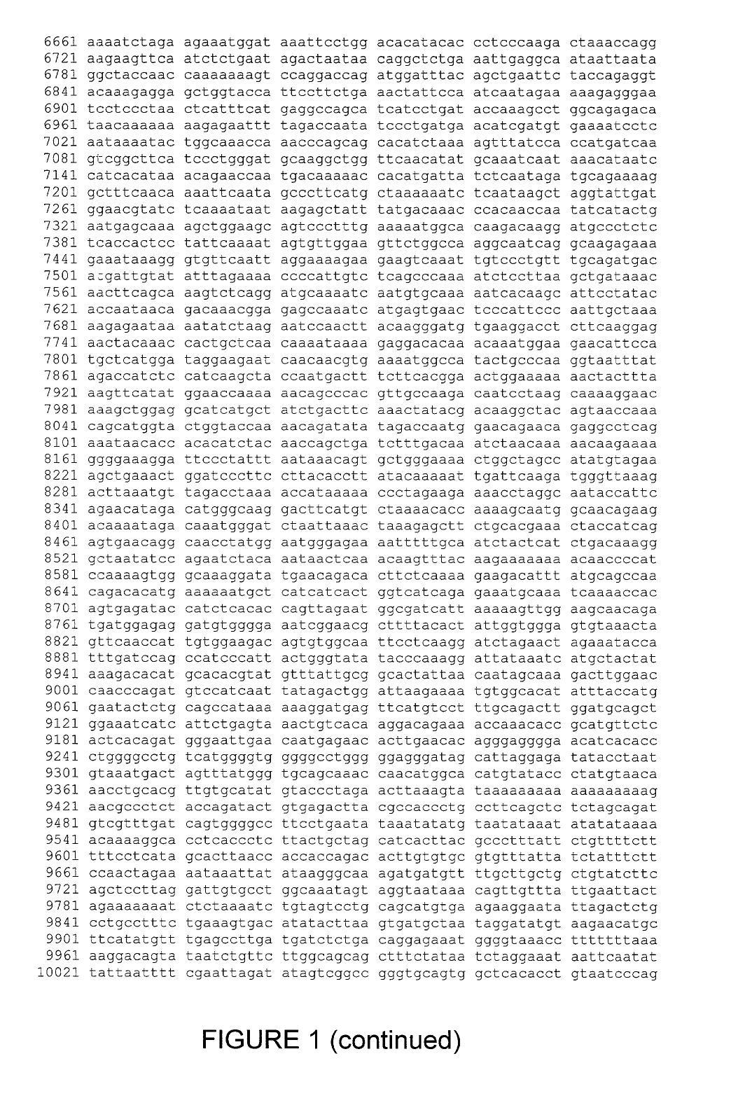

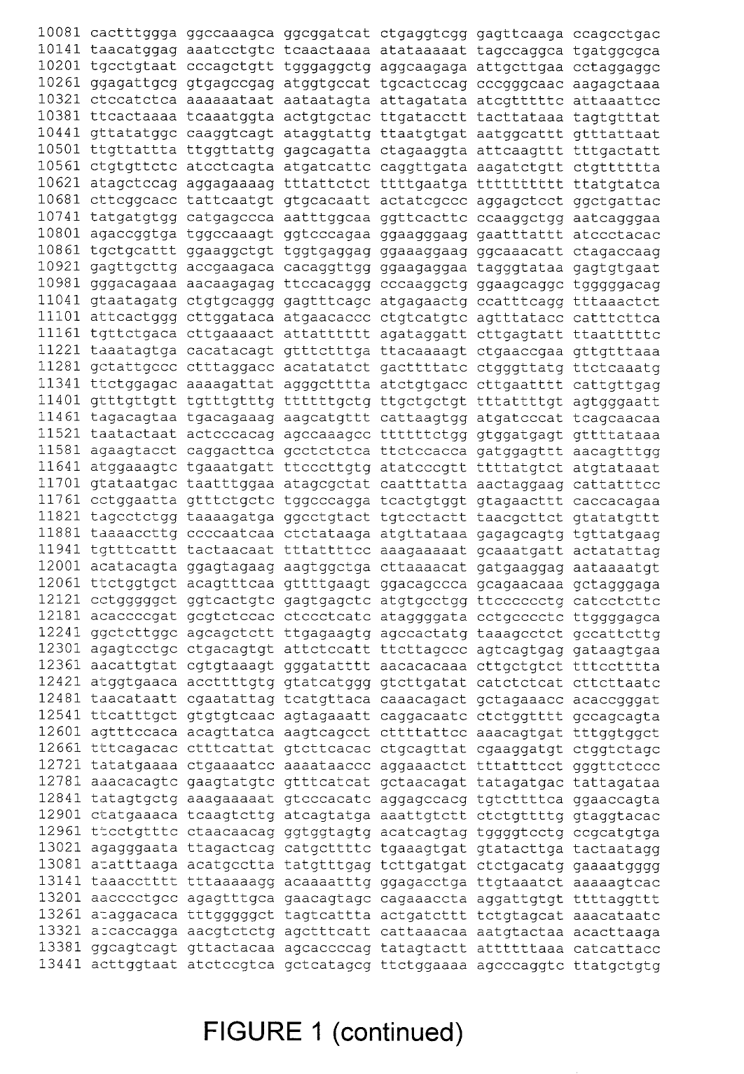

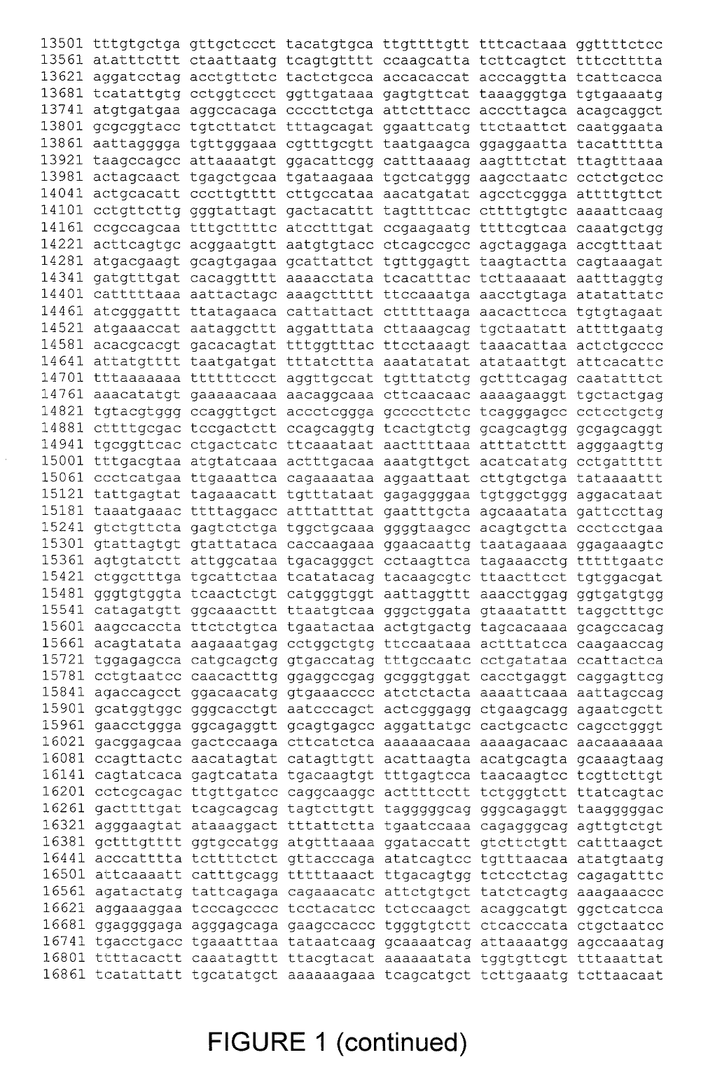

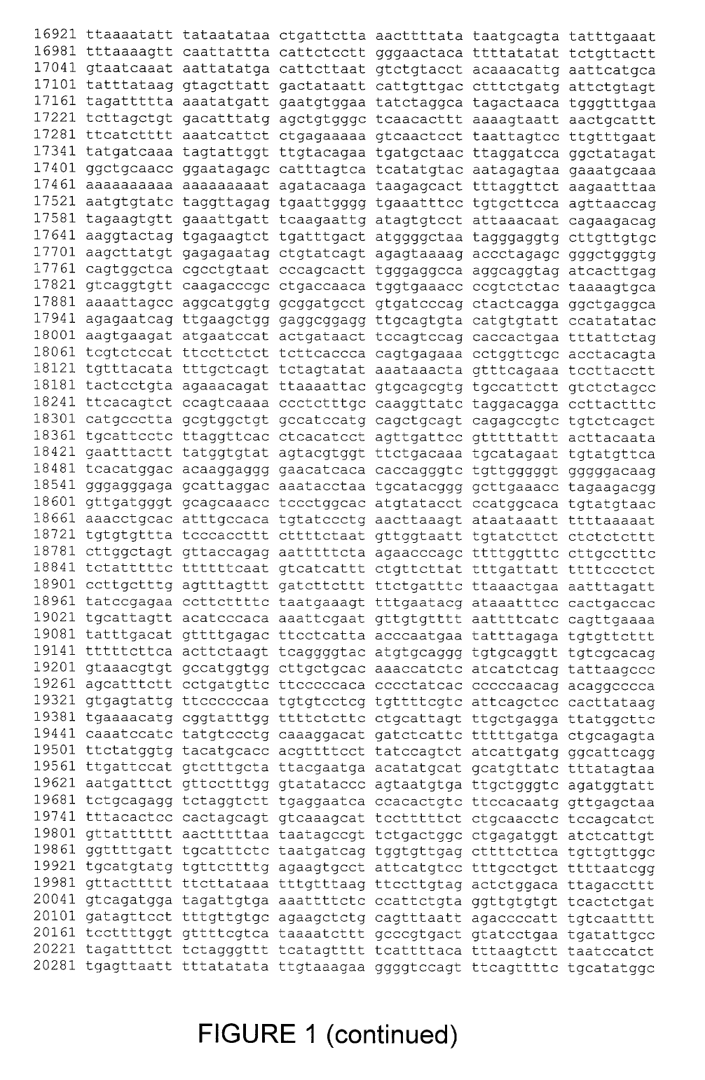

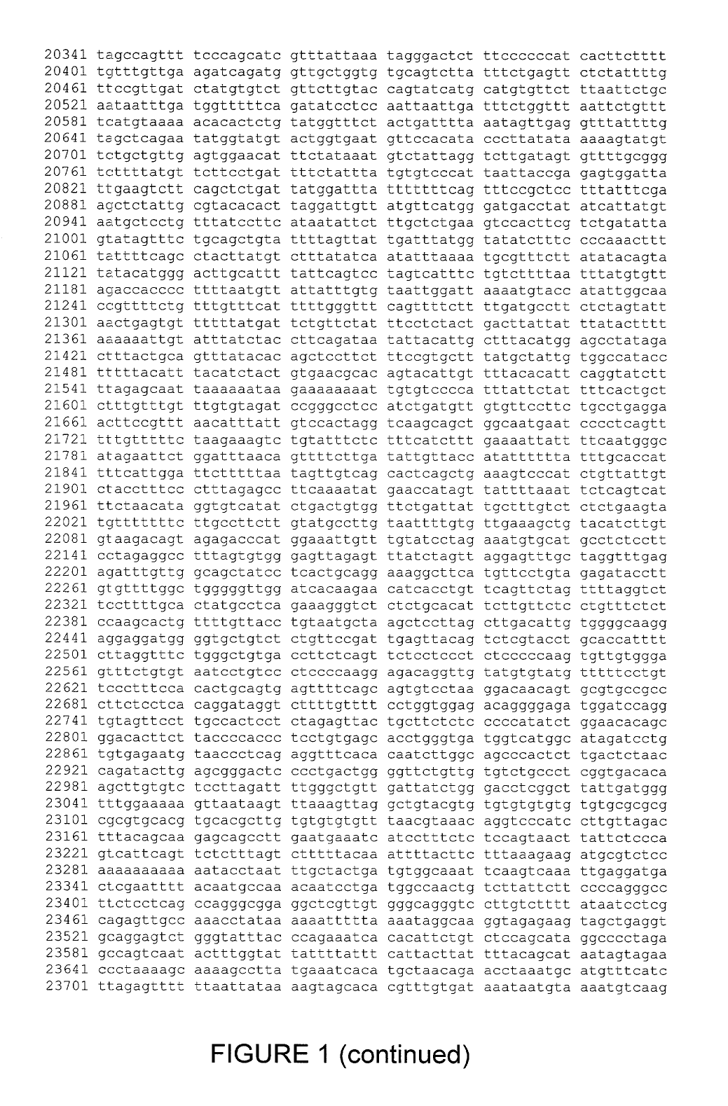

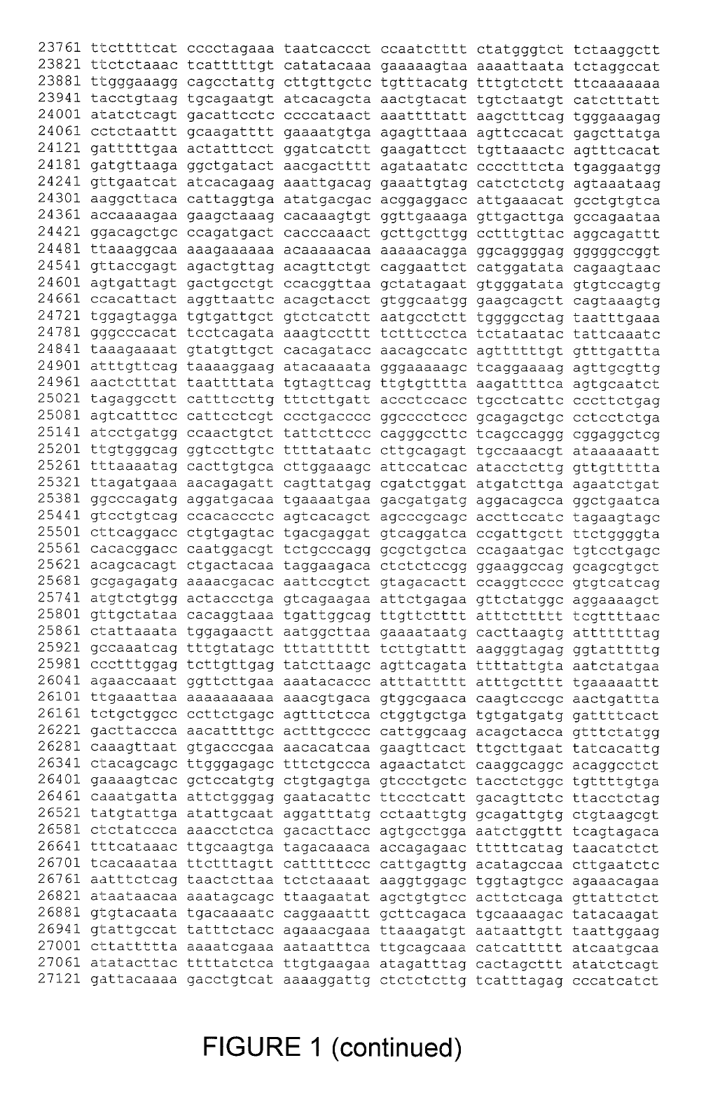

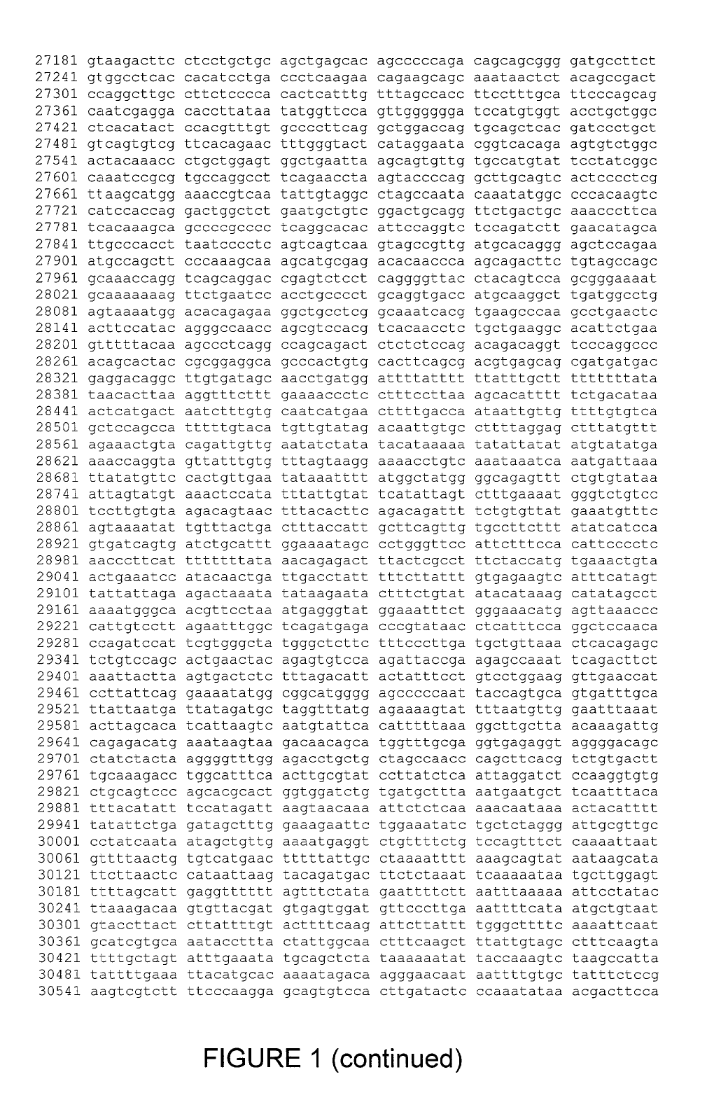

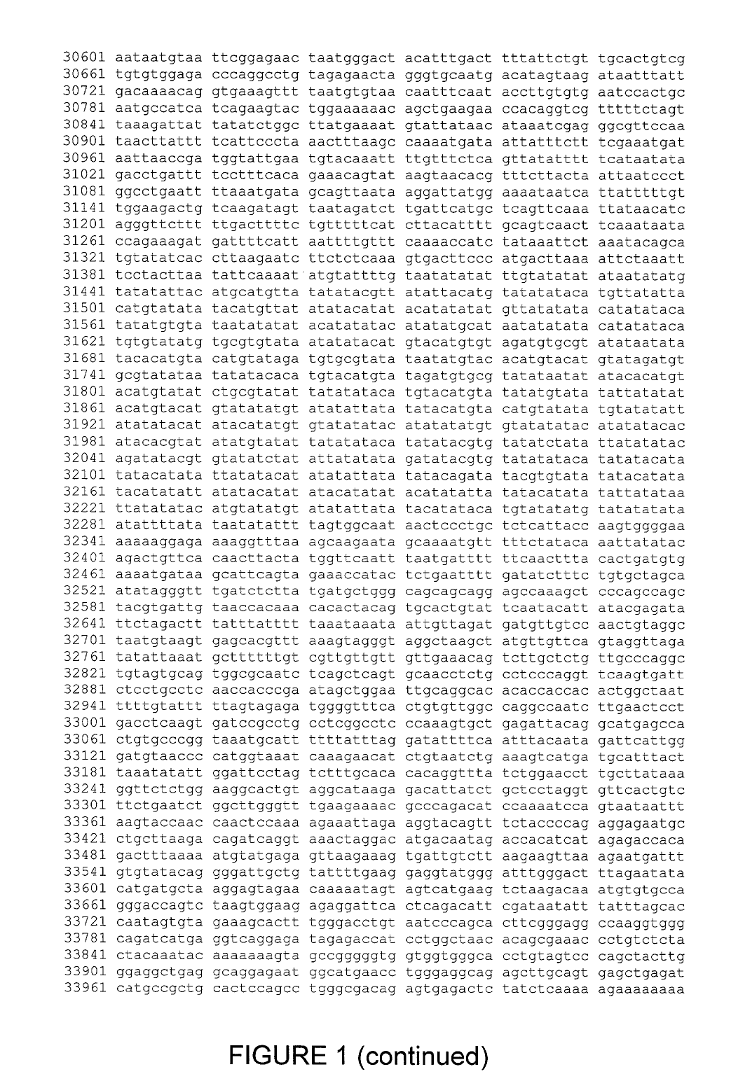

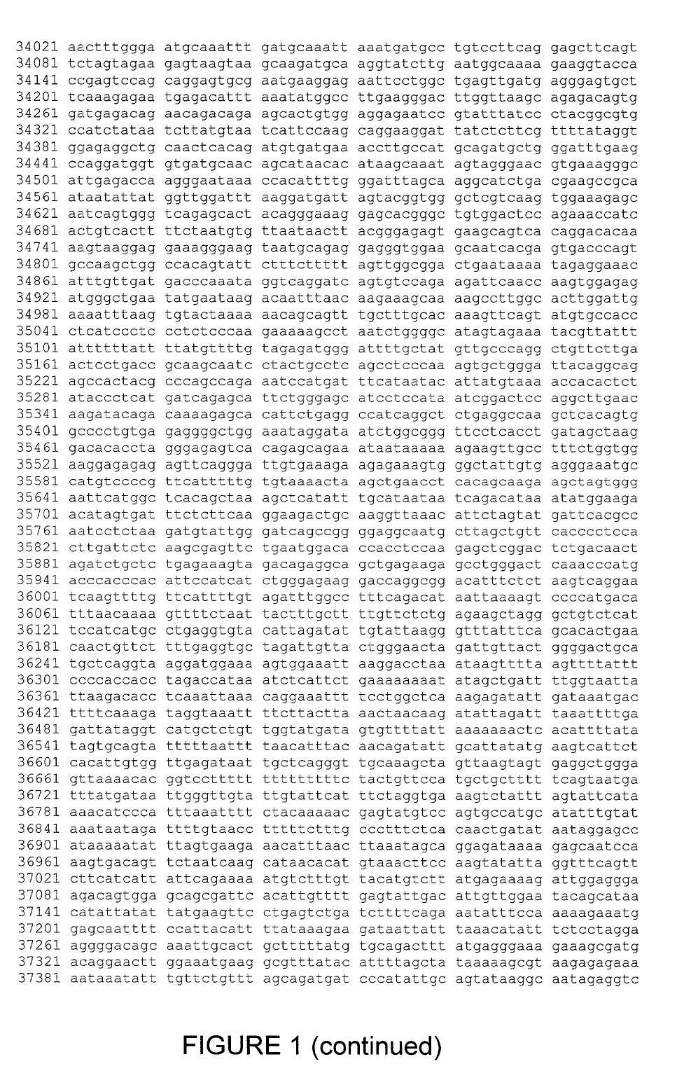

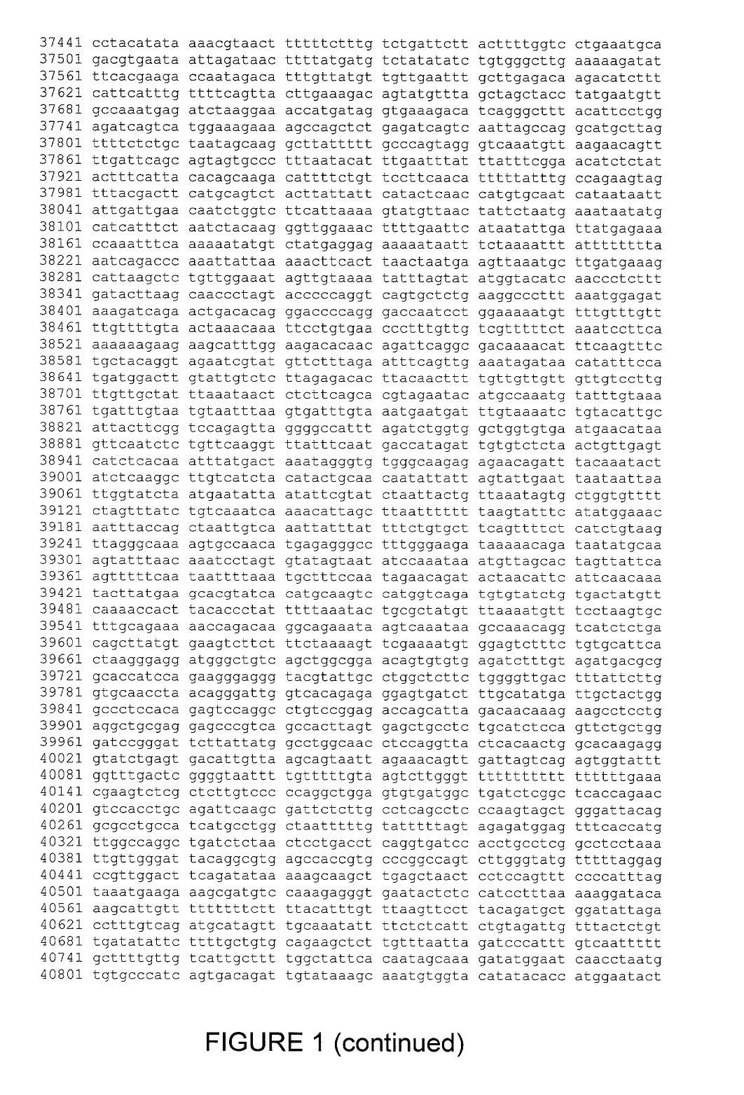

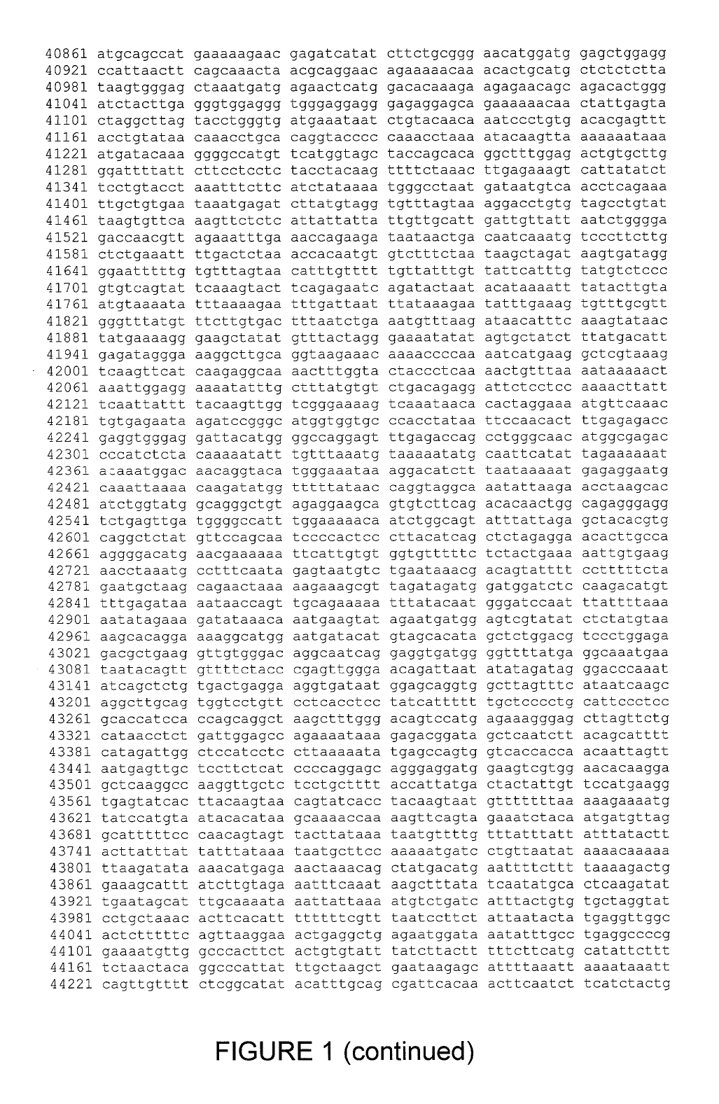

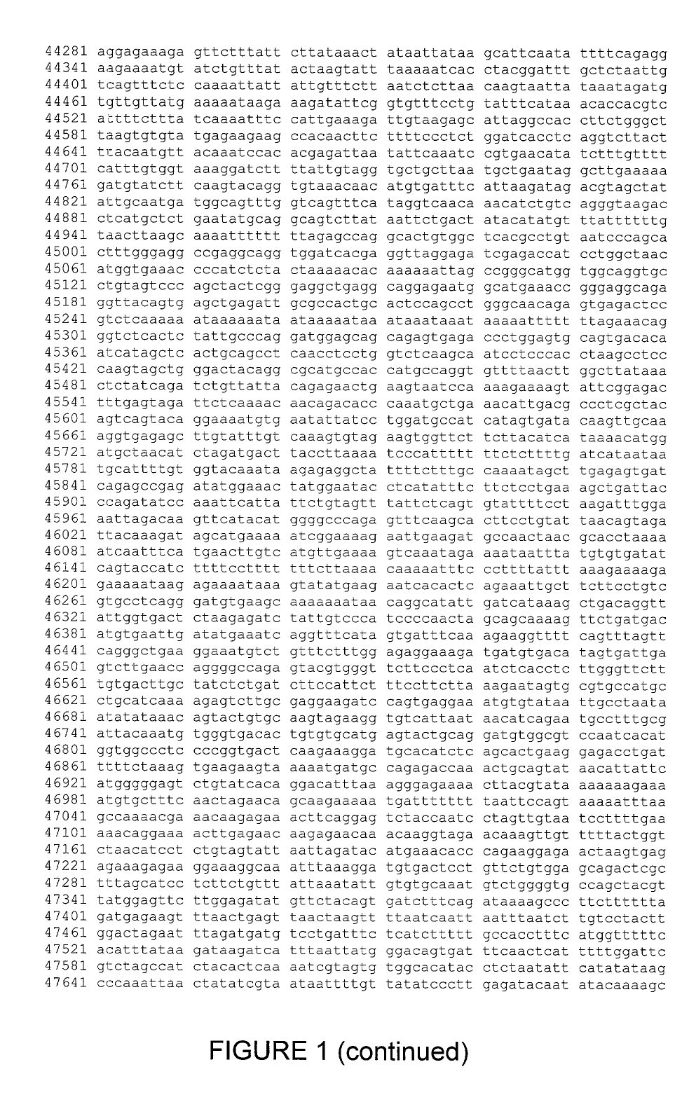

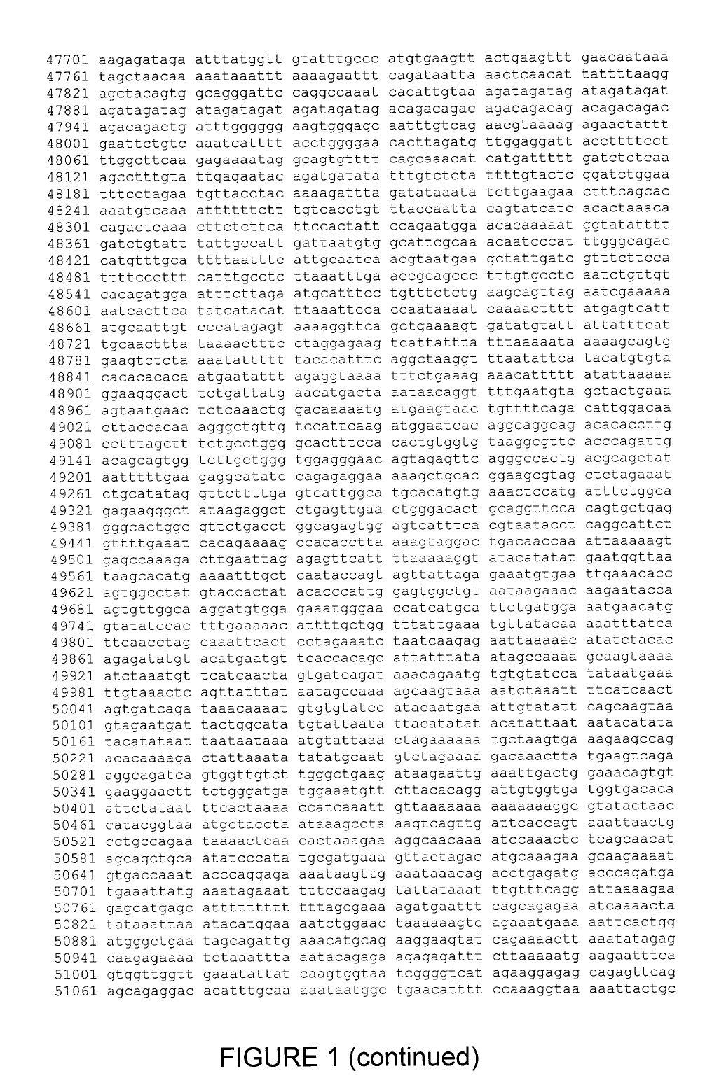

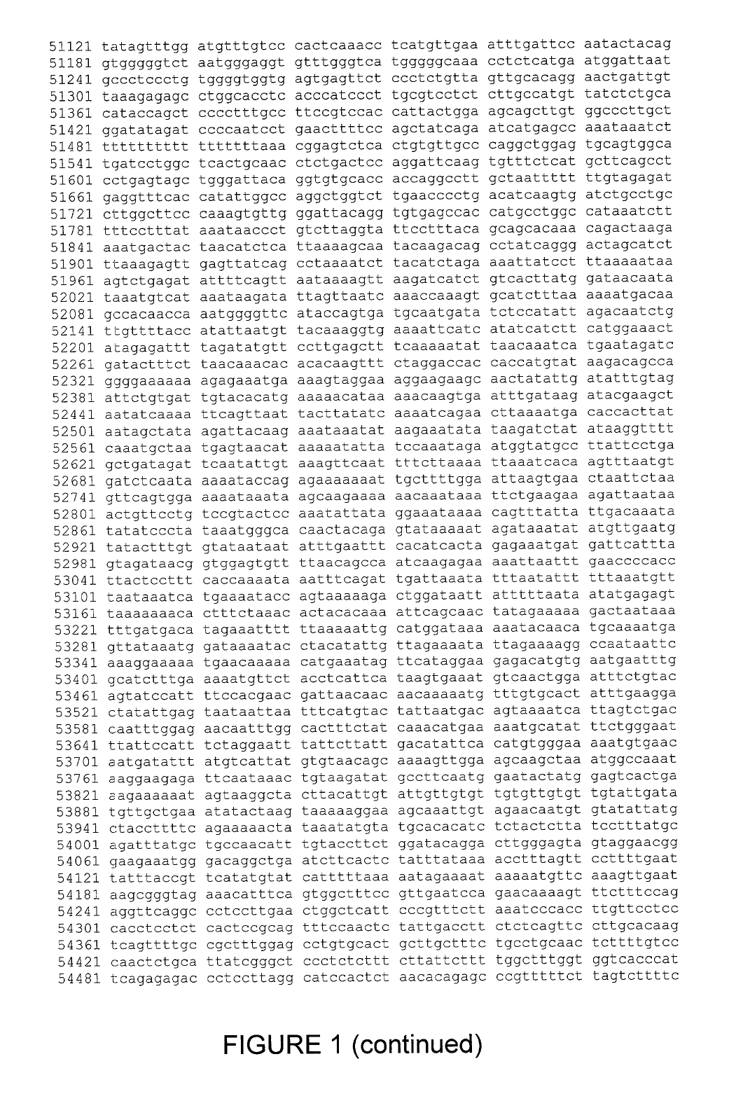

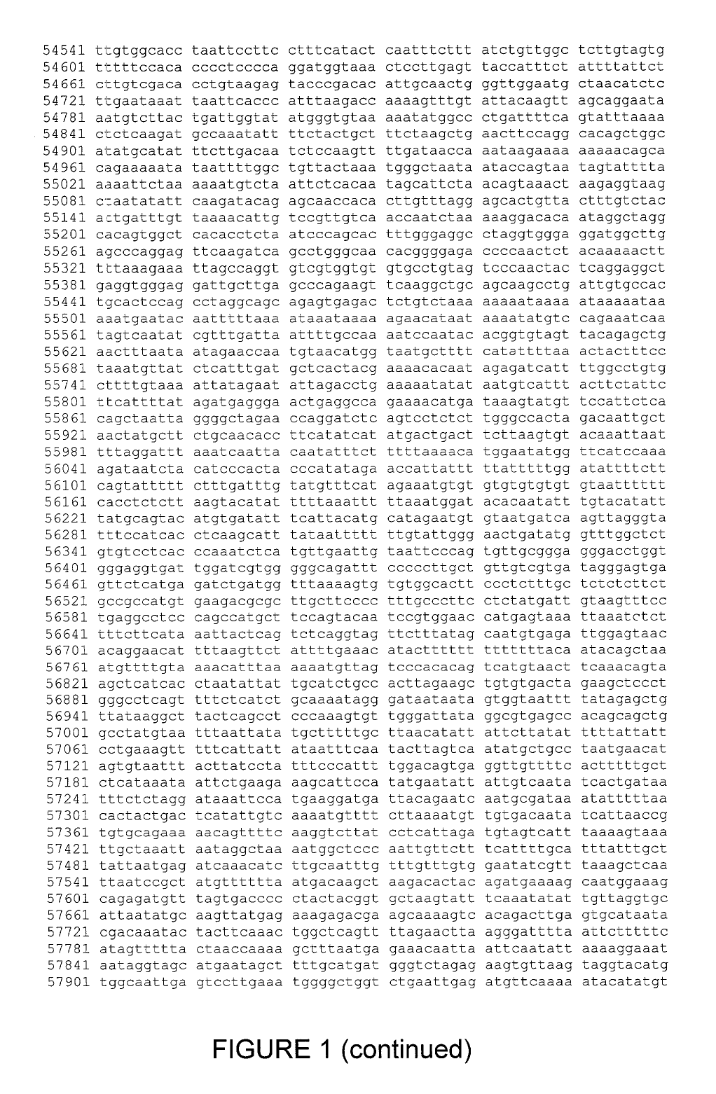

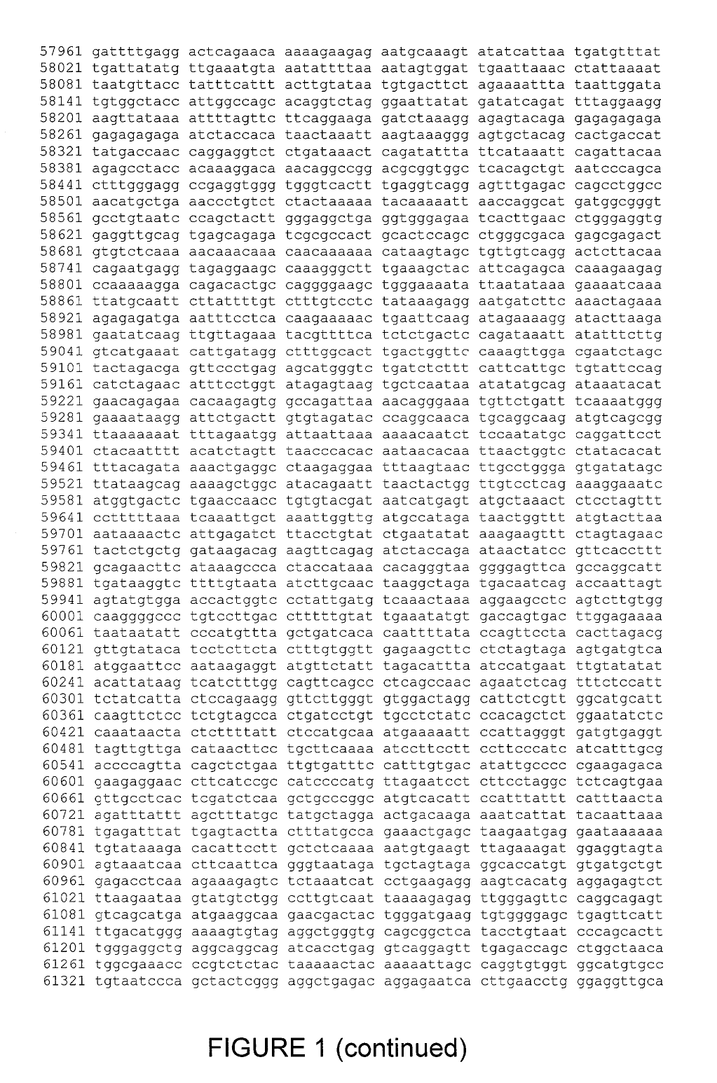

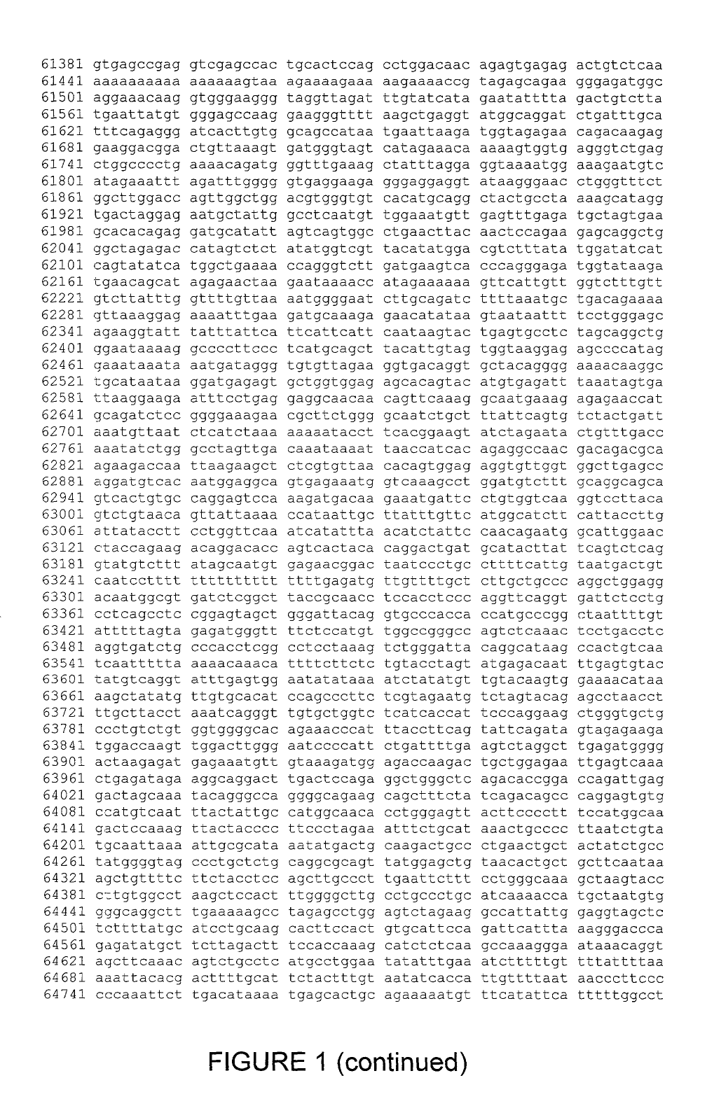

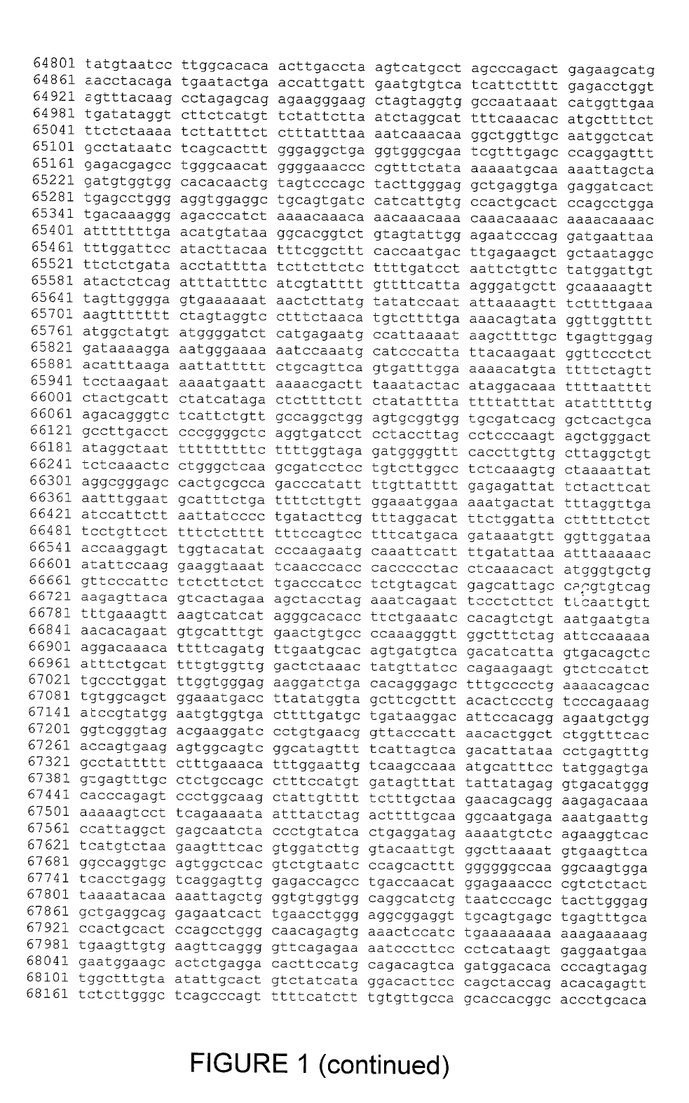

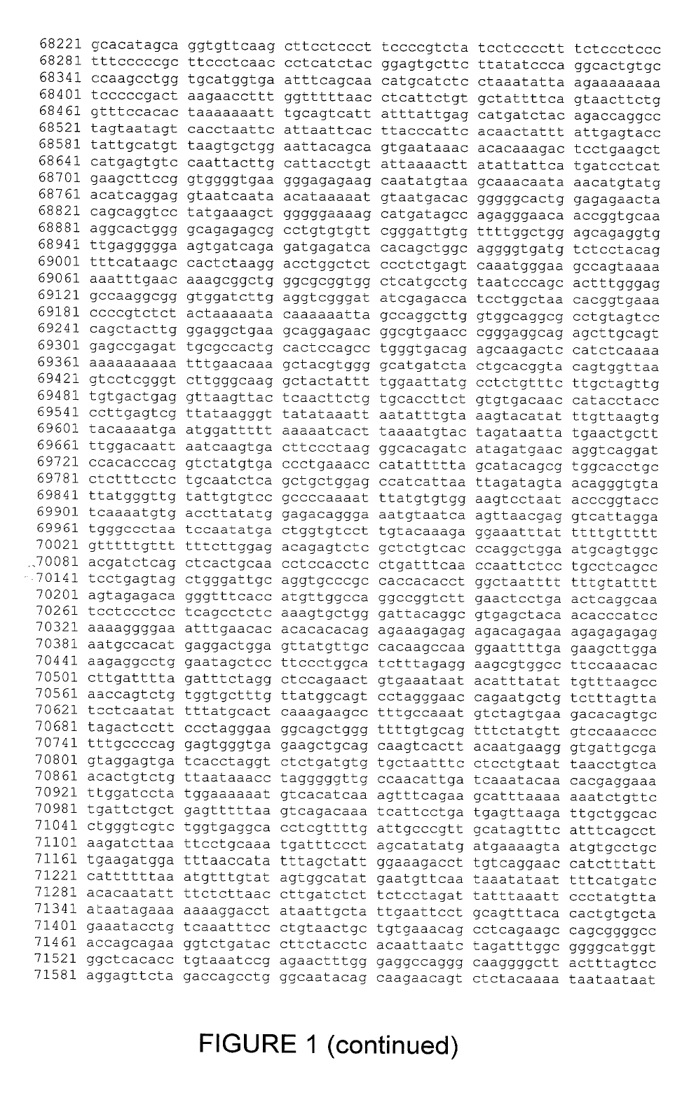

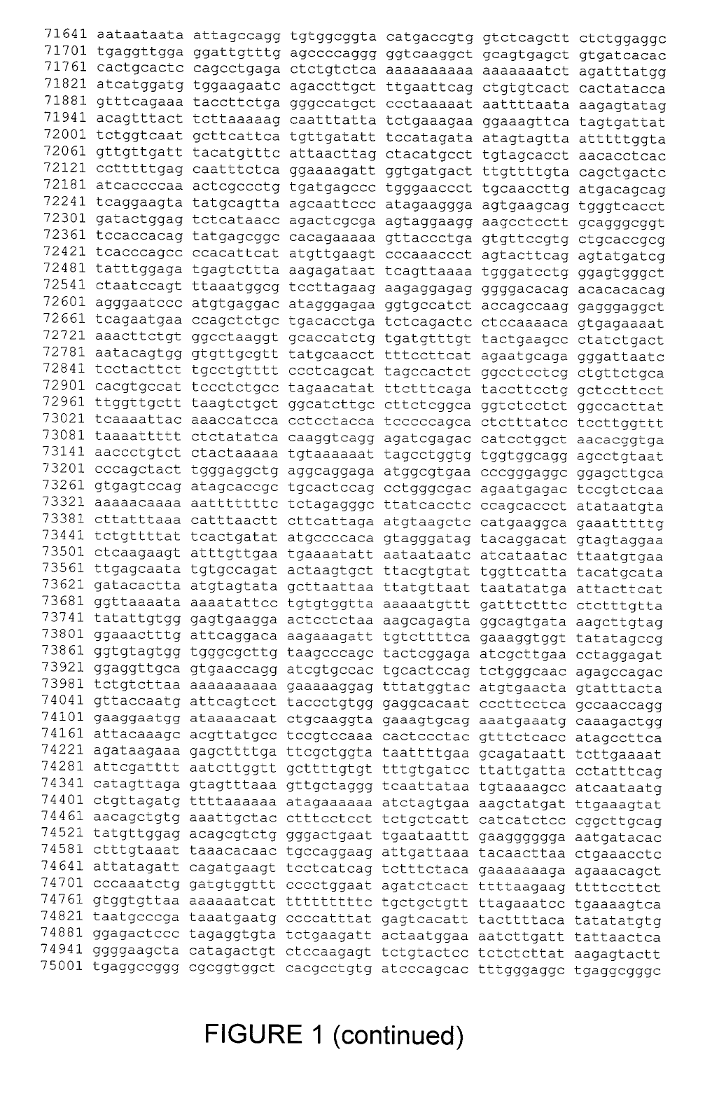

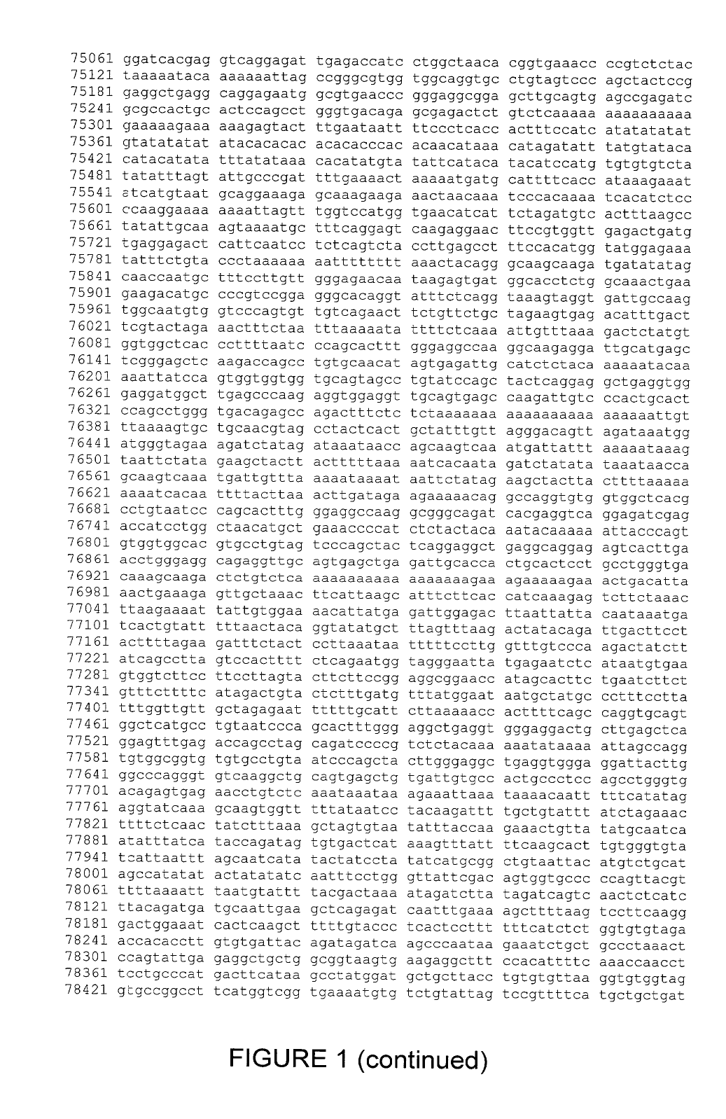

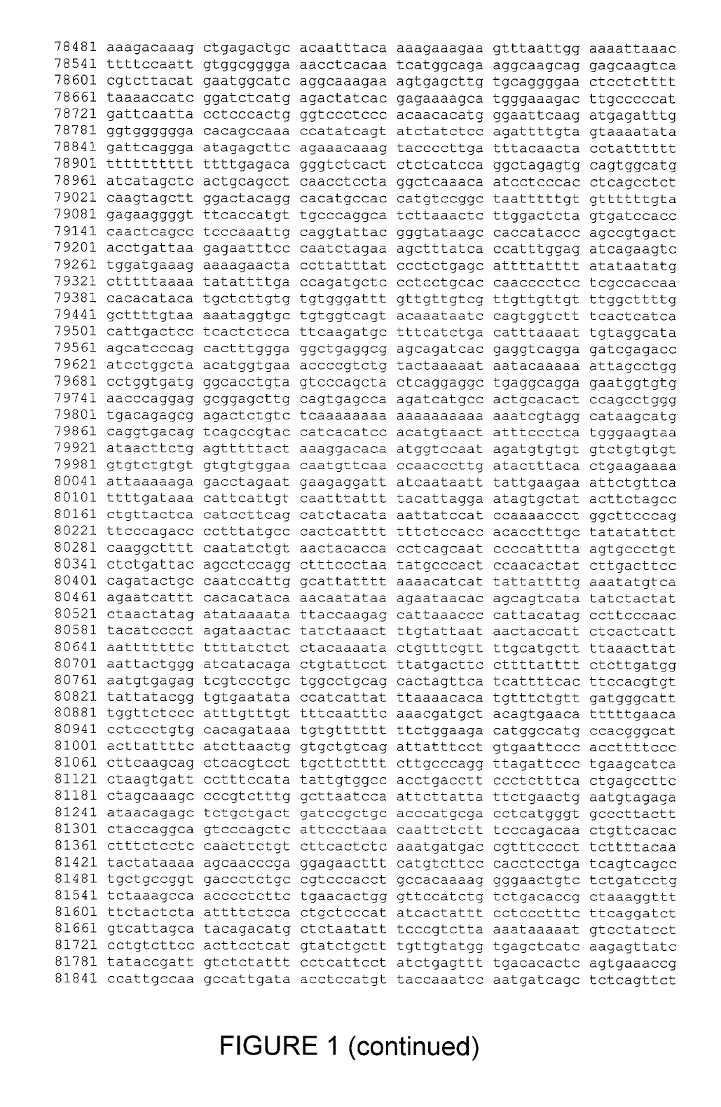

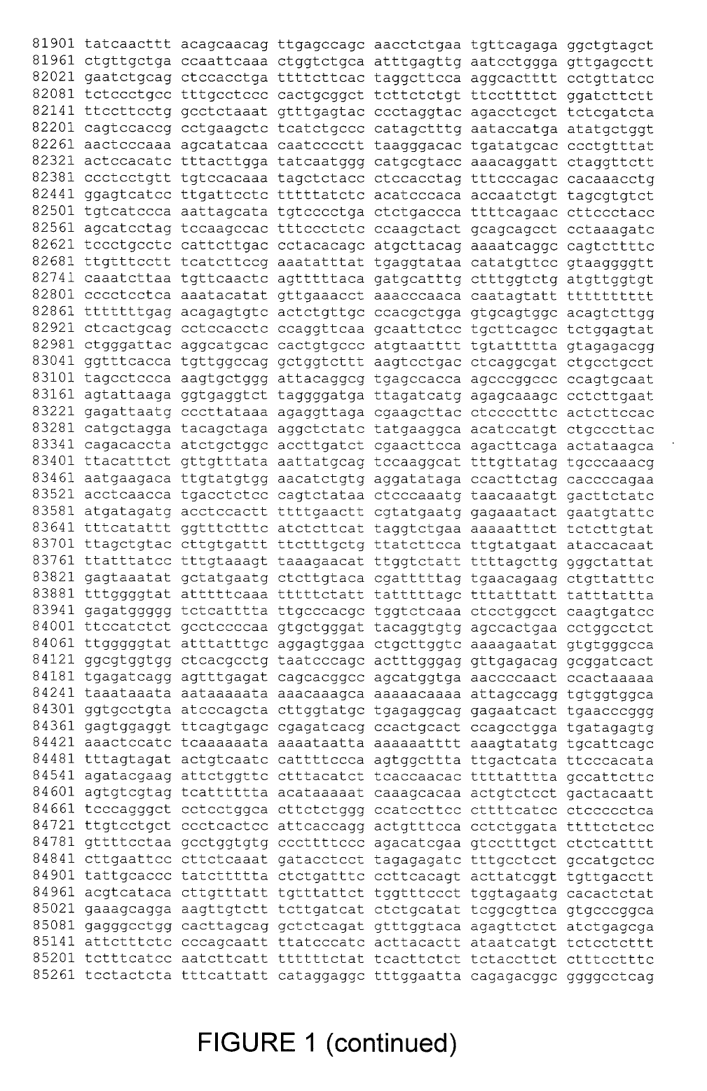

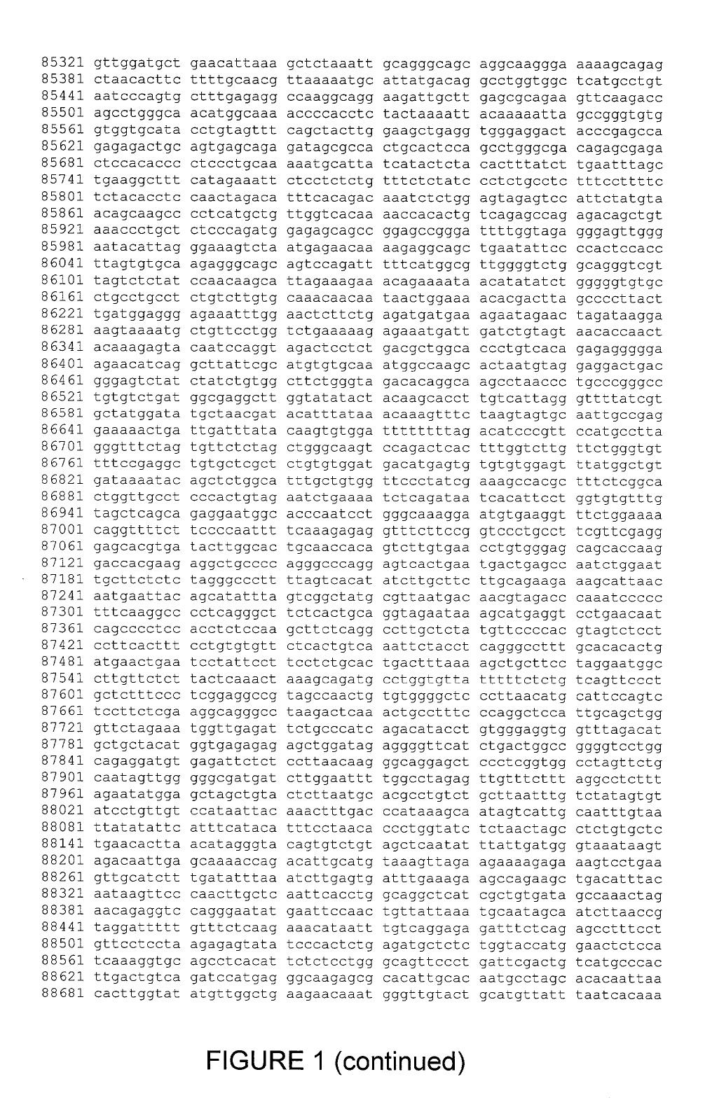

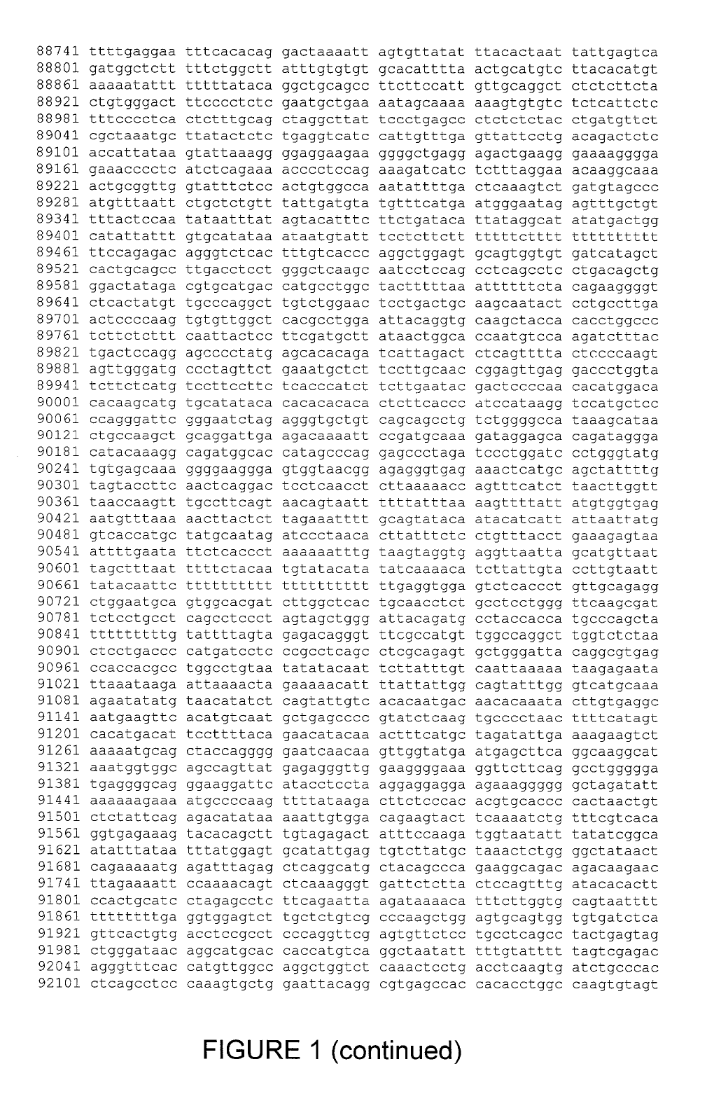

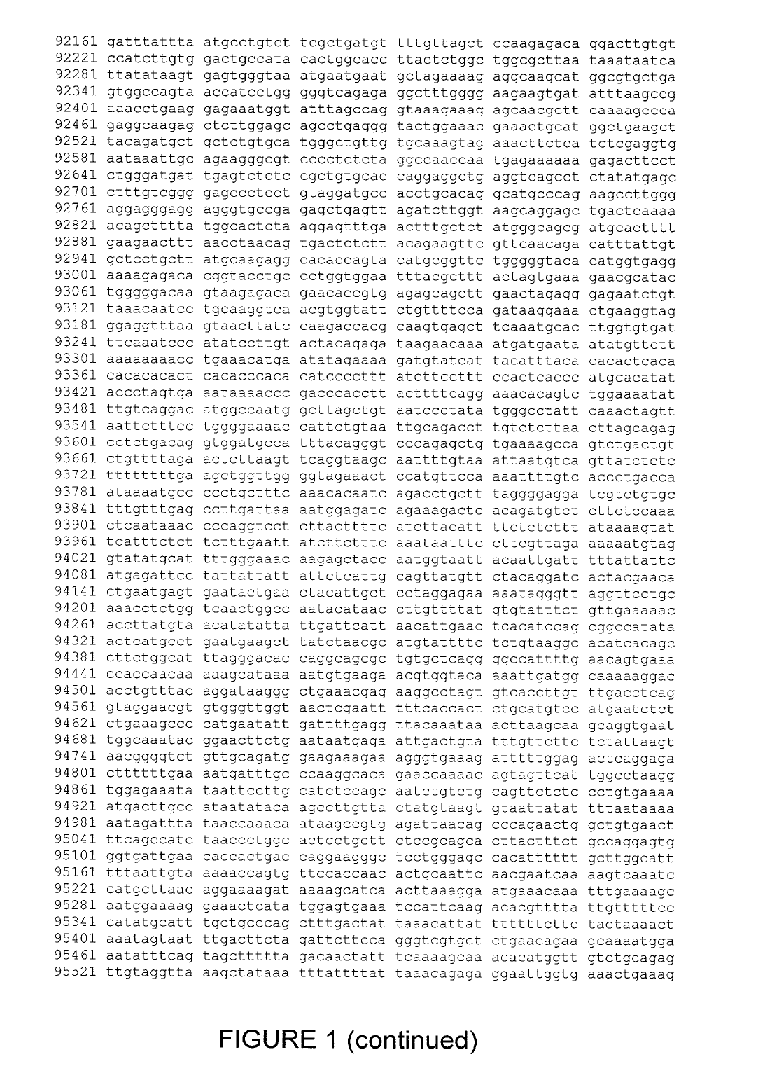

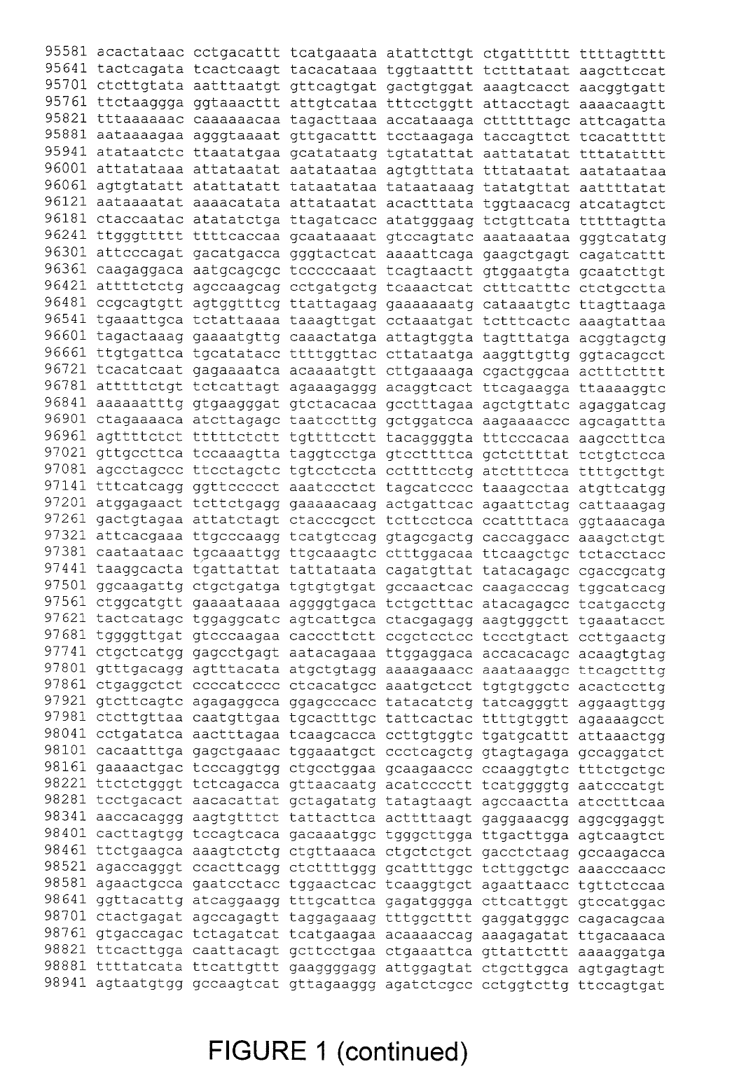

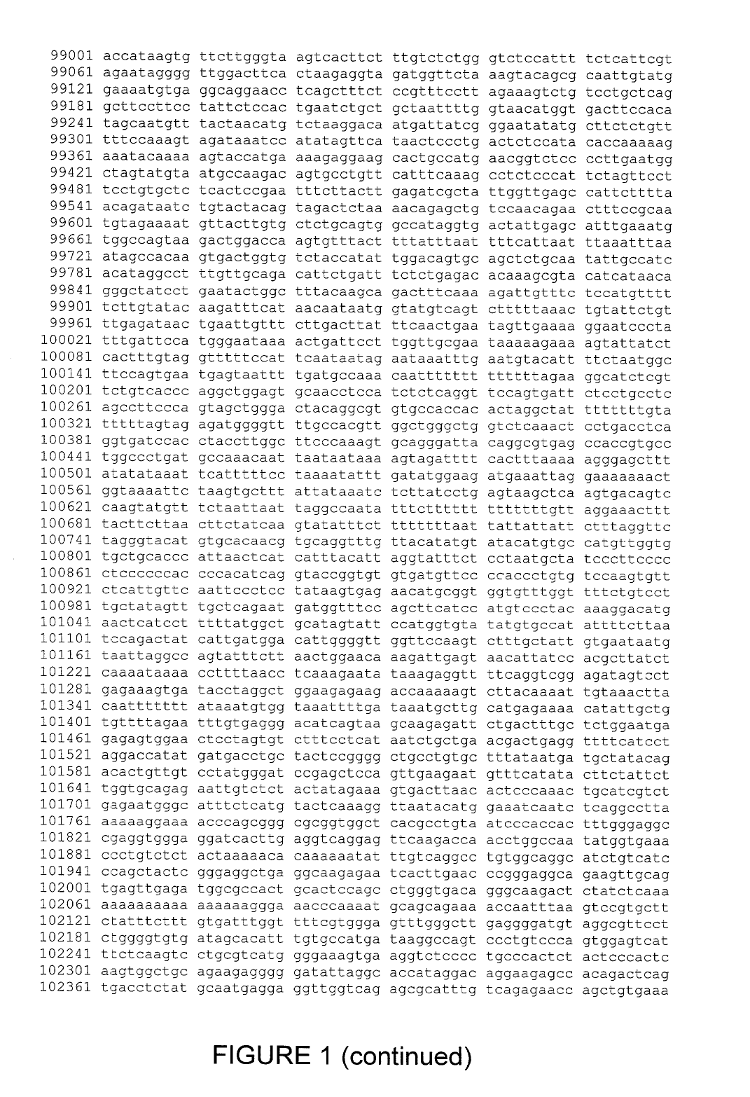

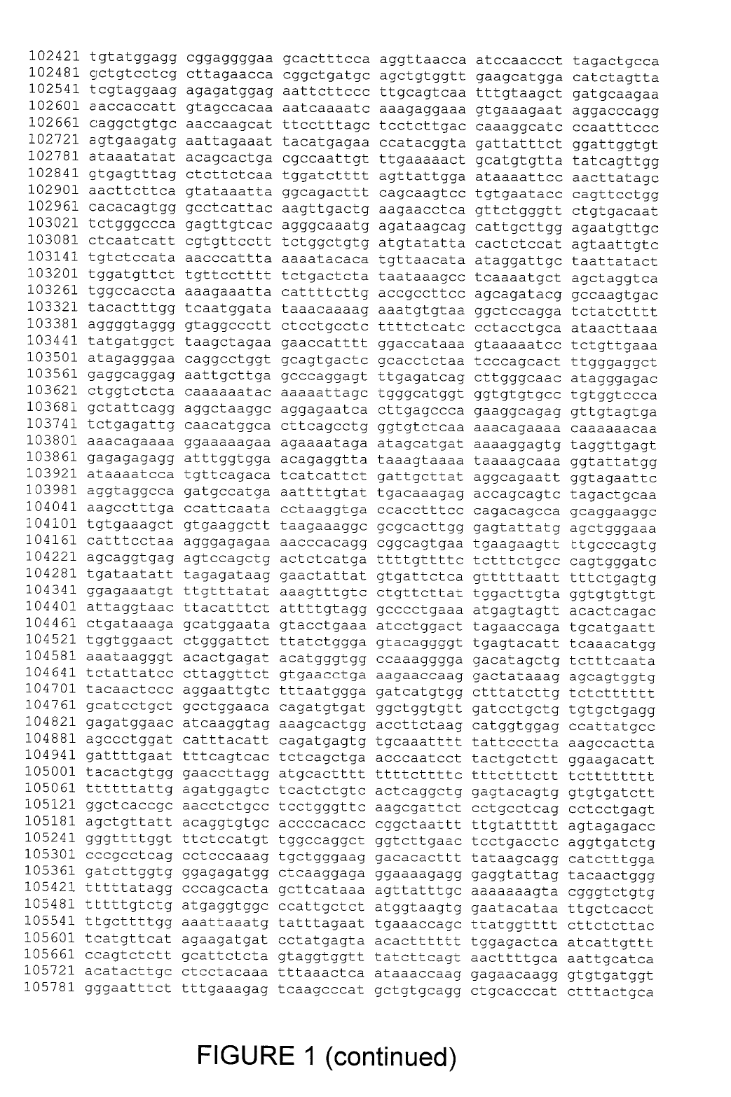

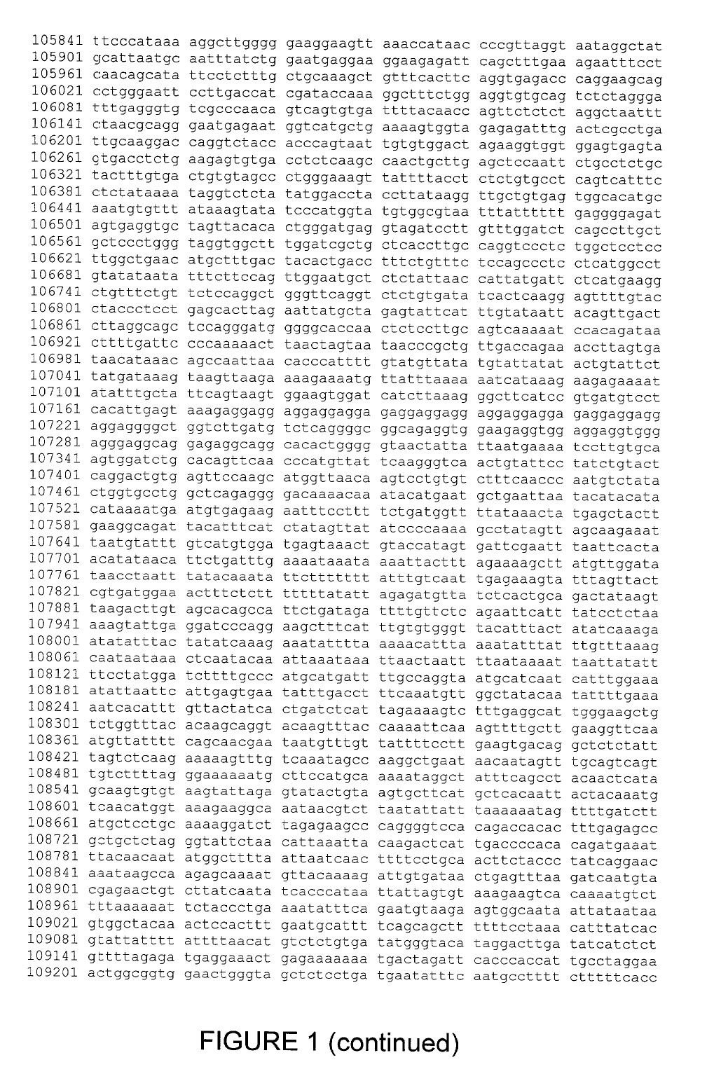

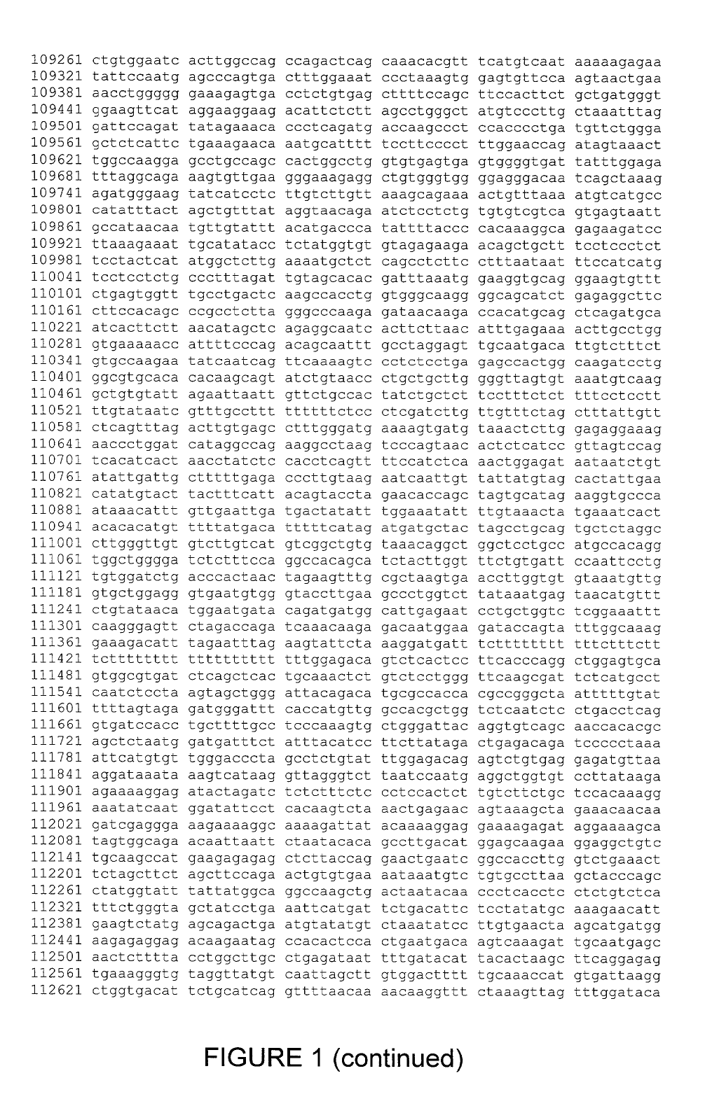

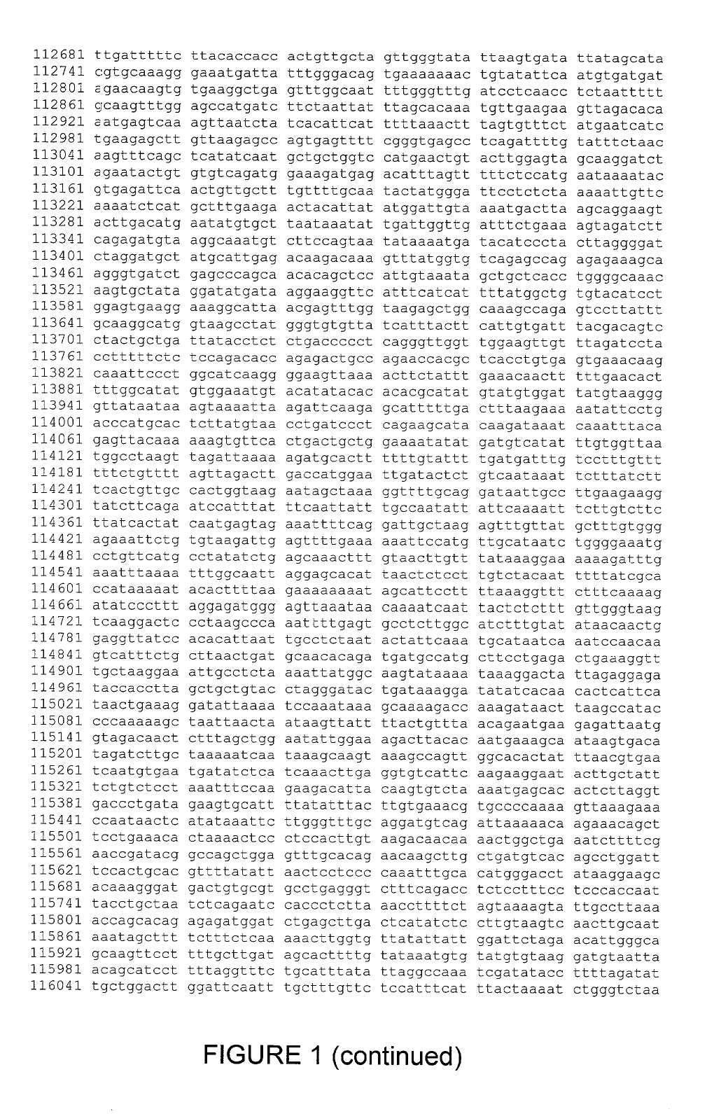

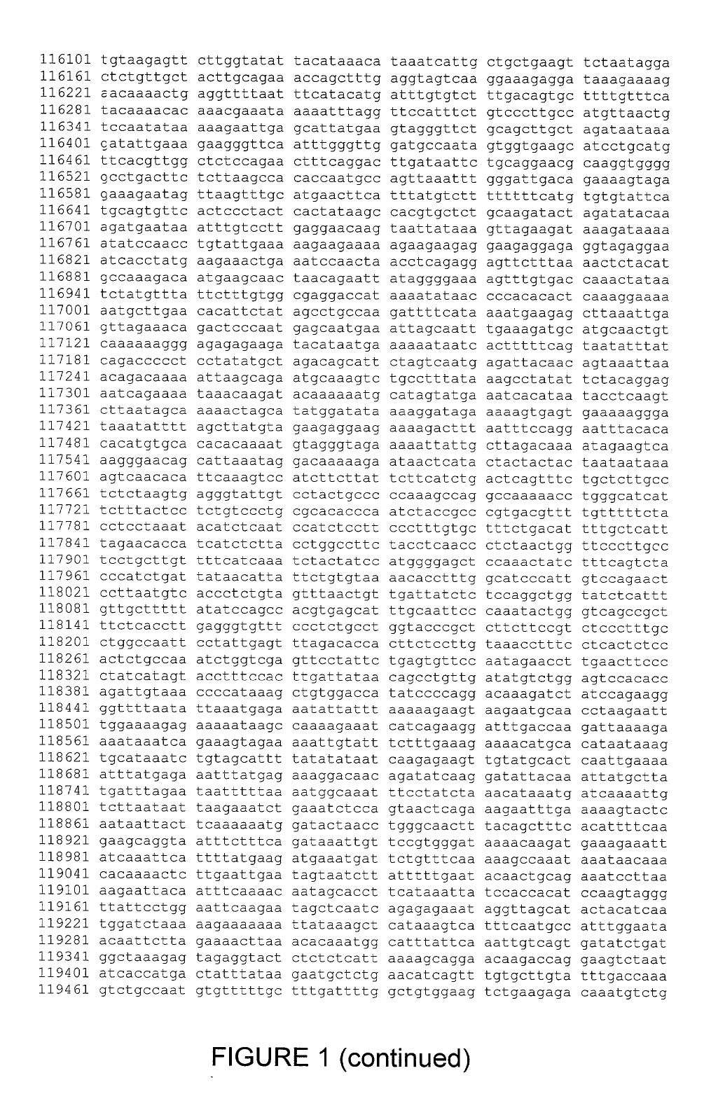

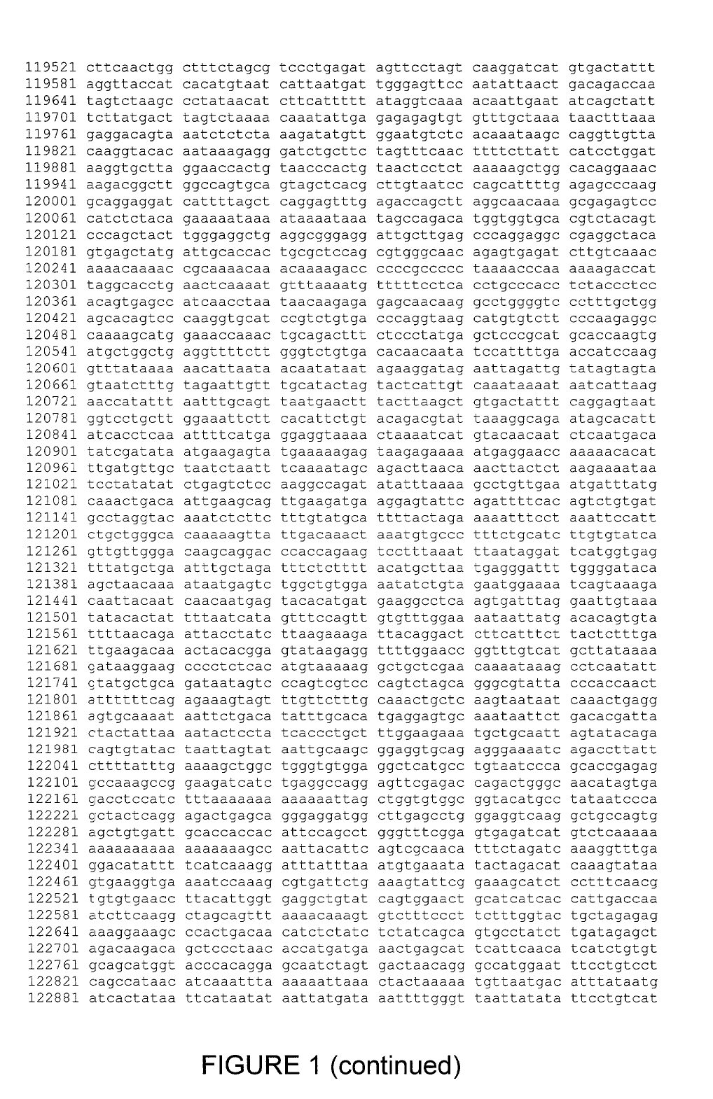

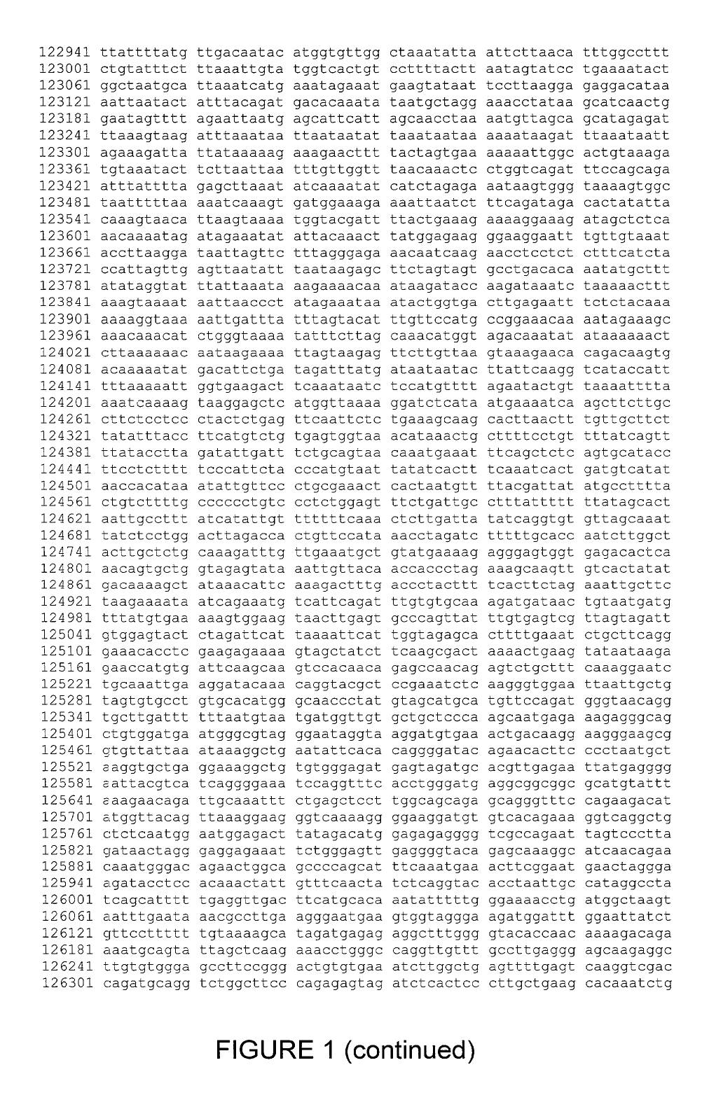

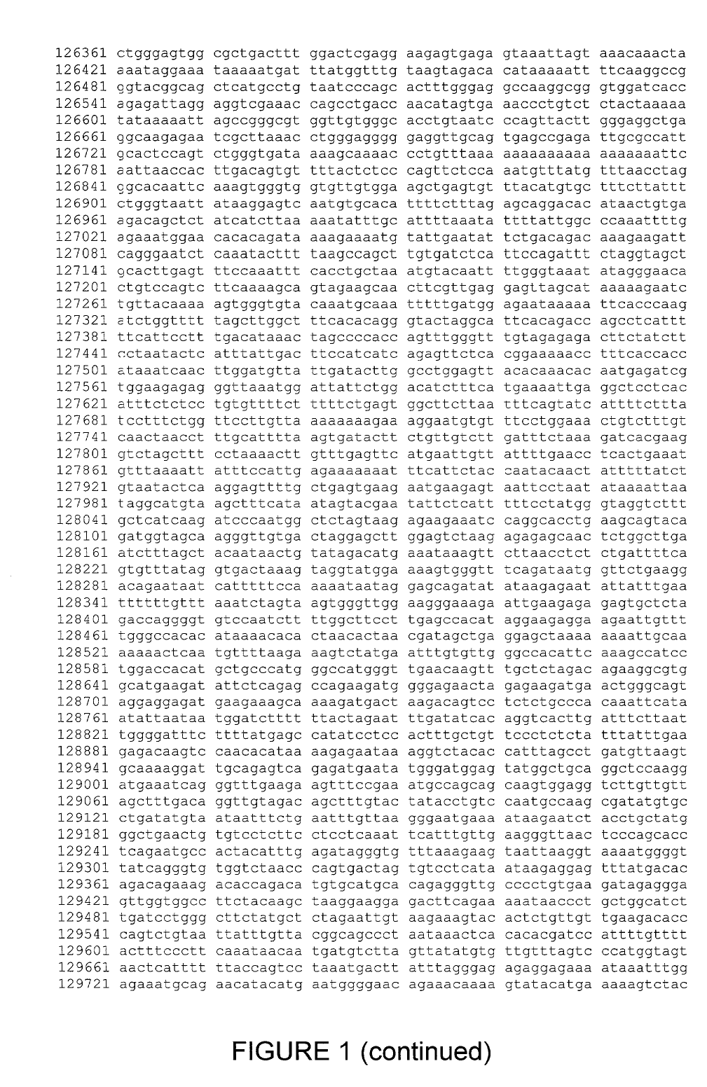

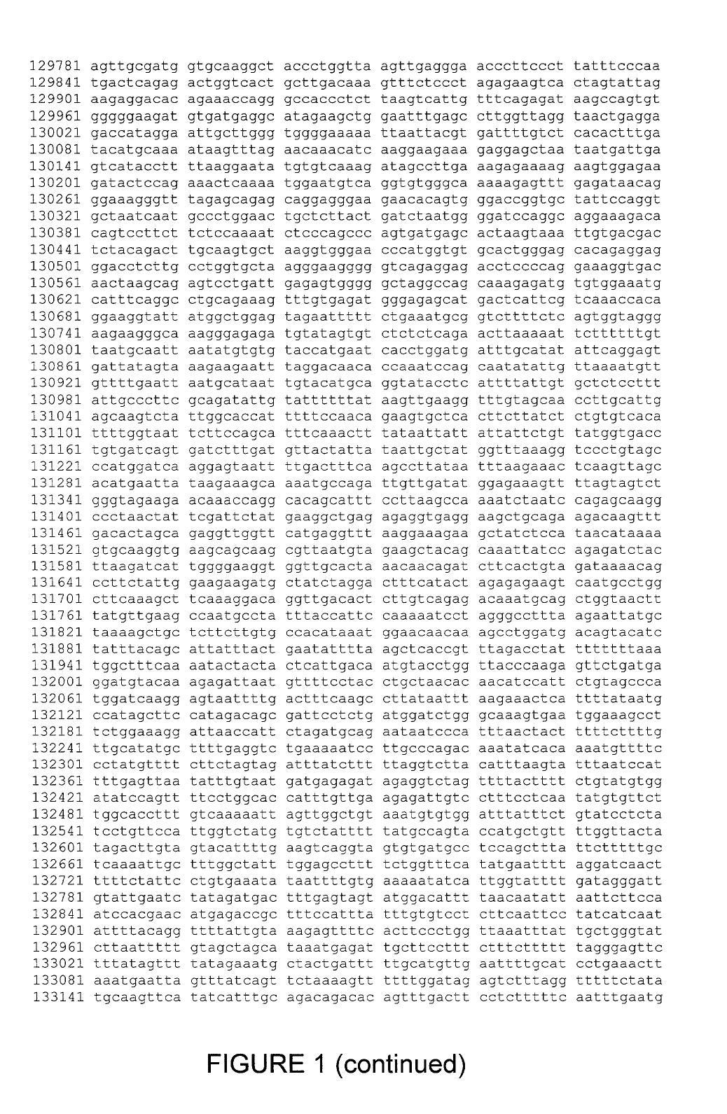

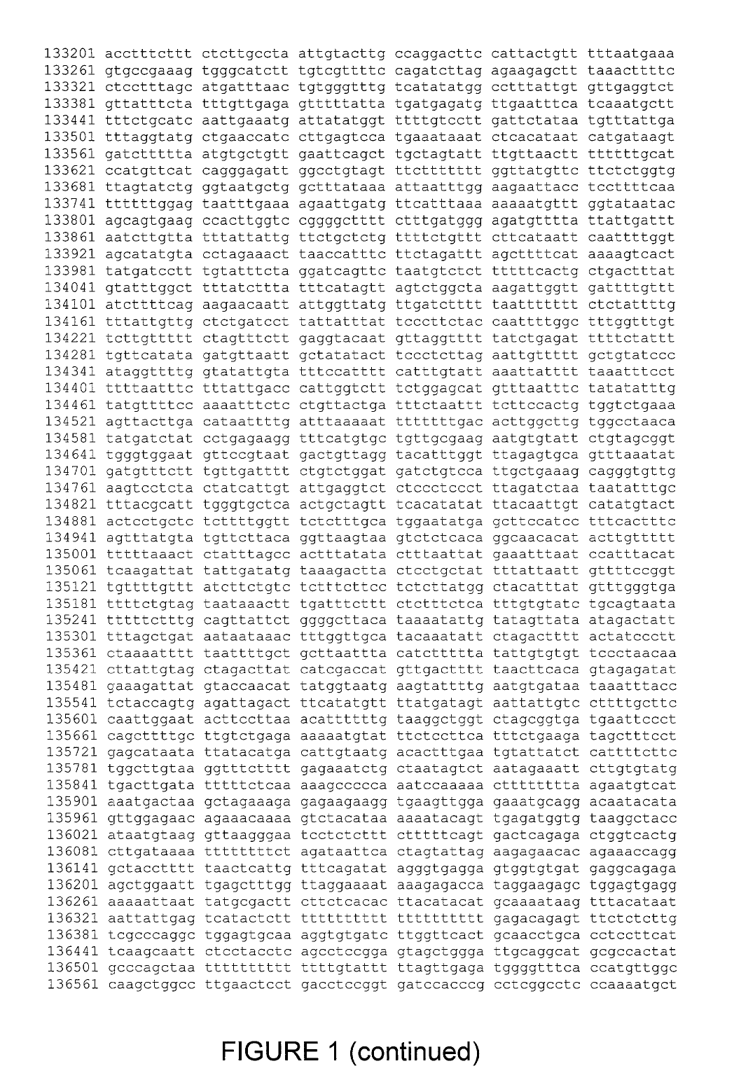

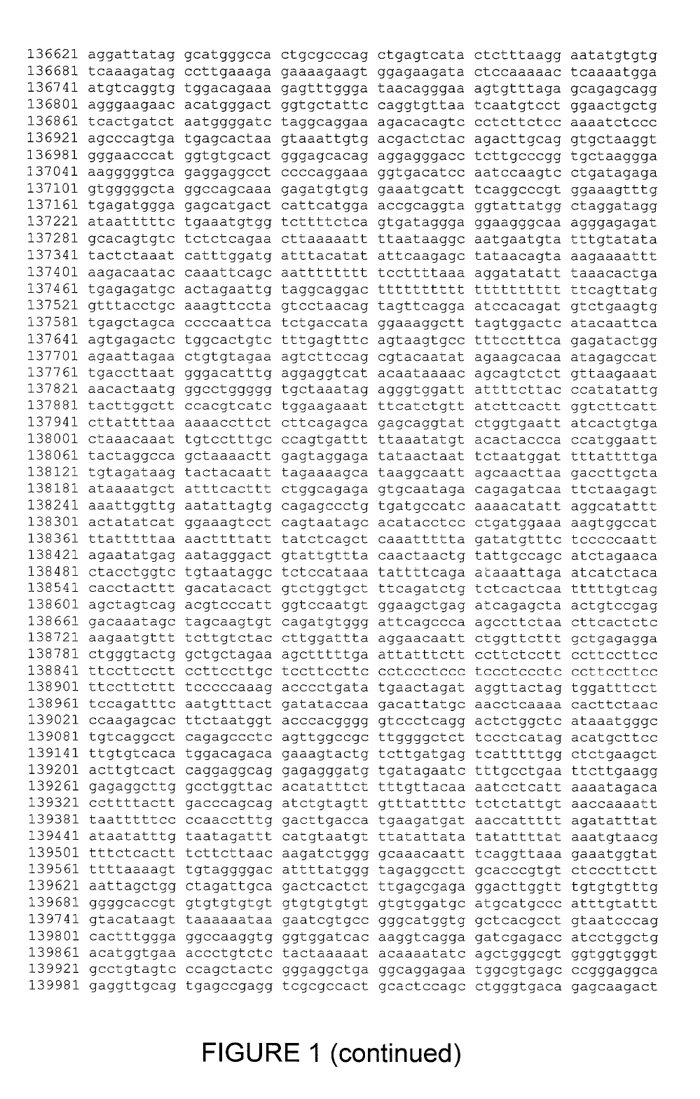

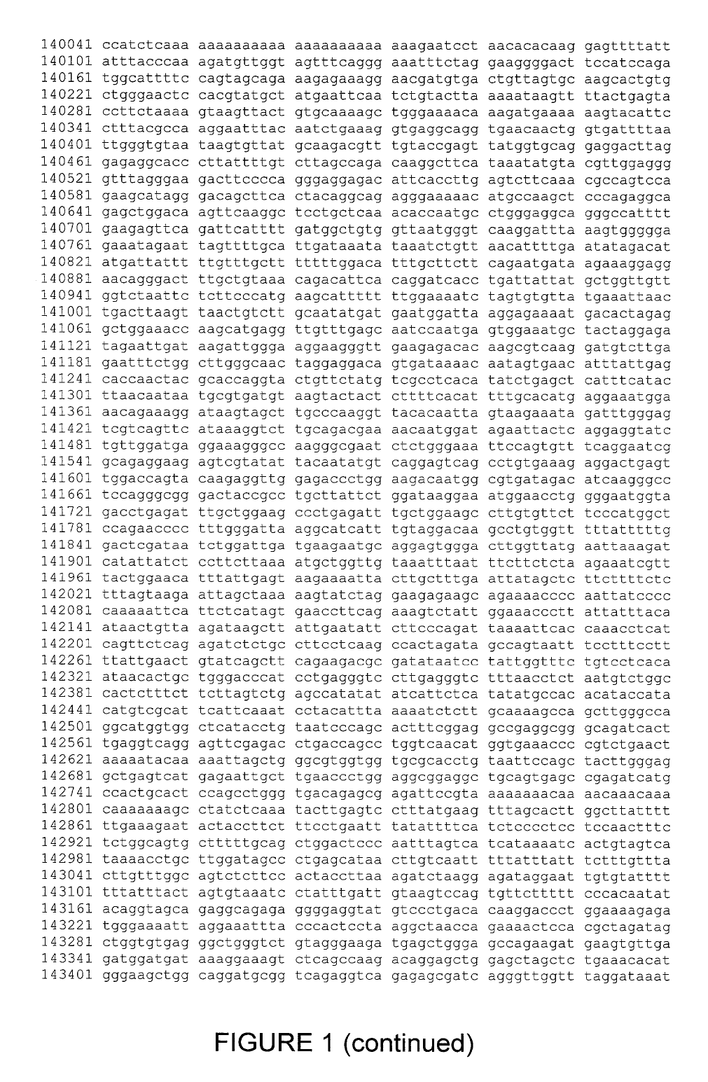

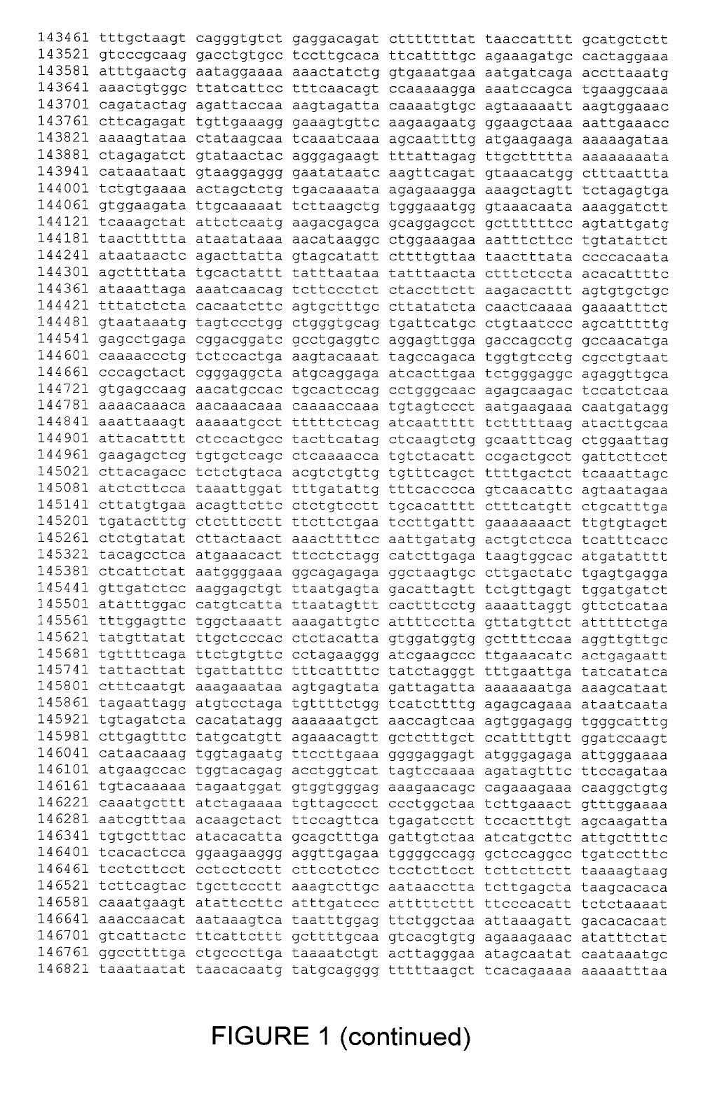

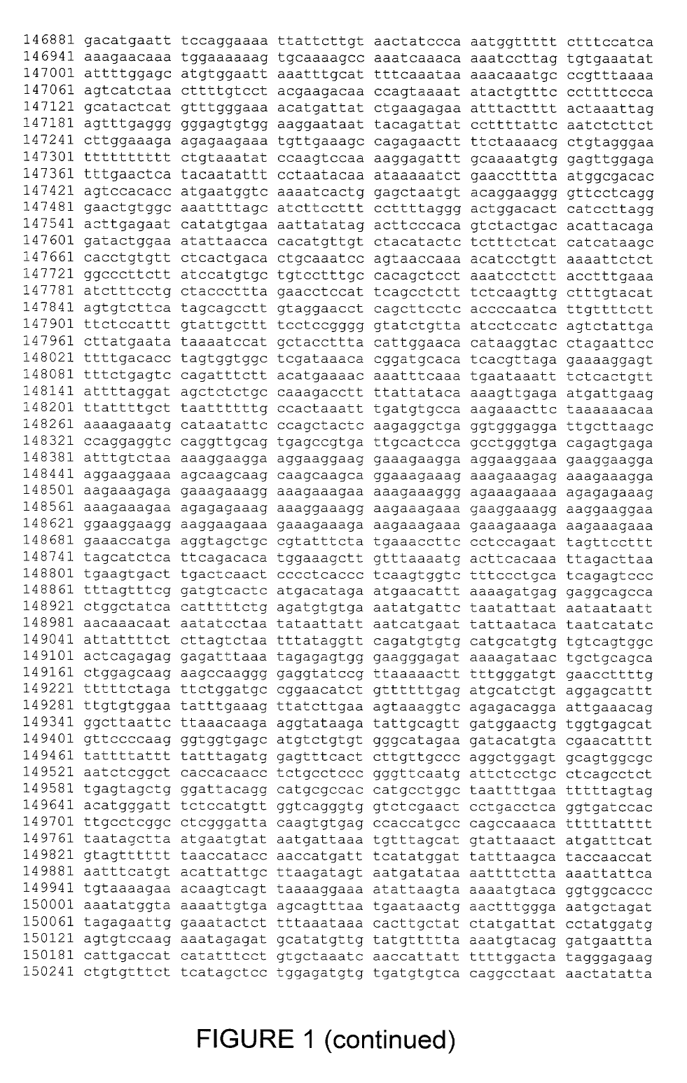

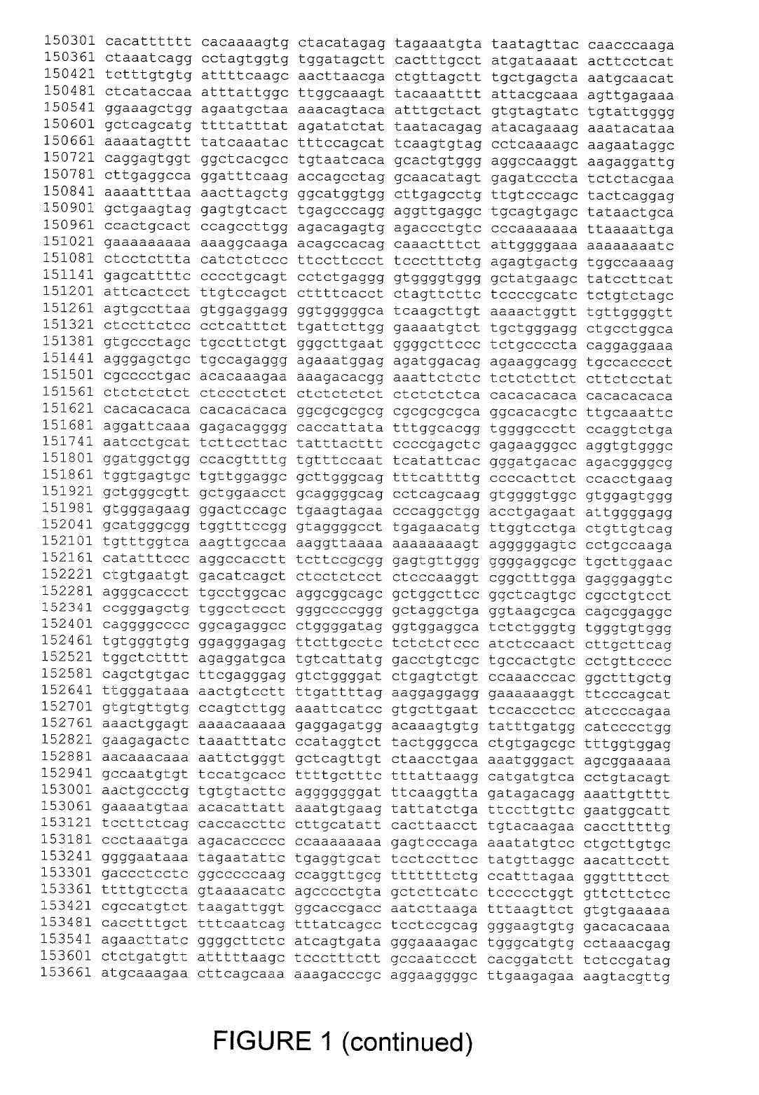

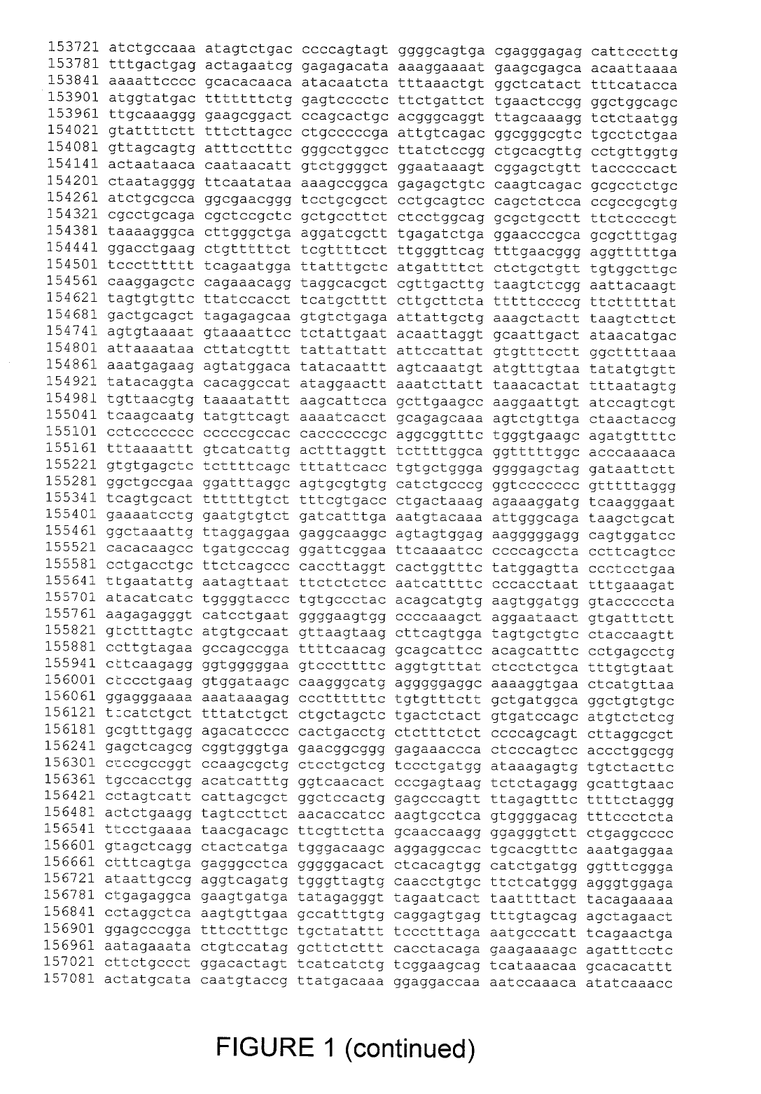

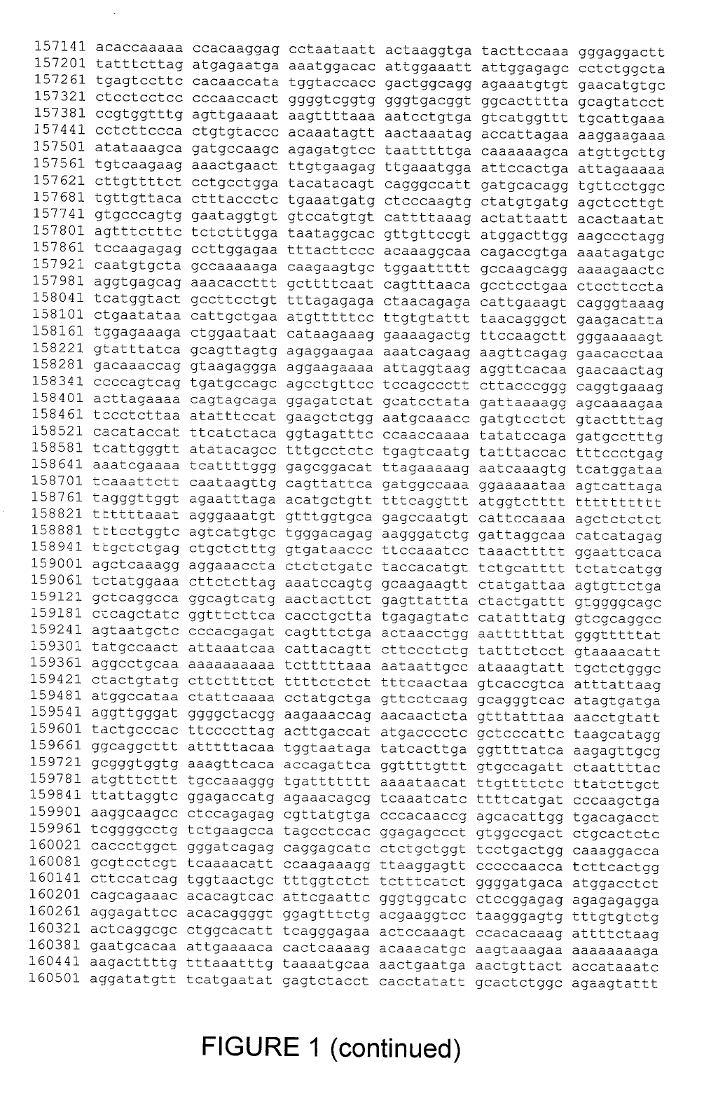

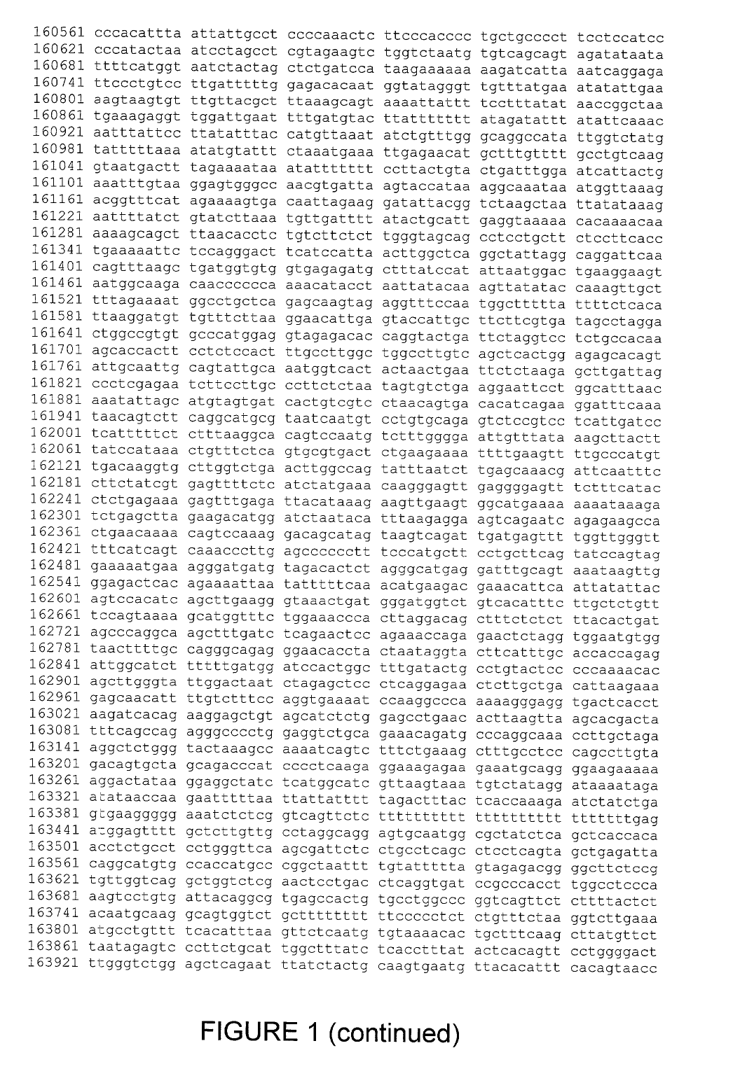

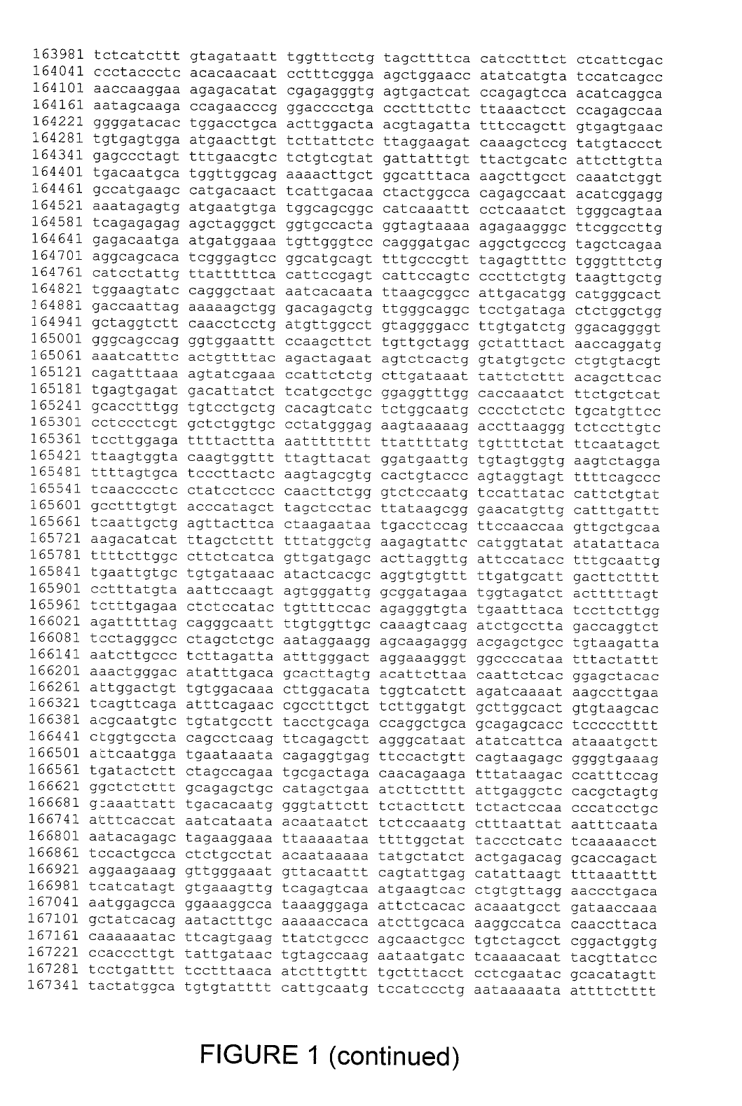

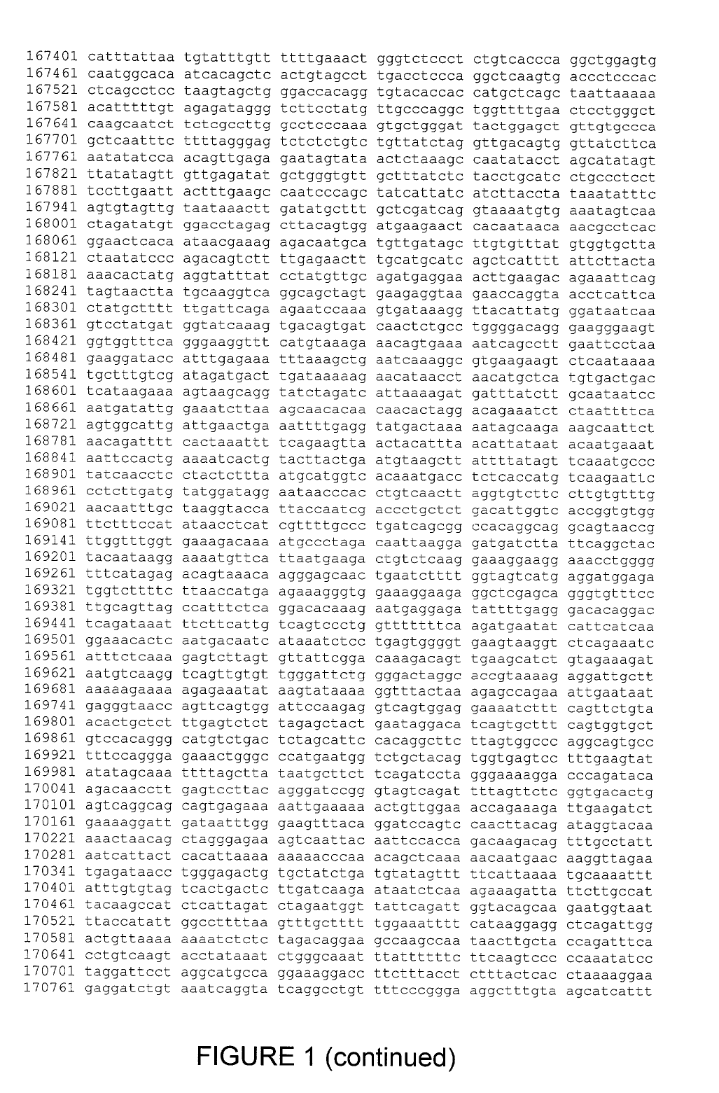

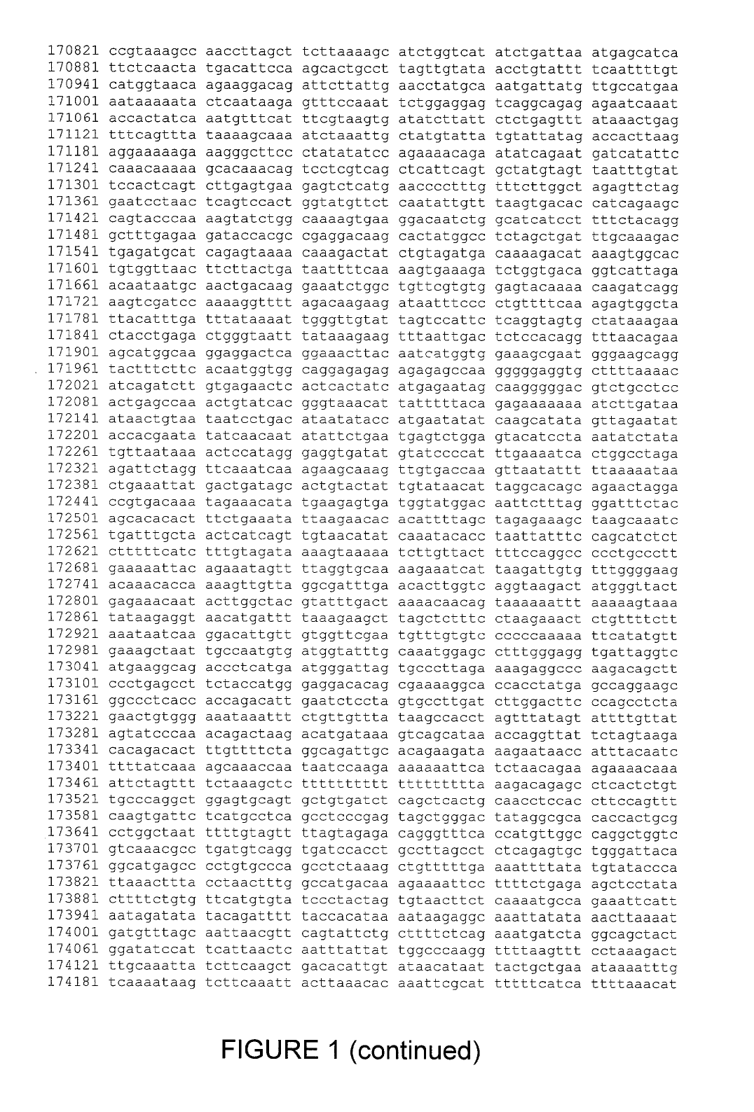

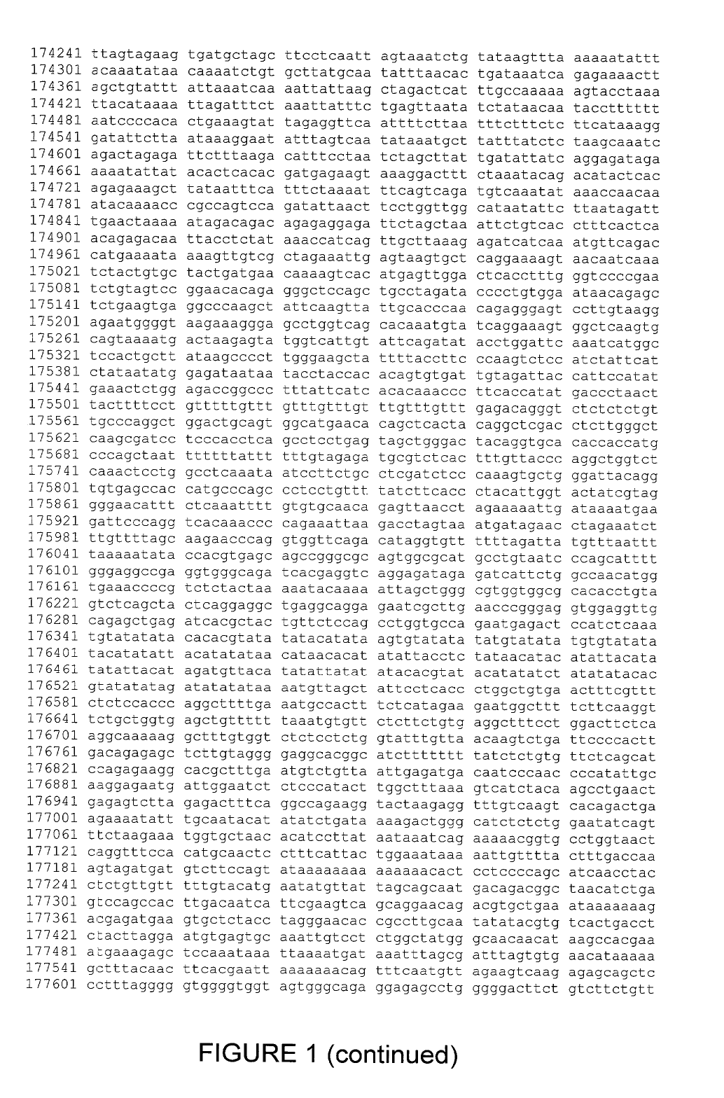

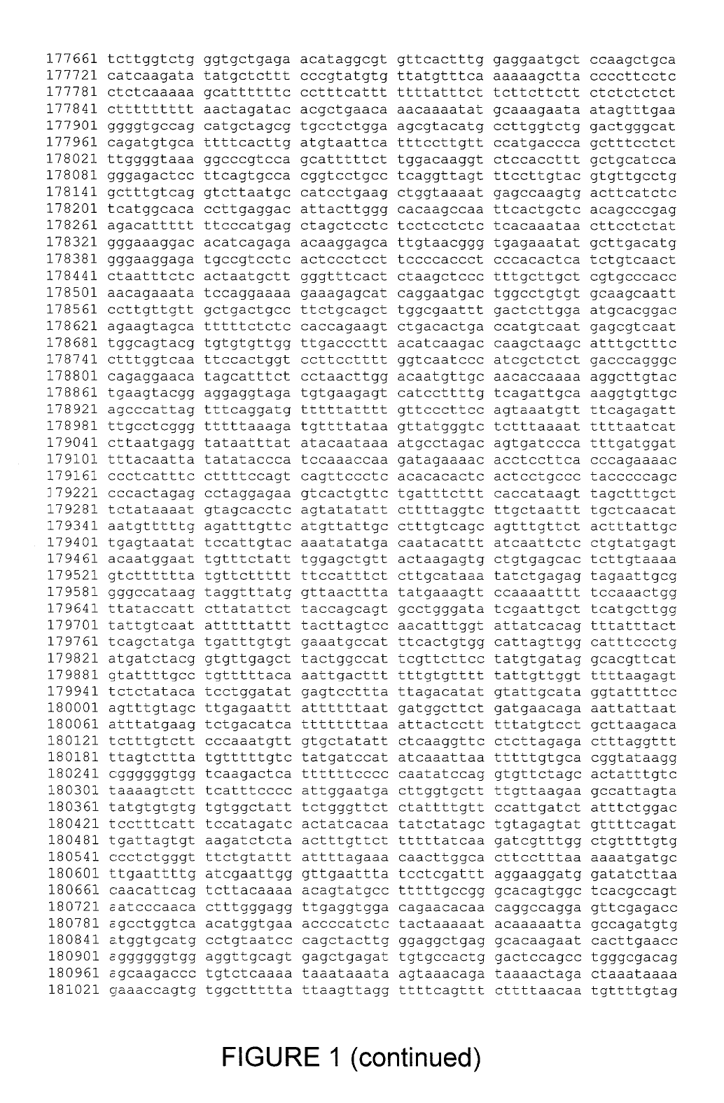

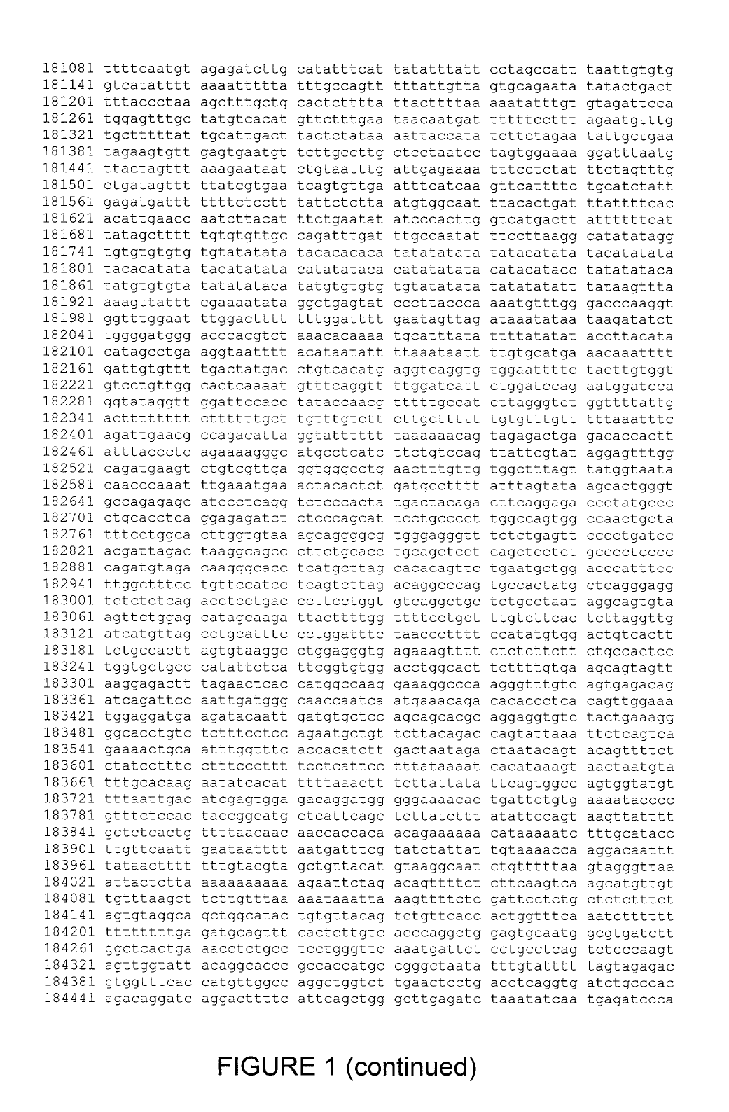

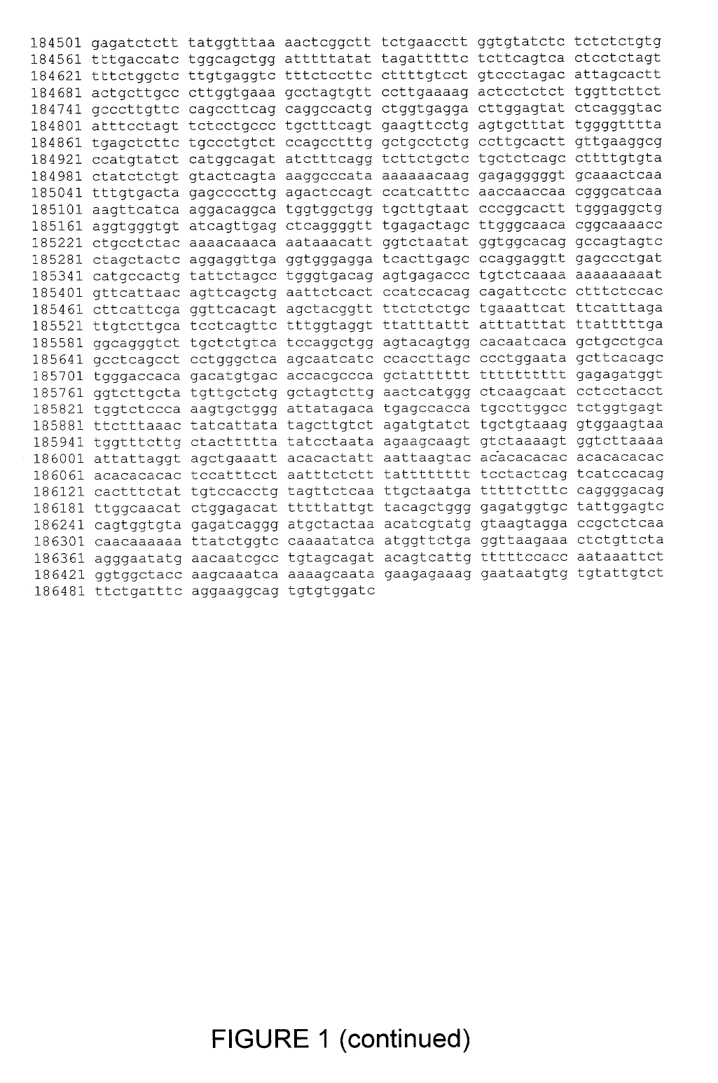

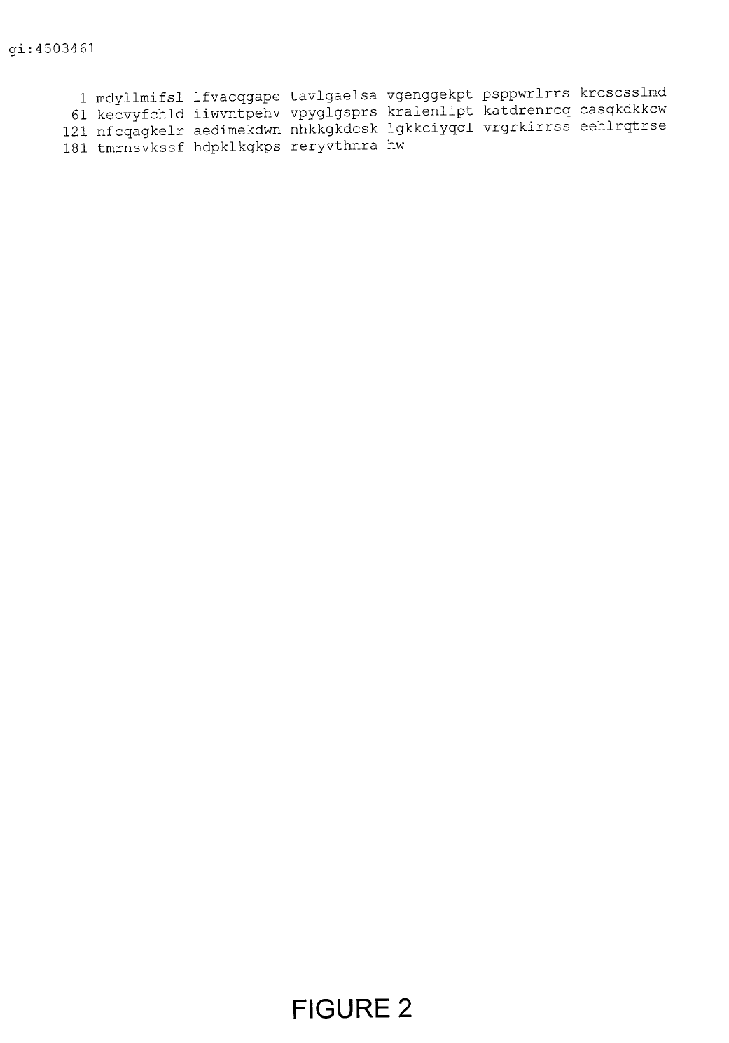

The polymorphisms are identified based on a change in the nucleotide sequence from a consensus sequence, or the “reference sequence.” As used herein, the reference sequence of EDN1 is the nucleotide sequence of SEQ ID NO:1 which corresponds to GI 2791272 (see FIG. 1).

To identify the location of the polymorphisms of the present invention, a specific nucleotide residue in a reference sequence is listed for the polymorphism, where nucleotide residue number 1 is the first (i.e., 5′) nucleotide in each reference sequence. Column 7 lists the reference sequence and polymorphic nucleotide residue for the polymorphisms. Column 3 describes the type of variant, e.g., either non-coding or missense.

The nucleic acid molecules of the invention can be double- or single-stranded. Accordingly, the invention further provides for the complementary nucleic acid strands comprising the polymorphisms listed in Table 1.

The invention further provides allele-specific oligonucleotides that hybridize to a gene comprising a single nucleotide polymorphism or to the complement of the gene. Such oligonucleotides will hybridize to one polymorphic form of the nucleic acid molecules described herein but not to the other polymorphic form of the sequence. Thus such oligonucleotides can be used to determine the presence or absence of particular alleles of the polymorphic sequences described herein. These oligonucleotides can be probes or primers.

Not only does the present invention provide polymorphisms in linkage disequilibrium with the polymorphisms of Table 1, it also provides methods for revealing the existence of yet other polymorphic regions in the human EDN1 gene. For example, the polymorphism studies described herein can also be applied to populations in which other vascular diseases or disorders are prevalent.

Other aspects of the invention are described below or will be apparent to one of skill in the art in light of the present disclosure.

Definitions

For convenience, the meaning of certain terms and phrases employed in the specification, examples, and appended claims are provided below.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene, including the EDN1 gene, can differ from each other in a single nucleotide, or several nucleotides, and can include substitutions, deletions, and insertions of nucleotides. An allele of a gene can also be a form of a gene containing one or more mutations.

The term “allelic variant of a polymorphic region of an EDN1 gene” refers to an alternative form of the EDN1 gene having one of several possible nucleotide sequences found in that region of the gene in the population.

“Biological activity” or “bioactivity” or “activity” or “biological function”, which are used interchangeably, for the purposes herein when applied to EDN1, means an effector or antigenic function that is directly or indirectly performed by an EDN1 polypeptide (whether in its native or denatured conformation), or by a fragment thereof. Biological activities include modulation of the development of atherosclerotic plaque leading to vascular disease and other biological activities, whether presently known or inherent. An EDN1 bioactivity can be modulated by directly affecting an EDN1 protein effected by, for example, changing the level of effector or substrate level. Alternatively, an EDN1 bioactivity can be modulated by modulating the level of an EDN1 protein, such as by modulating expression of an EDN1 gene. Antigenic functions include possession of an epitope or antigenic site that is capable of cross-reacting with antibodies that bind a native or denatured EDN1 polypeptide or fragment thereof.

Biologically active EDN1 polypeptides include polypeptides having both an effector and antigenic function, or only one of such functions. EDN1 polypeptides include antagonist polypeptides and native EDN1 polypeptides, provided that such antagonists include an epitope of a native EDN1 polypeptide. An effector function of EDN1 polypeptide can be the ability to bind to a ligand of an EDN1 molecule.

As used herein the term “bioactive fragment of an EDN1 protein” refers to a fragment of a full-length EDN1 protein, wherein the fragment specifically mimics or antagonizes the activity of a wild-type EDN1 protein. The bioactive fragment preferably is a fragment capable of binding to a second molecule, such as a ligand.

The term “an aberrant activity” or “abnormal activity”, as applied to an activity of a protein such as EDN1, refers to an activity which differs from the activity of the normal or reference protein or which differs from the activity of the protein in a healthy subject, e.g., a subject not afflicted with a disease associated with an EDN1 allelic variant. An activity of a protein can be aberrant because it is stronger than the activity of its wild-type counterpart. Alternatively, an activity of a protein can be aberrant because it is weaker or absent relative to the activity of its normal or reference counterpart. An aberrant activity can also be a change in reactivity. For example an aberrant protein can interact with a different protein or ligand relative to its normal or reference counterpart. A cell can also have aberrant EDN1 activity due to overexpression or underexpression of the EDN1 gene. Aberrant EDN1 activity can result from a mutation in the gene, which results, e.g., in lower or higher binding affinity of a ligand to the EDN1 protein encoded by the mutated gene. Aberrant EDN1 activity can also result from an abnormal EDN1 5′ upstream regulatory element activity.

“Cells,” “host cells” or “recombinant host cells” are terms used interchangeably herein. It is understood that such terms refer not only to the particular cell but to the progeny or derivatives of such a cell. Because certain modifications may occur in succeeding generations due to either mutation or environmental influences, such progeny may not, in fact, be identical to the parent cell, but are still included within the scope of the term as used herein.

As used herein, the term “course of clinical therapy” refers to any chosen method to treat, prevent, or ameliorate a vascular disease, e.g., CAD or MI, symptoms thereof, or related diseases or disorders. Courses of clinical therapy include, but are not limited to, lifestyle changes (e.g., changes in diet or environment), administration of medication, use of medical devices, such as, but not limited to, a defibrillator, a stent, a device used in coronary revascularization, a pacemaker, or any combination thereof, and surgical procedures such as percutaneous transluminal coronary balloon angioplasty (PTCA) or laser angioplasty, or other surgical intervention, such as, for example, coronary bypass grafting (CABG), or any combination thereof.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “genetic profile” refers to the information obtained from identification of the specific allelic variants of a subject. For example, an EDN1 genetic profile refers to the specific allelic variants of a subject within the EDN1 gene. For example, one can determine a subject's EDN1 genetic profile by determining the identity of one or more of the nucleotides present at nucleotide residues 157790 and 159908 of SEQ ID NO:1 (the EDN1 gene), or the complements thereof, or by determining the amino acid present at amino acid residue 198 of SEQ ID NO:2 (the EDN1 protein). The genetic profile of a particular disease can be ascertained through identification of the identity of allelic variants in one or more genes which are associated with the particular disease.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The term “a homolog of a nucleic acid” refers to a nucleic acid having a nucleotide sequence having a certain degree of homology with the nucleotide sequence of the nucleic acid or complement thereof. For example, a homolog of a double stranded nucleic acid having SEQ ID NO:N is intended to include nucleic acids having a nucleotide sequence which has a certain degree of homology with SEQ ID NO:N or with the complement thereof. Preferred homologs of nucleic acids are capable of hybridizing to the nucleic acid or complement thereof.

The term “hybridization probe” or “primer” as used herein is intended to include oligonucleotides which hybridize bind in a base-specific manner to a complementary strand of a target nucleic acid. Such probes include peptide nucleic acids, and described in Nielsen et al., (1991) Science 254:1497-1500. Probes and primers can be any length suitable for specific hybridization to the target nucleic acid sequence. The most appropriate length of the probe and primer may vary depending on the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range form about 5 nucleotides to about 30 nucleotides in length. For example, probes and primers can be 5, 6, 8, 10, 12, 14, 16, 18, 20, 22, 24, 25, 26, 28 or 30 nucleotides in length. The probe or primer of the invention comprises a sequence that flanks and/or preferably overlaps, at least one polymorphic site occupied by any of the possible variant nucleotides. The nucleotide sequence of an overlapping probe or primer can correspond to the coding sequence of the allele or to the complement of the coding sequence of the allele.

The term “vascular disease or disorder” as used herein refers to any disease or disorder effecting the vascular system, including the heart and blood vessels. A vascular disease or disorder includes any disease or disorder characterized by vascular dysfunction, including, for example, intravascular stenosis (narrowing) or occlusion (blockage), due to the development of atherosclerotic plaque and diseases and disorders resulting therefrom. Examples of vascular diseases and disorders include, without limitation, atherosclerosis, CAD, MI, ischemia, stroke, peripheral vascular diseases, venous thromboembolism and pulmonary embolism.

The term “interact” as used herein is meant to include detectable interactions between molecules, such as can be detected using, for example, a binding or hybridization assay. The term interact is also meant to include “binding” interactions between molecules. Interactions may be, for example, protein-protein, protein-nucleic acid, protein-small molecule or small molecule-nucleic acid in nature.

The term “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “linkage” describes the tendency of genes, alleles, loci or genetic markers to be inherited together as a result of their location on the same chromosome. It can be measured by percent recombination between the two genes, alleles, loci, or genetic markers. The term “linkage disequilibrium,” also referred to herein as “LD,” refers to a greater than random association between specific alleles at two marker loci within a particular population. In general, linkage disequilibrium decreases with an increase in physical distance. If linkage disequilibrium exists between two markers, then the genotypic information at one marker can be used to make probabilistic predictions about the genotype of the second marker.

The term “locus” refers to a specific position in a chromosome. For example, a locus of an EDN1 gene refers to the chromosomal position of the EDN1 gene.

The term “modulation” as used herein refers to both upregulation, (i.e., activation or stimulation), for example by agonizing; and downregulation (i.e., inhibition or suppression), for example by antagonizing of a bioactivity (e.g. expression of a gene).

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

The term “mutated gene” refers to an allelic form of a gene that differs from the predominant form in a population. A mutated gene is capable of altering the phenotype of a subject having the mutated gene relative to a subject having the predominant form of the gene. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenine”, “cytidine”, “guanine”, and “thymidine” and/or “A”, “C”, “G”, and “T”, respectively, are used. It is understood that if the nucleic acid is RNA, a nucleotide having a uracil base is uridine.

The term “nucleotide sequence complementary to the nucleotide sequence set forth in SEQ ID NO:N” refers to the nucleotide sequence of the complementary strand of a nucleic acid strand having SEQ ID NO:N. The term “complementary strand” is used herein interchangeably with the term “complement.” The complement of a nucleic acid strand can be the complement of a coding strand or the complement of a non-coding strand. When referring to double stranded nucleic acids, the complement of a nucleic acid having SEQ ID NO:N refers to the complementary strand of the strand having SEQ ID NO:N or to any nucleic acid having the nucleotide sequence of the complementary strand of SEQ ID NO:N. When referring to a single stranded nucleic acid having the nucleotide sequence SEQ ID NO:N, the complement of this nucleic acid is a nucleic acid having a nucleotide sequence which is complementary to that of SEQ ID NO:N. The nucleotide sequences and complementary sequences thereof are always given in the 5′ to 3′ direction. The term “complement” and “reverse complement” are used interchangeably herein.

A “non-human animal” of the invention can include mammals such as rodents, non-human primates, sheep, goats, horses, dogs, cows, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which an exogenous sequence is found, or in which an exogenous sequence is expressed in some but not all cells of the animal. The term “tissue-specific chimeric animal” indicates that an exogenous sequence is present and/or expressed or disrupted in some tissues, but not others.

The term “oligonucleotide” is intended to include and single- or double stranded DNA or RNA. Oligonucleotides can be naturally occurring or synthetic, but are typically prepared by synthetic means. Preferred oligonucleotides of the invention include segments of EDN1 gene sequence or their complements, which include and/or flank any one of the polymorphic sites shown in Table 1. The segments can be between 5 and 250 bases, and, in specific embodiments, are between 5-10, 5-20, 10-20, 10-50, 20-50 or 10-100 bases. For example, the segments can be 21 bases. The polymorphic site can occur within any position of the segment or a region next to the segment. The segments can be from any of the allelic forms of the EDN1 gene sequences shown in Table 1.

The term “operably-linked” is intended to mean that the 5′ upstream regulatory element is associated with a nucleic acid in such a manner as to facilitate transcription of the nucleic acid from the 5′ upstream regulatory element.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurring most frequently in a selected population is often referred to as the reference and/or wildtype form. Other allelic forms are typically designated or alternative or variant alleles. Diploid organisms may be homozygous or heterozygous for allelic forms. A diallelic or biallelic polymorphism has two forms. A trialleleic polymorphism has three forms.

A “polymorphic gene” refers to a gene having at least one polymorphic region.

The term “primer” as used herein, refers to a single-stranded oligonucleotide which acts as a point of initiation of template-directed DNA synthesis under appropriate conditions (e.g., in the presence of four different nucleoside triphosphates and as agent for polymerization, such as DNA or RNA polymerase or reverse transcriptase) in an appropriate buffer and at a suitable temperature. The length of a primer may vary but typically ranges from 15 to 30 nucleotides. A primer need not match the exact sequence of a template, but must be sufficiently complementary to hybridize with the template.

The term “primer pair” refers to a set of primers including an upstream primer that hybridizes with the 3′ end of the complement of the DNA sequence to be amplified and a downstream primer that hybridizes with the 3′ end of the sequence to be amplified.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

A “regulatory element”, also termed herein “regulatory sequence” is intended to include elements which are capable of modulating transcription from a 5′ upstream regulatory sequence, including, but not limited to a basic promoter, and include elements such as enhancers and silencers. The term “enhancer”, also referred to herein as “enhancer element”, is intended to include regulatory elements capable of increasing, stimulating, or enhancing transcription from a 5′ upstream regulatory element, including a basic promoter. The term “silencer”, also referred to herein as “silencer element” is intended to include regulatory elements capable of decreasing, inhibiting, or repressing transcription from a 5′ upstream regulatory element, including a basic promoter. Regulatory elements are typically present in 5′ flanking regions of genes. Regulatory elements also may be present in other regions of a gene, such as introns. Thus, it is possible that an EDN1 gene has regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of an operably linked DNA sequence preferentially in specific cells (e.g., cells of a specific tissue). Gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, e.g., a molecule, such as a protein, hormone, cytokine, heavy metal, phorbol ester, cyclic AMP (cAMP), or retinoic acid.

Regulatory elements are typically bound by proteins, e.g., transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a postranslational modification, such as the attachment of a phosphate group. The activity of a transcription factor is frequently modulated by a postranslational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, e.g., in public databases, e.g., TFMATRIX Transcription Factor Binding Site Profile database.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect.

As used herein, the term “specifically hybridizes” or “specifically detects” refers to the ability of a nucleic acid molecule of the invention to hybridize to at least approximately 6, 12, 20, 30, 40, 50, 60, 70, 80, 90, 100, 110, 120, 130 or 140 consecutive nucleotides of either strand of an EDN1 gene.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence which has been genetic-engineered into a cell. Daughter cells deriving from a cell in which a transgene has been introduced are also said to contain the transgene (unless it has been deleted). A transgene can encode, e.g., a polypeptide, or an antisense transcript, partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). Alternatively, a transgene can also be present in an episome. A transgene can include one or more transcriptional regulatory sequence and any other nucleic acid, (e.g. intron), that may be necessary for optimal expression of a selected nucleic acid.

A “transgenic animal” refers to any animal, preferably a non-human animal, e.g. a mammal, bird or an amphibian, in which one or more of the cells of the animal contain heterologous nucleic acid introduced by genetic engineering, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a protein, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treatment”, or “treating” as used herein, is defined as the application or administration of a therapeutic agent to a subject, implementation of lifestyle changes (e.g., changes in diet or environment), administration of medication, use of medical devices, such as, but not limited to, stents, defibrillators, and angioplasty devices, or any combination thereof or, surgical procedures such as percutaneous transluminal coronary balloon angioplasty (PTCA) or laser angioplasty, defibrillators, implantation of a stent, or other surgical intervention, such as, for example, coronary bypass grafting (CABG), or any combination thereof, or application or administration of a therapeutic agent to an isolated tissue or cell line from a subject, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. The medical devices described in the methods of the invention can also be used in combination with a modulator of EDN1 gene expression or EDN1 polypeptide activity. “Modulators of EDN1 gene expression,” as used herein include, for example, EDN1 nucleic acid molecules, antisense EDN1 nucleic acid molecules, ribozymes, or a small molecules. “Modulators of EDN1 polypeptide activity” include, for example, EDN1-specific antibodies or EDN1 proteins or polypeptides.

As used herein, the term “vector” refers to a nucleic acid molecule capable of transporting or replicating another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively-linked are referred to herein as “expression vectors”. In general, expression vectors of utility in recombinant DNA techniques are often in the form of “plasmids” which refer generally to circular double stranded DNA circles which, in their vector form are not physically linked to the host chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Polymorphisms of the Invention

The nucleic acid molecules of the present invention include specific allelic variants of the EDN1 gene, which differ from the reference sequence set forth in SEQ ID NO:1, or at least a portion thereof, having a polymorphic region. The preferred nucleic acid molecules of the present invention comprise EDN1 sequences having the polymorphisms shown in Table 1 (SEQ ID NOs:3 and 4), and those in linkage disequilibrium therewith. The invention further comprises isolated nucleic acid molecules complementary to nucleic acid molecules comprising the polymorphisms of the present invention. Nucleic acid molecules of the present invention can function as probes or primers, e.g., in methods for determining the allelic identity of an EDN1 polymorphic region. The nucleic acids of the invention can also be used, either in combination with each other or in combination with other SNPs in the EDN1 gene or other genes, to determine whether a subject is or is not at risk of developing a disease associated with a specific allelic variant of an EDN1 polymorphic region, e.g., a vascular disease or disorder. The nucleic acids of the invention can further be used to prepare or express EDN1 polypeptides encoded by specific alleles, such as mutant alleles. Such nucleic acids can be used in gene therapy. Polypeptides encoded by specific EDN1 alleles, such as mutant EDN1 polypeptides, can also be used in therapy or for preparing reagents, e.g., antibodies, for detecting EDN1 proteins encoded by these alleles. Accordingly, such reagents can be used to detect mutant EDN1 proteins.

As described herein, allelic variants of the human EDN1 gene which are associated with vascular disease have been identified. The invention is intended to encompass the allelic variants as well as those in linkage disequilibrium which can be identified, e.g., according to the methods described herein. “Linkage disequilibrium” refers to an association between specific alleles at two marker loci within a particular population. In general, linkage disequilbrium decreases with an increase in physical distance. If linkage disequilbrium exists between two markers, then the genotypic information at one marker can be used to make predictions about the genotype of the second marker.

The invention also provides isolated nucleic acids comprising at least one polymorphic region of an EDN1 gene having a nucleotide sequence which differs from the reference nucleotide sequence set forth in SEQ ID NO:1. Preferred nucleic acids can have a polymorphic region in an upstream regulatory element, an exon, an intron, or in the 3′ UTR.

The nucleic acid molecules of the invention can be single stranded DNA (e.g., an oligonucleotide), double stranded DNA (e.g., double stranded oligonucleotide) or RNA. Preferred nucleic acid molecules of the invention can be used as probes or primers. Primers of the invention refer to nucleic acids which hybridize to a nucleic acid sequence which is adjacent to the region of interest or which covers the region of interest and is extended. As used herein, the term “hybridizes” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions vary according to the length of the involved nucleotide sequence but are known to those skilled in the art and can be found or determined based on teachings in Current Protocols in Molecular Biology, Ausubel et al., eds., John Wiley & Sons, Inc. (1995), sections 2, 4 and 6. Additional stringent conditions and formulas for determining such conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions for hybrids that are at least basepairs in length includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions for such hybrids includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions for such hybrids includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.) 81.5+16.6(log₁₀[Na⁺])+0.41 (%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

A primer or probe can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which specifically hybridizes to a polymorphic region of an EDN1 gene, and which by hybridization or absence of hybridization to the DNA of a subject or the type of hybrid formed will be indicative of the identity of the allelic variant of the polymorphic region of the EDN1 gene.

Numerous procedures for determining the nucleotide sequence of a nucleic acid molecule, or for determining the presence of mutations in nucleic acid molecules include a nucleic acid amplification step, which can be carried out by, e.g., polymerase chain reaction (PCR). Accordingly, in one embodiment, the invention provides primers for amplifying portions of an EDN1 gene, such as portions of exons and/or portions of introns. In a preferred embodiment, the exons and/or sequences adjacent to the exons of the human EDN1 gene will be amplified to, e.g., detect which allelic variant, if any, of a polymorphic region is present in the EDN1 gene of a subject. Preferred primers comprise a nucleotide sequence complementary a specific allelic variant of an EDN1 polymorphic region and of sufficient length to selectively hybridize with an EDN1 gene, or a combination thereof. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of an EDN1 gene. In an even more preferred embodiment, the primer is capable of hybridizing to an EDN1 nucleotide sequence, complements thereof, allelic variants thereof, or complements of allelic variants thereof. For example, primers comprising a nucleotide sequence of at least about 15 consecutive nucleotides, at least about 25 nucleotides or having from about 15 to about 20 nucleotides set forth in SEQ ID NOs:3 or 4, or the complement thereof are provided by the invention. Primers having a sequence of more than about 25 nucleotides are also within the scope of the invention. Preferred primers of the invention are primers that can be used in PCR for amplifying each of the exons of an EDN1 gene.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to EDN1 nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified. A forward primer can be a primer having a nucleotide sequence or a portion of the nucleotide sequence shown in Table 1 (SEQ ID NOs:3 or 4). A reverse primer can be a primer having a nucleotide sequence or a portion of the nucleotide sequence that is complementary to a nucleotide sequence shown in Table 1 (SEQ ID NOs:3 or 4).

Yet other preferred primers of the invention are nucleic acids which are capable of selectively hybridizing to an allelic variant of a polymorphic region of an EDN1 gene. Thus, such primers can be specific for an EDN1 gene sequence, so long as they have a nucleotide sequence which is capable of hybridizing to an EDN1 gene. Preferred primers are capable of specifically hybridizing to the allelic variant listed in Table 1 (SEQ ID NOs:3 or 4). Such primers can be used, e.g., in sequence specific oligonucleotide priming as described further herein.

Other preferred primers used in the methods of the invention are nucleic acids which are capable of hybridizing to the reference sequence of an EDN1 gene, thereby detecting the presence of the reference allele of an allelic variant or the absence of a variant allele of an allelic variant in an EDN1 gene. Such primers can be used in combination, e.g., primers specific for the variant polynucleotide of the EDN1 gene can be used in combination. The sequences of primers specific for the reference sequences comprising the EDN1 gene will be readily apparent to one of skill in the art.

The EDN1 nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a region of an EDN1 gene which is polymorphic (SEQ ID NOs:3 or 4). In an even more preferred embodiment of the invention, the probes are capable of hybridizing specifically to one allelic variant of an EDN1 gene having a nucleotide sequence which differs from the nucleotide sequence set forth in SEQ ID NO:1. Such probes can then be used to specifically detect which allelic variant of a polymorphic region of an EDN1 gene is present in a subject. The polymorphic region can be located in the 3′ UTR, 5′ upstream regulatory element, exon, or intron sequences of an EDN1 gene.

Particularly, preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of an EDN1 gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In a preferred embodiment of the invention, the isolated nucleic acid, which is used, e.g., as a probe or a primer, is modified, so as to be more stable than naturally occurring nucleotides. Exemplary nucleic acid molecules which are modified include phosphoramidate, phosphothioate and methylphosphonate analogs of DNA (see also U.S. Pat. Nos. 5,176,996; 5,264,564; and 5,256,775).

The nucleic acids of the invention can also be modified at the base moiety, sugar moiety, Dr phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), hybridization-triggered cleavage agents. (See, e.g., Krol et al., 1988, BioTechniques 6:958-976) or intercalating agents (see, e.g., Zon, 1988, Pharm. Res. 5:539-549). To this end, the nucleic acid of the invention may be conjugated to another molecule, e.g., a peptide, hybridization triggered cross-linking agent, transport agent, hybridization-triggered cleavage agent, etc.

The isolated nucleic acid comprising an EDN1 intronic sequence may comprise at least one modified base moiety which is selected from the group including but not limited to 5-fluoroiracil, 5-bromouracil, 5-chlorouracil, 5-iodouracil, hypoxanthine, xantine, 4-acetylcyitidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocyticline, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an a-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The invention also provides vectors and plasmids comprising the nucleic acids of the invention. For example, in one embodiment, the invention provides a vector comprising at least a portion of the EDN1 gene comprising a polymorphic region. Thus, the invention provides vectors for expressing at least a portion of the newly identified allelic variants of the human EDN1 gene reference sequence, as well as other allelic variants, comprising a nucleotide sequence which is different from the nucleotide sequence disclosed in GI 2791272. The allelic variants can be expressed in eukaryotic cells, e.g., cells of a subject, e.g., a mammalian subject, or in prokaryotic cells.

In one embodiment, the vector comprising at least a portion of an EDN1 allele is introduced into a host cell, such that a protein encoded by the allele is synthesized. The EDN1 protein produced can be used, e.g., for the production of antibodies, which can be used, e.g., in methods for detecting mutant forms of EDN1. Alternatively, the vector can be used for gene therapy, and be, e.g., introduced into a subject to produce EDN1 protein. Host cells comprising a vector having at least a portion of an EDN1 gene are also within the scope of the invention.

Polypeptides of the Invention

The present invention provides isolated EDN1 polypeptides, such as EDN1 polypeptides which are encoded by specific allelic variants of EDN1, including those identified herein, e.g., an END1 polypeptide comprising an asparagine at amino acid residue 198 of SEQ ID NO:2. The amino acid sequence of the EDN1 protein has been deduced. The EDN1 gene encodes a 212 amino acid protein and is described in, for example, Inoue A, et al. (1989) J. Biol. Chem. 264 (25), 14954-14959, incorporated herein by reference.

In one embodiment, the EDN1 polypeptides are isolated from, or otherwise substantially free of other cellular proteins. The term “substantially free of other cellular proteins” (also referred to herein as “contaminating proteins”) or “substantially pure or purified preparations” are defined as encompassing preparations of EDN1 polypeptides having less than about 20% (by dry weight) contaminating protein, and preferably having less than about 5% contaminating protein. It will be appreciated that functional forms of the subject polypeptides can be prepared, for the first time, as purified preparations by using a cloned gene as described herein.

Preferred EDN1 proteins of the invention have an amino acid sequence which is at least about 60%, 70%, 80%, 85%, 90%, or 95% identical or homologous to the amino acid sequence of SEQ ID NO:2. Even more preferred EDN1 proteins comprise an amino acid sequence which is at least about 95%, 96%, 97%, 98%, or 99% homologous or identical to the amino acid sequence of SEQ ID NO:2. Such proteins can be recombinant proteins, and can be, e.g., produced in vitro from nucleic acids comprising a specific allele of an EDN1 polymorphic region. For example, recombinant polypeptides preferred by the present invention can be encoded by a nucleic acid which comprises a sequence which is at least 85% homologous and more preferably 90% homologous and most preferably 95% homologous with a nucleotide sequence set forth in SEQ ID NO:1 and comprises an allele of a polymorphic region that differs from that set forth in SEQ ID NO: 1. Polypeptides which are encoded by a nucleic acid comprising a sequence that is at least about 98-99% homologous with the sequence of SEQ ID NO:1 and comprises an allele of a polymorphic region that differs from that set forth in SEQ ID NO:1 are also within the scope of the invention.

In a preferred embodiment, an EDN1 protein of the present invention is a mammalian EDN1 protein. In an even more preferred embodiment, the EDN1 protein is a human protein.

The invention also provides peptides that preferably are capable of functioning in one of either role of an agonist or antagonist of at least one biological activity of a wild-type (“normal”) EDN1 protein of the appended sequence listing. The term “evolutionarily related to,” with respect to amino acid sequences of EDN1 proteins, refers to both polypeptides having amino acid sequences found in human populations, and also to artificially produced mutational variants of human EDN1 polypeptides which are derived, for example, by combinatorial mutagenesis.

Full length proteins or fragments corresponding to one or more particular motifs and/or domains or to arbitrary sizes, for example, at least 5, 10, 25, 50, 75 and 100, amino acids in length of EDN1 protein are within the scope of the present invention.

Isolated EDN1 peptides or polypeptides can be obtained by screening peptides recombinantly produced from the corresponding fragment of the nucleic acid encoding such peptides. In addition, such peptides and polypeptides can be chemically synthesized using techniques known in the art such as conventional Merrifield solid phase f-Moc or t-Boc chemistry. For example, an EDN1 peptide or polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptides or polypeptides which can function as either agonists or antagonists of a wild-type (e.g., “normal”) EDN1 protein.

In general, peptides and polypeptides referred to herein as having an activity (e.g., are “bioactive”) of an EDN1 protein are defined as peptides and polypeptides which mimic or antagonize all or a portion of the biological/biochemical activities of an EDN1 protein having SEQ ID NO:2, such as the ability to bind ligands. Other biological activities of the subject EDN1 proteins are described herein or will be reasonably apparent to those skilled in the art. According to the present invention, a peptide or polypeptide has biological activity if it is a specific agonist or antagonist of a naturally-occurring form of an EDN1 protein.

Assays for determining whether an EDN1 protein or variant thereof, has one or more biological activities are well known in the art.

Other preferred proteins of the invention are those encoded by the nucleic acids set forth in the section pertaining to nucleic acids of the invention. In particular, the invention provides fusion proteins, e.g., EDN1-immunoglobulin fusion proteins. Such fusion proteins can provide, e.g., enhanced stability and solubility of EDN1 proteins and may thus be useful in therapy. Fusion proteins can also be used to produce an immunogenic fragment of an EDN1 protein. For example, the VP6 capsid protein of rotavirus can be used as an immunologic carrier protein for portions of the EDN1 polypeptide, either in the monomeric form or in the form of a viral particle. The nucleic acid sequences corresponding to the portion of a subject EDN1 protein to which antibodies are to be raised can be incorporated into a fusion gene construct which includes coding sequences for a late vaccinia virus structural protein to produce a set of recombinant viruses expressing fusion proteins comprising EDN1 epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of an EDN1 protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of an EDN1 polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of EDN1 proteins can also be expressed and presented by bacterial cells.

Fusion proteins can also facilitate the expression of proteins including the EDN1 polypeptides of the present invention. For example, EDN1 polypeptides can be generated as glutathione-S-transferase (GST-fusion) proteins. Such GST-fusion proteins can be easily purified, as for example by the use of glutathione-derivatized matrices (see, for example, Current Protocols in Molecular Biology, eds. Ausubel et al. (N.Y.: John Wiley & Sons, 1991)) and used subsequently to yield purified EDN1 polypeptides.

The present invention further pertains to methods of producing the subject EDN1 polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant EDN1 polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant EDN1 polypeptide is a fusion protein containing a domain which facilitates its purification, such as GST fusion protein.

Moreover, it will be generally appreciated that, under certain circumstances, it may be advantageous to provide homologs of one of the subject EDN1 polypeptides which function in a limited capacity as one of either an EDN1 agonist (mimetic) or an EDN1 antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of EDN1 proteins.

Homologs of each of the subject EDN1 proteins can be generated by mutagenesis, such as by discrete point mutation(s), and/or by truncation. For instance, mutation can give rise to homologs which retain substantially the same, or merely a subset, of the biological activity of the EDN1 polypeptide from which it was derived. Alternatively, antagonistic forms of the protein can be generated which are able to inhibit the function of the naturally occurring form of the protein, such as by competitively binding to an EDN1 receptor.

The recombinant EDN1 polypeptides of the present invention also include homologs of EDN1 polypeptides which differ from the EDN1 protein having SEQ ID NO:2, such as versions of the protein which are resistant to proteolytic cleavage, as for example, due to mutations which alter ubiquitination or other enzymatic targeting associated with the protein.

EDN1 polypeptides may also be chemically modified to create EDN1 derivatives by forming covalent or aggregate conjugates with other chemical moieties, such as glycosyl groups, lipids, phosphate, acetyl groups and the like. Covalent derivatives of EDN1 proteins can be prepared by linking the chemical moieties to functional groups on amino acid side-chains of the protein or at the N-terminus or at the C-terminus of the polypeptide.

Modification of the structure of the subject EDN1 polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the EDN1 polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e., isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing =cysteine and methionine. (see, for example, Biochemistry, 2^{nd} ed., Ed. by L. Stryer, W H Freeman and Co.: 1981). Whether a change in the amino acid sequence of a peptide results in a functional EDN1 homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

Methods

The invention further provides predictive medicine methods, which are based, at least in part, on the discovery of EDN1 polymorphic regions which are associated with specific physiological states and/or diseases or disorders, e.g., vascular diseases or disorders such as CAD and MI. These methods can be used alone, or in combination with other predictive medicine methods, including the identification and analysis of known risk factors associated with vascular disease, e.g., phenotypic factors such as, for example, obesity and diabetes, and family history.

For example, information obtained using the diagnostic assays described herein (in combination with each other or in combination with information of another genetic defect which contributes to the same disease, e.g., a vascular disease or disorder) is useful for diagnosing or confirming that a subject has an allele of a polymorphic region which is associated with a particular disease or disorder, e.g., a vascular disease or disorder, or a combination of alleles which are associated with a particular disease or disorder, e.g., at least one copy of the variant allele at nucleotide position 157790 of GI 2791272 (C) in combination with at least one copy of the reference allele at nucleotide position 159908 (G) of GI 2791272, or the complements thereof, or at least one copy of the reference allele at nucleotide position 157790 (T) of GI 2791272 in combination with at least one copy of the variant allele at nucleotide position 159908 (T) of GI 2791272, or the complements thereof. Moreover, the information obtained using the diagnostic assays described herein, in combination with each other or in combination with information of another genetic defect which contributes to the same disease, e.g., a vascular disease or disorder, can be used to predict whether or not a subject will benefit from further diagnostic evaluation for a vascular disease or disorder. Such further diagnostic evaluation includes, but is not limited to, cardiovascular imaging, such as angiography, cardiac ultrasound, coronary angiogram, magnetic resonance imagery, nuclear imaging, CT scan, myocardial perfusion imagery, or electrocardiogram, genetic analysis, e.g., identification of additional polymorphisms e.g., which contribute to the same disease, familial health history analysis, lifestyle analysis, or exercise stress tests, either alone or in combination. Furthermore, the diagnostic information obtained using the diagnostic assays described herein (in combination with each other or in combination with information of another genetic defect which contributes to the same disease, e.g., a vascular disease or disorder), may be used to identify which subject will benefit from a particular clinical course of therapy useful for preventing, treating, ameliorating, or prolonging onset of the particular vascular disease or disorder in the particular subject. Clinical courses of therapy include, but are not limited to, administration of medication, non-surgical intervention, surgical procedures such as percutaneous transluminal coronary angioplasty, laser angioplasty, implantation of a stent, coronary bypass grafting, implantation of a defibrillator, implantation of a pacemaker, and any combination thereof, and use of surgical and non-surgical medical devices used in the treatment of vascular disease, such as, for example, a defibrillator, a stent, a device used in coronary revascularization, a pacemaker, and any combination thereof. Medical devices may also be used in combination with a modulator of EDN1 gene expression or EDN1 polypeptide activity.

Alternatively, the information, in combination with each other, or, preferably, in combination with information of another genetic defect which contributes to the same disease, e.g., a vascular disease or disorder, can be used prognostically for predicting whether a non-symptomatic subject is likely to develop a disease or condition which is associated with one or more specific alleles of EDN1 polymorphic regions in a subject. Based on the prognostic information, a health care provider can recommend a particular further diagnostic evaluation which will benefit the subject, or a particular clinical course of therapy, as described above.

In addition, knowledge of the identity of one or more particular EDN1 alleles in a subject (the EDN1 genetic profile), preferably, the alleles at nucleotide positions 157790 and 159908 of SEQ ID NO:1, or the complements thereof, allows customization of further diagnostic evaluation and/or a clinical course of therapy for a particular disease. For example, a subject's EDN1 genetic profile or the genetic profile of a disease or disorder associated with a specific allele of an EDN1 polymorphic region, e.g., a vascular disease or disorder, can enable a health care provider: 1) to more efficiently and cost-effectively identify means for further diagnostic evaluation, including, but not limited to, further genetic analysis, familial health history analysis, or use of vascular imaging devices or procedures; 2) to more effectively prescribe a drug that will address the molecular basis of the disease or condition; 3) to more efficiently and cost-effectively identify an appropriate clinical course of therapy, including, but not limited to, lifestyle changes, medications, surgical or non-surgical medical devices, surgical or non-surgical intervention or procedures, or any combination thereof; and 4) to better determine the appropriate dosage of a particular drug or duration of a particular course of clinical therapy. For example, the expression level of EDN1 proteins, alone or in conjunction with the expression level of other genes known to contribute to the same disease, can be measured in many subjects at various stages of the disease to generate a transcriptional or expression profile of the disease. Expression patterns of individual subjects can then be compared to the expression profile of the disease to determine the appropriate drug, dose to administer to the subject, or course of clinical therapy.

The ability to target populations expected to show the highest clinical benefit, based on the EDN1 or disease genetic profile, can enable: 1) the repositioning of marketed drugs, medical devices and surgical procedures for use in treating, preventing, or ameliorating vascular diseases or disorders, or diagnostics, such as vascular imaging devices or procedures, with disappointing market results; 2) the rescue of drug candidates whose clinical development has been discontinued as a result of safety or efficacy limitations, which are subject subgroup-specific; 3) an accelerated and less costly development for drug candidates and more optimal drug labeling (e.g., since the use of EDN1 as a marker is useful for optimizing effective dose); and 4) an accelerated, less costly, and more effective selection of a particular course of clinical therapy suited to a particular subject.

These and other methods are described in further detail in the following sections.

A. Prognostic and Diagnostic Assays

The present methods provide means for determining if a subject has or is or is not at risk of developing a disease, condition or disorder that is associated a specific EDN1 allele or combinations thereof, e.g., a vascular disease or a disease or disorder resulting therefrom.

The present invention provides methods for determining the molecular structure of an EDN1 gene, such as a human EDN1 gene, or a portion thereof. In one embodiment, determining the molecular structure of at least a portion of an EDN1 gene comprises determining the identity of the allelic variant of at least one polymorphic region of an EDN1 gene (determining the presence or absence of the allelic variant of SEQ ID NOs:3 and/or 4, or the complement thereof). A polymorphic region of an EDN1 gene can be located in an exon, an intron, at an intron/exon border, or in the 5′ upstream regulatory element of the EDN1 gene.

The invention provides methods for determining whether a subject has or is at risk of developing, a disease or disorder associated with a specific allelic variant of a polymorphic region of an EDN1 gene. Such diseases can be associated with aberrant EDN1 activity, e.g., a vascular disease or disorder.

Analysis of one or more EDN1 polymorphic regions in a subject can be useful for predicting whether a subject has or is likely to develop a vascular disease or disorder, e.g., CAD, MI, atherosclerosis, ischemia, stroke, peripheral vascular diseases, venous thromboembolism and pulmonary embolism.

In preferred embodiments, the methods of the invention can be characterized as comprising detecting, in a sample of cells from the subject, the presence or absence of a specific allelic variant of one or more polymorphic regions of an EDN1 gene. The allelic differences can be: (i) a difference in the identity of at least one nucleotide or (ii) a difference in the number of nucleotides, which difference can be a single nucleotide or several nucleotides. The invention also provides methods for detecting differences in an EDN1 gene such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of an EDN1 gene prior to identifying the allelic variant. Amplification can be performed, e.g., by PCR an(/or LCR (see Wu and Wallace, (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad Sci. USA 86:1173-1177), Q-Beta Replicase (Lizardi, P. M. et al., 1988, Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of an EDN1 gene and detect allelic variants, e.g., mutations, by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of an EDN1 gene in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an EDN1 allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of EDN1 allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of EDN1. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and can be used to detect specific allelic variants of a polymorphic region of an EDN1 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microliter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in an EDN1 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. (French Patent 2,650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J. Hum. Genet. 52:46-59 (1993)).

For determining the identity of the allelic variant of a polymorphic region located in the coding region of an EDN1 gene, yet other methods than those described above can be used. For example, identification of an allelic variant which encodes a mutated EDN1 protein can be performed by using an antibody specifically recognizing the mutant protein in, e.g., immunohistochemistry or immunoprecipitation. Antibodies to wild-type EDN1 or mutated forms of EDN1 proteins can be prepared according to methods known in the art.

Alternatively, one can also measure an activity of an EDN1 protein, such as binding to an EDN1 ligand. Binding assays are known in the art and involve, e.g. obtaining cells from a subject, and performing binding experiments with a labeled lipid, to determine whether binding to the mutated form of the protein differs from binding to the wild-type of the protein.

Antibodies directed against reference or mutant EDN1 polypeptides or allelic variant thereof, which are discussed above, may also be used in disease diagnostics and prognostics. Such diagnostic methods, may be used to detect abnormalities in the level of EDN1 polypeptide expression, or abnormalities in the structure and/or tissue, cellular, or subcellular location of an EDN1 polypeptide. Structural differences may include, for example, differences in the size, electronegativity, or antigenicity of the mutant EDN1 polypeptide relative to the normal EDN1 polypeptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of EDN1 polypeptides. In situ detection may be accomplished by removing a histological specimen from a subject, and applying thereto a labeled antibody of the present invention. The antibody (or fragment) is preferably applied by overlaying the labeled antibody (or fragment) onto a biological sample. Through the use of such a procedure, it is possible to determine not only the presence of the EDN1 polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-EDN1 polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific EDN1 allelic variant.

Sample nucleic acid to be analyzed by any of the above-described diagnostic and prognostic methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

B. Pharmacogenomics

Knowledge of the identity of the allele of the EDN1 gene polymorphic region in a subject (the more EDN1 genetic profile), alone or in conjunction with information of other genetic defects associated with the same disease (the genetic profile of the particular disease) also allows selection and customization of the therapy, e.g., a particular clinical course of therapy and/or further diagnostic evaluation for a particular disease to the subject's genetic profile. For example, subjects having specific alleles of an EDN1 gene in combination, may or may not exhibit symptoms of a particular disease or be predisposed to developing symptoms of a particular disease. Further, if those subjects are symptomatic, they may or may not respond to a certain drug, e.g., a specific therapeutic used in the treatment or prevention of a vascular disease or disorder, e.g., CAD or MI, such as, for example, beta blocker drugs, calcium channel blocker drugs, or nitrate drugs, but may respond to another. Furthermore, they may or may not respond to other treatments, including, for example, use of medical devices for treatment of vascular disease, or surgical and/or non-surgical procedures or courses of treatment. Moreover, if a subject does or does not exhibit symptoms of a particular disease, the subject may or may not benefit from further diagnostic evaluation, including, for example, use of vascular imaging devices or procedures. Thus, generation of an EDN1 genetic profile, (e.g., categorization of alterations in an EDN1 gene which are associated with the development of a particular disease), from a population of subjects, who are symptomatic for a disease or condition that is caused by or contributed to by a defective and/or deficient EDN1 gene and/or protein (an EDN1 genetic population profile) and comparison of a subject's EDN1 profile to the population profile, permits the selection or design of drugs that are expected to be safe and efficacious for a particular subject or subject population (i.e., a group of subjects having the same genetic alteration), as well as the selection or design of a particular clinical course of therapy or further diagnostic evaluations that are expected to be safe and efficacious for a particular subject or subject population.

For example, an EDN1 population profile can be performed by determining the EDN1 profile, e.g., the identity of EDN1 alleles, in a subject population having a disease, which is associated with one or more specific alleles of EDN1 polymorphic regions. Optionally, the EDN1 population profile can further include information relating to the response of the population to an EDN1 therapeutic, using any of a variety of methods, including, monitoring: 1) the severity of symptoms associated with the EDN1 related disease; 2) EDN1 gene expression level; 3) EDN1 mRNA level; and/or 4) EDN1 protein level, and dividing or categorizing the population based on particular EDN1 alleles. The EDN1 genetic population profile can also, optionally, indicate those particular EDN1 alleles which are present in subjects that are either responsive or non-responsive to a particular therapeutic, clinical course of therapy, or diagnostic evaluation. This information or population profile, is then useful for predicting which individuals should respond to particular drugs, particular clinical courses of therapy, or diagnostic evaluations based on their individual EDN1 genetic profile.

In a preferred embodiment, the EDN1 profile is a transcriptional or expression level profile and is comprised of determining the expression level of EDN1 proteins, alone or in conjunct ion with the expression level of other genes known to contribute to the same disease at various stages of the disease.

Pharmacogenomic studies can also be performed using transgenic animals. For example, one can produce transgenic mice, e.g., as described herein, which contain a specific allelic variant of an EDN1 gene. These mice can be created, e.g., by replacing their wild-type EDN1 gene with an allele of the human EDN1 gene. The response of these mice to specific EDN1 particular therapeutics, clinical courses of treatment, and/or diagnostic evaluations can then be determined.

(i) Diagnostic Evaluation

In one embodiment, the polymorphisms of the present invention are used to determine the most appropriate diagnostic evaluation and to determine whether or not a subject will benefit from further diagnostic evaluation. For example, if a subject has at least one copy of the variant allele at nucleotide position 157790 of GI 2791272 (C) in combination with at least one copy of the reference allele at nucleotide position 159908 (G) of GI 2791272, or the complements thereof, or at least one copy of the reference allele at nucleotide position 157790 (T) of GI 2791272 in combination with at least one copy of the variant allele at nucleotide position 159908 (T) of GI 2791272, or the complements thereof, as described herein, that subject is more likely to have or to be at a higher than normal risk of developing a vascular disease such as CAD or MI.

Thus, in one embodiment, the invention provides methods for classifying a subject who has, or is at risk for developing, a vascular disease or disorder as a candidate for further diagnostic evaluation for a vascular disease or disorder comprising the steps of determining the EDN1 genetic profile of the subject, comparing the subject's EDN1 genetic profile to an EDN1 genetic population profile, and classifying the subject based on the identified genetic profiles as a subject who is a candidate for further diagnostic evaluation for a vascular disease or disorder

In a preferred embodiment, the subject's EDN1 genetic profile is determined by identifying the nucleotides present at nucleotide positions 157790 and 159908 of the reference sequence GI 2791272 of the EDN1 gene, or the complements thereof.

Methods of further diagnostic evaluation include use of vascular imaging devices or procedures such as, for example, angiography, cardiac ultrasound, coronary angiogram, magnetic resonance imagery, nuclear imaging, CT scan, myocardial perfusion imagery, or electrocardiogram, or may include genetic analysis, familial health history analysis, lifestyle analysis, exercise stress tests, or any combination thereof.

In another embodiment, the invention provides methods for selecting an effective vascular imaging device as a diagnostic tool for a vascular disease or disorder comprising the steps of determining the EDN1 genetic profile of the subject; comparing the subject's EDN1 genetic profile to an EDN1 genetic population profile; and selecting an effective vascular imaging device or procedure as a diagnostic tool for a vascular disease or disorder. In a preferred embodiment, the vascular imaging device is selected from the group consisting of angiography, cardiac ultrasound, coronary angiogram, magnetic resonance imagery, nuclear imaging, CT scan, myocardial perfusion imagery, electrocardiogram, or any combination thereof.

(ii) Clinical Course of Therapy

In another aspect, the polymorphisms of the present invention are used to determine the most appropriate clinical course of therapy for a subject who has or is at risk of a vascular disease or disorder, and will aid in the determination of whether the subject will benefit from such clinical course of therapy, as determined by identification of the polymorphisms of the invention. If a subject has at least one copy of the variant allele at nucleotide position 157790 of GI 2791272 (C) in combination with at least one copy of the reference allele at nucleotide position 159908 (G) of GI 2791272, or the complements thereof, or at least one copy of the reference allele at nucleotide position 157790 (T) of GI 2791272 in combination with at least one copy of the variant allele at nucleotide position 159908 (T) of GI 2791272, or the complements thereof, that subject is more likely to have or to be at a higher than normal risk of developing a vascular disease such as CAD or MI.

Thus, in one aspect, the invention relates to the SNPs identified as described herein, in combination, as well as to the use of these SNPs, and others in these genes, particularly those nearby in linkage disequilibrium with these SNPs, in combination, for prediction of a particular clinical course of therapy for a subject who has, or is at risk for developing, a vascular disease. In one embodiment, the invention provides a method for determining whether a subject will benefit from a particular course of therapy by determining the presence of the polymorphisms of the invention. For example, the determination of the polymorphisms of the invention, in combination with each other, or in combination with other polymorphisms in the EDN1 gene or other genes, will aid in the determination of whether an individual will benefit from surgical revascularization and/or will benefit by the implantation of a stent following surgical revascularization, and will aid in the determination of the likelihood of success or failure of a particular clinical course of therapy.

In one embodiment, the invention provides methods for classifying a subject who has, or is at risk for developing, a vascular disease or disorder as a candidate for a particular clinical course of therapy for a vascular disease or disorder comprising the steps of determining the EDN1 genetic profile of the subject; comparing the subject's EDN1 genetic profile to an EDN1 genetic population profile; and classifying the subject based on the identified genetic profiles as a subject who is a candidate for a particular clinical course of therapy for a vascular disease or disorder.

In another embodiment, the invention provides methods for selecting an effective clinical course of therapy to treat a subject who has, or is at risk for developing, a vascular disease or disorder comprising the steps of: determining the EDN1 genetic profile of the subject; comparing the subject's EDN1 genetic profile to an EDN1 genetic population profile; and selecting an appropriate clinical course of therapy for treatment of a subject who has, or is at risk for developing, a vascular disease or disorder. An appropriate clinical course of therapy may include, for example, a lifestyle change, including, for example, a change in diet or environment. Other clinical courses of therapy include, but are not limited to, use of surgical procedures or medical devices. Surgical procedures for the treatment of vascular disorders, includes, for example, surgical revascularization, such as angioplasty, e.g., percutaneous transluminal coronary balloon angioplasty (PTCA), or laser angioplasty, or coronary bypass grafting (CABG). Medical devices used in the treatment or prevention of vascular diseases or disorders, include, for example, devices used in angioplasty, such as balloon angioplasty or laser angioplasty, a device used in coronary revascularization, or a stent, a defibrillator, a pacemaker, or any combination thereof. Medical devices may also be used in combination with modulators of EDN1 gene expression or EDN1 protein activity.

C. Monitoring Effects of EDN1 Therapeutics During Clinical Trials

The present invention provides a method for monitoring the effectiveness of treatment of a subject with an EDN1 therapeutic e.g., a modulator or agent (e.g., an agonist, antagonist, such as, for example, a peptidomimetic, protein, peptide, nucleic acid, ribozyme, small molecule, or other drug candidate identified, e.g., by the screening assays described herein) comprising the steps of (i) obtaining a preadministration sample from a subject prior to administration of the agent; (ii) detecting the level of expression or activity of an EDN1 protein, mRNA or gene in the preadministration sample; (iii) obtaining one or more post-administration samples from the subject; (iv) detecting the level of expression or activity of the EDN1 protein, mRNA or gene in the post-administration samples; (v) comparing the level of expression or activity of the EDN1 protein, mRNA, or gene in the preadministration sample with those of the EDN1 protein, mRNA, or gene in the post administration sample or samples; and (vi) altering the administration of the agent to the subject accordingly. For example, increased administration of the agent may be desirable to increase the expression or activity of EDN1 to higher levels than detected, i.e., to increase the effectiveness of the agent. Alternatively, decreased administration of the agent may be desirable to decrease expression or activity of EDN1 to lower levels than detected, i.e., to decrease the effectiveness of the agent.

Cells of a subject may also be obtained before and after administration of an EDN1 therapeutic to detect the level of expression of genes other than EDN1, to verify that the EDN1 therapeutic does not increase or decrease the expression of genes which could be deleterious. This can be done, e.g., by using the method of transcriptional profiling. Thus, mRNA from cells exposed in vivo to an EDN1 therapeutic and mRNA from the same type of cells that were not exposed to the EDN1 therapeutic could be reverse transcribed and hybridized to a chip containing DNA from numerous genes, to thereby compare the expression of genes in cells treated and not treated with an EDN1 therapeutic. If, for example an EDN1 therapeutic turns on the expression of a proto-oncogene in a subject, use of this particular EDN1 therapeutic may be undesirable.

D. Methods of Treatment

The present invention provides for both prophylactic and therapeutic methods of treating a subject having or likely to develop a disorder associated with specific EDN1 alleles and/or aberrant EDN1 expression or activity, e.g., vascular diseases or disorders.

i) Prophylactic Methods

In one aspect, the invention provides a method for preventing a disease or disorder associated with a specific EDN1 allele such as a vascular disease or disorder, e.g., CAD or MI, and medical conditions resulting therefrom, by administering to the subject an agent which counteracts the unfavorable biological effect of the specific EDN1 allele. Subjects at risk for such a disease can be identified by a diagnostic or prognostic assay, e.g., as described herein. Administration of a prophylactic agent can occur prior to the manifestation of symptoms associated with specific EDN1 alleles, such that a disease or disorder is prevented or, alternatively, delayed in its progression. Depending on the identity of the EDN1 allele in a subject, a compound that counteracts the effect of this allele is administered. The compound can be a compound modulating the activity of EDN1, e.g., an EDN1 inhibitor. The treatment can also be a specific lifestyle change, e.g., a change in diet or an environmental alteration. In particular, the treatment can be undertaken prophylactically, before any other symptoms are present. Such a prophylactic treatment could thus prevent the development of aberrant vascular activity, e.g., the production of atherosclerotic plaque leading to, e.g., CAD or MI. The prophylactic methods are similar to therapeutic methods of the present invention and are further discussed in the following subsections.

(ii) Therapeutic Methods

The invention further provides methods of treating a subject having a disease or disorder associated with a specific allelic variant of a polymorphic region of an EDN1 gene. Preferred diseases or disorders include vascular diseases and disorders, and disorders resulting therefrom (e.g., such as, for example, atherosclerosis, CAD, MI, ischemia, stroke, peripheral vascular diseases, venous thromboembolism and pulmonary embolism).

In one embodiment, the method comprises (a) determining the identity of one or more of the allelic variants of an EDN1 gene, or preferably, the identity of the nucleotides at nucleotide residues 157790 and 159908 of SEQ ID NO:1, or the complements thereof; and (b) administering to the subject a compound that compensates for the effect of the specific allelic variant(s). The polymorphic region can be localized at any location of the gene, e.g., in a regulatory element (e.g., in a 5′ upstream regulatory element), in an exon, (e.g., coding region of an exon), in an intron, at an exon/intron border, or in the 3′ UTR. Thus, depending on the site of the polymorphism in the EDN1 gene, a subject having a specific variant of the polymorphic region which is associated with a specific disease or condition, can be treated with compounds which specifically compensate for the effect of the allelic variant.

In a preferred embodiment, the identity of the nucleotides present at the nucleotide residue 157790 and 159908 of SEQ ID NO:1 (the EDN1 gene), or the complement thereof is determined. If a subject has at least one copy of the variant allele at nucleotide position 157790 of GI 2791272 (C) in combination with at least one copy of the reference allele at nucleotide position 159908 (G) of GI 2791272, or the complements thereof, or at least one copy of the reference allele at nucleotide position 157790 (T) of GI 2791272 in combination with at least one copy of the variant allele at nucleotide position 159908 (T) of GI 2791272, or the complements thereof, that subject is at a higher than normal risk of developing a vascular disease such as CAD or MI.

A mutation can be a substitution, deletion, and/or addition of at least one nucleotide relative to the wild-type allele (i.e., the reference sequence). Depending on where the mutation is located in the EDN1 gene, the subject can be treated to specifically compensate for the mutation. For example, if the mutation is present in the coding region of the gene and results in a more active EDN1 protein, the subject can be treated, e.g., by administration to the subject of a modulator, e.g., a therapeutic or course of clinical treatment which treat, prevents, or ameliorates a vascular disease or disorder. Normal EDN1 protein can also be used to counteract or compensate for the endogenous mutated form of the EDN1 protein. Normal EDN1 protein can be directly delivered to the subject or indirectly by gene therapy wherein some cells in the subject are transformed or transfected with an expression construct encoding wild-type EDN1 protein. Nucleic acids encoding reference human EDN1 protein are set forth in SEQ ID NO:1.

Yet in another embodiment, the invention provides methods for treating a subject having a mutated EDN1 gene, in which the mutation is located in a regulatory region of the gene. Such a regulatory region can be localized in the 5′ upstream regulatory element of the gene, in the 5′ or 3′ untranslated region of an exon, or in an intron. A mutation in a regulatory region can result in increased production of EDN1 protein, decreased production of EDN1 protein, or production of EDN1 having an aberrant tissue distribution. The effect of a mutation in a regulatory region upon the EDN1 protein can be determined, e.g., by measuring the EDN1 protein level or mRNA level in cells having an EDN1 gene having this mutation and which, normally (i.e., in the absence of the mutation) produce EDN1 protein. The effect of a mutation can also be determined in vitro. For example, if the mutation is in the 5′ upstream regulatory element, a reporter construct can be constructed which comprises the mutated 5′ upstream regulatory element linked to a reporter gene, the construct transfected into cells, and comparison of the level of expression of the reporter gene under the control of the mutated 5′ upstream regulatory element and under the control of a wild-type 5′ upstream regulatory element. Such experiments can also be carried out in mice transgenic for the mutated 5′ upstream regulatory element. If the mutation is located in an intron, the effect of the mutation can be determined, e.g., by producing transgenic animals in which the mutated EDN1 gene has been introduced and in which the wild-type gene may have been knocked out. Comparison of the level of expression of EDN1 in the mice transgenic for the mutant human EDN1 gene with mice transgenic for a wild-type human EDN1 gene will reveal whether the mutation results in increased, or decreased synthesis of the EDN1 protein and/or aberrant tissue distribution of EDN1 protein. Such analysis could also be performed in cultured cells, in which the human mutant EDN1 gene is introduced and, e.g., replaces the endogenous wild-type EDN1 gene in the cell. Thus, depending on the effect of the mutation in a regulatory region of an EDN1 gene, a specific treatment can be administered to a subject having such a mutation. Accordingly, if the mutation results in increased EDN1 protein levels, the subject can be treated by administration of a compound which reduces EDN1 protein production, e.g., by reducing EDN1 gene expression or a compound which inhibits or reduces the activity of EDN1.

A correlation between drug responses and specific alleles of EDN1 can be shown, for example, by clinical studies wherein the response to specific drugs of subjects having different allelic variants of a polymorphic region of an EDN1 gene is compared. Such studies can also be performed using animal models, such as mice having various alleles of a human EDN1 gene and in which, e.g., the endogenous EDN1 gene has been inactivated such as by a knock-out mutation. Test drugs are then administered to the mice having different human EDN1 alleles and the response of the different mice to a specific compound is compared. Accordingly, the invention provides assays for identifying the drug which will be best suited for treating a specific disease or condition in a subject. For example, it will be possible to select drugs which will be devoid of toxicity, or have the lowest level of toxicity possible for treating a subject having a disease or condition.

Other Uses for the Nucleic Acid Molecules of the Invention

The identification of different alleles of EDN1 can also be useful for identifying an individual among other individuals from the same species. For example, DNA sequences can be used as a fingerprint for detection of different individuals within the same species (Thompson, J. S. and Thompson, eds., Genetics in Medicine, W B Saunders Co., Philadelphia, Pa. (1991)). This is useful, for example, in forensic studies and paternity testing, as described below.

A. Forensics

Determination of which specific allele occupies a set of one or more polymorphic sites in an individual identifies a set of polymorphic forms that distinguish the individual from others in the population. See generally National Research Council, The Evaluation of Forensic DNA Evidence (Eds. Pollard et al., National Academy Press, DC, 1996). The more polymorphic sites that are analyzed, the lower the probability that the set of polymorphic forms in one individual is the same as that in an unrelated individual. Preferably, if multiple sites are analyzed, the sites are unlinked. Thus, the polymorphisms of the invention can be used in conjunction with known polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of polymorphic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers is the same in the sample as in the suspect, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. For example, in biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation: p(ID)=(x²).

These calculations can be extended for any number of polymorphic forms at a given locus. For example, the probability of identity p(ID) for a 3-allele system where the alleles have the frequencies in the population of x, y, and z, respectively, is equal to the sum of the squares of the genotype frequencies: P(ID)=X⁴+(2xy)²+(2yz)²+(2XZ)²+Z⁴+y⁴.

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus: cum p(ID)=p(ID1)p(ID2)p(ID3) . . . p(IDn).

The cumulative probability of non-identity for n loci (i.e., the probability that two random individuals will be difference at 1 or more loci) is given by the equation: cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

B. Paternity Testing

The object of paternity testing is usually to determine whether a male is the father of a child. In most cases, the mother of the child is known, and thus, it is possible to trace the mother's contribution to the child's genotype. Paternity testing investigates whether the part of the child's genotype not attributable to the mother is consistent to that of the putative father. Paternity testing can be performed by analyzing sets of polymorphisms in the putative father and in the child.

If the set of polymorphisms in the child attributable to the father does not match the set of polymorphisms of the putative father, it can be concluded, barring experimental error, that that putative father is not the real father. If the set of polymorphisms in the child attributable to the father does match the set of polymorphisms of the putative father, a statistical calculation can be performed to determine the probability of a coincidental match.

The probability of parentage exclusion (representing the probability that a random male will have a polymorphic form at a given polymorphic site that makes him incompatible as the father) is given by the equation (see WO 95/12607): p(exc)=xy(1−xy), where x and y are the population frequencies of alleles A and B of a biallelic polymorphic site.

(At a triallelic site p(exc)=xy(1−xy)+yz(1−yz)+xz(1−xz)+3xyz(1−xyz)), where x, y, and z and the respective populations frequencies of alleles A, B, and C).

The probability of non-exclusion is: p(non-exc)=1−p(exc).

The cumulative probability of non-exclusion (representing the values obtained when n loci are is used) is thus:

Cum p(non-exc)=p(non-exc1)p(non-exc2)p(non-exc3) . . . p(non-excn).

The cumulative probability of the exclusion for n loci (representing the probability that a random male will be excluded: cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his or her father.

C. Kits

As set forth herein, the invention provides methods, e.g., diagnostic and therapeutic methods, e.g., for determining the type of allelic variant of a polymorphic region present in an EDN1 gene, such as a human EDN1 gene. In preferred embodiments, the methods use probes or primers comprising nucleotide sequences which are complementary to a polymorphic region of an EDN1 gene (SEQ ID NOs:3 or 4). In a preferred embodiment, the methods use probes or primers comprising nucleotide sequences which are complementary to a polymorphic region of an EDN1 gene. Accordingly, the invention provides kits for performing these methods. In a preferred embodiment, the kit comprises probes or primers comprising nucleotide sequences which are complementary to one or more of the variant alleles at nucleotide position 157790 and/or 159908 of SEQ ID NO: 1, or the complements thereof. For example, if a subject has at least one copy of the variant allele (C) at nucleotide position 157790 of GI 2791272, in combination with at least one copy of the reference allele (G) at nucleotide position 159908 of GI 2791272, or the complements thereof, or at least one copy of the reference allele (T) at nucleotide position 157790 of GI 2791272, in combination with at least one copy of the variant allele (T) at nucleotide position 159908 of GI 2791272, or the complements thereof, that subject is more likely to have or to be at a higher than normal risk of developing a vascular disease such as CAD or MI.

In a preferred embodiment, the invention provides a kit for determining whether a subject has or is at risk of developing a disease or condition associated with a specific allelic variant of an EDN1 polymorphic region. In an even more preferred embodiment, the disease or disorder is characterized by an abnormal EDN1 activity. In an even more preferred embodiment, the invention provides a kit for determining whether a subject has or is or is not at risk of developing a vascular disease, e.g., atherosclerosis, CAD, MI, ischemia, stroke, peripheral vascular diseases, venous thromboembolism and pulmonary embolism.

A preferred kit provides reagents for determining whether a subject is likely to develop a vascular disease, e.g., CAD or MI.

Preferred kits comprise at least one probe or primer which is capable of specifically hybridizing under stringent conditions to an EDN1 sequence or polymorphic region and instructions for use. The kits preferably comprise at least one of the above described nucleic acids. Preferred kits for amplifying at least a portion of an EDN1 gene comprise at least two primers, at least one of which is capable of hybridizing to an allelic variant sequence.

The kits of the invention can also comprise one or more control nucleic acids or reference nucleic acids, such as nucleic acids comprising an EDN1 intronic sequence. For example, a kit can comprise primers for amplifying a polymorphic region of an EDN1 gene and a control DNA corresponding to such an amplified DNA and having the nucleotide sequence of a specific allelic variant. Thus, direct comparison can be performed between the DNA amplified from a subject and the DNA having the nucleotide sequence of a specific allelic variant. In one embodiment, the control nucleic acid comprises at least a portion of an EDN1 gene of an individual who does not have a vascular disease, or a disease or disorder associated with an aberrant EDN1 activity.

Yet other kits of the invention comprise at least one reagent necessary to perform the assay. For example, the kit can comprise an enzyme. Alternatively the kit can comprise a buffer or any other necessary reagent.

D. Electronic Apparatus Readable Media and Arrays

Electronic apparatus readable media comprising polymorphisms of the present invention is also provided. As used herein, “electronic apparatus readable media” and “computer readable media,” which are used interchangeably herein, refer to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the polymorphisms of the present invention.

A variety of software programs and formats can be used to store the polymorphisms information of the present invention on the electronic apparatus readable medium. For example, the polymorphic sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the markers of the present invention.

By providing the polymorphisms of the invention in readable form, in combination, one can routinely access the polymorphism information for a variety of purposes. For example, one skilled in the art can use the sequences of the polymorphisms of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a vascular disease or a pre-disposition to a vascular disease, wherein the method comprises the steps of determining the presence or absence of a polymorphism and based on the presence or absence of the polymorphism, determining whether the subject has a vascular disease or a pre-disposition to a vascular disease and/or recommending a particular clinical course of therapy or diagnostic evaluation for the vascular disease or pre-vascular disease condition.

The present invention further provides in an electronic system and/or in a network, a method for determining whether a subject has a vascular disease or a pre-disposition to vascular disease associated with a polymorphism as described herein wherein the method comprises the steps of determining the presence or absence of the polymorphism, and based on the presence or absence of the polymorphism, determining whether the subject has a vascular disease or a pre-disposition to a vascular disease, and/or recommending a particular treatment for the vascular disease or pre-vascular disease condition. The method may further comprise the step of receiving phenotypic information associated with the subject and/or acquiring from a network phenotypic information associated with the subject.

The present invention also provides in a network, a method for determining whether a subject has vascular disease or a pre-disposition to vascular disease associated with a polymorphism, said method comprising the steps of receiving information associated with the polymorphism, receiving phenotypic information associated with the subject, acquiring information from the network corresponding to the polymorphism and/or vascular disease, and based on one or more of the phenotypic information, the polymorphism, and the acquired information, determining whether the subject has a vascular disease or a pre-disposition to a vascular disease. The method may further comprise the step of recommending a particular treatment for the vascular disease or pre-vascular disease condition.

The present invention also provides a method for determining whether a subject has a vascular disease or a pre-disposition to a vascular disease, said method comprising the steps of receiving information associated with the polymorphism, receiving phenotypic information associated with the subject, acquiring information from the network corresponding to the polymorphism and/or vascular disease, and based on one or more of the phenotypic information, the polymorphism, and the acquired information, determining whether the subject has vascular disease or a pre-disposition to vascular disease. The method may further comprise the step of recommending a particular treatment for the vascular disease or pre-vascular disease condition.

E. Personalized Health Assessment

Methods and systems of assessing personal health and risk for disease, e.g., vascular disease, in a subject, using the polymorphisms and association of the instant invention are also provided. The methods provide personalized health care knowledge to individuals as well as to their health care providers, as well as to health care companies. It will be appreciated that the term “health care providers” is not limited to physicians but can be any source of health care. The methods and systems provide personalized information including a personal health assessment report that can include a personalized molecular profile, e.g., an EDN1 genetic profile, a health profile, or both. Overall, the methods and systems as described herein provide personalized information for individuals and patient management tools for healthcare providers and/or subjects using a variety of communications networks such as, for example, the Internet. U.S. patent application Ser. No. 60/266,082, filed Feb. 1, 2001, entitled “Methods and Systems for Personalized Health Assessment,” further describes personalized health assessment methods, systems, and apparatus, and is expressly incorporated herein by reference.

In one aspect, the invention provides an Internet-based method for assessing a subject's risk for vascular disease, e.g., CAD or MI. In one embodiment, the method comprises obtaining a biological sample from a subject, analyzing the biological sample to determine the presence or absence of a polymorphic region of EDN1, and providing results of the analysis to the subject via the Internet, wherein the presence of a polymorphic region of EDN1 indicates an increased or decreased risk for vascular disease. In another embodiment, the method comprises analyzing data from a biological sample from a subject relating to the presence or absence of a polymorphic region of EDN1 and providing results of the analysis to the subject via the Internet, wherein the presence of a polymorphic region of EDN1 indicates an increased or decreased risk for vascular disease.

It will be appreciated that the phrase “wherein the presence of a polymorphic region of EDN1 indicates an increased risk for vascular disease” includes an increased or higher than normal risk of developing a vascular disease indicated by a subject having at least one copy of the variant allele (C) at nucleotide position 157790 of GI 2791272, in combination with at least one copy of the reference allele (G) at nucleotide position 159908 of GI 2791272. or the complements thereof, or at least one copy of the reference allele (T) at nucleotide position 157790 of GI 2791272, in combination with at least one copy of the variant allele (T) at nucleotide position 159908 of GI 2791272, or the complements thereof.

The terms “Internet” and/or “communications network” as used herein refer to any suitable communication link, which permits electronic communications. It should be understood that these terms are not limited to “the Internet” or any other particular system or type of communication link. That is, the terms “Internet” and/or “communications network” refer to any suitable communication system, including extra-computer system and intra-computer system communications. Examples of such communication systems include internal busses, local area networks, wide area networks, point-to-point shared and dedicated communications, infra-red links, microwave links, telephone links, CATV links, satellite and radio links, and fiber-optic links. The terms “Internet” and/or “communications network” can also refer to any suitable communications system for sending messages between remote locations, directly or via a third party communication provider such as AT&T. In this instance, messages can be communicated via telephone or facsimile or computer synthesized voice telephone messages with or without voice or tone recognition, or any other suitable communications technique.

In another aspect, the methods of the invention also provide methods of assessing a subject's risk for vascular disease, e.g., CAD or MI. In one embodiment, the method comprises obtaining information from the subject regarding the polymorphic region of an EDN1 gene, through e.g., obtaining a biological sample from the individual, analyzing the sample to obtain the subject's EDN1 genetic profile, representing the EDN1 genetic profile information as digital genetic profile data, electronically processing the EDN1 digital genetic profile data to generate a risk assessment report for vascular disease, and displaying the risk assessment report on an output device, where the presence of a polymorphic region of EDN1 indicates an increased risk for vascular disease. In another embodiment, the method comprises analyzing a subject's EDN1 genetic profile, representing the EDN1 genetic profile information as digital genetic profile data, electronically processing the EDN1 digital genetic profile data to generate a risk assessment report for vascular disease, and displaying the risk assessment report on an output device, where the presence of a polymorphic region of EDN1 indicates an increased risk for vascular disease, e.g., CAD or MI. Additional health information may be provided and can be utilized to generate the risk assessment report. Such information includes, but is not limited to, information regarding one or more of age, sex, ethnic origin, diet, sibling health, parental health, clinical symptoms, personal health history, blood test data, weight, and alcohol use, drug use, nicotine use, and blood pressure.

The EDN1 digital genetic profile data may be transmitted via a communications network, e.g., the Internet, to a medical information system for processing.

In yet another aspect the invention provides a medical information system for assessing a subject's risk for vascular disease comprising a means for obtaining information from the subject regarding the polymorphic region of an EDN1 gene, through e.g., obtaining a biological sample from the individual to obtain an EDN1 genetic profile, a means for representing the EDN1 genetic profile as digital molecular data, a means for electronically processing the EDN1 digital genetic profile to generate a risk assessment report for vascular disease, and a means for displaying the risk assessment report on an output device, where the presence of a polymorphic region of EDN1 indicates an increased risk for vascular disease.

In another aspect, the invention provides a computerized method of providing medical advice to a subject comprising obtaining information from the subject regarding the polymorphic region of an EDN1 gene, through e.g., obtaining a biological sample from the subject, analyzing the subject's biological sample to determine the subject's EDN1 genetic profile, and, based on the subject's EDN1 genetic profile, determining the subject's risk for vascular disease. Medical advice may be then provided electronically to the subject, based on the subject's risk for vascular disease. The medical advice may comprise, for example, recommending one or more of the group consisting of: further diagnostic evaluation, use of medical or surgical devices, administration of medication, or lifestyle change. Additional health information may also be obtained from the subject and may also be used to provide the medical advice.

In another aspect, the invention includes a method for self-assessing risk for a vascular disease. The method comprises providing information from the subject regarding the polymorphic region of an EDN1 gene, through e.g., providing a biological sample for genetic analysis, and accessing an electronic output device displaying results of the genetic analysis, thereby self-assessing risk for a vascular disease, where the presence of a polymorphic region of EDN1 indicates an increased risk for vascular disease.

In another aspect, the invention provides a method of self-assessing risk for vascular disease comprising providing information from the subject regarding the polymorphic region of an EDN1 gene, through e.g., providing a biological sample, accessing EDN1 digital genetic profile data obtained from the biological sample, the EDN1 digital genetic profile data being displayed via an output device, where the presence of a polymorphic region of EDN1 indicates an increased risk for vascular disease.

An output device may be, for example, a CRT, printer, or website. An electronic output device may be accessed via the Internet.

The biological sample may be obtained from the individual at a laboratory company. In one embodiment, the laboratory company processes the biological sample to obtain EDN1 genetic profile data, represents at least some of the EDN1 genetic profile data as digital genetic profile data, and transmits the EDN1 digital genetic profile data via a communications network to a medical information system for processing. The biological sample may also be obtained from the subject at a draw station. A draw station processes the biological sample to obtain EDN1 genetic profile data and transfers the data to a laboratory company. The laboratory company then represents at least some of the EDN1 genetic profile data as digital genetic profile data, and transmits the EDN1 digital genetic profile data via a communications network to a medical information system for processing.

In another aspect, the invention provides a method for a health care provider to generate a personal health assessment report for an individual. The method comprises counseling the individual to provide a biological sample and authorizing a draw station to take a biological sample from the individual and transmit molecular information from the sample to a laboratory company, where the molecular information comprises the presence or absence of a polymorphic region of EDN1. The health care provider then requests the laboratory company to provide digital molecular data corresponding to the molecular information to a medical information system to electronically process the digital molecular data and digital health data obtained from the individual to generate a health assessment report, receives the health assessment report from the medical information system, and provides the health assessment report to the individual.

In still another aspect, the invention provides a method of assessing the health of an individual. The method comprises obtaining health information from the individual using an input device (e.g., a keyboard, touch screen, hand-held device, telephone, wireless input device, or interactive page on a website), representing at least some of the health information as digital health data, obtaining a biological sample from the individual, and processing the biological sample to obtain molecular information, where the molecular information comprises the presence or absence of a polymorphic region of EDN1. At least some of the molecular information and health data is then presented as digital molecular data and electronically processed to generate a health assessment report. The health assessment report is then displayed on an output device. The health assessment report can comprise a digital health profile of the individual. The molecular data can comprise protein sequence data, and the molecular profile can comprise a proteomic profile. The molecular data can also comprise information regarding one or more of the absence, presence, or level, of one or more specific proteins, polypeptides, chemicals, cells, organisms, or compounds in the individual's biological sample. The molecular data may also comprise, e.g., nucleic acid sequence data, and the molecular profile may comprise, e.g., a genetic profile.

In yet another embodiment, the method of assessing the health of an individual further comprises obtaining a second biological sample or a second health information at a time after obtaining the initial biological sample or initial health information, processing the second biological sample to obtain second molecular information, processing the second health information, representing at least some of the second molecular information as digital second molecular data and second health information as digital health information, and processing the molecular data and second molecular data and health information and second health information to generate a health assessment report. In one embodiment, the health assessment report provides information about the individual's predisposition for vascular disease, e.g., CAD or MI, and options for risk reduction.

Options for risk reduction comprise, for example, one or more of diet, exercise, one or more vitamins, one or more drugs, cessation of nicotine use, and cessation of alcohol use. wherein the health assessment report provides information about treatment options for a particular disorder. Treatment options comprise, for example, one or more of diet, one or more drugs, physical therapy, and surgery. In one embodiment, the health assessment report provides information about the efficacy of a particular treatment regimen and options for therapy adjustment.

In another embodiment, electronically processing the digital molecular data and digital health data to generate a health assessment report comprises using the digital molecular data and/or digital health data as inputs for an algorithm or a rule-based system that determines whether the individual is at risk for a specific disorder, e.g., a vascular disorder, such as CAD or MI. Electronically processing the digital molecular data and digital health data may also comprise using the digital molecular data and digital health data as inputs for an algorithm or a rule-based system based on one or more databases comprising stored digital molecular data and/or digital health data relating to one or more disorders, e.g., vascular disorders, such as CAD or MI.

In another embodiment, processing the digital molecular data and digital health data comprises using the digital molecular data and digital health data as inputs for an algorithm or a rule-based system based on one or more databases comprising: (i) stored digital molecular data and/or digital health data from a plurality of healthy individuals, and (ii) stored digital molecular data and/or digital health data from one or more pluralities of unhealthy individuals, each plurality of individuals having a specific disorder. At least one of the databases can be a public database. In one embodiment, the digital health data and digital molecular data are transmitted via, e.g., a communications network, e.g., the Internet, to a medical information system for processing.

A database of stored molecular data and health data, e.g., stored digital molecular data and/or digital health data, from a plurality of individuals, is further provided. A database of stored digital molecular data and/or digital health data from a plurality of healthy individuals, and stored digital molecular data and/or digital health data from one or more pluralities of unhealthy individuals, each plurality of individuals having a specific disorder, e.g., a vascular disorder, is also provided.

The new methods and systems of the invention provide healthcare providers with access to ever-growing relational databases that include both molecular data and health data that is linked to specific disorders, e.g., vascular disorders. In addition public medical knowledge is screened and abstracted to provide concise, accurate information that is added to the database on an ongoing basis. In addition, new relationships between particular SNPs, e.g., SNPs associated with vascular disease, or genetic mutations and specific discords are added as they are discovered.

T he present invention is further illustrated by the following examples which should not be construed as limiting in any way. The contents of all cited references (including, without limitation, literature references, issued patents, published patent applications and database records including Genbank™ records) as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Detection of Polymorphic Regions in the Human EDN1 Gene: Variant Allele Discovery, Validation, and Genotyping

This example describes the detection of polymorphic regions in the human EDN1 gene through use of denaturing high performance liquid chromatography (DHPLC), variant detector arrays, polymerase chain reaction (PCR), and direct sequencing. Cell lines derived from an ethnically diverse population were obtained and used for single nucleotide polymorphism (SNP) discovery by methods described in Cargill, et al. (1999) Nature Genetics 22:231-238.

Genomic sequence representing the coding and partial regulatory regions of genes were amplified by polymerase chain reaction and screened via two independent methods: denaturing high performance liquid chromatography (DHPLC) or variant detector arrays (Affymetrix™). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). Generally, the analysis was carried out as described in O'Donovan et al. ((1998) Genomics 52:44-49). PCR products having product sizes ranging from about 150-400 bp were generated using the primers and PCR conditions described in Example 2. Two PCR reactions were pooled together for DHPLC analysis (4 ul of each reaction for a total of 8 ul per sample). DHPLC was performed on a DHPLC system purchased from Transgenomic, Inc. The gradient was created by mixing buffers A (0.1M TEAA) and B (0.1M TEAA, 25% Acetontitrile). WAVEmaker™ software was utilized to predict a melting temperature and calculate a buffer gradient for mutation analysis of a given DNA sequence. The resulting chromatograms were analyzed to identify base pair alterations or deletions based on specific chromatographic profiles.

Detection of Polymorphic Regions in the Human EDN1 Gene by SSCP

Genomic DNA from an ethnically diverse population (as described by Cargill, et al. (1999) Nature Genetics 22:231-238) were subjected to PCR in 25 μl reactions (1×PCR Amplitaq polymerase buffer, 0.1 mM dNTPs, 0.8 μM 5′ primer, 0.8 μM 3′ primer, 0.75 units of Amplitaq polymerase, 50 ng genomic DNA) using each of the above described pairs of primers under the following cycle conditions: 94° C. for 2 min, 35×[94° C. for 40 sec, 57° C. for 30 sec, 72° C. for 1 min], 72° C. 5 min, 4° C. hold.

The amplified genomic DNA fragments were then analyzed by SSCP (Orita et al. (1989) PNAS USA 86:2766, see also Cotton (1993) Mutat Res 285:125-144; and Hayashi (1992) Genet Anal Tech Appl 9:73-79). From each 25 μl PCR reaction, 3 μl was taken and added to 7 μl of loading buffer. The mixture was heated to 94° C. for 5 min and then immediately cooled in a slurry of ice-water. 3-4 μl were then loaded on a 10% polyacrylamide gel either with 10% glycerol or without 10% glycerol, and then subjected to electrophoresis either overnight at 4 Watts at room temperature, overnight at 4 Watts at 4° C. (for amplifying a 5′ upstream regulatory element), or for 5 hours at 20 Watts at 4° C. The secondary structure of single-stranded nucleic acids varies according to sequence, thus allowing the detection of small differences in nucleic acid sequence between similar nucleic acids. At the end of the electrophoretic period, the DNA was analyzed by gently overlaying a mixture of dyes onto the gel (1×the manufacturer's recommended concentration of SYBR Green I™ and SYBR Green II™ in 0.5×TBE buffer (Molecular Probes™)) for 5 min, followed by rinsing in distilled water and detection in a Fluoroimager 575™ (Molecular Dynamics™).

Direct Sequencing of PCR Products

To determine the sequences of the polymorphisms identified as described above, the region containing the polymorphism was reamplified using the identified flanking primers. The genomic DNA from the subject was subjected to PCR in 50 μl reactions (1×PCR Amplitaq polymerase buffer, 0.1 mM dNTPs, 0.8 μM 5′ primer, 0.8 μM 3′ primer, 0.75 units of Amplitaq polymerase, 50 ng genomic DNA) using each of the pairs of primers under the following cycle conditions: 94° C. for 2 min, 35×[94° C. for 40 sec, 57° C. for 30 sec, 72° C. for 1 min], 72° C. 5 min, 4° C. hold. The newly amplified products were then purified using the Qiagen Qiaquick PCR purification kit according to the manufacturer's protocol, and subjected to sequencing using the aforementioned primers which were utilized for amplification.

Case-Control Population

A total of 352 U.S. Caucasian subjects with premature coronary artery disease were identified in 15 participating medical centers, fulfilling the criteria of either myocardial infarction, surgical or percutaneous revascularization, or a significant coronary artery lesion (e.g., at least a 70% stenosis in a major epicardial artery) diagnosed before age 45 in men or age 50 in women and having a living sibling who met the same criteria. These cases were compared with a random sample of 418 Caucasian controls drawn from the general U.S. population in Atlanta, Ga. Controls representing a general, unselected population were identified through random-digit dialing in the Atlanta, Ga. area. Subjects ranging in age from 20 years to 70 years were invited to participate in the study. The subjects answered a health questionnaire, had anthropometric measures taken, and blood drawn for measurement of serum markers and extraction of DNA.

Statistical Analysis

All analyses were done using the SAS statistical package (Version 8.0, SAS Institute Inc., Cary, N.C.). Differences between cases and controls were assessed with a chi-square statistic for categorical covariates and the Wilcoxon statistic for continuous covariates. Association between each SNP and two outcomes, CAD and MI, was measured by comparing genotype frequencies between controls and all CAD cases and the subset of cases with MI. Significance was determined using a continuity-adjusted chi-square or Fisher's exact test for each genotype compared to the homozygotes wild-type for that locus. Odds ratios were calculated and presented with 95% confidence intervals.

Genotype groups were pooled for subsequent analysis of the top loci. Pooling allows the best model for each locus (dominant, codominant, or recessive) to be tested. Models were chosen based on significant differences between genotypes within a locus. A recessive model was chosen when the homozygous variant differed significantly from both the heterozygous and homozygous wildtype, and the latter two did not differ from each other. A codominant model was chosen when homozygous variant genotypes differed from both heterozygous and homozygous wild-type, and the latter two differed significantly from each other. A dominant model was chosen when no significant difference was observed between heterozygous and homozygous variant genotypes.

Multivariate logistic regression was used to adjust for sex, presence of hypertension, diabetes and body mass index using the LOGISTC procedure in SAS. Height and weight, measured at the time of enrollment, were used to calculate body mass index for each subject. Presence of hypertension and non-insulin-dependent diabetes was measures by self-report (controls) and medical record confirmation (cases).

Results

The first SNP in the EDN1 gene, G456a4, is a change from T to C at nucleotide position 157790 in genomic sequence GI 2791272. This SNP is non-coding and therefore does not change an amino acid sequence of EDN1 (SEQ ID NO:2). The second SNP is a change from a G to a T at nucleotide position 159908 in the genomic sequence GI: 2791272. This variant results in the change of an amino acid from lysine (K) to asparagine (N) at amino acid position 198 of the amino acid sequence of EDN1 (SEQ ID NO:2) (see Table 1, below). These two SNPs were in strong linkage disequilibrium with each other (D′=0.56, p<0.0001).

TABLE 1
SNPs Examined in the EDN1 Gene

| | | | | | | 7 | | |
|---|---|---|---|---|---|---|---|---|
| | | 3 | 4 | | | Genbank | 8 | |
| 1 | 2 | Type of | Geno- | 5 | 6 | Accession/nt | Flanking | 9 |
| Gene | PolyID | variant | types | Ref | Var | position | sequence | SEQ ID NO. |
| EDN1 | G456a4 | Non- | CC | T | C | GI: 2791272 | TTAAAGAC | 3 |
| | | coding | CT | | | nt. 157790 | TATTAAT[c] | |
| | | | TT | | | | ACACTAAT | |
| | | | | | | | ATAGTTT | |
| EDN1 | G456a3 | Missense | TT | G | T | GI: 2791272 | CAAGCTG | 4 |
| | | (K/N) | GT | | | nt. 159908 | AAAGGCA | |
| | | | GG | | | | A[t]CCCTC | |
| | | | | | | | CAGAGAG | |
| | | | | | | | CGT | |

When these two SNPs were analyzed singly, no association with CAD or MI was revealed (see Table 2, below).

TABLE 2
Analysis of Each SNP Individually

| | | | CAD | MI | Odds ratio | Odds ratio | P value | P value |
|---|---|---|---|---|---|---|---|---|
| SNP | genotype | controls | cases | cases | CAD (95% Cl) | MI (95% Cl) | CAD | MI |
| | CC | 15 | 6 | 10 | 1.36 (.65, 2.82) | 1.52 (.66, 3.50) | | |
| | CT | 142 | 111 | 56 | 0.99 (.72, 1.37) | 0.90 (.61, 1.33) | | |
| G456a4 | TT | 221 | 174 | 97 | 1.00 | 1.00 | 0.70 | 0.47 |
| | TT | 13 | 21 | 10 | 2.00 (.98, 4.10) | 1.73 (.73, 4.06) | | |
| | GT | 125 | 105 | 55 | 1.04 (.76, 1.44) | 0.99 (.67, 1.46) | | |
| G456a3 | GG | 238 | 192 | 106 | 1.00 | 1.00 | 0.16 | 0.43 |

However, when the two SNPs were analyzed together as described herein, an association with vascular disease, e.g., CAD and MI was revealed (see Table 3, below).

TABLE 3
Analysis of Both END1 SNPs Together

| | | | | Odds | | | Odds |
|---|---|---|---|---|---|---|---|
| G456a4 | G456a3 | CAD | | ratio | MI | | ratio |
| allele C | allele T | cases | Controls | CAD | cases | Controls | MI |
| − | − | 117 | 179 | 1.00 | 67 | 179 | 1.00 |
| − | + | 40 | 18 | 3.40 | 19 | 18 | 2.82 |
| + | − | 46 | 35 | 2.01 | 24 | 35 | 1.83 |
| + | + | 66 | 106 | 0.95 | 35 | 106 | 0.88 |

P values for the association of the combination of these two SNPs with CAD and MI were p < .0001 (CAD) and p = .003 (MI).

Individuals who carried at least one copy of either variant allele (allele C for the G456a4 SNP; allele T for the G456a3 SNP), but not both, were at increased risk of CAD and MI. Comparing individuals who were carriers of either variant allele (−+) or (+−) to those with both variants (++) or neither variant (−−) gave an odds ratio of 2.53 for CAD (p=0.000002) and an odds ratio of 2.27 for MI (p=0.0004).

These results suggest that two different haplotypes in the EDN1 gene are associated with CAD/MI. The first haplotype is comprised of allele C for the SNP G456a4 and allele G for the SNP G456a3. The second haplotype is comprised of allele T for the SNP G456a4 and allele T for the SNP G456a3. These two haplotypes each result in increased risk of CAD/MI relative to all other haplotypes of these two SNPs. Without intending to be limited by theory, the true causative variant(s) which underlies this increased risk may be located in another position in the END1 gene and be in linkage disequilibrium with both of the risk haplotypes defined here. Alternatively, two or more causative variants may exist in the END1 gene, each being represented by a distinct haplotype (two of which are described here) with which it is in linkage disequilibrium.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for identifying a subject as a candidate for a particular clinical course of therapy to treat a vascular disease or disorder comprising the steps of:
a) determining the identity of the nucleotides present at nucleotide positions 157790 and 159908 of SEQ ID NO: 1, or the complements thereof; and
b) identifying the subject as a candidate for a particular clinical course of therapy based on the identity the nucleotides present at nucleotide positions 157790 and 159908 of SEQ ID NO: 1, or the complements thereof.

2. The method of claim 1, wherein determining the identity of the nucleotides is by obtaining a nucleic acid sample from the subject.

3. The method of claim 1, wherein the clinical course of therapy is use of a medical device.

4. The method of claim 1, wherein the clinical course of therapy is use of a surgical procedure.

5. The method of claim 3, wherein said medical device is selected from the group consisting of: a defibrillator, a stent, a device used in coronary revascularization, a pacemaker, and any combination thereof.

6. The method of claim 3, wherein said medical device is used in combination with a modulator of EDN1 gene expression or EDN1 polypeptide activity.

7. The method of claim 4, wherein said surgical procedure is selected from the group consisting of: percutaneous transluminal coronary angioplasty, laser angioplasty, implantation of a stent, coronary bypass grafting, implantation of a defibrillator, implantation of a pacemaker, and any combination thereof.

8. A method for identifying a subject who is a candidate for further diagnostic evaluation for a vascular disease or disorder comprising the steps of:
a) determining the identity of the nucleotides present at nucleotide positions 157790 and 159908 of SEQ ID NO:1, or the complements thereof; and
b) identifying the subject as a subject who is a candidate for further diagnostic evaluation for a vascular disease or disorder based on the identity of the nucleotides present at nucleotide positions 157790 and 159908 of SEQ ID NO: 1, or the complements thereof.

9. The method of claim 8, wherein determining the identity of the nucleotides is by obtaining a nucleic acid sample from the subject.

10. The method of claim 8, wherein said further diagnostic evaluation consists of use of one or more vascular imaging devices.

11. The method of claim 10, wherein said vascular imaging device is selected from the group consisting of: angiography, cardiac ultrasound, coronary angiogram, magnetic resonance imagery, nuclear imaging, CT scan, myocardial perfusion imagery, electrocardiogram, and any combination thereof.

12. The method of claim 8, wherein further diagnostic evaluation is selected from the group consisting of: genetic analysis, familial health history analysis, lifestyle analysis, exercise stress tests, and any combination thereof.

13. A method for selecting a clinical course of therapy to treat a subject who is at risk for developing a vascular disease or disorder comprising the steps of:
a) determining the identity of the nucleotides present at nucleotide positions 157790 and 159908 of SEQ ID NO: 1, or the complements thereof; and
b) selecting a clinical course of therapy for treatment of a subject who is at risk for developing a vascular disease or disorder based on the identity of the nucleotides present at nucleotide positions 157790 and 159908 of SEQ ID NO:1, or the complements thereof.

14. The method of claim 13, wherein determining the identity of the nucleotides is by obtaining a nucleic acid sample from the subject.

15. The method of claim 13, wherein the clinical course of therapy comprises use of a medical device for treating a vascular disease or disorder.

16. The method of claim 15, wherein said medical device is selected from the group consisting of: a defibrillator, a stent, a device used in coronary revascularization, a pacemaker, and any combination thereof.

17. The method of claim 15, wherein said medical device is used in combination with a modulator of modulators of EDN1 gene expression or EDN1 polypeptide activity.

18. The method of claim 13, wherein said clinical course of therapy is use of a surgical procedure.

19. The method of claim 18, wherein said surgical procedure is selected from the group consisting of: percutaneous transluminal coronary angioplasty, laser angioplasty, implantation of a stent, coronary bypass grafting, implantation of a defibrillator, implantation of a pacemaker, and any combination thereof.

20. A method for determining whether a subject will benefit from implantation of a stent comprising the steps of:
a) determining the identity of the nucleotides present at nucleotide positions 157790 and 159908 of SEQ ID NO: 1, or the complements thereof; and
b) determining whether a subject will benefit from implantation of a stent based on the identity of the nucleotides present at nucleotide positions 157790 and 159908 of SEQ ID NO: 1, or the complements thereof.

21. The method of claim 20, wherein determining the identity of the nucleotides is by obtaining a nucleic acid sample from the subject.

22. A method for determining whether a subject will benefit from use of a vascular imaging procedure comprising the steps of:
a) determining the identity of the nucleotides present at nucleotide positions 157790 and 159908 of SEQ ID NO: 1, or the complements thereof; and
b) determining whether a subject will benefit from use of a vascular imaging procedure based on the identity of the nucleotides present at nucleotide positions 157790 and 159908 of SEQ ID NO: 1, or the complements thereof.

23. The method of claim 22, wherein determining the identity of the nucleotides is by obtaining a nucleic acid sample from the subject.

24. The method of claim 22, wherein said vascular imaging procedure is selected from the group consisting of angiography, cardiac ultrasound, coronary angiogram, magnetic resonance imagery, nuclear imaging, CT scan, myocardial perfusion imagery, electrocardiogram, and any combination thereof.

25. A method for determining whether a subject will benefit from a surgical procedure comprising the steps of:
a) determining the identity of the nucleotides present at nucleotide positions 157790 and 159908 of SEQ ID NO: 1, or the complements thereof; and
b) determining whether a subject will benefit from a surgical procedure based on the identity of the nucleotides present at nucleotide positions 157790 and 159908 of SEQ ID NO: 1, or the complements thereof.

26. The method of claim 25, wherein determining the identity of the nucleotides is by obtaining a nucleic acid sample from the subject.

27. The method of claim 25, wherein said surgical procedure is selected from the group consisting of percutaneous transluminal coronary angioplasty, laser angioplasty, implantation of a stent, coronary bypass grafting, implantation of a defibrillator, implantation of a pacemaker, and any combination thereof.

28. A method for selecting an effective vascular imaging device as a diagnostic tool in a subject comprising the steps of:
a) determining the identity of the nucleotides present at nucleotide positions 157790 and 159908 of SEQ ID NO: 1, or the complements thereof, and
b) selecting an effective vascular imaging device as a diagnostic tool for said subject based on the identity of the nucleotides present at nucleotide positions 157790 and 159908 of SEQ ID NO: 1, or the complements thereof.

29. The method of claim 28, wherein determining the identity of the nucleotides is by obtaining a nucleic acid sample from the subject.

30. The method of claim 28, wherein said vascular imaging device is selected from the group consisting of: angiography, cardiac ultrasound, coronary angiogram, magnetic resonance imagery, nuclear imaging, CT scan, myocardial perfusion imagery, electrocardiogram, and any combination thereof.

31. A computer readable medium for storing instructions for performing a computer implemented method for determining whether or not a subject has a predisposition to a vascular disease or disorder, said instructions comprising the functionality of:
obtaining information from the subject indicative of the presence or absence of the polymorphic region of an EDN1 gene, and
based on the presence or absence of the polymorphic region of an EDN1 gene, determining whether or not the subject has a predisposition to a vascular disease or disorder.

32. A computer readable medium for storing instructions for performing a computer implemented method for identifying a predisposition to a vascular disease or disorder, said instructions comprising the functionality of:
obtaining information regarding the presence or absence of the polymorphic region of an EDN1 gene, and
based on the presence or absence of the polymorphic region of an EDN1 gene, identifying a predisposition to a vascular disease or disorder.

33. An electronic system comprising a processor for determining whether or not a subject has a predisposition to a vascular disease or disorder, said processor implementing the functionality of:
obtaining information from the subject indicative of the presence or absence of the polymorphic region of an EDN1 gene, and
based on the presence or absence of the polymorphic region of an EDN1 gene, determining whether or not the subject has the predisposition to a vascular disease or disorder.

34. An electronic system comprising a processor for performing a method for identifying a predisposition to a vascular disease or disorder in a subject, said processor implementing the functionality of:
obtaining information from the subject indicative of the presence or absence of the polymorphic region of an EDN1 gene, and
based on the presence or absence of the polymorphic region of an EDN1 gene, performing a method for identifying a predisposition to a vascular disease or disorder associated with the polymorphic region.

35. The electronic system of claims 33 or 34, wherein said processor further implements the functionality of receiving phenotypic information associated with the subject.

36. The electronic system of claims 33 or 34, wherein said processor further implements the functionality of acquiring from a network phenotypic information associated with the subject.

37. A network system for identifying a predisposition to a vascular disease or disorder in response to information submitted by an individual, said system comprising means for:
receiving data from the individual regarding the presence or absence of the polymorphic region of an EDN1 gene, and
based on the presence or absence of the polymorphic region, determining whether or not the subject has the predisposition to the vascular disease or disorder associated with the polymorphic region.

38. A network system for identifying whether or not a subject has a predisposition to a vascular disease or disorder, said system comprising means for:
receiving information from the subject regarding the polymorphic region of an EDN1 gene,
receiving phenotypic information associated with the subject,
acquiring additional information from the network, and
based on one or more of the phenotypic information, the polymorphic region, and the acquired information, determining whether or not the subject has a pre-disposition to a vascular disease or disorder associated with a polymorphic region of an EDN1 gene.

39. The system of claims 37 or 38, wherein the network system comprises a server and a work station operatively connected to said server via the network.

40. A method for determining whether a subject has a pre-disposition to a vascular disease or disorder associated with a polymorphic region of an EDN1 gene, said method comprising the steps of:
receiving information associated with the polymorphic region of an EDN1 gene,
receiving phenotypic information associated with the subject,
acquiring information from the network corresponding to an EDN1 gene, and
based on one or more of the phenotypic information, the polymorphic region, and the acquired information, determining whether the subject has a pre-disposition to a vascular disease or disorder associated with a polymorphic region of an EDN1 gene.

41. A method for diagnosing or aiding in the diagnosis of a vascular disease or disorder in a subject comprising the steps of determining the EDN1 genetic profile of the subject, thereby diagnosing or aiding in the diagnosis of a vascular disease or disorder.

42. The method of claim 41, wherein determining the subject's EDN1 genetic profile comprises determining the identity of the nucleotides present at nucleotide positions 157790 and 159908 of SEQ ID NO: 1, or the complements thereof.

43. The method of claim 41, further comprising utilizing a vascular imaging device to diagnose or aid in the diagnosis of a vascular disease or disorder.

44. The method of claim 43, wherein the vascular imaging device is selected from the group consisting of: angiography, cardiac ultrasound, coronary angiogram, magnetic resonance imagery, nuclear imaging, CT scan, myocardial perfusion imagery, electrocardiogram, and any combination thereof.

45. A method for selecting the appropriate drug to administer to a subject who has, or is at risk of developing, a vascular disease or disorder, comprising determining the molecular structure of at least a portion of an EDN1 gene of the subject.

46. The method of claim 45, wherein determining the molecular structure comprises determining the identities of the allelic variants of at least one polymorphic region of the EDN1 gene of the subject.

47. The method of claim 45, wherein determining the molecular structure comprises determining the identities of the allelic variants of at least one polymorphic region of the EDN1 gene of the subject.

48. A method for treating a subject having a disease or condition associated with specific allelic variants of an EDN1 gene, comprising the steps of:
(a) determining the identity of EDN1 allelic variants associated with vascular disease; and
(b) administering to the subject a compound that modulates EDN1 gene expression or protein activity.

49. The method of claim 48, wherein the specific allelic variant comprises a nucleotide sequence selected from the group consisting of SEQ ID NO:3 or SEQ ID NO:4, or the complement thereof.

50. A method of diagnosing or aiding in the diagnosis of a vascular disease in a subject comprising the steps of:
(a) obtaining a nucleic acid sample from the subject; and
(b) determining the identity of the nucleotides at nucleotide positions 157790 and 159908 of SEQ ID NO: 1, or the complement thereof,
wherein the presence at least one copy of a C at nucleotide position 157790 of GI 2791272 in combination with at least one copy of a G at nucleotide position 159908 of GI 2791272, or the complements thereof, or at least one copy of T at nucleotide position 157790 of GI 2791272 in combination with at least one copy of T at nucleotide position 159908 of GI 2791272, or the complements thereof, is indicative of increased likelihood of a vascular disease in the subject as compared with a subject having any other combination of these alleles.

51. The method of claim 50, wherein the vascular disease is selected from the group consisting of atherosclerosis, coronary artery disease, myocardial infarction, ischemia, stroke. peripheral vascular diseases, venous thromboembolism and pulmonary embolism.

52. The method of claim 51, wherein the vascular disease is myocardial infarction.

53. The method of claim 51, wherein the vascular disease is coronary artery disease.

54. A method for predicting the likelihood that a subject will have a vascular disease, comprising the steps of:
(a) obtaining a nucleic acid sample from the subject; and
(b) determining the identity of the nucleotides at nucleotide positions 157790 and 159908 of SEQ ID NO: 1, or the complement thereof,
wherein the presence of at least one copy of a C at nucleotide position 157790 of GI 2791272, in combination with at least one copy of a G at nucleotide position 159908 of GI 2791272, or the complements thereof, or at least one copy of T at nucleotide position 157790 of GI 2791272, in combination with at least one copy of T at nucleotide position 159908 of GI 2791272, or the complements thereof, is indicative of increased likelihood of a vascular disease in the subject as compared with a subject having any other combination of these alleles.

55. The method of claim 54, wherein the vascular disease is selected from the group consisting of atherosclerosis, coronary artery disease, myocardial infarction, ischemia, stroke, peripheral vascular diseases, venous thromboembolism and pulmonary embolism.

56. The method of claim 55, wherein the vascular disease is myocardial infarction.

57. The method of claim 55, wherein the vascular disease is coronary artery disease.

58. An isolated nucleic acid molecule comprising a nucleotide sequence comprising at least two allelic variants of a polymorphic region of an EDN1 gene, or the complements thereof, and allelic variants in linkage disequilibrium therewith, wherein the allelic variants differ from the reference sequence set forth in SEQ ID NO:1, and wherein the allelic variants are associated with vascular disease.

59. A kit comprising probes or primers which are capable of hybridizing to the nucleic acid molecule of claim 58.

60. The kit of claim 59, wherein the probes or primers comprise a nucleotide sequence from about 15 to about 30 nucleotides.

61. The kit of claim 60, wherein the probes or primers are labeled.

62. A method for determining the identity of one or more allelic variants of a polymorphic region of an EDN1 gene in a nucleic acid obtained from a subject, comprising contacting a sample nucleic acid from the subject with probes or primers having sequences which are complementary to an EDN1 gene sequence, wherein the sample comprises an EDN1 gene sequence, thereby determining the identity of one or more of the allelic variants.

63. The method of claim 62, wherein the probes or primers are capable of hybridizing to an allelic variant of a polymorphic region, and wherein the allelic variant differs From the reference sequence set forth in SEQ ID NO:1.

64. The method of claim 63, wherein determining the identity of the allelic variant comprises determining the identity of at least one nucleotide of the polymorphic region of an EDN1 gene.

65. The method of claim 63, wherein determining the identity of the allelic variant consists of determining the nucleotide content of the polymorphic region.

66. The method of claim 63, wherein determining the nucleotide content comprises sequencing the nucleotide sequence.

67. The method of claim 63, wherein determining the identity of the allelic variant comprises performing a restriction enzyme site analysis.

68. The method of claim 63, wherein determining the identity of the allelic variant is carried out by single-stranded conformation polymorphism.

69. The method of claim 63, wherein determining the identity of the allelic variant is carried out by allele specific hybridization.

70. The method of claim 63, wherein determining the identity of the allelic variant is carried out by primer specific extension.

71. The method of claim 63, wherein determining the identity of the allelic variant is carried out by an oligonucleotide ligation assay.

72. The method of claim 63, wherein the probe or primer comprises a nucleotide sequence from about 15 to about 30 nucleotides.

73. An Internet-based method for assessing a subject's risk for vascular disease, the method comprising:
a) analyzing biological information from a subject indicative of the presence or absence of a polymorphic region of EDN1;
b) providing results of the analysis to the subject via the Internet, wherein the presence of a polymorphic region of EDN1 indicates an increased risk for vascular disease.

74. A method of assessing a subject's risk for vascular disease, the method comprising:
a) obtaining biological information from the individual;
b) analyzing the information to obtain the subject's EDN1 genetic profile;
c) representing the EDN1 genetic profile information as digital genetic profile data;
d) electronically processing the EDN1 digital genetic profile data to generate a risk assessment report for vascular disease, wherein the presence of a polymorphic region of EDN1 indicates an increased risk for vascular disease; and
e) displaying the risk assessment report on an output device.

75. A method of assessing a subject's risk for vascular disease, the method comprising:
a) obtaining the subject's EDN1 genetic profile information as digital genetic profile data;
b) electronically processing the EDN1 digital genetic profile data to generate a risk assessment report for vascular disease, wherein the presence of a polymorphic region of EDN1 indicates an increased risk for vascular disease; and
c) displaying the risk assessment report on an output device.

76. The method of claims 74 or 75, further comprising the step of using the risk assessment report to provide medical advice.

77. The method of claims 74 or 75, wherein additional health information is provided.

78. The method of claim 77, wherein the additional health information comprises information regarding one or more of age, sex, ethnic origin, diet, sibling health, parental health, clinical symptoms, personal health history, blood test data, weight, and alcohol use, drug use, nicotine use, and blood pressure.

79. The method of claim 75, wherein the EDN1 digital genetic profile data are transmitted via a communications network to a medical information system for processing.

80. The method of claim 79, wherein the communications network is the Internet.

81. A medical information system for assessing a subject's risk for vascular disease comprising:
a) means for obtaining biological information from the individual to obtain an EDN1 genetic profile;
b) means for representing the EDN1 genetic profile as digital molecular data;
c) means for electronically processing the EDN1 digital genetic profile to generate a risk assessment report for vascular disease; and
d) means for displaying the risk assessment report on an output device, wherein the presence of a polymorphic region of EDN1 indicates an increased risk for vascular disease.

82. A medical information system for assessing a subject's risk for vascular disease comprising:
a) means for representing the subject's EDN1 genetic profile data as digital molecular data;
b) means for electronically processing the EDN1 digital genetic profile to generate a risk assessment report for vascular disease; and
c) means for displaying the risk assessment report on an output device, wherein the presence of a polymorphic region of EDN1 indicates an increased risk for vascular disease.

83. A computerized method of providing medical advice to a subject comprising:
a) analyzing biological information from a subject to determine the subject's EDN1 genetic profile;
b) based on the subject's EDN1 genetic profile, determining the subject's risk for vascular disease;
c) based on the subject's risk for vascular disease, electronically providing medical advice to the subject.

84. A computerized method of providing medical advice to a subject comprising:
a) based on the subject's EDN1 genetic profile, determining the subject's risk for vascular disease;
b) based on the subject's risk for vascular disease, electronically providing medical advice to the subject.

85. The method of any of claims 83 or 84, wherein the medical advice comprises one or more of the group consisting of further diagnostic evaluation, administration of medication, or lifestyle change.

86. The method of claims 83 or 84, wherein additional health information is obtained from the subject.

87. The method of claim 86, wherein the additional health information comprises information regarding one or more of age, sex, ethnic origin, diet, sibling health, parental health, clinical symptoms, personal health history, blood test data, weight, and alcohol use, drug use, nicotine use, and blood pressure.

88. A method for self-assessing risk for a vascular disease comprising
a) providing biological information for genetic analysis;
b) accessing an electronic output device displaying results of the genetic analysis, thereby self-assessing risk for a vascular disease, wherein the presence of a polymorphic region of EDN1 indicates an increased risk for vascular disease.

89. A method for self-assessing risk for a vascular disease comprising accessing an electronic output device displaying results of a genetic analysis of a biological sample, wherein the presence of a polymorphic region of EDN1 indicates an increased risk for vascular disease, thereby self-assessing risk for a vascular disease.

90. A method of self-assessing risk for vascular disease, the method comprising
a) providing biological information;
b) accessing EDN1 digital genetic profile data obtained from the biological information, the EDN1 digital genetic profile data being displayed via an output device, wherein the presence of a polymorphic region of EDN1 indicates an increased risk for vascular disease.

91. A method of self-assessing risk for vascular disease, the method comprising accessing EDN1 digital genetic profile data obtained from biological information, the EDN1 digital genetic profile data being displayed via an output device, wherein the presence of a polymorphic region of EDN1 indicates an increased risk for vascular disease.

92. The method of claims 89 or 91, wherein the electronic output device is accessed via the Internet.

93. The method of claims 89 or 91, wherein additional health information is provided.

94. The method of claim 93, wherein the additional health information comprises information regarding one or more of age, sex, ethnic origin, diet, sibling health, parental health, clinical symptoms, personal health history, blood test data, weight, and alcohol use, drug use, nicotine use, and blood pressure.

95. The method of any of claims 88, 89, 90, or 91, wherein the biological information is obtained from a sample from an individual at a laboratory company.

96. The method of claim 95, wherein the laboratory company processes the biological sample to obtain EDN1 genetic profile data, represents at least some of the EDN1 genetic profile data as digital genetic profile data, and transmits the EDN1 digital genetic profile data via a communications network to a medical information system for processing.

97. The method of any of claims 88, 89, 90, or 91, wherein the biological information is obtained from a sample from an individual at a draw station, wherein the draw station processes the biological sample to obtain EDN1 genetic profile data, and transfers the data to a laboratory company.

98. The method of claim 97, wherein the laboratory company represents at least some of the EDN1 genetic profile data as digital genetic profile data, and transmits the EDN1 digital genetic profile data via a communications network to a medical information system for processing.

99. A method for a health care provider to generate a personal health assessment report for an individual, the method comprising counseling the individual to provide a biological sample; authorizing a draw station to take a biological sample from the individual and transmit molecular information from the sample to a laboratory company, wherein the molecular information comprises the presence or absence of a polymorphic region of EDN1; requesting the laboratory company to provide digital molecular data corresponding to the molecular information to a medical information system to electronically process the digital molecular data and digital health data obtained from the individual to generate a health assessment report; receiving the health assessment report from the medical information system; and providing the health assessment report to the individual.

100. A method for a health care provider to generate a personal health assessment report for an individual, the method comprising requesting a laboratory company to provide digital molecular data corresponding to the molecular information derived from a biological sample from the individual to a medical information system to electronically process the digital molecular data and digital health data obtained to generate a health assessment report; receiving the health assessment report from the medical information system; and providing the health assessment report to the individual.

101. A method of assessing the health of an individual, the method comprising: obtaining health information from the individual using an input device; representing at least some of the health information as digital health data; obtaining biological information from the individual, wherein the information comprises the presence or absence of a polymorphic region of EDN1; representing at least some of the information as digital molecular data; electronically processing the digital molecular data and digital health data to generate a health assessment report; and displaying the health assessment report on an output device.

102. The method of claim 101, wherein electronically processing the digital molecular data and digital health data to generate a health assessment report comprises using the digital molecular data and digital health data as inputs for an algorithm or a rule-based system that determines whether the individual is at risk for a specific disorder.

103. The method of claim 101, wherein the individual has or is at risk of developing vascular disease, and wherein electronically processing the digital molecular data and digital health data to generate a health assessment report comprises using the digital molecular data and digital health data as inputs for an algorithm or a rule-based system that determines the individual's prognosis.

104. The method of claim 101, wherein electronically processing the digital molecular data and digital health data comprises using the digital molecular data and digital health data as inputs for an algorithm or a rule-based system based on one or more databases comprising stored digital molecular data and/or digital health data relating to one or more disorders.

105. The method of claim 101, wherein electronically processing the digital molecular data and digital health data comprises using the digital molecular data and digital health data as inputs for an algorithm or a rule-based system based on one or more databases comprising (i) stored digital molecular data and/or digital health data from a plurality of healthy individuals, and (ii) stored digital molecular data and/or digital health data from one or more pluralities of unhealthy individuals, each plurality of individuals having a specific disorder.

106. The method of either of claims 104 or 105, wherein at least one of the databases is a public database.

107. The method of claim 101, wherein the digital health data and digital molecular data are transmitted via a communications network to a medical information system for processing.

108. The method of claim 107, wherein the communications network is the Internet.

109. The method of claim 107, wherein the input device is a keyboard, touch screen, hand-held device, telephone, wireless input device, or interactive page on a website.

110. The method of claim 101, wherein the health assessment report comprises a digital molecular profile of the individual.

111. The method of claim 101, wherein the health assessment report comprises a digital health profile of the individual.

112. The method of claim 101, wherein the molecular data comprises nucleic acid sequence data, and the molecular profile comprises a genetic profile.

113. The method of claim 101, wherein the molecular data comprises protein sequence data, and the molecular profile comprises a proteomic profile.

114. The method of claim 101, wherein the molecular data comprises information regarding one or more of the absence, presence, or level, of one or more specific proteins, polypeptides, chemicals, cells, organisms, or compounds in the individual's biological sample.

115. The method of claim 101, wherein the health information comprises information relating to one or more of age, sex, ethnic origin, diet, sibling health, parental health, clinical symptoms, personal health history, blood test data, weight, and alcohol use, drug use, nicotine use, and blood pressure.

116. The method of claim 101 , wherein the health information comprises current and historical health information.

117. The method of claim 101, further comprising obtaining a second set of biological information at a time after obtaining the first set of biological information; processing the second set of biological information to obtain a second set of information; representing at least some of the second set of information as digital second molecular data; and processing the molecular data and second molecular data to generate a health assessment report.

118. The method of claim 117, further comprising obtaining second health information at a time after obtaining the health information; representing at least some of the second health information as digital second health data and processing the molecular data, health data, second molecular data, and second health data to generate a health assessment report.

119. The method of claim 101, wherein the health assessment report provides information about the individual's predisposition for vascular disease and options for risk reduction.

120. The method of claim 119, wherein the options for risk reduction comprise one or more of diet, exercise, one or more vitamins, one or more drugs, cessation of nicotine use, and cessation of alcohol use.

121. The method of claim 101, wherein the health assessment report provides information about treatment options for a particular disorder.

122. The method of claim 121, wherein the treatment options comprise one or more of diet, one or more drugs, physical therapy, and surgery.

123. The method of claim 101, wherein the health assessment report provides information about the efficacy of a particular treatment regimen and options for therapy adjustment.

124. The method of claim 101, further comprising storing the molecular data.

125. The method of claim 124, further comprising building a database of stored molecular data from a plurality of individuals.

126. The method of claim 101, further comprising storing the molecular data and health data.

127. The method of claim 126, further comprising building a database of stored molecular data and health data from a plurality of individuals.

128. The method of claim 126, further comprising building a database of stored digital molecular data and/or digital health data from a plurality of healthy individuals, and stored digital molecular data and/or digital health data from one or more pluralities of unhealthy individuals, each plurality of individuals having a specific disorder.

129. The method of claim 128, further comprising building a database of stored molecular data and health data from a plurality of individuals.

130. The method of claim 128, further comprising building a database of stored digital molecular data and/or digital health data from a plurality of healthy individuals, and stored digital molecular data and/or digital health data from one or more pluralities of unhealthy individuals, each plurality of individuals having a specific disorder.


SEQUENCE LISTING (4 sequences)

SEQ ID NO: 1 - DNA, 186510 bp, Homo sapiens
gatcatacctctcctctgctcagaggttctcatcggttcccatctgtctagaaaataaaatgtgaacacttgaccttgatcaaaatcctttgtaatctgctgattacctacttctctacccttctcatttctctcttttacctaccctcatgcttgaatttcctgcctacttaacattgatcgctcttcacctttacacccctcacttaaaaccccagccctcactccccacacaaacgtggctcccagtgccctggtcaaaatctcactttccttcaagatccagccaagtccactgctgtcatgaagccttctctgattttccttttccacacctcttgcaggaaacaattcttcccttctctggactcctctagtattttacaatttcctccctgaaagcatttaatccccctcttgttctgagattattcacgcactcacctgagctcccaccacagtgattcagcatgtcatgagcagagccagtgagtgcttaacgtgtgctcaccagaagcacttagcgcagtgccttgcactgctcagcagctccatatttgtggaataaagcgtttgtttacttctccaaagtgtttattattccgaaaaggttaaatggctgtttttttttaaaaggtgttaacaaatttaagtatttatctctgaccaattttgaggaagaataaacctgttcatgtgttctaactcatcagagaatagaaatatgtttttgtgaaacttgtttttaacccaaggatttattgacaaaagaaaaaaaaaattgaaaaagtattctgctttttaaaatgccctgaattaaacatttacgtgcttgctatatttttcttaacatgtatgagtttggtctagagaaaatttccaaagagaaagtgatgaggacatcattgaatatataggtaaaaattcaaaggcatttaatccctgtaacacacgaaatgaatcagcctgaatttacttataaatatacaagagaaatgactcacgtcttcagtgatacagagcaattcagttgtaaaaagctaggaaaagtggaaaagtgtgtaagagtttcagcaagtacgttcattacccactgactagaaaagggacaaggtaaagctattattatagtatatactcatgtatatcttcttcaagaagatgagtcttataaaaattaatatgtctgtagaattctctaaattgttaagggaaggtagtacttatatcttaaatttgcataggagaattatagtttctgttaattatcatggattgtttacatttgctgactgattgacctacctatgtatttattcattgttgtattttcatctaattccaaaagcatttgaacctggtcttagattattactcaaagttgatatcctaattccctatttagattagaacttctgataaacagcgtagttttacctttttcctttcagtttacttttttttaaaactactgtatttgtgggtagtgtgctcaaagtttttttttttacagtgtttgtcaaataatagtgaagtagaacacagaggtggagttttgccaagtgctgtgtcatgctgtgctagagacctaacattctgtgttgctacaccagcagaatttattcctctccttttgctttattataggcttgcagtgtaagtttaaatatagtaaacagttgttgttttaaacaaatgatcttgttttattgtgcttttctgtgcttttaaaacatatttggagatttttttacatacaaaaaagtacaaagtattttgttgtgtgtactcatgtattaacagcagagttgattataaatattttctcatatttgcttcacgttcttttcacaaattcaatatattttttaaaaaccagctcagcaaggttattgctgctgaggggcagcttccgttactccccactccttgccactctcctgtccctctcagaggagctctcactgcactgggggttttcctttcagttcattcaacgtacatgtatatatctatgaatagtatgtgattttatgtttgtattttaaatttgcataaacagtaccactttgtatgtagatacctagatatttgctttataattttacattgttctccagaagagactttccatttcctcacctctttaattatacctaatattgttagaatttaaatttttggccaatttgatggatttgtttgttgtctttctggtttattgattactaagattgggtctcattattctttaaaacaaacttttactccattaaactgatgatagctggagatgggattttgcttagcaattctgcacatgtccctgtttgaggggacaagtcagaagcctgcagcatgatgctgtgtctggagggtgcactgtttctagccttccactagcccagagtggtaccagctgttccttttcagtcttctgtgctggctcctcctcacctccccagcctctcacataggccccagggctgtggcctttgatctcttctcttcctgtctgtgctcactttgtaggtagtctcatccggtctcctgacttcagataccatttctgtgctgatgatgccccagttcattccacatgtgggtatcattctgacttaccccaccagtctctgctggggtgtctaattagcatctcaggtctgatgtgtcccagatccctggttcaccccttacctctaagcacacttcccccatagtcttccgccatccttccatctgcttggccaaaaatcttgaagtcatccttgatgcccatctttctcacgtgttgcctctaatctaccagtaaaaccttttggctcttcacttcaaaatgtcttcagaatccatccacttttaatcacctccaacaccccaccccagccacatcctctctactaaattgcttctggaatagctcctgattggtctctgtggttctaccctgttcctttacagactatcttccacatagcagccaaagtggccttgtaacaattgtgtcattcctttgcttaaaagccccaggggtttcctgactcatttggagcactggtcaggcacccaccaggagctctgtcttcctccttaccatctccctgaggctcagctccccaaccttcctccctcgcgtgctcagctgcagccacacgggtctcctgcctctttcttgaacacccgcagcaggcccctgtctcagggactttgcactcctttcctctgttaagaatgccctctaggaggattccattccaagatggccaaataggaacagctccagtctgcagttcccagcgtgattgacgcagaagacggatgatttctgcatttccaactgaggtacctggttcatctcattgggactggttggacagtgggtgcagcccacagagggtgagccaaagcagggcaaggcatcgcctcacccaggaagcacaaggggtcaggggatttccctttcctagccaagggacgctgtgacagacagtacctggaaaaacgggacactcctgcccaaatactgcacttttccaacagtcttagcaaatggcacaccagaagattatatcccgcatctggcttggtggatcccatgcccacagagccttgctcactgctagcacagcagtctgagatctacctgtgaggcagcagcctggcagagggagtggcatctgccattgctgaggcttgagtaggtaaacaaagaggctggggaagctcaaactgggcagagcccaccgcagctcagcaaggcagactgcctctgtagtctccacctctgggggcagggcatagctgagcaaaagacagcagaaacttctgcaggcttaaacattcctgtctgacagctctgaagagagcagtggttctcccagcatggtgtttgagctcagagaaaggacagactgcctcctcaagtgggtccctgacccccgtgtagcctaactaggagacacctccccgtaggggccgactgacacctcatacaggcgggtacccctctggggcgaagcttccagaggaaggatcaggcaccaatatttgccgttctgcaatatttgctgttcttcagcctctgctggtgatacccaggcatacagggcctggagtggacctccagcaaactccaacagatgtgcagctgagggacctgactgttaggaggaaaactaacaaacaaaaggattagcatcaacatcaacaaaaagaacatccacaccaaaaccccatttgatgttggtgggtcaccaacatcaaagaccaaaggtagataaaatcacaatgatggggagaaaccagaccagaacatctgaaaattctgaaaaccagagaccctcttctcttccaaaagatcgcagctcctcgccagcaatggaacaaagctggatggagaatgactttgatgagctgacagaagtaggcttcagaaggtcagtaataacaaacttttccaagctaaaggaggatgtttgaacccattgcaaggaagctaaaaaccttgaaaaaagcttagacaaatggctaactagaataaacagtgtagagaagaccttaaatgacctgatggagcaaaactgtggcacgagaactacgtgacgcatacacaagcttcaatagctgatttgatcaagtggaagaaagggtatcagtgattgaagatcaaattaatgaaataaagcgagaagagaaggttagagaaaaaagagtagaaagaaacgaacaaagcctccaagaaatatgggactatgtgaaaaaaccaaatctacatttgattggtgtacctgaaagtgacggggagaatggaaccaagttggaaaacactcttagggatattatccaggagaacttccccaacctagcaaggcaggccaatattcaaatttaggaaatacagagaacaccacaaagatactccttgagaagagcaaccgcaagacacataattgtcagattcaccaaggttgaaatgaagggaaaaatgttaaggacagccagagagaaaggtcgggtcacccacaaagggaaacccatcagacttaacagcagatctctcggcagaaactctacaagccagaagagagtgggggctggtattcagcattcttaaagaaaagaattttcaacccagaatttcatatctagccaaactaagcctcataagtgaaggagaaaaaaaaatcctttatagacaagcaaatgctgagagattttgtcaccaccagacctgccttacgagagttcctgaaggaagcactaaacatggaaaggaacaaccagtaccagctactgcaaaaacatgccaaattgtaaagaccatcaatgcttggaagaaactgcatcaactaacgggcaaaataaccagctaacatagtgacgggatcaaattcacacataacaatattaacctttaatgtaaatgggctaaatgcctcaattaaaagacacaggcaaattggataaagagacaagacccatcagtgtgctgtattcaggagacccatctcacatgcagtgacacacatagactcaaaataaagggatggaagaagatctaccaagcaaatggaaagcaaaaaaaaaaaaaaaaaaaaaaaaaaaaagcaggggttgcaatcctagtctctgataaaacagactaaaccagtaaagatcagaagagacaaagaaggccattacataatggcaaagggatcaattcaacaagaagagctaactatcctaaatatatatgcacccaatacaggagcacccagattcataaagcaagtccttagagacctacaaagagacttagactcccatacaataataatgggagactttaacaccccactgtcagtattagacagatcaacaagacagaaggttaacaaggatatctaggacttgaactccgctctgcaccaagcagacctaagagacatctacagaactctccaccacaaatcaagagaatatacattcttctcagcaccacatcgcccttattctaaaattgaccacatagttggaagtaaagcactcctcagcaaatgtaaaagaacagaaatcacaacaaactgtctctcagaccacagtgcaatccaattagaactcaggattaagaaactcactcaaaaccgcacaactacatggaaactgaacaacatgctcctgaatggctgatgggtaagtaatgaaatgaaggcagaaataaagatgttctttgaaaccagtgagaaccaagacacaacatatcagaatctctgggacatatttaaagcagtgtgtagagggacatttatagcactaaatgcccacaagagaaagcaggaaagatgtaaaatcaacacctagcatcaaaattaaaggaactagagaagcaagagcaaacaaattcaaaagccagcagaaggcaagaaataactaagatcagagcagaactgaaggagatagagacacaaaaaaacccttgaaaaaatcaatgaatccaggagctggttttttgaaaagatcaacaaaattgatagactgctagcaagactaataaagaagaaaagagagaagaatcaaacagacacaataaaaaatgataaaggggctatcaccactgatcccacagaatacaaactatcatcagagaatactataaacacctctatgcaaataaactagaaaatctagaagaaatggataaattcctggacacatacaccctcccaagactaaaccaggaagaagttcaatctctgaatagactaataacaggctctgaaattgaggcaataattaataggctaccaaccaaaaaaagtccaggaccagatggatttacagctgaattctaccagaggtacaaagaggagctggtaccattccttctgaaactattccaatcaatagaaaaagagggaatcctccctaactcatttcatgaggccagcatcatcctgataccaaagcctggcagagacataacaaaaaaaagagaattttagaccaatatccctgatgaacatcgatgtgaaaatcctcaataaaatactggcaaaccaaacccagcagcacatctaaaagtttatccaccatgatcaagtcggcttcatccctgggatgcaaggctggttcaacatatgcaaatcaataaacataatccatcacataaacagaaccaatgacaaaaaccacatgattatctcaatagatgcagaaaaggctttcaacaaaattcaatagcccttcatgctaaaaaatctcaataagctaggtattgatggaacgtatctcaaaataataagagctatttatgacaaacccacaaccaatatcatactgaatgagcaaaagctggaagcagtccctttgaaaaatggcacaagacaaggatgccctctctcaccactcctattcaaaatagtgttggaagttctggccaaggcaatcaggcaagagaaagaaataaagggtgttcaattaggaaaagaagaagtcaaattgtccctgtttgcagatgacatgattgtatatttagaaaaccccattgtctcagcccaaaatctccttaagctgataaacaacttcagcaaagtctcaggatgcaaaatcaatgtgcaaaaatcacaagcattcctatacaccaataacagacaaacggagagccaaatcatgagtgaactcccattcccaattgctaaaaagagaataaaatatctaagaatccaacttacaagggatgtgaaggacctcttcaaggagaactacaaaccactgctcaacaaaataaaagaggacacaaacaaatggaagaacattccatgctcatggataggaagaatcaacaacgtgaaaatggccatactgcccaaggtaatttatagaccatctccatcaagctaccaatgactttcttcacggaactggaaaaaaactactttaaagttcatatggaaccaaaaaacagcccacgttgccaagacaatcctaagcaaaaggaacaaagctggaggcatcatgctatctgacttcaaactatacgacaaggctacagtaaccaaacagcatggtactggtaccaaaacagatatatagaccaatggaacagaacagaggcctcagaaataacaccacacatctacaaccagctgatctttgacaaatctaacaaaaacaagaaaaggggaaaggattccctatttaataaacagtgctgggaaaactggctagccatatgtagaaagctgaaactggatcccttccttacaccttatacaaaaattgattcaagatgggttaaagacttaaatgttagacctaaaaccataaaaaccctagaagaaaacctaggcaataccattcagaacatagacatgggcaaggacttcatgtctaaaacaccaaaagcaatggcaacagaagacaaaatagacaaatgggatctaattaaactaaagagcttctgcacgaaactaccatcagagtgaacaggcaacctatggaatgggagaaaatttttgcaatctactcatctgacaaagggctaatatccagaatctacaaataactcaaacaagtttacaagaaaaaaaacaaccccatccaaaagtgggcaaaggatatgaacagacacttctcaaaagaagacatttatgcagccaacagacacatgaaaaaatgctcatcatcactggtcatcagagaaatgcaaatcaaaaccacagtgagataccatctcacaccagttagaatggcgatcattaaaaagttggaagcaacagatgatggagaggatgtggggaaatcggaacgcttttacactattggtgggagtgtaaactagttcaaccattgtggaagacagtgtggcaattcctcaaggatctagaactagaaataccatttgatccagccatcccattactgggtatatacccaaaggattataaatcatgctactataaagacacatgcacacgtatgtttattgcggcactattaacaatagcaaagacttggaaccaacccagatgtccatcaattatagactggattaagaaaatgtggcacatatttaccatggaatactctgcagccataaaaaaggatgagttcatgtcctttgcagacttggatgcagctggaaatcatcattctgagtaaactgtcacaaggacagaaaaccaaacaccgcatgttctcactcacagatgggaattgaacaatgagaacacttgaacacagggaggggaacatcacaccctggggcctgtcatggggtgggggcctgggggagggatagcattaggagatatacctaatgtaaatgactagtttatgggtgcagcaaaccaacatggcacatgtataccctatgtaacaaacctgcacgttgtgcatatgtaccctagaacttaaagtataaaaaaaaaaaaaaaaaagaacgccctctaccagatactgtgagacttacgccaccctgccttcagctctctagcagatgtcgtttgatcagtggggccttcctgaatataaatatatgtaatataaatatatataaaaacaaaaggcacctcaccctcttactgctagcatcacttacgccctttattctgttttctttttcctcatagcacttaaccaccaccagacacttgtgtgcgtgtttattatctatttcttccaactagaaaataaattatataagggcaaagatgatgttttgcttgctgctgtatcttcagctccttaggattgtgcctggcaaatagtaggtaataaacagttgtttattgaattactagaaaaaaatctctaaaatctgtagtcctgcagcatgtgaagaaggaatattagactctgcctgcctttctgaaagtgacatatacttaagtgatgctaataggatatgtaagaacatgcttcatatgtttgagccttgatgatctctgacaggagaaatggggtaaacctttttttaaaaaggacagtataatctgttcttggcagcagctttctataatctaggaaataattcaatattattaattttcgaattagatatagtcggccgggtgcagtggctcacacctgtaatcccagcactttgggaggccaaagcaggcggatcatctgaggtcgggagttcaagaccagcctgactaacatggagaaatcctgtctcaactaaaaatataaaaattagccaggcatgatggcgcatgcctgtaatcccagctgtttgggaggctgaggcaagagaattgcttgaacctaggaggcggagattgcggtgagccgagatggtgccattgcactccagcccgggcaacaagagctaaactccatctcaaaaaaataataataatagtaattagatataatcgtttttcattaaattccttcactaaaatcaaatggtaactgtgctacttgatacctttacttataaatagtgtttatgttatatggccaaggtcagtataggtattgttaatgtgataatggcatttgtttattaatttgttatttattggttattggagcagattactagaaggtaattcaagttttttgactattctgtgttctcatcctcagtaatgatcattccaggttgataaagatctgttctgttttttaatagctccagaggagaaaagtttattctctttttgaatgattttttttttttatgtatcacttcggcacctattcaatgtgtgcacaattactatcgcccaggagctcctggctgattactatgatgtggcatgagcccaaatttggcaaggttcacttcccaaggctggaatcagggaaagaccggtgatggccaaagtggtcccagaaggaagggaaggaatttatttatccctacactgctgcatttggaaggctgttggtgaggagggaaaggaagggcaaacattctagaccaaggagttgcttgaccgaagacacacaggttggggaagaggaatagggtataagagtgtgaatgggacagaaaaacaagagagttccacagggcccaaggctgggaagcaggctgggggacaggtaatagatgctgtgcaggggagtttcagcatgagaactgccatttcaggtttaaactctattcactgggcttggatacaatgaacacccctgtcatgtcagtttatacccatttcttcatgttctgacacttgaaaactattattttttagataggattcttgagtattttaatttttctaaatagtgacacatacagtgtttctttgattacaaaagtctgaaccgaagttgtttaaagctattgcccctttaggaccacatatatctgacttttatcctgggttatgttctcaaatgttctggagacaaaagattatagggcttttaatctgtgacccttgaattttcattgttgaggtttgttgtttgtttgtttgttttttgctgttgctgctgttttattttgtagtgggaatttagacagtaatgacagaaagaagcatgtttcattaagtggatgatcccattcagcaacaataatactaatactcccacagagccaaagccttttttctgggtggatgagtgttttataaaagaagtacctcaggacttcagcctctctcattctccaccagatggagtttaacagtttggatggaaagtctgaaatgattttcccttgtgatatcccgttttttatgtctatgtataaatgtataatgactaatttggaaatagcgctatcaatttattaaactaggaagcattatttcccctggaattagtttctgctctggcccaggatcactgtggtgtagaactttcaccacagaatagcctctggtaaaagatgaggcctgtacttgtcctactttaacgcttctgtatatgttttaaaaccttgccccaatcaactctataagaatgttataaagagagcagtgtgttatgaagtgtttcattttactaacaattttattttccaaagaaaaatgcaaatgattactatattagacatacagtaggagtagaagaagtggctgacttaaaacatgatgaaggagaataaaatgtttctggtgctacagtttcaagttttgaagtggacagcccagcagaacaaagctagggagacctgggggctggtcactgtcgagtgagctcatgtgcctggttcccccctgcatcctcttcacaccccgatgcgtctccacctccctcatcataggggatacctgcccctcttggggagcaggctcttggcagcagctcttttgagaagtgagccactatgtaaagcctctgccattcttgagagtcctgcctgacagtgtattctccattttcttagcccagtcagtgaggataagtgaaaacattgtatcgtgtaaagtgggatattttaacacacaaacttgctgtcttttccttttaatggtgaacaaccttttgtggtatcatggggtcttgatatcatctctcatcttcttaatctaacataattcgaatattagtcatgttacacaaacagactgctagaaaccacaccgggatttcatttgctgtgtgtcaacagtagaaattcaggacaatcctctggttttgccagcagtaagtttccacaacagttatcaaagtcagcctcttttattccaaacagtgattttggtggcttttcagacacctttcattatgtcttcacacctgcagttatcgaaggatgtctggtctagctatatgaaaactgaaaatccaaaataacccaggaaactcttttatttcctgggttctcccaaacacagtcgaagtatgtcgtttcatcatgctaacagattatagatgactattagataatatagtgctgaaagaaaaatgtcccacatcaggagccacgtgtcttttcaggaaccagtactatgaaacatcaagtcttgatcagtatgaaaattgtcttctctgttttggtaggtacacttcctgtttcctaacaacagggtggtagtgacatcagtagtggggtcctgccgcatgtgaagagggaatattagactcagcatgcttttctgaaagtgatgtatacttgatactaataggatatttaagaacatgccttatatgtttgagtcttgatgatctctgacatggaaaatggggtaaacctttttttaaaaaggacaaaatttgggagacctgattgtaaatctaaaaagtcacaacccctgccagagtttgcagaacagtagccagaaacctaaggattgtgtttttaggtttataggacacatttgggggcttagtcatttaactgatcttttctgtagcataaacataatcatcaccaggaaacgtctctgagctttcattcattaaacaaaatgtactaaacacttaagaggcagtcagtgttactacaaagcaccccagtatagtacttattttttaaacatcattaccacttggtaatatctccgtcagctcatagcgttctggaaaaagcccaggtcttatgctgtgtttgtgctgagttgctcccttacatgtgcattgttttgtttttcactaaaggttttctccatatttctttctaattaatgtcagtgttttccaagcattatcttcagtcttttccttttaaggatcctagacctgttctctactctgccaaccacaccatacccaggttatcattcaccatcatattgtgcctggtccctggttgataaagagtgttcattaaagggtgatgtgaaaatgatgtgatgaaaggccacagaccccttctgaattctttaccacccttagcaacagcaggctgcgcggtacctgtcttatcttttagcagatggaattcatgttctaattctcaatggaataaattaggggatgttgggaaacgtttgcgtttaatgaagcaggaggaattatacatttttataagccagccattaaaatgtggacattcggcatttaaaagaagtttctatttagtttaaaactagcaacttgagctgcaatgataagaaatgctcatgggaagcctaatccctctgctccactgcacattcccttgttttcttgccataaaacatgatatagcctcgggaattttgttctcctgttcttggggtattagtgactacattttagttttcaccttttgtgtcaaaattcaagccgccagcaatttgcttttcatcctttgatccgaagaatgttttcgtcaacaaatgctggacttcagtgcacggaatgttaatgtgtaccctcagccgccagctaggagaaccgtttaatatgacgaagtgcagtgagaagcattattcttgttggagtttaagtacttacagtaaagatgatgtttgatcacaggttttaaaacctatatcacatttactcttaaaaataatttaggtgcatttttaaaaattactagcaaagctttttttccaaatgaaacctgtagaatatattatcatcgggatttttatagaacacattattactctttttaagaaacacttccatgtgtagaatatgaaaccataataggctttaggatttatacttaaagcagtgctaatattattttgaatgacacgcacgtgacacagtattttggtttacttcctaaagttaaacattaaactctgccccattatgtttttaatgatgattttatctttaaaatatatatatataattgtattcacattctttaaaaaaattttttccctaggttgccattgtttatctggctttcagagcaatatttctaaacatatgtgaaaaacaaaaacaggcaaacttcaacaacaaaagaaggttgctactgagtgtacgtgggccaggttgctaccctcgggagccccttctctcagggagcccctcctgctgcttttgcgactccgactcttccagcaggtgtcactgtctggcagcagtgggcgagcaggttgcggttcacctgactcatcttcaaataataacttttaaaatttatctttagggaagttgtttgacgtaaatgtatcaaaactttgacaaaaatgttgctacatcatatgcctgatttttccctcatgaattgaaattcacagaaaataaaggaattaatcttgtgctgatataaaattttattgagtattagaaacatttgtttataatgagaggggaatgtggctgggaggacataattaaatgaaacttttaggaccatttatttatgaatttgctaagcaaatatagattccttaggtctgttctagagtctctgatggctgcaaaggggtaagccacagtgcttaccctcctgaagtattagtgtgtattatacacaccaagaaaggaacaattgtaatagaaaaggagaaagtcagtgtatcttattggcataatgacagggctcctaagttcatagaaacctgtttttgaatcctggctttgatgcattctaatcatatacagtacaagcgtcttaacttccttgtggacgatgggtgtggtatcaactctgtcatgggtggtaattaggtttaaacctggagggtgatgtggcatagatgttggcaaactttttaatgtcaagggctggatagtaaatattttaggctttgcaagccacctattctctgtcatgaatactaaactgtgactgtagcacaaaagcagccacagacagtatataaagaaatgagcctggctgtgttccaataaaactttatccacaagaaccagtggagagccacatgcagctggtgaccatagtttgccaatccctgatataaccattactcacctgtaatcccaacactttgggaggccgaggcgggtggatcacctgaggtcaggagttcgagaccagcctggacaacatggtgaaaccccatctctactaaaaattcaaaaattagccaggcatggtggcgggcacctgtaatcccagctactcgggaggctgaagcaggagaatcgcttgaacctgggaggcagaggttgcagtgagccaggattatgccactgcactccagcctgggtgacggagcaagactccaagacttcatctcaaaaaaacaaaaaaagacaacaacaaaaaaaccagttactcaacatagtatcatagttgttacattaagtaacatgcagtagcaaagtaagcagtatcacagagtcatatatgacaagtgttttgagtccataacaagtcctcgttcttgtcctcgcagacttgttgatcccaggcaaggcacttttcctttctgggtcttttatcagtacgacttttgattcagcagcagtagtcttgtttagggggcaggggcagaggttaagggggacagggaagtatataaaggacttttattcttatgaatccaaacagagggcagagttgtctgtgctttgttttggtgccatggatgtttaaaaggataccattgtcttctgttcatttaagctacccattttatcttttctctgttacccagaatatcagtcctgtttaacaaatatgtaatgattcaaaattcatttgcaggtttttaaactttgacagtggtctcctctagcagagatttcagatactatgtattcagagacagaaacatcattctgtgcttatctcagtgaaagaaacccaggaaaggaatcccagcccctcctacatcctctccaagctacaggcatgtggctcatccaggaggggagaagggagcagagaagccaccctgggtgtcttctcacccatactgctaatcctgacctgacctgaaatttaatataatcaaggcaaaatcagattaaaatggagccaaatagttttacacttcaaatagtttttacgtacataaaaaatatatggtgttcgttttaaattattcatattatttgcatatgctaaaaaagaaatcagcatgcttcttgaaatgtcttaacaatttaaaatatttataatataactgattcttaaacttttatataatgcagtatatttgaaattttaaaagttcaattatttacattctccttgggaactacattttatatattctgttacttgtaatcaaataattatatgacattcttaatgtctgtacctacaaacattgaattcatgcatatttataaggtagcttattgactataattcattgttgacctttctgatgattctgtagttagatttttaaaatatgattgaatgtggaatatctaggcatagactaacatgggtttgaatcttagctgtgacatttatgagctgtgggctcaacactttaaaagtaattaactgcatttttcatcttttaaatcattctctgagaaaaagtcaactccttaattagtccttgtttgaattatgatcaaatagtattggtttgtacagaatgatgctaacttaggatccaggctatagatggctgcaaccggaatagagccatttagtcatcatatgtacaatagagtaagaaatgcaaaaaaaaaaaaaaaaaaaaaatagatacaagataagagcacttttaggttctaagaatttaaaatgtgtatctaggttagagtgaattggggtgaaatttcctgtgcttccaagttaaccagtagaagtgttgaaattgatttcaagaattgatagtgtcctattaaacaatcagaagacagaaggtactagtgagaagtcttgatttgactatggggctaatagggaggtgcttgttgtgcaagcttatgtgagagaatagctgtatcagtagagtaaaagaccctagagcgggctgggtgcagtggctcacgcctgtaatcccagcactttgggaggccaaggcaggtagatcacttgaggtcaggtgttcaagacccgcctgaccaacatggtgaaaccccgtctctactaaaagtgcaaaaattagccaggcatggtggcggatgcctgtgatcccagctactcaggaggctgaggcaagagaatcagttgaagctgggaggcggaggttgcagtgtacatgtgtattccatatatacaagtgaagatatgaatccatactgataacttccagtccagcaccactgaatttattctagtcgtctccatttccttctcttcttcacccacagtgagaaacctggttcgcacctacagtatgtttacatatttgctcagttctagtatataaataaactagtttcagaaatccttacctttactcctgtaagaaacagatttaaaattacgtgcagcgtgtgccattcttgtctctagccttcacagtctccagtcaaaaccctctttgccaaggttatctaggacaggaccttactttccatgcccttagcgtggctgtgccatccatgcagctgcagtcagagccgtctgtctcagcttgcattcctcttaggttcacctcacatcctagttgattccgtttttatttacttacaatagaatttactttatggtgtatagtacgtggtttctgacaaatgcatagaattgtatgttcatcacatggacacaaggaggggaacatcacacaccagggtctgttgggggtgggggacaaggggagggagagcattaggacaaatacctaatgcatacggggcttgaaacctagaagacgggttgatgggtgcagcaaacctccctggcacatgtatacctccatggcacatgtatgtaacaaacctgcacatttgccacatgtatccctgaacttaaagtataataaattttttaaaaattgtgtgtttatcccacctttcttttctaatgttggtaatttgtatcttctctctctctttcttggctagtgttaccagagaatttttctaagaacccagcttttggtttccttgcctttctctatttttcttttttcaatgtcatcatttctgttcttattttgattattttttccctctccttgctttgagtttagtttgatcttctttttctgatttcttaaactgaaaatttagatttatccgagaaccttcttttctaatgaaagttttgaatacgataaatttcccactgaccactgcattagttacatcccacaaaattcgaatgttgtgttttaattttcatccagttgaaaatatttgacatgttttgagacttcctcattaacccaatgaatatttagagatgtgttcttttttttcttcaacttctaagttcaggggtacatgtgcagggtgtgcaggtttgtcgcacaggtaaacgtgtgccatggtggcttgctgcacaaaccatctcatcatctcagtattaagcccagcatttcttcctgatgttcttcccccacacccctatcaccccccaacagacaggccccagtgagtattgttccccccaatgtgtcctcgtgttttcgtcattcagctcccacttataagtgaaaacatgcggtatttggttttctcttcctgcattagtttgctgaggattatggcttccaaatccatctatgtccctgcaaaggacatgatctcattctttttgatgactgcagagtattctatggtgtacatgcaccacgttttccttatccagtctatcattgatgggcattcaggttgattccatgtctttgctattacgaatgaacatatgcatgcatgttatctttatagtaaaatgatttctgttcctttgggtatatacccagtaatgtgattgctgggtcagatggtatttctgcagaggtctaggtctttgaggaatcaccacactgtcttccacaatggttgagctaatttacactcccactagcagtgtcaaagcattcctttttctctgcaacctctccagcatctgttattttttaactttttaataatagccgttctgactggcctgagatggtatctcattgtggttttgatttgcatttctctaatgatcagtggtgttgagcttttcttcatgttgttggctgcatgtatgtgttcttttgagaagtgcctattcatgtcctttgcctgctttttaatcgggttactttttttcttataaatttgtttaagttccttgtagactctggacattagacctttgtcagatggatagattgtgaaaattttctcccattctgtaggttgtgtgttcactctgatgatagttccttttgttgtgcagaagctctgcagtttaattagaccccatttgtcaatttttccttttggtgttttcgtcataaaatctttgcccgtgactgtatcctgaatgatattgcctagattttcttctagggttttcatagtttttcattttacatttaagtctttaatccatcttgagttaatttttatatatattgtaaagaaggggtccagtttcagttttctgcatatggctagccagttttcccagcatcgtttattaaatagggactctttccccccatcacttctttttgtttgttgaagatcagatggttgctggtgtgcagtcttatttctgagttctctattttgttccgttgatctatgtgtctgttcttgtaccagtatcatgcatgtgttctttaattctgcaataatttgatggtttttcagatatcctccaattaattgatttctggtttaattctgttttcatgtaaaaacacactctgtatggtttctactgattttaaatagttgaggtttattttgtagctcagaatatggtatgtactggtgaatgttccacatacccttatataaaaagtatgttctgctgttgagtggaacatttctataaatgtctattaggtcttgatagtgttttgcgggtcttttatgttcttcctgattttctatttatgtgtcccattaattaccgagagtggattattgaagtcttcagctctgattatggatttatttttttcagtttccgctcctttatttcgaagctctattgcgtacacacttaggattgttatgttcatgggatgacctatatcattatgtaatgctcctgtttatccttcataatattctttgctctgaagtccacttcgtctgatattagtatagtttctgcagctgtattttagttattgatttatggtatatctttccccaaacttttattttcagcctacttatgtctttatatcaatatttaaaatgcgtttcttatatacagtatatacatgggacttgcattttattcagtcctagtcatttctgtcttttaatttatgtgttagaccaccccttttaatgttattatttgtgtaattggattaaaatgtaccatattggcaaccgttttctgtttgtttcatttttgggtttcagttttcttttgatgccttctctagtattaactgagtgttttttatgattctgttctatttcctctactgacttattatttatacttttaaaaaattgtatttatctaccttcagataatattacattgctttacatggagcctatagactttactgcagtttatacacagctccttctttccgtgctttatgctattgtggccatacctttttacatttacatctactgtgaacgcacagtacattgttttacacattcaggtatcttttagagcaattaaaaaataagaaaaaaaattgtgtccccatttattctattttcactgctctttgtttgtttgtgtagatccgggcctccatctgatgttgtgttccttctgcctgaggaacttccgtttaacatttattgtccactaggtcaagcagctggcaatgaatcccctcagtttttgtttttctaagaaagtctgtatttctctttcatctttgaaaattattttcaatgggcatagaattctggatttaacagttttcttgatattgttaccatattttttatttgcaccattttcattggattctttttaatagttgtcagcactcagctgaaagtcccatctgttattgtctacctttccctttagagccttcaaaatatgaaccatagttattttaaattctcagtcatttctaacataggtgtcatatctgactgtggttctgattattgctttgtctctctgaagtatgtttttttcttgccttcttgtatgccttgtaattttgtgttgaaagctgtacatcttgtgtaagacagtagagacccatggaaattgtttgtatcctagaaatgtgcatgcctctccttcctagaggcctttagtgtgggagttagagtttatctagttaggagtttgctaggtttgagagatttgttggcagctatcctcactgcaggaaaggcttcatgttcctgtagagataccttgtgttttggctgggggttggatcacaagaacatcacctgttcagttctagttttaggtcttccttttgcactatgcctcagaaagggtctctctgcacattcttgttctcctgtttctctccaagcactgttttgttacctgtaatgctaagctccttagcttgacattgtggggcaaggaggaggatggggtgctgtctctgttccgattgagttacagtctcgtacctgcaccattttcttaggtttctgggctgtgaccttctcagttctcctccctctcccccaagtgttgtgggagtttctgtgtaatcctgtccctccccaaggagacaggttgtatgtgtatgtttttcctgttccctttccacactgcagtgagttttcagcagtgtcctaaggacaacagtgcgtgccgcccttctcctcacaggataggtcttttgttttcctggtggagacaggggagatggatccaggtgtagttccttgccactcctctagagttactgcttctctcccccatatctggaacacagcggacacttcttaccccaccctcctgtgagcacctgggtgatggtcatggcatagatcctgtgtgagaatgtaaccctcagaggtttcacacaatcttggcagcccactcttgactctaaccagatacttgagcgggactcccctgactggggttctgttgtgtctgccctcggtgacacaagcttgtgtctccttagattttgggctgttgattatctgggacctcggcttattgatgggtttggaaaaagttaataagtttaaagttaggctgtacgtgtgtgtgtgtgtgtgcgcgcgcgcgtgcacgtgcacgcttgtgtgtgtgtttaacgtaaacaggtcccatccttgttagactttacagcaagagcagccttgaatgaaatcatcctttctctccagtaacttattctcccagtcattcagttctctttagtctttttacaaattttacttctttaaagaagatgcgtctccaaaaaaaaaaaatacctaatttgctactgatgtggcaaattcaagtcaaattgaggatgactcgaattttacaatgccaaacaatcctgatggccaactgtcttattcttccccagggccttctcctcagccagggcggaggctcgttgtgggcagggtccttgtcttttataatcctcgcagagttgccaaacctataaaaaatttttaaaataggcaaggtagagaagtagctgaggtgcaggagtctgggtatttacccagaaatcacacattctgtctccagcataggcccctagagccagtcaatactttggtattattttatttcattacttattttacagcataatagtagaaccctaaaagcaaaagccttatgaaatcacatgctaacagaacctaaatgcatgtttcatcttagagttttttaattataaaagtagcacacgtttgtgataaataatgtaaaatgtcaagttcttttcatcccctagaaataatcaccctccaatcttttctatgggtcttctaaggcttttctctaaactcatttttgtcatatacaaagaaaaagtaaaaaattaatatctaggccatttgggaaaggcagcctattgcttgttgctctgtttacatgtttgtctcttttcaaaaaaatacctgtaagtgcagaatgtatcacagctaaactgtacattgtctaatgtcatctttattatatctcagtgacattcctcccccataactaaattttattaagctttcagtgggaaagagcctctaatttgcaagattttgaaaatgtgaagagtttaaaagttccacatgagcttatgagatttttgaaactatttcctggatcatcttgaagattccttgttaaactcagtttcacatgatgttaagaggctgatactaacgacttttagataatatccccctttctatgaggaatgggttgaatcatatcacagaagaaattgacaggaaattgtagcatctctctgagtaaataagaaggcttacacattaggtgaatatgacgacacggaggaccattgaaacatgcctgtgtcaaccaaaagaagaagctaaagcacaaagtgtggttgaaagagttgacttgagccagaataaggacagctgcccagatgactcacccaaactgcttgcttggcctttgttacaggcagatttttaaaggcaaaaagaaaaaaacaaaaacaaaaaaacaggaggcaggggaggggggccggtgttaccgagtagactgttagacagttctgtcaggaattctcatggatatacagaagtaacagtgattagtgactgcctgtccacggttaagctatagaatgtgggatatagtgtccagtgccacattactaggttaattcacagctacctgtggcaatgggaagcagcttcagtaaagtgtggagtaggatgtgattgctgtctcatcttaatgcctctttggggcctagtaatttgaaagggcccacattcctcagataaaagtccttttctttcctcatctataatactattcaaatctaaagaaaatgtatgttgctcacagataccaacagccatcagttttttgtgtttgatttaatttgttcagtaaaaggaagatacaaaatagggaaaaagctcaggaaaagagttgcgttgaactctttattaattttatatgtagttcagttgtgttttaaagattttcaagtgcaatcttagaggccttcatttccttgtttcttgattaccctccacctgcctcattccccttctgagagtcatttcccattcctcgtccctgaccccggcccctcccgcagagctgccctcctctgaatcctgatggccaactgtcttattcttccccagggccttctcagccagggcggaggctcgttgtgggcagggtccttgtcttttataatccttgcagagttgccaaacgtataaaaaatttttaaaatagcacttgtgcacttggaaagcattccatcacatacctcttggttgtttttattagatgaaaaacagagattcagttatgagcgatctggatatgatcttgaagaatctgatggcccagatgaggatgacaatgaaaatgaagacgatgatgaggacagccaggctgaatcagtcctgtcagccacaccctcagtcacagctagcccgcagcaccttccatctagaagtagccttcaggaccctgtgagtactgacgaggatgtcaggatcaccgattgcttttctggggtacacacggacccaatggacgttctgcccagggcgctgctcaccagaatgactgtcctgagcacagcacagtctgactacaataggaagacactctctccggggaaggccaggcagcgtgctgcgagagatgaaaacgacacaattccgtctgtagacacttccaggtccccgtgtcatcagatgtctgtggactaccctgagtcagaagaaattctgagaagttctatggcaggaaaagctgttgctataacacaggtaaatgattggcagttgttcttttatttcttttttcgttttaacctattaaatatggagaacttaatggcttaagaaaataatgcacttaagtgatttttttaggccaaatcagtttgtatagctttatttttttcttgtatttaagggtagagggtatttttgccctttggagtcttgttgagtatcttaagcagttcagatattttattgtaaatctatgaaagaaccaaatggttcttgaaaaatacacccatttatttttatttgctttttgaaaaatttttgaaattaaaaaaaaaaaaaaacgtgacagtggcgaacacaagtcccgcaactgatttatctgctggccccttctgagcagtttctccactggtgctgatgtgatgatggattttcactgacttacccaaacattttgcactttgcccccattggcaagacagctaccagtttctatggcaaagttaatgtgacccgaaaacacatcaagaagttcactttgcttgaattatcacattgctacagcagcttgggagagctttctgcccaagaactatctcaaggcaggcacaggcctctgaaaagtcacgctccatgtgctgtgagtgagtccctgctctacctctggctgttttgtgacaaatgattaattctgggaggaatacattcttccctcattgacagttctcttacctctagtatgtattgaatattgcaataggatttatgcctaattgtggcagattgtgctgtaagcgtctctatcccaaaacctctcagacacttaccagtgcctggaaatctggttttcagtagacatttcataaacttgcaagtgatagacaaacaaccagagaactttttcatagtaacatctcttcacaaataattctttagttcatttttccccattgagttgacatagccaacttgaatctcaatttctcagtaactcttaatctctaaaataaggtggagctggtagtgccagaaacagaaataataacaaaaatagcagcttaagaatatagctgtgtccacttctcagagttattctctgtgtacaatatgacaaaatccaggaaatttgcttcagacatgcaaaagactatacaagatgtattgccattatttctaccagaaacgaaattaaagatgtaataattgtttaattggaagcttatttttaaaaatcgaaaaataatttcattgcagcaaacatcatttttatcaatgcaaatatacttacttttatctcattgtgaagaaatagatttagcactagctttatatctcagtgattacaaaagacctgtcataaaaggattgctctctcttgtcatttagagcccatcatctgtaagacttcctcctgctgcagctgagcacagcccccagacagcagcggggatgccttctgtggcctcaccacatcctgaccctcaagaacagaagcagcaaataactctacagccgactccaggcttgccttctccccacactcatttgtttagccaccttcctttgcattcccagcagcaatcgaggacaccttataatatggttccagttggggggatccatgtggtacctgctggcctcacatactccacgtttgtgccccttcaggctggaccagtgcagctcacgatccctgctgtcagtgtcgttcacagaactttgggtactcataggaatacggtcacagaagtgtctggcactacaaaccctgctggagtggctgaattaagcagtgttgtgccatgtattcctatcggccaaatccgcgtgccaggccttcagaacctaagtaccccaggcttgcagtcactcccctcgttaagcatggaaaccgtcaatattgtaggcctagccaatacaaatatggccccacaagtccatccaccaggactggctctgaatgctgtcggactgcaggttctgactgcaaacccttcatcacaaagcagccccgcccctcaggcacacattccaggtctccagatcttgaacatagcattgcccaccttaatcccctcagtcagtcaagtagccgttgatgcacagggagctccagaaatgccagcttcccaaagcaaagcatgcgagacacaacccaagcagacttctgtagccagcgcaaaccaggtcagcaggaccgagtctcctcaggggttacctacagtccagcgggaaaatgcaaaaaaagttctgaatccacctgcccctgcaggtgaccatgcaaggcttgatggcctgagtaaaatggacacagagaaggctgcctcggcaaatcacgtgaagcccaagcctgaactcacttccatacagggccaaccagcgtccacgtcacaacctctgctgaaggcacattctgaagtttttacaaagccctcaggccagcagactctctctccagacagacaggttcccaggcccacagcactaccgcggaggcagcccactgtgcacttcagcgacgtgagcagcgatgatgacgaggacaggcttgtgatagcaacctgatggattttattttttatttgctttttttttatataacacttaaaggtttctttgaaaaccctcctttccttaaagcacatttttctgacataaactcatgactaatctttgtgcaatcatgaacttttgaccaataattgttgttttgtgtcagctccagccatttttgtacatgttgtatagacaattgtgccttttaggagctttatgtttagaaactgtacagattgttgaatatctatatacataaaaatatattatatatgtatatgaaaaccaggtagttatttgtgtttagtaaggaaaacctgtcaaataaatcaaatgattaaattatatgttccactgttgaatataaattttatggctatggggcagagtttctgtgtataaattagtatgtaaactccatatttattgtattcatattagtctttgaaaatgggtctgtcctccttgtgtaagacagtaactttacacttcagacagattttctgtgttatgaaatgtttcagtaaaatattgtttactgactttaccattgcttcagttgtgccttctttatatcatccagtgatcagtgatctgcatttggaaaatagccctgggttccattctttccacattcccctcaacccttcattttttttataaacagagactttactcgcctttctaccatgtgaaactgtaactgaaatccatacaactgattgacctatttttcttatttgtgagaagtcatttcatagttattattagaagactaaatatataagaatactttctgtatatacataaagcatatagcctaaaatgggcaacgttcctaaatgagggtatggaaatttctgggaaacatgagttaaaccccattgtccttagaatttggctcagatgagacccgtataacctcatttccaggctccaacaccagatccattcgtgggctatgggctcttctttcccttgatgctgttaaactcacagagctctgtccagcactgaactacagagtgtccaagattaccgaagagccaaattcagacttctaaattacttaagtgactctctttagacattactatttcctgtcctggaaggttgaaccatccttattcaggaaaatatggcggcatggggagcccccaattaccagtgcagtgatttgcattattaatgattatagatgctaggtttatgagaaaagtattttaatgttggaatttaaatacttagcacatcattaagtcaatgtattcacatttttaaaggcttgcttaacaaagattgcagagacatgaaataagtaagacaacagcatggtttgcgaggtgagaggtaggggacagcctatctactaaggggtttggagacctgctgctagccaacccagcttcacgtctgtgactttgcaaagacctggcatttcaacttgcgtatccttatctcaattaggatctccaaggtgtgctgcagtcccagcacgcactggtggatctgtgatgctttaaatgaatgcttcaatttacatttacatatttccatagattaagtaacaaaattctctcaaaaacaataaaactacatttttatattctgagatagctttggaaagaattctggaaatatctgctctagggattgcgttgccctatcaataatagctgttgaaaatgaggtctgttttctgtccagtttctcaaaattaatgttttaactgtgtcatgaactttttattgcctaaaattttaaagcagtataataagcatattcttaactccataattaagtacagatgacttctctaaattcaaaaataatgcttggagtttttagcattgaggttttttagtttctatagaattttcttaatttaaaaaattcctatacttaaagacaagtgttacgatgtgagtggatgttcccttgaaattttcataatgctgtaatgtaccttactcttattttgtacttttcaagattcttattttgggcttttcaaaattcaatgcatcgtgcaaatacctttactattggcaactttcaagctttattgtagcctttcaagtattttgctagtatttgaaatatgcagctctataaaaaatattaccaaagtctaagccattatattttgaaattacatgcacaaaatagacaagggaacaataattttgtgctatttctccgaagtcgtcttttcccaaggagcagtgtccacttgatactcccaaatataaacgacttccaaataatgtaattcggagaactaatgggactacatttgacttttattctgttgcactgtcgtgtgtggagacccaggcctgtagagaactagggtgcaatgacatagtaagataatttattgacaaaacaggtgaaagttttaatgtgtaacaatttcaataccttgtgtgaatccactgcaatgccatcatcagaagtactggaaaaaacagctgaagaaccacaggtcgtttttctagttaaagattattatatctggcttatgaaaatgtattataacataaatcgagggcgttccaataacttattttcattccctaaactttaagccaaaatgataattatttctttcgaaatgataattaaccgatggtattgaatgtacaaattttgtttctcagttatatttttcataatatagacctgattttcctttcacagaaacagtataagtaacacgtttcttactaattaatccctggcctgaattttaaatgatagcagttaataaggattatggaaaataatcattatttttgttggaagactgtcaagatagttaatagatcttgattcatgctcagttcaaattataacatcagggttctttttgacttttctgtttttcatcttacattttgcagtcaacttcaaataataccagaaagatgattttcattaattttgtttcaaaaccatctataaattctaaatacagcatgtatatcaccttaagaatcttctctcaaagtgacttcccatgacttaaaattctaaatttcctacttaatattcaaaatatgtattttgtaatatatatttgtatatatataatatatgtatatattacatgcatgttatatatacgttatattacatgtatatatacatgttatattacatgtatatatacatgttatatatacatatacatatatatgttatatatacatatatacatatatgtgtataatatatatacatatatacatatatgcataatatatatacatatatacatgtgtatatgtgcgtgtataatatatacatgtacatgtgtagatgtgcgtatataatatatacacatgtacatgtatagatgtgcgtatataatatgtacacatgtacatgtatagatgtgcgtatataatatatacacatgtacatgtatagatgtgcgtatataatatatacacatgtacatgtatatctgcgtatattatatatacatgtacatgtatatatgtatatattatatatacatgtacatgtatatatgtatatattatatatacatgtacatgtatatatgtatatattatatatacatatacatatgtgtatatatacatatatatgtgtatatatacatatatacacatacacgtatatatgtatattatatatacatatatacgtgtatatctatattatatatacagatatacgtgtatatctatattatatatagatatacgtgtatatatacatatatacatatatacatatattatatacatatatattatatatacagatatacgtgtatatatacatatatacatatattatatacatatatacatatatacatatattatatacatatatattatataattatatatacatgtatatgtatatattatatacatatacatgtatatatgtatatatataatattttatataatatattttagtggcaataactccctgctctcattaccaagtggggaaaaaaaggagaaaaggtttaaagcaagaatagcaaaatgtttttctatacaaattatatacagactgttcacaacttactatggttcaatttaatgattttttcaactttacactgatgtgaaaatgataagcattcagtagaaaccatactctgaattttgatatctttctgtgctagcaatatagggtttgatctcttatgatgctgggcagcagcaggagccaaagctcccagccagctacgtgattgtaaccacaaacacactacagtgcactgtattcaatacattatacgagatattctagactttatttatttttaaataaataattgttagatgatgttgtccaactgtaggctaatgtaagtgagcacgtttaaagtagggtaggctaagctatgttgttcagtaggttagatatattaaatgcttttttgtcgttgttgttgttgaaacagtcttgctctgttgcccaggctgtagtgcagtggcgcaatctcagctcagtgcaacctctgcctcccaggttcaagtgattctcctgcctcaaccacccgaatagctggaattgcaggcacacaccaccacactggctaatttttgtatttttagtagagatggggtttcactgtgttggccaggccaatcttgaactcctgacctcaagtgatccgcctgcctcggcctcccaaagtgctgagattacaggcatgagccactgtgcccggtaaatgcattttttatttaggatattttcaatttacaatagattcattgggatgtaaccccatggtaaatcaaagaacatctgtaatctgaaagtcatgatgcatttacttaaatatattggattcctagtctttgcacacacaggtttatctggaaccttgcttataaaggttctctggaaggcactgtaggcataagagacattatctgctcctaggtgttcactgtcttctgaatctggcttgggtttgaagaaaacgcccagacatccaaaatccagtaataatttaagtaccaaccaactccaaaagaaattagaaggtacagtttctaccccagaggagaatgcctgcttaagacagatcaggtaaactaggacatgacaatagaccacatcatagagaccacagactttaaaaatgtatgagagttaagaaagtgattgtcttaagaagttaaagaatgatttgtgtatacaggggattgctgtattttgaaggaggtatgggatttgggactttagaatatacatgatgctaaggagtagaacaaaaatagtagtcatgaagtctaagacaaatgtgtgccagggaccagtctaagtggaagagaggattcactcagacattcgataatatttatttagcaccaatagtgtagaaagcactttgggacctgtaatcccagcacttcgggaggccaaggtgggcagatcatgaggtcaggagatagagaccatcctggctaacacagcgaaaccctgtctctactacaaatacaaaaaaagtagccgggggtggtggtgggcacctgtagtcccagctacttgggaggctgaggcaggagaatggcatgaacctgggaggcagagcttgcagtgagctgagatcatgccgctgcactccagcctgggcgacagagtgagactctatctcaaaaagaaaaaaaaaactttgggaatgcaaatttgatgcaaattaaatgatgcctgtccttcaggagcttcagttctagtagaagagtaagtaagcaagatgcaaggtatcttgaatggcaaaagaaggtaccaccgagtccagcaggagtgcgaatgaaggagaattcctggctgagttgatgagggagtgcttcaaagagaatgagacatttaaatatggccttgaagggacttggttaagcagagacagtggatgagacagaacagacagaagcactgtggaggagaatccgtatttatccctacggcgtgccatctataatcttatgtaatcattccaagcaggaaggattatctcttcgttttataggtggagaggctgcaactcacagatgtgatgaaaccttgccatgcagatgctgggatttgaagccaggatggtgtgatgcaacagcataacacataagcaaatagtagggaacgtgaaagggcattgagaccaagggaataaaccacattttgggatttagcaaggcatctgacgaagccgcaataatattatggttggatttaaggatgattagtacggtgggctcgtcaagtggaaagagcaatcagtgggtcagagcactacagggaaaggagcacgggctgtggactccagaaaccatcactgtcacttttctaatgtgttaataacttacgggagagtgaagcagtcacaggacacaaaagtaaggaggaaagggaagtaatgcagaggagggtggaagcaatcacgagtgacccagtgccaagctggccacagtattctttctttttagttggcggactgaataaaatagaggaaacatttgttgatgacccaaataggtcaggatcagtgtccagaagattcaaccaagtggagagatgggctgaatatgaataagacaatttaacaagaaagcaaaagccttggcacttggattgaaaatttaagtgtactaaaaaacagcagtttgctttgcacaaagttcagtatgtgccaccctcatccctccctctcccaagaaaaagcctaatctggggcatagtagaaatacgttatttattttttattttatgttttgtagagatgggattttgctatgttgcccaggctgttcttgaactcctgaccgcaagcaatcctactgcctcagcctcccaaagtgctgggattacaggcagagccactacgcccagccagaaatccatgatttcataatacattatgtaaaaccacactctataccctcatgatcagagcattctgggagcatcctccataatcggactccaggcttgaacaagatacagacaaaagagcacattctgaggccatcaggctctgaggccaagctcacagtggcccctgtgagaggggctggaaataggataatctggcgggttcctcacctgatagctaaggacacacctagggagagtcacagagcagaaataataaaaaagaagttgcctttctggtggaaggagagagagttcagggattgtgaaagaagagaaagtgggctattgtgagggaaatgccatgtccccgttcatttttgtgtaaaactaagctgaacctcacagcaagaagctagtgggaattcatggctcacagctaaagctcatatttgcataataatcagacataaatatggaagaacatagtgatttctcttcaaggaagactgcaaggttaaacattctagtatgattcacgccaatcctctaagatgtattgggatcagccggggaggcaatgcttagctgttcacccctccacttgattctcaagcgagttctgaatggacaccacctccaagagctcggactctgacaactagatctgctctgagaaagtagacagaggcagctgagaagagcctgggactcaaacccatgacccacccacattccatcatctgggagaaggaccaggcggacatttctctaagtcaggaatcaagttttgttcattttgtagatttggcctttcagacataattaaaagtccccatgacatttaacaaaagttttctaattactttgcttttgttctctgagaagctagggctgtctcattccatcatgcctgaggtgtacattagatattgtattaagggtttatttcagcacactgaacaactgttcttttgaggtgctagattgttactgggaactagattgttactggggactgcatgctcaggtaaggatggaaaagtggaaattaaggacctaaataagttttaagttttatttccccaccacctagaccataaatctcattctgaaaaaaaatatagctgattttggtaattattaagacacctcaaattaaacaggaaattttcctggctcaaagagatattgataaatgacttttcaaagataggtaaattttcttacttaaactaacaagatattagatttaaattttgagattataggtcatgctctgttggtatgatagtgttttattaaaaaaactcacattttatatagtgcagtatttttaattttaacatttacaacagatattgcattatatgaagtcattctcacattgtggttgagataattgctcagggttgcaaagctagttaagtagtgaggctgggagttaaaacacggtccttttttttttttttctactgttccatgctgctttttcagtaatgatttatgataattgggttgtattgtattcatttctaggtgaaagtctatttagtattcataaaacatcccatttaaattttctacaaaaacgagtatgtccagtgccatgcatatttgtataaataatagattttgtaacctttttctttgccctttctcacaactgatataataggagccataaaaatatttagtgaagaaacatttaacttaaatagcaggagataaaagagcaatccaaagtgacagttctaatcaagcataacacatgtaaacttccaagtatattaggtttcagttcttcatcattattcagaaaaatgtctttgttacatgtcttatgagaaaagattggagggaagacagtggagcagcgattcacattgttttgagtattgacattgttggaatacagcataacatattatattatgaagttcctgagtctgatcttttcagaaatatttccaaaaagaaatggagcaattttccattacattttataaagaagataattatttaaacatatttctcctaggaaggggacagcaaattgcactgctttttatgtgcagactttatgagggaaagaaagcgatgacaggaacttggaaatgaaggcgtttatacattttagctataaaaagcgtaagagagaaaaataaatatttgttctgtttagcagatgatcccatattgcagtataaggcaatagaggtccctacatataaaacgtaacttttttctttgtctgattcttacttttggtcctgaaatgcagacgtgaataattagataacttttatgatgtctatatatctgtgggcttgaaaaagatatttcacgaagaccaatagacatttgttatgttgttgaatttgcttgagacaagacatctttcattcatttgttttcagttacttgaaagacagtatgtttagctagctacctatgaatgttgccaaatgagatctaaggaaaccatgataggtgaaagacatcagggctttacattcctggagatcagtcatggaaagaaaagccagctctgagatcagtcaattagccaggcatgcttagttttctctgctaatagcaaggcttatttttgcccagtagggtcaaatgttaagaacagttttgattcagcagtagtgccctttaatacatttgaatttatttatttcggaacatctctatactttcattacacagcaagacattttctgttccttcaacatttttatttgccagaagtagtttacgacttcatgcagtctacttattattcatactcaaccatgtgcaatcataataattattgattgaacaatctggtcttcattaaaagtatgttaactattctaatgaaataatatgcatcatttctaatctacaagggttggaaacttttgaattcataatattgattatgagaaaccaaatttcaaaaaatatgtctatgaggagaaaaataatttctaaaatttattttttttaaatcagacccaaattattaaaaacttcacttaactaatgaagttaaatgcttgatgaaagcattaagctctgttggaaatagttgtaaaatatttagtatatggtacatcaaccctctttgatacttaagcaaccctagtacccccaggtcagtgctctgaaggccctttaaatggagataaagatcagaactgacacagggaccccagggaccaatcctggaaaaatgttttgtttgttttgttttgtaactaaacaaattcctgtgaaccctttgttgtcgtttttctaaatccttcaaaaaaagaagaagcatttggaagacacaacagattcaggcgacaaaacatttcaagtttctgctacaggtagaatcgtatgttctttagaatttcagttgaaatagataacatatttccatgatggacttgtattgtctcttagagacacttacaacttttgttgttgttgttgtccttgttgttgctatttaaataactctcttcagcacgtagaatacatgccaaatgtatttgtaaatgatttgtaatgtaatttaagtgatttgtaaatgaatgatttgtaaaatctgtacattgcattacttcggtccagagttaggggccatttagatctggtggctggtgtgaatgaacataagttcaatctctgttcaaggtttatttcaatgaccatagattgtgtctctaactgttgagtcatctcacaaatttatgactaaatagggtgtgggcaagagagaacagatttacaaatactatctcaaggcttgtcatctacatactgcaacaatattattagtattgaattaataattaattggtatctaatgaatattaatattcgtatctaattactgttaaatagtgctggtgttttctagtttatctgtcaaatcaaaacattagcttaatttttttaagtatttcatatggaaacaatttaccagctaattgtcaaattatttattttctgtgcttcagttttctcatctgtaagttagggcaaaagtgccaacatgagagggcctttgggaagataaaaacagataatatgcaaagtatttaacaaatcctagtgtatagtaatatccaaataaatgttagcactagttattcaagtttttcaataattttaaatgctttccaatagaacagatactaacattcattcaacaaatacttatgaagcacgtatcacatgcaagtccatggtcagatgtgtatctgtgactatgttcaaaaccacttacaccctatttttaaatactgcgctatgtttaaaatgtttcctaagtgctttgcagaaaaaccagacaaggcagaaataagtcaaataagccaaacaggtcatctctgacagcttatgtgaagtcttctttctaaaagttcgaaaatgtggagtctttctgtgcattcactaagggaggatgggctgtcagctggcggaacagtgtgtgagatctttgtagatgacgcggcaccatccagaagggagggtacgtattgcctggctcttctggggttgactttattcttggtgcaacctaacagggattggtcacagagaggagtgatctttgcatatgattgctactgggccctccacagagtccaggcctgtccggagaccagcattagacaacaaagaagcctcctgaggctgcgaggagcccgtcagccacttagtgagctgcctctgcatctccagttctgctgggatccgggattcttattatggcctggcaacctccaggttactcacaactggcacaagagggtatctgagtgacattgttaagcagtaattagaaacagttgattagtcagagtggtatttggtttgactcggggtaattttgtttttgtaagtcttgggtttttttttttttttttgaaacgaagtctcgctcttgtcccccaggctggagtgtgatggctgatctcggctcaccagaacgtccacctgcagattcaagcgattctcttgcctcagcctcccaagtagctgggattacaggcgcctgccatcatgcctggctaatttttgtatttttagtagagatggagtttcaccatgttggccaggctgatctctaactcctgacctcaggtgatccacctgcctcggcctcctaaattgttgggattacaggcgtgagccaccgtgcccggccagtcttgggtatgtttttaggagccgttggacttcagatataaaaagcaagcttgagctaactcctccagtttccccatttagtaaatgaagaaagcgatgtccaaagagggtgaatactctccatcctttaaaaaggatacaaagcattgtttttttttcttttacatttgtttaagttccttacagatgctggatattagacctttgtcagatgcatagtttgcaaatattttctctcattctgtagattgtttactctgttgatatattcttttgctgtgcagaagctcttgtttaattagatcccatttgtcaatttttgcttttgttgtcattgcttttggctattcacaatagcaaagatatggaatcaacctaatgtgtgcccatcagtgacagattgtataaagcaaatgtggtacatatacaccatggaatactatgcagccatgaaaaagaacgagatcatatcttctgcgggaacatggatggagctggaggccattaacttcagcaaactaacgcaggaacagaaaaacaaacactgcatgctctctcttataagtgggagctaaatgatgagaactcatggacacaaagaagagaacagcagacactgggatctacttgagggtggagggtgggaggagggagaggagcagaaaaaacaactattgagtactaggcttagtacctgggtgatgaaataatctgtacaacaaatccctgtgacacgagtttacctgtataacaaacctgcacaggtacccccaaacctaaaatacaagttaaaaaaataaaatgatacaaaggggccatgttcatggtagctaccagcacaggctttggagactgtgcttgggattttattcttcctcctctacctacaagttttctaaacttgagaaagtcattatatcttcctgtacctaaatttcttcatctataaaatgggcctaatgataatgtcaacctcagaaattgctgtgaataaatgagatcttatgtaggtgtttagtaaaggacctgtgtagcctgtattaagtgttcaaagttctctcattattattattgttgcattgattgttattaatctggggagaccaacgttagaaatttgaaaccagaagataataactgacaatcaaatgtcccttcttgctctgaaattttgactctaaaccacaatgtgtctttctaataagctagataagtgataggggaatttttgtgtttagtaacatttgtttttgttatttgttattcatttgtatgtctcccgtgtcagtattcaaagtacttcagagaatcagatactaatacataaaatttatacttgtaatgtaaaatatttaaaagaatttgattaatttataaagaatatttgaaagtgtttgcgttgggtttatgtttcttgtgactttaatctgaaatgtttaagataacatttcaaagtataactatgaaaagggaagctatatgtttactagggaaaatatatagtgctatctttatgacattgagatagggaaaggcttgcaggtaagaaacaaaaccccaaaatcatgaaggctcgtaaagtcaagttcatcaagaggcaaaactttggtactaccctcaaaactgtttaaaataaaaactaaattggaggaaaatatttgctttatgtgtctgacagaggattctcctccaaaacttatttcaattattttacaagttggtcgggaaaagtcaaataacacactaggaaaatgttcaaactgtgagaataagatccgggcatggtggtgcccacctataattccaacactttgagagaccgaggtgggaggattacatggggccaggagtttgagaccagcctgggcaacatggcgagaccccatctctacaaaaatatttgtttaaatgtaaaaatatgcaattcatattagaaaaaatataaatggacaacaggtacatgggaaataaaggacatctttaataaaaatgagaggaatgcaaattaaaacaagatatggtttttataaccaggtaggcaaatattaagaacctaagcacatctggtatggcagggctgtagaggaagcagtgtcttcagacacaactggcagagggaggtctgagttgatggggccatttggaaaaacaatctggcagtatttattagagctacacgtgcaggctctatgttccagcaatccccactcccttacatcagctctagaggaacacttgccaaggggacatgaacgaaaaaattcattgtgtggtgtttttctctactgaaaaattgtgaagaacctaaatgcctttcaatagagtaatgtctgaataaacgacagtattttcctttttctagaatgctaagcagaactaaaaagaaagcgttagatagatggatggatctccaagacatgttttgagataaaataaccagttgcagaaaaatttatacaatgggatccaatttattttaaaaatatagaaagatataaacaaatgaagtatagaatgatggagtcgtatatctctatgtaaaagcacaggaaaaggcatggaatgatacatgtagcacatagctctggacgtccctggagagacgctgaaggttgtgggacaggcaatcaggaggtgatggggttttatgaggcaaatgaataatacagttgttttctacccgagttgggaacagattaatatatagatagggacccaaatatcagctctgtgactgaggaaggtgataatggagcaggtggcttagtttcataatcaagcaggcttgcagtggtcctgttcctcacctcctatcattttttgctcccctgcattccctccgcaccatccaccagcaggctaagctttgggacagtccatgagaaagggagcttagttctgcataacctctgattggagccagaaaataaagagacggatagctcaatcttacagcattttcatagattggctccatcctccttaaaaatatgagccagtggtcaccaccaacaattagttaatgagttgctccttctcatccccaggagcagggaggatggaagtcgtggaacacaaggagctcaaggccaaggttgctctcctgcttttaccattatgactactattgttccatgaaggtgagtatcacttacaagtaacagtatcacctacaagtaatgtttttttaaaaagaaaatgtatccatgtaatacacataagcaaaaccaaaagttcagtagaaatctacaatgatgttaggcatttttcccaacagtagttacttataaataatgttttgtttatttattatttatacttacttatttattatttataaataatgcttccaaaaatgatcctgttaatataaaacaaaaattaagatataaaacatgagaaactaaacagctatgacatgaattttcttttaaaagactggaaagcatttatcttgtagaaatttcaaataagctttatatcaatatgcactcaagatattgaatagcatttgcaaaataaattattaaaatgtctgatcatttactgtgtgctaggtatcctgctaaacacttcacattttttttcgtttaatccttctattaatactatgaggttggcactctttttcagttaaggaaactgaggctgagaatggataaatatttgcctgaggccccggaaaatgttggcccacttctactgtgtatttatcttactttttcttcatgcatattcttttctaactacaggcccattatttgctaagctgaataagagcattttaaattaaaataaattcagttgttttctcggcatatacatttgcagcgattcacaaacttcaatcttcatctactgaggagaaagagttctttattcttataaactataattataagcattcaatattttcagaggaagaaaatgtatctgtttatactaagtatttaaaaatcacctacggatttgctctaattgtcagtttctccaaaattattattgtttcttaatctcttaacaagtaattataaatagatgtgttgttatgaaaaataagaaagatattcggtgtttcctgtatttcataaacaccacgtcattttctttatcaaaatttccattgaaagattgtaagagcattaggccaccttctgggcttaagtgtgtatgagaagaagccacaacttcttttccctctggatcacctcaggtcttactttacaatgttacaaatccacacgagattaatattcaaatccgtgaacatatctttgttttcatttgtggtaaaggatcttttattgtaggtgctgcttaatgctgaataggcttgaaaaagatgtatcttcaagtacaggtgtaaacaacatgtgatttcattaagatagacgtagctatattgcaatgatggcagtttggtcagtttcataggtcaacaaacatctgtcagggtaagacctcatgctctgaatatgcaggcagtcttataattctgactatacatatgtttattttttgtaacttaagcaaaattttttttagagccaggcactgtggctcacgcctgtaatcccagcactttgggaggccgaggcaggtggatcacgaggttaggagatcgagaccatcctggctaacatggtgaaaccccatctctactaaaaacacaaaaaattagccgggcatggtggcaggtgcctgtagtcccagctactcgggaggctgaggcaggagaatggcatgaaaccgggaggcagaggttacagtgagctgagattgcgccactgcactccagcctgggcaacagagtgagactccgtctcaaaaaataaaaaataataaaaataaataaataaataaaaatttttttagaaacagggtctcactctattgcccaggatggagcagcagagtgagaccctggagtgcagtgacacaatcatagctcactgcagcctcaacctcctggtctcaagcaatcctcccacctaagcctcccaagtagctgggactacaggcgcatgccaccatgccaggtgttttaacttggcttataaactctatcagatctgttattacagagaactgaagtaatccaaaagaaaagtattcggagactttgagtagattctcaaaacaacagacacccaaatgctgaaacattgacgccctcgctacagtcagtacaggaaaatgtgaatattatcctggatgccatcatagtgatacaagttgcaaaggtgagagcttgtatttgtcaaagtgtagaagtggttcttcttacatcataaaacatggatgctaacatctagatgacttaccttaaaatcccatttttttctcttttgatcataataatgcattttgtggtacaaataagagaggctattttctttgccaaaatagcttgagagtgatcagagccgagatatggaaactatggaatacctcatatttcttctcctgaaagctgattacccagatatccaaattcattattctgtagtttattctcagtgtattttcctaagatttggaaattagacaagttcatacatggggcccagagtttcaagcacttcctgtattaacagtagattacaaagatagcatgaaaaatcggaaaagaattgaagatgccaactaacgcacctaaaaatcaatttcatgaacttgtcatgttgaaaagtcaaatagaaaataatttatgtgtgatatcagtaccatcttttcctttttttcttaaaacaaaaatttcccttttatttaaagaaaagagaaaaataagagaaaataaagtatatgaagaatcacactcagaaattgcttcttcctgtcgtgcctcagggatgtgaagcaaaaaaataacaggcatattgatcataaagctgacaggttattggtgactctaagagatctattgtcccatccccaactagcagcaaaagttctgatgacatgtgaattgatatgaaatcaggtttcatagtgatttcaaagaaggttttcagtttagttcagggctgaaggaaatgtctgtttctttggagaggaaagatgatgtgacatagtgattgagtcttgaaccaggggccagagtacgtgggttcttccctcaatctcacctcttgggttctttgtgacttgctatctctgatcttccattctttccttcttaaagaatagtgcgtgccatgcctgcatcaaaagagtcttgcgaggaagatccagtgaggaaatgtgtataattgcctaataatatataaacagtactgtgcaagtagaaggtgtcattaataacatcagaatgcctttgcgattacaaatgtgggtgacactgtgtgcatgagtactgcaggatgtggcgtccaatcacatggtggccctccccggtgactcaagaaaggatgcacatctcagcactgaaggagacctgatttttctaaagtgaagaagtaaaaatgatgccagagaccaaactgcagtataacattattcatgggggagtctgtatcacaggacatttaaagggagaaaacttacgtataaaaaaagaaaatgtgctttcaactagaacagcaagaaaaatgattttttttaattccagtaaaaatttaagccaaaacgaaacaagagaaacttcaggagtctaccaatcctagttgtaatccttttgaaaaacaggaaaacttgagaacaagagaacaaacaaggtagaacaaagttgtttttactggtctaacatcctctgtagtattaattagatacatgaaacacccagaaggagaactaagtgagagaaagagaaggaaaggcaaatttaaaggatgtgactcctgttctgtggagcagactcgctttagcatcctcttctgtttattaaatattgtgtgcaaatgtctggggtgccagctacgttatggagttcttggagatatgttctacagtgatctttcagataaaagcccttcttttttagatgagaagtttaactgagttaactaagttttaatcaattaatttaatcttgtcctacttggactagaatttagatgatgtcctgatttctcatctttttgccacctttcatggtttttcacatttataagataagatcatttaattatgggacagtgatttcaactcatttttggattcgtctagccatctacactcaaaatcgtagtgtggcacatacctctaatattcatatataagcccaaattaactatatcgtaataattttgttatatcccttgagatacaatatacaaaagcaagagatagaatttatggttgtatttgcccatgtgaagttactgaagtttgaacaataaatagctaacaaaaataaatttaaaagaatttcagataattaaactcaacattattttaaggagctacagtggcagggattccaggccaaatcacattgtaaagatagatagatagatagatagatagatagatagatagatagatagatagacagacagacagacagacagacagacagacagacagactgatttggggggaagtgggagcaatttgtcagaacgtaaaagagaactatttgaattctgtcaaatcattttacctggggaacacttagatgttggaggattaccttttcctttggcttcaagagaaaataggcagtgttttcagcaaacatcatgatttttgatctctcaaagcctttgtattgagaatacagatgatatatttgtctctattttgtactcggatctggaatttcctagaatgttacctacaaaagatttagatataaatatcttgaagaactttcagcacaaatgtcaaaattttttctttgtcacctgtttaccaattacagtatcatcacactaaacacagactcaaacttctcttcattccactattccagaatggaacacaaaaatggtatattttgatctgtatttattgccattgattaatgtggcattcgcaaacaatcccatttgggcagaccatgtttgcattttaatttcattgcaatcaacgtaatgaagctattgatcgtttcttccattttccctttcatttgcctcttaaatttgaaccgcagccctttgtgcctcaatctgttgtcacagatggaatttcttagaatgcatttcctgtttctctgaagcagttagaatcgaaaaaaatcacttcatatcatacatttaaattccaccaataaaatcaaaacttttatgagtcattatgcaattgtcccatagagtaaaaggttcagctgaaaagtgatatgtattattatttcattgcaactttataaaactttcctaggagaagtcattatttatttaaaaataaaaagcagtggaagtctctaaaatattttttacacatttcaggctaaggtttaatattcatacatgtgtacacacacacaatgaatatttagaggtaaaatttctgaaagaaacatttttatattaaaaaggaagggacttctgattatgaacatgactaaataacaggttttgaatgtagctactgaaaagtaatgaactctcaaactggacaaaaatgatgaagtaactgttttcagacattggacaacttaccacaaagggctgttgtccattcaagatggaatcacaggcaggcagacacaccttgcctttagctttctgcctggggcactttccacactgtggtgtaaggcgttcacccagattgacagcagtggtcttgctgggtggagggaacagtagagttcagggccactgacgcagctataatttttgaagaggcatatccagagaggaaaaagctgcacggaagcgtagctctagaaatctgcatataggttcttttgagtcattggcatgcacatgtgaaactccatgatttctggcagagaagggctataagaggctctgagttgaactgggacactgcaggttccacagtgctgaggggcactggcgttctgacctggcagagtggagtcatttcacgtaatacctcaggcattctgttttgaaatcacagaaaagccacaccttaaaagtaggactgacaaccaaattaaaaagtgagccaaagacttgaattagagagttcattttaaaaaggtatacatatatgaatggttaataagcacatgaaaatttgctcaataccagtagttattagagaaatgtgaattgaaacaccagtggcctatgtaccactatacacccattggagtggctgtaataagaaacaagaataccaagtgttggcaaggatgtggagaaatgggaaccatcatgcattctgatggaaatgaacatggtatatccactttgaaaaacattttgctggtttattgaaatgttatacaaaaatttatcattcaacctagcaaattcactcctagaaatctaatcaagagaattaaaaacatatctacacagagatatgtacatgaatgttcaccacagcattatttataatagccaaaagcaagtaaaaatctaaatgttcatcaactagtgatcagataaacagaatgtgtgtatccatataatgaaattgtaaactcagttatttataatagccaaaagcaagtaaaaatctaaattttcatcaactagtgatcagataaacaaaatgtgtgtatccatacaatgaaattgtatattcagcaagtaagtagaatgattactggcatatgtattaatattacatatatacatattaataatacatatatacatataattaataataaaatgtattaaactagaaaaaatgctaagtgaaagaagccagacacaaaagactattaaatatatatgcaatgtctagaaaagacaaacttatgaagtcagaaggcagatcagtggttgtcttgggctgaagataagaattgaaattgactggaaacagtgtgaaggaactttctgggatgatggaaatgttcttacacaggattgtggtgatggtgacacaattctataatttcactaaaaccatcaaattgttaaaaaaaaaaaaaaggcgtatactaaccatacggtaaatgctacctaataaagcctaaagtcagttgattcaccagtaaattaactgcctgccagaataaaactcaacactaaagaaaggcaacaaaatccaaactctcagcaacatagcagctgcaatatcccatatgcgatgaaagttactagacatgcaaagaagcaagaaaatgtgaccaaatacccaggagaaaataagttgaaataaacagacctgagatgacccagatgatgaaattatgaaatagaaattttccaagagtattataaatttgtttcaggattaaaagaagagcatgagcattttttttttttagcgaaaagatgaatttcagcagagaaatcaaaactatataaattaaatacatggaaaatctggaactaaaaaagtcagaaatgaaaaattcactggatgggctgaatagcagattgaaacatgcagaaggaagtatcagaaaacttaaatatagagcaagagaaaatctaaatttaaatacagagaagagagatttcttaaaaatgaagaatttcagtggttggttgaaatattatcaagtggtaatcggggtcatagaaggagagcagagttcagagcagaggacacatttgcaaaaataatggctgaacattttccaaaggtaaaaattactgctatagtttggatgtttgtcccactcaaacctcatgttgaaatttgattccaatactacaggtgggggtctaatgggaggtgtttgggtcatgggggcaaacctctcatgaatggattaatgccctccctgtggggtggtgagtgagttctccctctgttagttgcacaggaactgattgttaaagagagcctggcacctcacccatcccttgcgtcctctcttgccatgttatctctgcacataccagctcccctttgccttccgtccaccattactggaagcagcttgtggcccttgctggatatagatccccaatcctgaacttttccagctatcagaatcatgagccaaataaatcttttttttttttttttttaaacggagtctcactgtgttgcccaggctggagtgcagtggcatgatcctggctcactgcaacctctgactccaggattcaagtgtttctcatgcttcagcctcctgagtagctgggattacaggtgtgcaccaccaggccttgctaatttttttgtagagatgaggtttcaccatattggccaggctggtcttgaacccctgacatcaagtgatctgcctgccttggcttcccaaagtgttgggattacaggtgtgagccaccatgcctggccataaatctttttcctttataaataaccctgtcttaggtattcctttacagcagcacaaacagactaagaaaatgactactaacatctcattaaaagcaatacaagacagcctatcagggactagcatctttaaagagttgagttatcagcctaaaatcttacatctagaaaattatcctttaaaaataaagtctgagatattttcagttaataaaagttaagatcatctgtcacttatggataacaatataaatgtcataaataagatattagttaatcaaaccaaagtgcatctttaaaaaatgacaagccacaaccaaatggggttcataccagtgatgcaatgatatctccatattagacaatctgttgttttaccatattaatgttacaaaggtgaaaattcatcatatcatcttcatggaaactatagagattttagatatgttccttgagctttcaaaaatattaacaaatcatgaatagatcgatactttcttaacaaacacacacaagtttctaggaccaccaccatgtataagacagccaggggaaaaaaagagaaatgaaaagtaggaaaggaagaagcaactatattgatatttgtagattctgtgattgtacacatgaaaaacataaaaacaagtgaatttgataagatacgaagctaatatcaaaattcagttaattacttatatcaaaatcagaacttaaaatgacaccacttataatagctataagattacaagaaataaatataagaaatatataagatctatataaggttttcaaatgctaatgagtaacataaaaatattatccaaatagaatggtatgccttattcctgagctgatagattcaatattgtaaagttcaattttcttaaaattaaatcacaagtttaatgtgatctcaataaaaataccagagaaaaaaattgcttttggaattaagtgaactaattctaagttcagtggaaaaataaataagcaagaaaaaacaaataaattctgaagaaagattaataaactgttcctgtccgtactccaaatattataggaaataaaacagtttattattgacaaatatatatccctataaatgggcacaactacagagtataaaaatagataaatatatgttgaatgtatactttgtgtataataatatttgaatttcacatcactagagaaatgatgattcatttagtagataacggtggagtgttttaacagccaatcaagagaaaaattaatttgaaccccaccttactcctttcaccaaaataaatttcagattgattaaatatttaatattttttaaatgtttaataaatcatgaaaataccagtaaaaagactggataattatttttaataatatgagagttaaaaaaacactttctaaacactacacaaaattcagcaactatagaaaaagactaataaatttgatgacatagaaattttttaaaaattgcatggataaaaaatacaacatgcaaaatgagttataaatggataaaatacctacatattgttagaaaatattagaaaaggccaataattcaaaggaaaaatgaacaaaaacatgaaatagttcataggaagagacatgtgaatgaatttggcatctttgaaaaatgttctacctcattcataagtgaaatgtcaactggaatttctgtacagtatccattttccacgaacgattaacaacaacaaaaatgtttgtgcactatttgaaggactatattgagtaataattaatttcatgtactattaatgacagtaaaatcattagtctgaccaatttggagaacaatttggcactttctatcaaacatgaaaaatgcatatttctgggaatttattccatttctaggaatttattcttattgacatattcacatgtgggaaaaatgtgaacaatgatatttatgtcattatgtgtaacagcaaaagttggaagcaagctaaatggccaaataaggaagagattcaataaactgtaagatatgccttcaatggaatactatggagtcactgaaagaaaaaatagtaaggctacttacattgtattgttgtgttgtgttgtgttgtattgatatgttgctgaaatatactaagtaaaaaggaaagcaaattgtagaacaatgtgtatattatgctaccttttcagaaaaactataaatatgtatgcacacatctctactcttatcctttatgcagatttatgctgccaacatttgtaccttctggatacaggacttgggagtagtaggaacgggaagaaatgggacaggctgaatcttcactctatttataaaacctttagttccttttgaattatttaccgttcatatgtatcatttttaaaaatagaaaataaaaatgttcaaagttgaataagcgggtagaaacatttcagtggctttccgttgaatccagaacaaaagtttctttccagaggttcaggccctccttgaactggctcattcccgtttcttaaatcccaccttgttcctcccacctcctctcactccgcagtttccaactctattgaccttctctcagttccttgcacaagtcagttttgccgctttggagcctgtgcactgcttgctttctgcctgcaactcttttgtcccaactctgcattatcgggctccctctctttcttattcttttggctttggtggtcacccattcagagagaccctccttaggcatccactctaacacagagcccgtttttcttagtcttttcttgtggcacctaattccttcctttcatactcaatttctttatctgttggctcttgtagtgtttttccacacccctccccaggatggtaaactccttgagttaccatttctattttattctcttgtcgacacctgtaagagtacccgacacattgcaactgggttggaatgctaacatctcttgaataaattaattcacccatttaagaccaaaagtttgtattacaagttagcaggaataaatgtcttactgattggtatatgggtgtaaaaatatggccctgattttcagtatttaaaactctcaagatgccaaatattttctactgctttctaagctgaacttccaggcacagctggcatatgcatatttcttgacaatctccaagttttgataaccaaataagaaaaaaaaacagcacagaaaaatataattttggctgttactaaatgggctaataataccagtaatagtattttaaaaattctaaaaaatgtctaattctcacaatagcattctaacagtaaactaagaggtaagctaatatattcaagatacagagcaaccacacttgtttaggagcactgttactttgtctacattgatttgttaaaacattgtccgttgtcaaccaatctaaaaaggacacaataggctaggcacagtggctcacacctctaatcccagcactttgggaggcctaggtgggaggatggcttgagcccaggagttcaagatcagcctgggcaacacggggagaccccaactctacaaaaactttttaaagaaattagccaggtgtcgtggtgtgtgcctgtagtcccaactactcaggaggctgaggtgggaggattgcttgagcccagaagttcaaggctgcagcaagcctgattgtgccactgcactccagcctaggcagcagagtgagactctgtctaaaaaaaataaaaataaaaataaaaatgaatacaatttttaaaataaataaaaagaacataataaaatatgtccagaaatcaatagtcaatatcgtttgattaattttgccaaaatccaatacacggtgtagttacagagctgaactttaataatagaaccaatgtaacatggtaatgcttttcatattttaaactactttcctaaatgttatctcatttgatgctcactacgaaaacacaatagagatcattttggcctgtgcttttgtaaaattatagaatattagacctgaaaaatatataatgtcatttacttctattcttcattttatagatgagggaactgaggccagaaaacatgataaagtatgttccattctcacagctaattaggggctagaaccaggatctcagtcctctcttgggccactagacaattgctaactatgcttctgcaacaccttcatatcatatgactgacttcttaagtgtacaaattaattttaggatttaaatcaattacaatatttctttttaaaacatggaatatggttcatccaaaagataatctacatcccactacccatatagaaccattatttttatttttggatattttcttcagtatttttctttgatttgtatgtttcatagaaatgtgtgtgtgtgtgtgtaattttttcacctctcttaagtacatatttttaaatttttaaatggatacacaatatttgtacatatttatgcagtacatgtgatatttcattacatgcatagaatgtgtaatgatcaagttagggtatttccatcacctcaagcatttataatttttttgtattgggaactgatatggtttggctctgtgtcctcacccaaatctcatgttgaattgtaattcccagtgttgcgggagggacctggtgggaggtgattggatcgtgggggcagatttcccccttgctgttgtcgtgatagggagtgagttctcatgagatctgatggtttaaaagtgtgtggcacttccctctttgctctctcttctgccgccatgtgaagacgcgcttgcttcccctttgcccttcctctatgattgtaagtttcctgaggcctcccagccatgcttccagtacaatccgtggaaccatgagtaaattaaatctcttttcttcataaattactcagtctcaggtagttctttatagcaatgtgagattggagtaacacaggaacattttaagttctattttgaaacatactttttttttttttacaatacagctaaatgttttgtaaaacatttaaaaaatgttagtcccacacagtcatgtaacttcaaacagtaagctcatcacctaatattattgcatctgccacttagaagctgtgtgactagaagctccctgggcctcagttttctcatctgcaaaatagggataataatagtggtaattttatagagctgttataaggcttactcagcctcccaaagtgttgggattataggcgtgagccacagcagctggcctatgtaatttaattatatgctttttgcttaacatattattcttatatttttattattcctgaaagtttttcattattataatttcaatacttagtcaatatgctgcctaatgaacatagtgtaatttacttatcctatttcccattttggacagtgaggttgttttcactttttgctctcataaataattctgaagaaagcattccatatgaatattattgtcaatatcactgataatttctctaggataaattccatgaaggatgattacagaatcaatgcgataaatatttttaacactactgactcatattgtcaaaatgttttcttaaaatgttgtgacaatatcattaaccgtgtgcagaaaaacagttttcaaggtcttatcctcattagatgtagtcatttaaaagtaaattgctaaattaataggctaaaatggctcccaattgttctttcattttgcatttatttgcttattaatgagatcaaacatcttgcaatttgtttgtttgtggaatatcgtttaaagctcaattaatccgctatgttttttaatgacaagctaagacactacagatgaaaagcaatggaaagcagagatgtttagtgaccccctactacggtgctaagtatttcaaatatattgttaggtgcattaatatgcaagttatgagaaagagacgaagcaaaagtcacagacttgagtgcataatacgacaaatactacttcaaactggctcagttttagaacttaagggattttaattctttttcatagtttttactaaccaaaagctttaatgagaaacaattaattcaatattaaaaggaaataataggtagcatgaatagcttttgcatgatgggtctagagaagtgttaagtaggtacatgtggcaattgagtccttgaaatggggctggtctgaattgagatgttcaaaaatacatatgtgattttgaggactcagaacaaaaagaagagaatgcaaagtatatcattaatgatgtttattgattatatgttgaaatgtaaatattttaaaatagtggattgaattaaacctattaaaattaatgttacctatttcatttacttgtataatgtgacttctagaaaatttataattggatatgtggctaccattggccagcacaggtctagggaattatatgatatcagattttaggaaggaagttataaaattttagttcttcaggaagagatctaaaggagagtacagagagagagagagagagagagaatctaccacataactaaattaagtaaagggagtgctacagcactgaccattatgaccaaccaggaggtctctgataaactcagatatttattcataaattcagattacaaagagcctaccacaaaggacaaacaggccggacgcggtggctcacagctgtaatcccagcactttgggaggccgaggtgggtgggtcactttgaggtcaggagtttgagaccagcctggccaacatgctgaaaccctgtctctactaaaaatacaaaaattaaccaggcatgatggcgggtgcctgtaatcccagctacttgggaggctgaggtgggagaatcacttgaacctgggaggtggaggttgcagtgagcagagatcgcgccactgcactccagcctgggcgacagagcgagactgtgtctcaaaaacaaacaaacaacaaaaaacataagtagctgttgtcaggactcttacaacagaatgaggtagaggaagccaaagggctttgaaagctacattcagagcacaaagaagagccaaaaaggacagacactgccaggggaagctgggaaaatattaatataaagaaaatcaaattatgcaattcttattttgtctttgtcctctataaagaggaatgatcttcaaactagaaaagagagatgaaatttcctcacaagaaaaactgaattcaagatagaaaaggatacttaagagaatatcaagttgttagaaatacgttttcatctctgactccagataaattatatttcttggtcatgaaatcattgataggctttggcacttgactggttccaaagttggacgaatctagctactagacgagttccctgagagcatgggtctgatctctttcattcattgctgtattccagcatctagaacatttcctggtatagagtaagtgctcaataaatatatgcagataaatacatgaacagagaacacaagagtggccagattaaaacagggaaatgttctgatttcaaaatggggaaaataaggattctgacttgtgtagatacccaggcaacatgcaggcaagatgtcagcggttaaaaaaattttagaatggattaattaaaaaaacaatcttccaatatgccaggattcctctacaattttacatctagtttaacccacacaataacacaattaactggtcctatacacattttacagataaaactgaggcctaagaggaatttaagtaacttgcctgggagtgatatagcttataagcagaaaagctggcatacagaatttaactactggttgtcctcagaaaggaaatcatggtgactctgaaccaacctgtgtacgataatcatgagtatgctaaactctcctagtttcctttttaaatcaaattgctaaattggttgatgccatagataactggtttatgtacttaaaataaaactcattgagatctttacctgtatctgaatatataaagaagtttctagtagaactactctgctggataagacagaagttcagagatctaccagaataactatccgttcacctttgcagaacttcataaagcccactaccataaacacagggtaaggggagttcagccaggcatttgataaggtcttttgtaataatcttgcaactaaggctagatgacaatcagaccaattagtagtatgtggaaccactggtccctattgatgtcaaactaaaaggaagcctcagtcttgtggcaaggggccctgtccttgacctttttgtattgaaatatgtgaccagtgacttggagaaaataataatattcccatgtttagctgatcacacaattttataccagttcctacacttagacggttgtatacatcctcttctactttgtggttgagaagcttcctctagtagaagtgatgtcaatggaattccaataagaggtatgttctatttagacatttaatccatgaatttgtatatatacattataagtcatctttggcagttcagccctcagccaacagaatctcagtttctccatttctatcattactccagaagggttcttgggtgtggactaggcattctcgttggcatgcattcaagttctcctctgtagccactgatcctgttgcctctatcccacagctctggaatatctccaaataactactcttttattctccatgcaaatgaaaaattccattagggtgatgtgaggttagttgttgacataacttcctgcttcaaaaatccttccttccttcccatcatcatttgcgaccccagttacagctctgaattgtgatttccatttgtgacatattgcccccgaagagacagaagaggaaccttcatccgccatccccatgttagaatcctcttcctaggctctcagtgaagttgcctcactcgatctcaagctgcccggcatgtcacattccatttatttcatttaactaagatttatttagctttatgctatgctaggaactgacaagaaaatcattattacaattaaatgagatttattgagtacttactttatgccagaaactgagctaagaatgaggaataaaaaatgtataaagacacattccttgctctcaaaaaatgtgaagtttagaaagatggaggtagtaagtaaatcaacttcaattcagggtaatagatgctagtagaggcaccatgtgtgatgctgtgagacctcaaagaaagagtctctaaatcatcctgaagaggaagtcacatgaggagagtctttaagaataagtatgtctggccttgtcaattaaaagagagttgggagttccaggcagagtgtcagcatgaatgaaggcaagaacgactactgggatgaagtgtggggagctgagttcattttgacatgggaaaagtgtagaggctgggtgcagcggctcatacctgtaatcccagcactttgggaggctgaggcaggcagatcacctgaggtcaggagtttgagaccagcctggctaacatggcgaaaccccgtctctactaaaaactacaaaaattagccaggtgtggtggcatgtgcctgtaatcccagctactcgggaggctgagacaggagaatcacttgaacctgggaggttgcagtgagccgaggtcgagccactgcactccagcctggacaacagagtgagagactgtctcaaaaaaaaaaaaaaaaaagtaaagaaaagaaaaagaaaaccgtagagcagaagggagatggcaggaaacaaggtgggaagggtaggttagatttgtatcatagaatattttagactgtcttatgaattatgtgggagccaaggaagggttttaagctgaggtatggcaggatctgatttgcatttcagagggatcacttgtggcagccataatgaattaagatggtagagaacagacaagaggaaggacggactgttaaagtgatgggtagtcatagaaacaaaaagtggtgagggtctgagctggcccctgaaaacagatgggtttgaaagctatttaggaggtaaaatggaaagaatgtcatagaaatttagatttgggggtgaggaagagggaggaggtataagggaacctgggtttctggcttggaccagttggctggacgtgggtgtcacatgcaggctactgcctaaaagcataggtgactaggagaatgctattggcctcaatgttggaaatgttgagtttgagatgctagtgaagcacacagaggatgcatattagtcagtggcctgaacttacaactccagaagagcaggctgggctagagaccatagtctctatatggtcgttacatatggacgtctttatatggatatcatcagtatatcatggctgaaaaccagggtcttgatgaagtcacccagggagatggtataagatgaacagcatagagaactaagaataaaaccatagaaaaaagttcattgttggtctttgttgtcttatttggttttgttaaaatggggaatcttgcagatcttttaaatgctgacagaaaagttaaaggagaaaatttgaagatgcaaagagaacatataagtaataattttcctgggagcagaaggtatttatttattcattcattcattcaataagtactgagtgcctctagcaggctgggaataaaaggccccttccctcatgcagcttacattgtagtggtaaggagagccccataggaaataaataaatgatagggtgtgttagaaggtgacaggtgctacaggggaaaacaaggctgcataataaggatgagagtgctggtggagagcacagtacatgtgagatttaaatagtgattaaggaagaatttcctgaggaggcaacaacagttcaaaggcaatgaaagagagaaccatgcagatctccggggaaagaacgcttctggggcaatctgctttattcagtgtctactgattaaatgttaatctcatctaaaaaaaataccttcacggaagtatctagaatactgtttgaccaaatatctgggcctagttgacaaataaaattaaccatcacagaggccaacgacagacgcaagaagaccaattaagaagctctcgtgttaacacagtggagaggtgttggtggcttgagccaggatgtcacaatggaggcagtgagaaatggtcaaagcctggatgtctttgcaggcagcagtcactgtgccaggagtccaaagatgacaagaaatgattcctgtggtcaaggtccttacagtctgtaacagttattaaaaccataattgcttatttgttcatggcatcttcattaccttgattataccttcctggttcaaatcatatttaacatctattccaacagaatggcattggaacctaccagaagacaggacaccagtcactacacaggactgatgcatacttattcagtctcaggtatgtctttatagcaatgtgagaacggactaatccctgccttttcattgtaatgactgtcaatccttttttttttttttttttgagatgttgttttgctcttgctgcccaggctggaggacaatggcgtgatctcggcttaccgcaacctccacctcccaggttcaggtgattctcctgcctcagcctccggagtagctgggattacaggtgcccaccaccatgcccggctaattttgtatttttagtagagatgggttttctccatgttggccgggccagtctcaaactcctgacctcaggtgatctgcccacctcggcctcctaaagtctgggattacaggcataagccactgtcaatcaatttttaaaaacaaacattttcttctctgtacctagtatgagacaatttgagtgtactatgtcaggtatttgagtggaatatataaaatctatatgttgtacaagtggaaaacataaaagctatatgttgtgcacatccagcccttctcgtagaatgtctagtacagagcctaacctttgcttacctaaatcagggttgtgctggtctcatcaccattcccaggaagctgggtgctgccctgtctgtggtggggcacagaaacccatttaccttcagtattcagatagtagagaagatggaccaagttggacttgggaatccccattctgattttgaagtctaggcttgagatggggactaagagatgagaaatgttgtaaagatggagaccaagactgctggagaattgagtcaaactgagatagaaggcaggacttgactccagaggctgggctcagacaccggaccagattgaggactagcaaatacagggccaggggcagaagcagctttctatcagacagcccaggagtgtgccatgtcaatttactattgccatggcaacacctgggagttacttcccctttccatggcaagactccaaagttactaccccttccctagaaatttctgcataaactgccccttaatctgtatgcaattaaaattgcgcataaatatgactgcaagactgccctgaactgctactatctgcctatggggtagccctgctctgcaggcgcagttatggagctgtaacactgctgcttcaataaagctgttttcttctacctccagcttgcccttgaattctttcctgggcaaagctaagtacccttgtggcctaagctccactttggggcttgcctgccctgcatcaaaaccatgctaatgtggggcaggctttgaaaaagcctagagcctggagtctagaaggccattattggaggtagctctcttttatgcatcctgcaagcacttccactgtgcattccagattcatttaaagggacccagagatatgcttcttagactttccaccaaagcatctctcaagccaaagggaataaacaggtagcttcaaacagtctgcctcatgcctggaatatatttgaaatctttttgttttattttaaaaattacacgacttttgcattctactttgtaatatcaccattgttttaataacccttccccccaaattcttgacataaaatgagcactgcagaaaaatgtttcatattcatttttggccttatgtaatccttggcacacaacttgacctaagtcatgcctagcccagactgagaagcatgaacctacagatgaatactgaaccattgattgaatgtgtcatcattcttttgagacctggtagtttacaagcctagagcagagaagggaagctagtaggtggccaataaatcatggttgaatgatataggtcttctcatgttctattcttaatctaggcattttcaaacacatgcttttctttctctaaaatcttatttctctttatttaaaatcaaacaaggctggttgcaatggctcatgcctataatctcagcactttgggaggctgaggtgggcgaatcgtttgagcccaggagtttgagacgagcctgggcaacatggggaaaccccgtttctataaaaaatgcaaaaattagctagatgtggtggcacacaactgtagtcccagctacttgggaggctgaggtgagaggatcacttgagcctgggaggtggaggctgcagtgatccatcattgtgccactgcactccagcctggatgacaaagggagacccatctaaaacaaacaaacaaacaaacaaacaaaacaaaacaaaacatttttttgaacatgtataaggcacggtctgtagtattggagaatcccaggatgaattaatttggattccatacttacaatttcggctttcaccaatgacttgagaagctgctaataggcttctctgataacctattttatcttcttctcttttgatcctaattctgttctatggattgtatactctcagatttattttcatcgtattttgttttcattaagggatgcttgcaaaaagtttagttggggagtgaaaaaataactcttatgtatatccaatattaaaagtttcttttgaaaaagtttttttctagtaggtcctttctaacatgtcttttgaaaacagtataggttggttttatggctatgtatggggatctcatgagaatgccattaaaataagcttttgctgagttggaggataaaaggaaatgggaaaaaatccaaatgcatcccattattacaagaatggttccctctacatttaagaaattatttttctgcagttcagtgatttggaaaaacatgtattttctagtttcctaagaataaaatgaattaaaacgactttaaatactacataggacaaattttaattttctactgcattctatcatagactcttttcttctatattttattttatttatatattttttgagacagggtctcattctgttgccaggctggagtgcggtggtgcgatcacggctcactgcagccttgacctcccggggctcaggtgatcctcctaccttagcctcccaagtagctgggactataggctaattttttttttcttttggtagagatggggtttcaccttgttgcttaggctgttctcaaactcctgggctcaagcgatcctcctgtcttggcctctcaaagtgctaaaattataggcgggagccactgcgccagacccatattttgttattttgagagattattctacttcataatttggaatgcatttctgattttcttgttggaaatggaaaaatgactattttaggttgaatccattcttaattatcccctgatacttcgtttaggacatttctggattactttttctcttcctgttccttttctctttttttccagtcctttcatgacagataaatgttggttggataaaccaaggagttggtacatatcccaagaatgcaaattcattttgatattaaatttaaaaacatattccaaggaaggtaaattcaacccacccaccccctacctcaaacactatgggtgctggttcccattctctcttctcttgacccatcctctgtagcatgagcattagccacgtgtcagaagagttacagtcactagaaagctacctagaaatcagaattccctcttctttcaattgtttttgaaagttaagtcatcatagggcacaccttctgaaatccacagtctgtaatgaatgtaaacacagaatgtgcatttgtgaactgtgccccaaagggttggctttctagattccaaaaaaggacaaacattttcagatgttgaatgcacagtgatgtcagacatcattagtgacagctcatttctgcattttgtggttggactctaaactatgttatcccagaagaagtgtctccatcttgccctggatttggtgggagaaggatctgacacagggagctttgcccctgaaaacagcactgtggcagctggaaatgaccttatatggtagcttcgctttacactccctgtcccagaaagatccgtatggaatgtggtgacttttgatgctgataaggacattccacaggagaatgctggggtcgggtagacgaaggatccctgtgaacggttacccattaacactggctctggtttcacaccagtgaagagtggcagtcggcatagttttcattagtcagacattataacctgagtttggcctatttttctttgaaacatttggaattgtcaagccaaaatgcatttcctatggagtgagtgagtttgcctctgccagcctttccatgtgatagtttattattatagaggtgacatgggcacccagagtccctggcaagctattgtttttctttgctaagaacagcaggaagagacaaaaaaaagtccttcagaaaataatttatctagacttttgcaaggcaatgagaaaatgaattgccattaggctgagcaatctaccctgtatcactgaggatagaaaatgtctcagaaggtcactcatgtctaagaagtttcacgtggatcttggtacaattgtggcttaaaatgtgaagttcaggccaggtgcagtggctcacgtctgtaatcccagcactttggggggccaaggcaagtggatcacctgaggtcaggagttggagaccagcctgaccaacatggagaaaccccgtctctacttaaaatacaaaaattagctgggtgtggtggcaggcatctgtaatcccagctacttgggaggctgaggcaggagaatcacttgaacctgggaggcggaggttgcagtgagctgagtttgcaccactgcactccagcctgggcaacagagtgaaactccatctgaaaaaaaaaaagaaaaagtgaagttgtgaagttcaggggttcagagaaaatcccttcccctcataagtgaggaatgaagaatggaagcactctgaggacacttccatgcagacagtcagatggacacacccagtagagtggctttgtaatattgcactgtctatcataggacacttcccagctaccagacacagagtttctcttgggctcagcccagtttttcatctttgtgttgccagcaccacggcaccctgcacagcacatagcaggtgttcaagcttcctcccttccccgtctatcctcccctttctccctccctttcccccgcttccctcaaccctcatctacggagtgcttcttatatcccaggcactgtgcccaagcctggtgcatggtgaatttcagcaacatgcatctcctaaatattaagaaaaaaaatcccccgactaagaacctttggtttttaacctcattctgtgctattttcagtaacttctggtttccacactaaaaaaatttgcagtcattatttattgagcatgatctacagaccaggcctagtaatagtcacctaattcattaattcacttacccattcacaactatttattgagtacctattgcatgttaagtgctggaattacagcagtgaataaacacacaaagactcctgaagctcatgagtgtccaattacttgcattacctgtattaaaacttatattattcatgatcctcatgaagcttccggtggggtgaagggagagaagcaatatgtaagcaaacaataaacatgtatgacatcaggaggtaatcaataacataaaaatgtaatgacacgggggcactggagagaactacagcaggtcctatgaaagctgggggaaaagcatgatagccagagggaacaaccggtgcaaaggcactggggcagagagcgcctgtgtgttcgggattgtgttttggctggagcagaggtgttgagggggaagtgatcagagatgagatcacacagctggcaggggtgatgtctcctacagtttcataagccactctaaggacctggctctccctctgagtcaaatgggaagccagtaaaaaaatttgaacaaagcggctgggcgcggtggctcatgcctgtaatcccagcactttgggaggccaaggcgggtggatcttgaggtcgggatatcgagaccatcctggctaacacggtgaaaccccgtctctactaaaaatacaaaaaattagccaggcttggtggcaggcgcctgtagtcccagctacttgggaggctgaagcaggagaacggcgtgaacccgggaggcagagcttgcagtgagccgagattgcgccactgcactccagcctgggtgacagagcaagactccatctcaaaaaaaaaaaaaatttgaacaaagctacgtggggcatgatctactgcacggtacagtggttaagtcctcgggtcttgggcaaggctactattttggaattatgcctctgtttcttgctagttgtgtgactgaggttaagttactcaacttctgtgcaccttctgtgtgacaaccatacctaccccttgagtcgttataagggttatataaattaatatttgtaaagtacatatttgttaagtgtacaaaatgaatggatttttaaaaatcacttaaaatgtactagataattatgaactgcttttggacaattaatcaagtgacttccctaagggcacagatcatagatgaacaggtcaggatccacacccaggtctatgtgaccctgaaacccatatttttagcatacagcgtggcacctgcctctttcctctgcaatctcagctgctggagccatcattaattagatagtaacagggtgtattatgggttgtattgtgtccgccccaaaatttatgtgtggaagtcctaatacccggtacctcaaaatgtgaccttatatggagacagggaaatgtaatcaagttaacgaggtcattaggatgggccctaatccaatatgactggtgtccttgtacaaagaggaaatttatttttgtttttgtttttgttttttcttggagacagagtctcgctctgtcacccaggctggaatgcagtggcacgatctcagctcactgcaacctccacctcctgatttcaaccaattctcctgcctcagcctcctgagtagctgggattgcaggtgcccgccaccacacctggctaatttttttgtattttagtagagacagggtttcaccatgttggccaggccggtcttgaactcctgaactcaggcaatcctccctcctcagcctctcaaagtgctgggattacaggcgtgagctacaacacccatccaaaaggggaaatttgaacacacacacacagagaaagagagagacagagaaagagagagagaatgccacatgaggactggagttatgttgccacaagccaaggaattttgagaagcttggaaagaggcctggaatagctccttccctggcatctttagaggaagcgtggccttccaaacaccttgattttagatttctaggctccagaactgtgaaataatacatttatattgtttaagccaaccagtctgtggtgctttgttatggcagtcctagggaaccagaatgctgtctttagttatcctcaatattttatgcactcaaagaagcctttgccaaatgtctagtgaagacacagtgctagactccttccctagggaaggcagctgggttttgtgcagtttctatgttgtccaaaccctttgccccaggagtgggtgagaagctgcagcaagtcacttacaatgaagggtgattgcgagtaggagtgatcacctaggtctctgatgtgtgctaatttcctcctgtaattaacctgtcaacactgtctgttaataaacctagggggttgccaacattgatcaaatacaacacgaggaaattggatcctatggaaaaaatgtcacatcaaagtttcagaagcatttaaaaaaatctgttctgattctgctgagtttttaagtcagacaaatcattcctgatgagttaagattgctggcacctgggtcgtctggtgaggcacctcgttttgattgcccgttgcatagtttcatttcagcctaagatcttaattcctgcaaatgatttccctagcatatatgatgaaaagtaatgtgcctgctgaagatggatttaaccatatttagctattggaaagaccttgtcaggaaccatctttattcattttttaaatgtttgtatagtggcatatgaatgttcaataaatataattttcatgatcacacaatattttctcttaaccttgatctcttctcctagattatttaaattccctatgttaataatagaaaaaaaggacctataattgctattgaattcctgcagtttacacactgtgctagaaatacctgtcaaatttccctgtaactgctgtgaaacagcctcagaagccagcggggccaccagcagaaggtctgataccttctacctcacaattaatctagatttggcggggcatggtggctcacacctgtaaatccgagaactttgggaggccagggcaaggggcttactttagtccaggagttctagaccagcctgggcaatacagcaagaacagtctctacaaaataataataataataataataattagccaggtgtggcggtacatgaccgtggtctcagcttctctggaggctgaggttggaggattgtttgagccccagggggtcaaggctgcagtgagctgtgatcacaccactgcactccagcctgagactctgtctcaaaaaaaaaaaaaaaaaatctagatttatggatcatggatgtggaagaatcagaccttgctttgaattcagctgtgtcactcactataccagtttcagaaataccttctgagggccatgctccctaaaaataattttaataaagagtatagacagtttacttcttaaaaagcaatttattatctgaaagaaggaaagttcatagtgattattctggtcaatgcttcattcatgttgatatttccatagataatagtagttaatttttggtagttgttgatttacatgtttcattaacttagctacatgccttgtagcacctaacacctcaccctttttgagcaatttctcaggaaaagattggtgatgactttgttttgtacagctgactcatcaccccaaactcgccctgtgatgagccctgggaacccttgcaaccttgatgacagcagtcaggaagtatatgcagttaagcaattcccatagaagggaagtgaagcagtgggtcacctgatactggagtctcataaccagactcgcgaagtaggaaggaagcctccttgcagggcggttccaccacagtatgagcggccacagaaaaagttaccctgagtgttccgtgctgcaccgcgtcacccagccccacattcatatgttgaagtcccaaaccctagtacttcagagtatgatcgtatttggagatgagtctttaaagagataattcagttaaaatgggatcctgggagtgggctctaatccagtttaaatggcgtccttagaagaagaggagagggggacacagacacacacagagggaatcccatgtgaggacatagggagaaggtgccatctaccagccaaggagggaggcttcagaatgaaccagctctgctgacacctgatctcagactcctccaaaacagtgagaaaataaacttctgtggcctaaggtgcaccatctgtgatgtttgttactgaagccctatctgactaatacagtgggtgttgcgtttatgcaaccttttccttcatagaatgcagagggattaatctcctacttcttgcctgttttccctcagcattagccactctggcctcctcgctgttctgcacacgtgccattccctctgcctagaacatatttctttcagataccttcctggctccttcctttggttgctttaagtctgctggcatcttgccttctcggcaggtctcctctggccacttattcaaaattacaaaccatccacctcctaccatcccccagcactctttatcctccttggttttaaaatttttctctatatcacaaggtcaggagatcgagaccatcctggctaacacggtgaaaccctgtctctactaaaaatgtaaaaaattagcctggtgtggtggcaggagcctgtaatcccagctacttgggaggctgaggcaggagaatggcgtgaacccgggaggcggagcttgcagtgagtccagatagcaccgctgcactccagcctgggcgacagaatgagactccgtctcaaaaaaacaaaaaatttttttctctagagggcttatcacctcccagcaccctatataatgtacttatttaaacatttaacttcttcattagaatgtaagctccatgaaggcagaaatttttgtctgttttattcactgatatatgccccacagtagggatagtacaggacatgtagtaggaactcaagaagtatttgttgaatgaaaatattaataataatcatcataatacttaatgtgaattgagcaatatgtgccagatactaagtgctttacgtgtattggttcattatacatgcatagatacacttaatgtagtatagcttaattaattatgttaattaatatatgaattacttcatggttaaaataaaaatattcctgtgtggttaaaaaatgtttgatttctttcctctttgttatatattgtgggagtgaaggaactcctctaaaagcagagtaggcagtgataaagcttgtagggaaactttgattcaggacaaagaaagatttgtcttttcagaaaggtggttatatagccgggtgtagtggtgggcgcttgtaagcccagctactcggagaatcgcttgaacctaggagatggaggttgcagtgaaccaggatcgtgccactgcactccagtctgggcaacagagccagactctgtcttaaaaaaaaaaaagaaaaaggagtttatggtacatgtgaactagtatttactagttaccaatgattcagtccttaccctgtgggaggcacaatcccttcctcagccaaccagggaaggaatggataaaacaatctgcaaggtagaaagtgcagaaatgaaatgcaaagactggattacaaagcacgttatgcctccgtccaaacactccctacgtttctcaccatagccttcaagataagaaagagcttttgattcgctggtataattttgaagcagataatttcttgaaaatattcgattttaatcttggttgcttttgtgttttgtgatccttattgattacctatttcagcatagttagagtagtttaaagttgctagggtcaattataatgtaaaagccatcaataatgctgttagatgttttaaaaaaatagaaaaaaatctagtgaaaagctatgatttgaaagtataacagctgtgaaattgctacctttcctccttctgctcattcatcatctcccggcttgcagtatgttggagacagcgtctggggactgaattgaataatttgaaggggggaaatgatacacctttgtaaattaaacacaactgccaggaagattgattaaatacaacttaactgaaacctcattatagattcagatgaagttcctcatcagtctttctacagaaaaaaagaagaaacagctcccaaatctggatgtggtttcccctggaatagatctcactttttaagaagttttccttctgtggtgttaaaaaaaatcattttttttttctgctgctgttttagaaatcctgaaaagtcataatgcccgataaatgaatgccccatttatgagtcacatttacttttacatatatatgtgggagactccctagaggtgtatctgaagattactaatggaaaatcttgatttattaactcaggggaagctacatagactgtctccaagagttctgtactcctctctcttataagagtactttgaggccgggcgcggtggctcacgcctgtgatcccagcactttgggaggctgaggcgggcggatcacgaggtcaggagattgagaccatcctggctaacacggtgaaaccccgtctctactaaaaatacaaaaaaattagccgggcgtggtggcaggtgcctgtagtcccagctactccggaggctgaggcaggagaatggcgtgaacccgggaggcggagcttgcagtgagccgagatcgcgccactgcactccagcctgggtgacagagcgagactctgtctcaaaaaaaaaaaaaaagaaaaagaaaaaagagtactttgaataattttccctcaccactttccatcatatatatatgtatatatatatacacacacacacacccacacaacataaacatagatatttatgtatacacatacatatatttatataaacacatatgtatattcatacatacatccatgtgtgtgtctatatatttagtattgcccgattttgaaaactaaaaatgatgcattttcaccataaagaaatatcatgtaatgcaggaaagagcaaagaagaaactaacaaatcccacaaaatcacatctccccaaggaaaaaaaattagtttggtccatggtgaacatcattctagatgtcactttaagcctatattgcaaagtaaaatgctttcaggagtcaagaggaacttccgtggttgagactgatgtgaggagactcattcaatcctctcagtctaccttgagcctttccacatggtatggagaaatatttctgtaccctaaaaaaaattttttttaaactacagggcaagcaagatgatatatagcaaccaatgctttccttgttgggagaacaataagagtgatggcacctctggcaaactgaagaagacatgccccgtccggagggcacaggtatttctcaggtaaagtaggtgattgccaagtggcaatgtggtcccagtgttgtcagaacttctgttctgctagaagtgagacatttgacttcgtactagaaactttctaatttaaaaatattttctcaaaattgtttaaagactctatgtggtggctcacccttttaatcccagcactttgggaggccaaggcaagaggattgcatgagctcgggagctcaagaccagcctgtgcaacatagtgagattgcatctctacaaaaaatacaaaaattatccagtggtggtggtgcagtagcctgtatccagctactcaggaggctgaggtgggaggatggcttgagcccaagaggtggaggttgcagtgagccaagattgtcccactgcactccagcctgggtgacagagccagactttctctctaaaaaaaaaaaaaaaaaaaaaaattgtttaaaagtgctgcaacgtagcctactcactgctatttgttagggacagttagataaatggatgggtagaaagatctatagataaataaccagcaagtcaaatgattatttaaaaataaagtaattctatagaagctacttactttttaaaaatcacaatagatctatatataaataaccagcaagtcaaatgattgtttaaaaataaaataattctatagaagctacttacttttaaaaaaaaatcacaattttacttaaacttgatagaagaaaaacaggccaggtgtggtggctcacgcctgtaatcccagcactttgggaggccaaggcgggcagatcacgaggtcaggagatcgagaccatcctggctaacatgctgaaaccccatctctactacaaatacaaaaaattacccagtgtggtggcacgtgcctgtagtcccagctactcaggaggctgaggcaggagagtcacttgaacctgggaggcagaggttgcagtgagctgagattgcaccactgcactcctgcctgggtgacaaagcaagactctgtctcaaaaaaaaaaaaaaaaaagaaagaaaaagaaactgacattaaactgaaagagttgctaaacttcattaagcatttcttcaccatcaaagagtcttctaaacttaagaaaattattgtggaaaacattatgagattggagacttaattattacaataaatgatcactgtatttttaactacaggtatatgctttagtttaagactatacagattgacttcctacttttagaagatttctactccttaaataatttttccttggtttgtcccaagactatcttatcagccttagtccacttttctcagaatggtagggaattatgagaatctcataatgtgaagtggtcttccttccttagtacttcttccggaggcggaaccatagcacttctgaatcttctgtttcttttcatagactgtactctttgatgtttatggaataatgctatgccctttccttatttggttgttgctagagaattttttgcattcttaaaaaccacttttcagccaggtgcagtggctcatgcctgtaatcccagcactttgggaggctgaggtgggaggactgcttgagctcaggagtttgagaccagcctagcagatccccgtctctacaaaaaatataaaaattagccaggtgtggcggtgtgtgcctgtaatcccagctacttgggaggctgaggtgggaggattacttgggcccagggtgtcaaggctgcagtgagctgtgattgtgccactgccctccagcctgggtgacagagtgagaacctgtctcaaataaataaagaaattaaataaaacaatttttcatatagaggtatcaaagcaagtggtttttataatcctacaagattttgctgtatttatctagaaacttttctcaactatctttaaagctagtgtaatatttaccaagaaactgttatatgcaatcaatatttatcataccagatagtgtgactcataaagtttattttcaagcacttgtgggtgtatcattaatttagcaatcatatactatcctatatcatgcggctgtaattacatgtctgcatagccatatatactatatatcaatttcctgggttattcgacagtggtgcccccagttacgtttttaaaatttaatgtattttacgactaaaatagatcttatagatcagtcaactctcatcttacagatgatgcaattgaagctcagagatcaatttgaaaagcttttaagtccttcaagggactggaaatcactcaagctttttgtaccctcactccttttttcatctctggtgtgtagaaccacaccttgtgtgattacagatagatcaagcccaataagaaatctgctgccctaaactccagtattgagaggctgctggcggtaagtgaagaggctttccacattttcaaaccaaccttcctgcccatgacttcataagcctatggatgctgcttacctgtgtgttaaggtgtggtaggtgccggccttcatggtcggtgaaaatgtgtctgtattagtccgttttcatgctgctgataaagacaaagctgagactgcacaatttacaaaagaaagaagtttaattggaaaattaaacttttccaattgtggcggggaaacctcacaatcatggcagaaggcaagcaggagcaagtcacgtcttacatgaatggcatcaggcaaagaaagtgagcttgtgcaggggaactcctctttttaaaaccatcggatctcatgagactatcacgagaaaagcatgggaaagacttgcccccatgattcaattacctcccactgggtccctcccacaacacatgggaattcaagatgagatttgggtggggggacacagccaaaccatatcagtatctatctccagattttgtagtaaaatatagattcagggaatagagcttcagaaacaaagtaccccttgatttacaactacctattttttttttttttttttttgagacagggtctcactctctcatccaggctagagtgcagtggcatgatcatagctcactgcagcctcaacctcctaggctcaaacaatcctcccacctcagcctctcaagtagcttggactacaggcacatgccaccatgtccggctaatttttgtgttttttgtagagaaggggtttcaccatgttgcccaggcatcttaaactcttggactctagtgatccacccaactcagcctcccaaattgcaggtattacgggtataagccaccatacccagccgtgactacctgattaagagaatttcccaatctagaaagctttatcaccatttggagatcagaagtctggatgaaagaaaagaactaccttatttatccctctgagcattttattttatataatatgctttttaaaatatattttgaccagatgctccctcctgcaccaacccctcctcgccaccaacacacatacatgctcttgtgtgtgggatttgttgttgtcgttgttgttgtttggcttttggcttttgtaaaaataggtgctgtggtcagtacaaataatccagtggtctttcactcatcacattgactcctcactctccattcaagatgctttcatctgacatttaaaattgtaggcataagcatcccagcactttgggaggctgaggcgagcagatcacgaggtcaggagatcgagaccatcctggctaacatggtgaaaccccgtctgtactaaaaataatacaaaaaattagcctggcctggtgatgggcacctgtagtcccagctactcaggaggctgaggcaggagaatggtgtgaacccaggaggcggagcttgcagtgagccaagatcatgccactgcacactccagcctgggtgacagagcgagactctgtctcaaaaaaaaaaaaaaaaaaaaatcgtaggcataagcatgcaggtgacagtcagccgtaccatcacatccacatgtaactatttccctcatgggaagtaaataacttctgagtttttactaaaggacacaatggtccaatagatgtgtgtgtctgtgtgtgtgtctgtgtgtgtgtggaacaatgttcaaccaacccttgatactttacactgaagaaaaattaaaaagagacctagaatgaagaggattatcaataatttattgaagaaattctgttcattttgataaacattcattgtcaatttattttacattaggaatagtgctatacttctagccctgttactcacatccttcagcatctacataaattatccatccaaaaccctggcttcccagttcccagacccctttatgcccactcatttttttctccaccacacctttgctatatattctcaaggcttttcaatatctgtaactacaccacctcagcaatccccattttaagtgccctgtctctgattacagcctccaggctttccctaatatgcccactccaacactatcttgacttcccagatactgccaatccattggcattattttaaaacatcattattattttgaaatatgtcaagaatcatttcacacatacaaacaatataaagaataacacagcagtcatatatctactatctaactatagatataaaatattaccaagagcattaaaccccattacatagccttcccaactacatcccctagataactactatctaaactttgtattaataactaccattctcactcattaatttttttcttttatctctctacaaaatactgtttcgttttgcatgcttttaaacttataattactgggatcatacagactgtattcctttatgacttccttttattttctcttgatggaatgtgagagtcgtccctgctggcctgcagcactagttcatcattttcacttccacgtgttattatacggtgtgaatataccatcattatttaaaacacatgtttctgttgatgggcatttggttctcccatttgtttgttttcaatttcaaacgatgctacagtgaacatttttgaacacctccctgtgcacagataaatgtgttttttttctggaagacatggccatgccacgggcatacttattttcatcttaactggtgctgtcagattatttcctgtgaattcccaccttttccccttcaagcagctcacgtccttgcttcttttcttgcccaggttagattccctgaagcatcactaagtgattcctttccatatattgtggccacctgaccttccctctttcactgagccttcctagcaaagccccgtctttggcttaatccaattcttattattctgaactgaatgtagagaataacagagctctgctgactgatccgctgcacccatgcgacctcatgggtgcccttacttctaccaggcagtcccagctcattccctaaacaattctctttcccagacaactgttcacacctttctcctccaacttctgtcttcactctcaaatgatgaccgtttccccttcttttacaatactataaaaagcaacccgaggagaactttcatgtcttcccacctcctgatcagtcagcctgctgccggtgaccctctgccgtcccacctgccacaaaaggggaactgtctctgatcctgtctaaagccaacccctcttctgaacactgggttccatctgtctgacaccgctaaaggtttttctactctaattttctccactgctcccatatcactatttcctccctttcttcaggatctgtcattagcatacagacatgctctaatatttcccgtcttaaaataaaaatgtcctatcctcctgtcttccacttcctcatgtatctgctttgttgtatggtgagctcatcaagagttatctataccgattgtctctatttcctcattcctatctgagttttgacacactcagtgaaaccgccattgccaagccattgataacctccatgttaccaaatccaatgatcagctctcagttcttatcaactttacagcaacagttgagccagcaacctctgaatgttcagagaggctgtagctctgttgctgaccaattcaaactggtctgcaatttgagttgaatcctgggagttgagccttgaatctgcagctccacctgattttcttcactaggcttccaaggcacttttcctgttatcctctccctgcctttgcctccccactgcggcttcttctctgtttccttttctggatcttcttttccttcctggcctctaaatgtttgagtacccctaggtacagacctcgcttctcgatctacagtccaccgcctgaagctctcatctgccccatagctttgaataccatgaatatgctggtaactcccaaaagcatatcaacaatcccctttaagggacactgatatgcacccctgtttatactccacatctttacttggatatcaatgggcatgcgtaccaaacaggattctaggttcttccctcctgtttgtccacaaatagctctaccctccacctagtttcccagaccacaaacctgggagtcatccttgattcctctttttatctcacatcccacaaccaatctgttagcgtgtcttgtcatcccaaattagcatatgtcccctgactctgacccattttcagaaccttccctaccagcatcctagtccaagccactttccctctcccaagctactgcagcagcctcctaaagatctccctgcctccattcttgaccctacacagcatgcttacagaaaatcaggccagtcttttcttgtttcctttcatcttccgaaatatttattgaggtataacatatgttccgtaaggggttcaaatcttaatgttcaactcagtttttacagatgcatttgctttggtctgatgttggtgtcccctcctcaaaatacatatgttgaaacctaaacccaacacaatagtatttttttttttttttttttgagacagagtgtcactctgttgcccacgctggagtgcagtggcacagtcttggctcactgcagcctccacctcccaggttcaagcaattctcctgcttcagcctctggagtatctgggattacaggcatgcaccactgtgcccatgtaatttttgtatttttagtagagacggggtttcaccatgttggccaggctggtctttaagtcctgacctcaggcgatctgcctgccttagcctcccaaagtgctgggattacaggcgtgagccaccaagcccggcccccagtgcaatagtattaagaggtgaggtcttaggggatgattagatcatgagagcaaagccctcttgaatgagattaatgcccttataaaagaggttagacgaagcttacctcccctttcactcttccaccatgctaggatacagctagaaggctctatctatgaaggcaacatccatgtctgcccttaccagacacctaatctgctggcaccttgatctcgaacttccaagacttcagaactataagcattacatttctgttgtttataaattatgcagtccaaggcattttgttatagtgcccaaacgaatgaagacattgtatgtggaacatctgtgaggatatagaccacttctagcaccccagaaacctcaaccatgacctctcccagtctataactcccaaatgtaacaaatgtgacttctatcatgatagatgacctccacttttttgaacttcgtatgaatggagaaatactgaatgtattctttcatatttggtttctttcatctcttcattaggtctgaaaaaaatttcttctcttgtatttagctgtaccttgtgatttttctttgctgttatcttccattgtatgaatataccacaatttatttatcctttgtaaagttaaagaacatttggtctattttttagcttggggctattatgagtaaatatgctatgaatgctcttgtacacgatttttagtgaacagaagctgttatttctttggggtatatttttcaaatttttctatttatttttagctttatttatttatttatttagagatgggggtctcattttattgcccacgctggtctcaaactcctggcctcaagtgatccttccatctctgcctccccaagtgctgggattacaggtgtgagccactgaacctggcctctttgggggtatatttatttgcaggagtggaactgcttggtcaaaagaatatgtgtgggccaggcgtggtggctcacgcctgtaatcccagcactttgggaggttgagacaggcggatcacttgagatcaggagtttgagatcagcacggccagcatggtgaaaccccaactccactaaaaataaataaataaataaaaataaaacaaagcaaaaaacaaaaattagccaggtgtggtggcaggtgcctgtaatcccagctacttggtatgctgagaggcaggagaatcacttgaacccggggagtggaggtttcagtgagccgagatcacgccactgcactccagcctggatgatagagtgaaactccatctcaaaaaataaaaataattaaaaaaattttaaagtatatgtgcattcagctttagtagatactgtcaatccattttcccaagtggctttattgactcatattcccacataagatacgaagattctggttcctttacatcttcaccaacacttttattttagccattcttcagtgtcgtagtcattttttaacataaaaatcaaagcacaaactgtctcctgactacaatttcccagggctcctcctggcacttctctgggccatccttcccttttcatccctccccctcattgtcctgctccctcactccattcaccaggactgtttccacctctggatattttctctccgttttcctaagcctggtgtgcccttttcccagacatcgaagtcctttgctctctcattttcttgaattcccttctcaaatgatacctccttagagagatctttgcctcctgccatgctcctattgcaccctatctttttactctgatttcccttcacagtacttatcggttgttgaccttacgtcatacacttgtttatttgtttattcttggtttcccttggtagaatgcacactctatgaaagcaggaaagttgtctttcttgatcatctctgcatattcggcgttcagtgcccggcagagggcctggcacttagcaggctctcagatgtttggtacaagagttctctatctgagcgaattctttctccccagcaattttatcccatcacttacacttataatcatgttctcctcttttctttcatccaatcttcattttttttctattcacttctcttctaccttctctttcctttctcctactctatttcattattcataggaggctttggaattacagagacggcggggcctcaggttggatgctgaacattaaagctctaaattgcagggcagcaggcaagggaaaaagcagagctaacacttcttttgcaacgttaaaaatgcattatgacaggcctggtggctcatgcctgtaatcccagtgctttgagaggccaaggcaggaagattgcttgagcgcagaagttcaagaccagcctgggcaacatggcaaaaccccacctctactaaaattacaaaaattagccgggtgtggtggtgcatacctgtagtttcagctacttggaagctgaggtgggaggactacccgagccagagagactgcagtgagcagagatagcgccactgcactccagcctgggcgacagagcgagactccacacccctccctgcaaaaatgcattatcatactctacactttatcttgaatttagctgaaggctttcatagaaattctcctctctgtttctctatccctctgcctctttccttttctctacacctccaactagacatttcacagacaaatctctggagtagagtccattctatgtaacagcaagcccctcatgctgttggtcacaaaaccacactgtcagagccagagacagctgtaaaccctgctctcccagatggagagcagccggagccgggattttggtagagggagttgggaatacattagggaaagtctaatgagaacaaaagaggcagctgaatattccccactccaccttagtgtgcaagagggcagcagtccagatttttcatggcgttggggtctggcagggtcgttagtctctatccaacaagcattagaaagaaacagaaaataacatatatctgggggtgtgcctgcctgcctctgtcttgtgcaaacaacaataactggaaaacacgacttagccccttacttgatggagggagaaatttggaactcttctgagatgatgaaagaatagaactagataaggaaagtaaaatgctgttcctggtctgaaaaagagaaatgattgatctgtagtaacaccaactacaaagagtacaatccaggtagactcctctgacgctggcaccctgtcacagagagggggaagaacatcaggcttattcgcatgtgtgcaaatggccaagcactaatgtaggaggactgacgggagtctatctatctgtggcttctgggtagacacaggcaagcctaaccctgcccgggcctgtgtctgatggcgaggcttggtatatactacaagcaccttgtcattagggttttatcgtgctatggatatgctaacgatacatttataaacaaagtttctaagtagtgcaattgccgaggaaaaactgattgatttatacaagtgtggattttttttagacatcccgttccatgccttagggtttctagtgttctctagctgggcaagtccagactcactttggtcttgttctgggtgttttccgaggctgtgctcgctctgtgtggatgacatgagtgtgtgtggagtttatggctgtgataaaatacagctctggcatttgctgtggttccctatcgaaagccacgctttctcggcactggttgcctcccactgtagaatctgaaaatctcagataatcacattcctggtgtgtttgtagctcagcagaggaatggcacccaatcctgggcaaaggaatgtgaaggtttctggaaaacaggttttcttccccaattttcaaagagaggtttcttccggtccctgccttcgttcgagggagcacgtgatacttggcactgcaaccacagtcttgtgaacctgtgggagcagcaccaaggaccacgaagaggctgccccagggcccaggagtcactgaatgactgagccaatctggaattgcttctctctagggcccttttagtcacatatcttgcttcttgcagaagaaagcattaacaatgaattacagcatatttagtcggctatgcgttaatgacaacgtagacccaaatccccctttcaaggcccctcagggcttctcactgcaggtagaataaagcatgaggtcctgaacaatcagcccctccacctctccaagcttctcaggccttgctctatgttccccacgtagtctcctccttcactttcctgtgtgttctcactgtcaaattctacctcagggcctttgcacacactgatgaactgaatcctattccttcctctgcactgactttaaaagctgcttcctaggaatggccttgttctcttactcaaactaaagcagatgcctggtgttatttttctctgtcagttccctgctctttccctcggaggccgtagccaactgtgtggggctcccttaacatgcattccagtctccttctcgaaggcagggcctaagactcaaactgcctttcccaggctccattgcagctgggttctagaaatggttgagattctgcccatcagacatacctgtgggaggtggtttagacatgctgctacatggtgagagagagctggatagaggggttcatctgactggccggggtcctggcagaggatgtgagattctctccttaacaagggcaggagctccctcggtggcctagttctgcaatagttgggggcgatgatcttggaattttggcctagagttgtttctttaggcctctttagaatatggagctagctgtactcttaatgcacgcctgtctgcttaatttgtctatagtgtatcctgttgtccataattacaaactttgacccataaagcaatagtcattgcaatttgtaattatatattcatttcatacatttcctaacaccctggtatctctaactagcctctgtgctctgaacacttaacatagggtacagtgtctgtagctcaatatttattgatgggtaaataagtagacaattgagcaaaaccagacattgcatgtaaagttagaagaaaagagaaagtcctgaagttgcatctttgatatttaaatcttgagtgatttgaaagaagccagaagctgacatttacaataagttcccaacttgctcaattcacctggcaggctcatcgctgtgatagccaaactagaacagaggtccagggaatatgaattccaactgttattaaatgcaatagcaatcttaaccgtaggatttttgtttctcaagaaacataatttgtcaggagagatttctcagagcctttcctgttcctcctaagagagtatatcccactctgagatgctctctggtaccatggaactctccatcaaaggtgcagcctcacattctctcctgggcagttccctgattcgactgtcatgcccacttgactgtcagatccatgagggcaagagcgcacattgcacaatgcctagcacacaattaacacttggtatatgttggctgaagaacaaatgggttgtactgcatgttatttaatcacaaattttgaggaatttcacacaggactaaaattagtgttatatttacactaattattgagtcagatggctctttttctggcttatttgtgtgtgcacattttaactgcatgtcttacacatgtaaaaatattttttttatacaggctgcagccttcttccattgttgcaggctctctcttctactgtgggactttcccctctcgaatgctgaaaatagcaaaaaaagtgtgtctctcattctctttcccctcactctttgcagctaggcttattccctgagccctctctctacctgatgttctcgctaaatgcttatactctctgaggtcatccattgtttgagttattcctgacagactctcaccattataagtattaaaggggaggaagaaggggctgaggagactgaagggaaaaggggagaaacccctcatctcagaaaacccctccagaaagatcatctctttaggaaacaaggcaaaactgcggttggtatttctccactgtggccaaatattttgactcaaagtctgatgtagcccatgtttaattctgctctgtttattgatgtatgtttcatgaatgggaatagagtttgctgttttactccaatataatttatagtacatttcttctgatacattataggcatatatgactggcatattatttgtgcatataaataatgtatttcctcttctttttttcttttttttttttttttccagagacagggtctcactttgtcacccaggctggagtgcagtggtgtgatcatagctcactgcagccttgacctcctgggctcaagcaatcctccagcctcagcctcctgacagctgggactatagacgtgcatgaccatgcctggctactttttaaattttttctacagaaggggtctcactatgttgcccaggcttgtctggaactcctgactgcaagcaatactcctgccttgaactccccaagtgtgttggctcacgcctggaattacaggtgcaagctaccacacctggccctcttctctttcaattactccttcgatgcttataactggcaccaatgtccaagatctttactgactccaggagcccctatgagcacacagatcattagactctcagttttactccccaagtagttgggatgccctagttctgaaatgctcttccttgcaaccggagttgaggaccctggtatcttctcatgtccttccttctcacccatcttcttgaatacgactccccaacacatggacacacaagcatgtgcatatacacacacacacactcttcacccatccataaggtccatgctccccagggattcgggaatctagagggtgctgtcagcagcctgtctggggccataaagcataactgccaagctgcaggattgaagacaaaattccgatgcaaagataggagcacagatagggacatacaaaggcagatggcaccatagcccaggagccctagatccctggatccctgggtatgtgtgagcaaaggggaagggagtggtaacggagagggtgagaaactcatgcagctattttgtagtaccttcaactcaggactcctcaacctcttaaaaaccagtttcatcttaacttggtttaaccaagtttgccttcagtaacagtaattttttatttaaaagttttattatgtggtgagaatgtttaaaaacttactcttagaaattttgcagtatacaatacatcattattaattatggtcaccatgctatgcaatagatccctaacacttatttctcctgtttacctgaaagagtaaattttgaatattctcaccctaaaaaatttgtaagtaggtgaggttaattagcatgttaattagctttaatttttctacaatgtatacatatatcaaaacatcttattgtaccttgtaatttatacaattcttttttttttttttttttttttgaggtggagtctcaccctgttgcagaggctggaatgcagtggcacgatcttggctcactgcaacctctgcctcctgggttcaagcgattctcctgcctcagcctccctagtagctgggattacagatgcctaccaccatgcccagctatttttttttgtattttagtagagacagggtttcgccatgttggccaggcttggtctctaactcctgaccccatgatcctcccgcctcagcctcgcagagtgctgggattacaggcgtgagccaccacgcctggcctgtaatatatacaattcttatttgtcaattaaaaataagagaatattaaataagaattaaaactagaaaaacattttattattggcagtatttgggtcatgcaaaagaatatatgtaacatatctcagtattgtcacacaatgacaacacaaatacttgtgaggcaatgaagttcacatgtcaatgctgagccccgtatctcaagtgcccctaacttttcatagtcacatgacattccttttacagaacatacaaactttcatgctagatattgaaaagaagtctaaaaatgcagctaccagggggaatcaacaagttggtatgaatgagcttcaggcaaggcataaatggtggcagccagttatgagagggttggaaggggaaaggttcttcaggcctgggggatgaggggcagggaaggattcatacctcctaaggaggaggaagaaaggggggctagatattaaaaaagaaaatgccccaagttttataagacttctcccacacgtgcaccccactaactgtctctattcagagacatataaaaattgtggacagaagtacttcaaaatctgtttcgtcacaggtgagaaagtacacagctttgtagagactatttccaagatggtaatatttatatcggcaatatttataatttatggagtgcatattgagtgtcttatgctaaactctggggctataactcagaaaaatgagatttagagctcaggcatgctacagcccagaaggcagacagacaagaacttagaaaattccaaaacagtctcaaagggtgattctcttactccagtttgatacacacttccactgcatcctagagcctcttcagaattaagataaaacatttcttggtgcagtaattttttttttttgaggtggagtcttgctctgtcgcccaagctggagtgcagtggtgtgatctcagttcactgtgacctccgcctcccaggttcgagtgttctcctgcctcagcctactgagtagctgggataacaggcatgcaccaccatgtcaggctaatatttttgtatttttagtcgagacagggtttcaccatgttggccaggctggtctcaaactcctgacctcaagtgatctgcccacctcagcctcccaaagtgctggaattacaggcgtgagccaccacacctggccaagtgtagtgatttatttaatgcctgtcttcgctgatgttttgttagctccaagagacaggacttgtgtccatcttgtggactgccatacactggcaccttactctggctggcgcttaataaataatcattatataagtgagtgggtaaatgaatgaatgctagaaaagaggcaagcatggcgtgctgagtggccagtaaccatcctgggggtcagagaggctttggggaagaagtgatatttaagccgaaacctgaaggagaaatggtatttagccaggtaaagaaagagcaacgcttcaaaagcccagaggcaagagctcttggagcagcctgagggtactggaaacgaaactgcatggctgaagcttacagatgctgctctgtgcatgggctgttgtgcaaagtagaaacttctcatctcgaggtgaataaattgcagaagggcgtcccctctctaggccaaccaatgagaaaaaagagacttcctctgggatgattgagtctctccgctgtgcaccaggaggctgaggtcagcctctatatgagcctttgtcggggagccctcctgtaggatgccacctgcacaggcatgcccagaagccttgggaggagggaggagggtgccgagagctgagttagatcttggtaagcaggagctgactcaaaaacagcttttatggcactctaaggagtttgaactttgctctatgggcagcgatgcacttttgaagaactttaacctaacagtgactctcttacagaagttcgttcaacagacatttattgtgctcctgcttatgcaagaggcacaccagtacatgcggttctgggggtacacatggtgaggaaaagagacacggtacctgccctggtggaatttacgctttactagtgaaagaacgcatactgggggacaagtaagagacagaacaccgtgagagcagcttgaactagagggagaatctgttaaacaatcctgcaaggtcaacgtggtattctgttttccagataaggaaactgaaggtagggaggtttaagtaacttatccaagaccacgcaagtgagcttcaaatgcacttggtgtgatttcaaatcccatatccttgtactacagagataagaacaaaatgatgaataatatgttcttaaaaaaaacctgaaacatgaatatagaaaagatgtatcattacatttacacacactcacacacacacactcacacccacacatcccctttatcttcctttccactcacccatgcacatataccctagtgaaataaaacccgacccaccttacttttcaggaaacacagtctggaaaatatttgtcaggacatggccaatggcttagctgtaatccctatatgggcctattcaaactagttaattctttcctggggaaaaccattctgtaattgcagaccttgtctcttaacttagcagagcctctgacaggtggatgccatttacagggtcccagagctgtgaaaagccagtctgactgtctgttttagaactcttaagttcaggtaagcaattttgtaaattaatgtcagttatctctcttttttttgaagctggttggggtagaaactccatgttccaaaattttgtcaccctgaccaataaaatgccccctgctttcaaacacaatcagacctgctttaggggaggatcgtctgtgctttgtttgagccttgattaaaatggagatcagaaagactcacagatgtctcttctccaaactcaataaaccccaggtcctcttacttttcatcttacattttctctctttataaaagtattcatttctcttctttgaattatcttctttcaaataatttccttcgttagaaaaaatgtaggtatatgcattttgggaaacaagagctaccaatggtaattacaattgatttttattattcatgagattcctattattattattctcattgcagttatgttctacaggatcactacgaacactgaatgagtgaatactgaactacattgctcctaggagaaaaatagggttaggttcctgcaaacctctggtcaactggccaatacataaccttgttttatgtgtatttctgttgaaaaacaccttatgtaacatatattattgattcattaacattgaactcacatccagcggccatataactcatgcctgaatgaagcttatctaacgcatgtattttctctgtaaggcacatcacagccttctggcatttagggacaccaggcagcgctgtgctcaggggccattttgaacagtgaaaccaccaacaaaaagcataaaaatgtgaagaacgtggtacaaaattgatggcaaaaaggacacctgtttacaggataagggctgaaacgagaaggcctagtgtcaccttgtttgacctcaggtaggaacgtgtgggttggtaactcgaatttttcaccactctgcatgtccatgaatctctctgaaagccccatgaatattgattttgaggttacaaataaacttaagcaagcaggtgaattggcaaatacggaacttctgaataatgagaattgactgtatttgttcttctctattaagtaacggggtctgttgcagatggaagaaagaaagggtgaaagatttttggagactcaggagacttttttgaaaatgatttgcccaaggcacagaaccaaaacagtagttcattggcctaaggtggagaaatataattccttgcatctccagcaatctgtctgcagttctctccctgtgaaaaatgacttgccataatatacaagccttgttactatgtaagtgtaattatattttaataaaaaatagatttataaccaaacaataagccgtgagattaacagcccagaactggctgtgaactttcagccatctaaccctggcactcctgcttctccgcagcacttactttctgccaggagtgggtgattgaacaccactgaccaggaagggctcctgggagccacattttttgcttggcatttttaattgtaaaaaccagtgttccaccaacactgcaattcaacgaatcaaaagtcaaatccatgcttaacaggaaaagataaaagcatcaacttaaaggaatgaaacaaatttgaaaagcaatggaaaaggaaactcatatggagtgaaatccattcaagacacgttttattgtttttcccatatgcatttgctgcccagctttgactattaaacattatttttttcttctactaaaactaaatagtaatttgacttctagattcttccagggtcgtgctctgaacagaagcaaaatggaaatatttcagtagctttttagacaactatttcaaaagcaaacacatggttgtctgcagagttgtaggttaaagctataaatttattttattaaacagagaggaattggtgaaactgaaagacactataaccctgacattttcatgaaataatattcttgtctgattttttttttagtttttactcagatatcactcaagttacacataaatggtaatttttctttataataagcttccatctcttgtataaatttaatgtgttcagtgatgactgtggataaagtcacctaacggtgattttctaagggaggtaaactttattgtcataatttcctggttattacctagtaaaacaagtttttaaaaaaccaaaaaacaatagacttaaaaccataaagacttttttagcattcagattaaataaaagaaagggtaaaatgttgacattttcctaagagataccagttcttcacatttttatataatctcttaatatgaagcatataatgtgtatattataattatatattttatattttattatataaaattataatataatataataaagtgtttatatttataatataatataataaagtgtatattatattatatttataatataatataataaagtatatgttataattttatataataaaatataaaacatataattataatatacactttatatggtaacacgatcatagtctctaccaatacatatatctgattagatcaccatatgggaagtctgttcatatttttagttattgggtttttttttcaccaagcaataaaatgtccagtatcaaataaataagggtcatatgattcccagatgacatgaccagggtactcataaaattcagagaagctgagtcagatcatttcaagaggacaaatgcagcgctcccccaaattcagtaacttgtggaatgtagcaatcttgtattttctctgagccaagcagcctgatgctgtcaaactcatctttcatttcctctgccttaccgcagtgttagtggtttcgttattagaaggaaaaaaatgcataaatgtcttagttaagatgaaattgcatctattaaaataaagttgatcctaaatgattctttcactcaaagtattaatagactaaaggaaaatgttgcaaactatgaattagtggtatagtttatgaacggtagctgttgtgattcatgcatataccttttggttaccttataatgaaaggttgttgggtacagccttcacatcaatgagaaaatcaacaaaatgttcttgaaaagacgactggcaaactttcttttatttttctgttctcattagtagaaagagggacaggtcactttcagaaggattaaaaggtcaaaaaatttggtgaagggatgtctacacaagcctttagaaagctgttatcagaggatcagctagaaaacaatcttagagctaatcctttggctggatccaaagaaaacccagcagatttaagttttctcttttttctctttgttttcctttacaggggtatttcccacaaaagcctttcagttgccttcatccaaagttataggtcctgagtccttttcagctcttttattctgtctccaagcctagcccttcctagctctgtcctcctaccttttcctgatcttttccattttgcttgttttcatcaggggttccccctaaatccctcttagcatcccctaaagcctaaatgttcatggatggagaacttcttctgagggaaaaacaagactgattcacagaattctagcattaaagaggactgtagaaattatctagtctacccgccttcttcctccaccattttacaggtaaacagaattcacgaaattgcccaaggtcatgtccaggtagcgactgcaccaggaccaaagctctgtcaataataactgcaaattggttgcaaagtcctttggacaattcaagctgctctacctacctaaggcactatgattattattattataatacagatgttattatacagagccgaccgcatgggcaagattgctgctgatgatgtgtgtgatgccaactcaccaagacccagtggcatcacgctggcatgttgaaaataaaaaggggtgacatctgctttacatacagagcctcatgacctgtactcatagctggaggcatcagtcattgcactacgagaggaagtgggctttgaaataccttggggttgatgtcccaagaacacccttcttccgctcctcctccctgtactccttgaactgctgctcatgggagcctgagtaatacagaaattggaggacaaccacacagcacaagtgtaggtttgacaggagtttacataatgctgtaggaaaagaaaccaaataaaggcttcagctttgctgaggctctccccatccccctcacatgccaaatgctccttgtgtggctcacactccttggtcttcagtcagagaggccaggagcccacctatacatctgtatcagggttaggaagttggctcttgttaacaatgttgaatgcactttgctattcactacttttgtggttagaaaagcctcctgatatcaaactttagaatcaagcaccaccttgtggtctgatgcatttattaaactggcacaatttgagagctgaaactggaaatgctccctcagctggtagtagagagccaggatctgaaaactgactcccaggtggctgcctggaagcaagaacccccaaggtgtctttctgctgcttctctgggttctcagaccagttaacaatgacatcccctttcatggggtgaatcccatgttcctgacactaacacattatgctagatatgtatagtaagtagccaacttaatcctttcaaaaccacagggaagtgtttcttattacttcaacttttaagtgaggaaacggaggcggaggtcacttagtggtccagtcacagacaaatggctgggcttggattgacttggaagtcaagtctttctgaagcaaaagtctctgctgttaaacactgctctgctgacctctaaggccaagaccaagaccagggtccacttcaggctcttttggggcattttggctcttggctgcaaacccaaccagaactgccagaatcctacctggaactcactcaaggtgctagaattaacctgttctccaaggttacattgatcaggaaggtttgcattcagagatggggacttcattggtgtccatggacctactgagatagccagagtttaggagaaagtttggcttttgaggatgggccagacagcaagtgaccagactctagatcattcatgaagaaacaaaaccagaaagagatatttgacaaacattcacttggacaattacagtgcttcctgaactgaaattcagttattctttaaaaggatgattttatcatattcattgtttgaaggggaggattggagtatctgcttggcaagtgagtagtagtaatgtgggccaagtcatgttagaagggagatctcgcccctggtcttgttccagtgataccataagtgttcttgggtaagtcacttctttgtctctgggtctccattttctcattcgtagaataggggttggacttcactaagaggtagatggttctaaagtacagcgcaattgtatggaaaatgtgaggcaggaacctcagctttctccgtttccttagaaagtctgtcctgctcaggcttccttcctattctccactgaatctgctgctaattttggtaacatggtgacttccacatagcaatgtttactaacatgtctaaggacaatgattatcgggaatatatgcttctctgtttttccaaagtagataaatccatatagttcataactccctgactctccatacaccaaaaagaaatacaaaaagtaccatgaaaagaggaagcactgccatgaacggtctccccttgaatggctagtatgtaatgccaagacagtgcctgttcatttcaaagcctctcccattctagttccttcctgtgctctcactccgaatttcttacttgagatcgctattggttgagccattcttttaacagataatctgtactacagtagactctaaaacagagctgtccaacagaactttccgcaatgtagaaaatgttacttgtgctctgcagtggccataggtgactattgagcatttgaaatgtggccagtaagactggaccaagtgtttacttttatttaattttcattaatttaaatttaaatagccacaagtgactggtgtctaccatattggacagtgcagctctgcaatattgccatcacataggcctttgttgcagacattctgatttctctgagacacaaagcgtacatcataacagggctatcctgaatactggctttacaagcagactttcaaaagattgtttctccatgtttttcttgtatacaagatttcataacaataatggtatgtcagtctttttaaactgtattctgtttgagataactgaattgtttcttgacttatttcaactgaatagttgaaaaggaatccctatttgattccatgggaataaaactgattccttggttgcgaataaaaagaaaagtattatctcactttgtaggtttttccattcaataatagaataaatttgaatgtacattttctaatggcttccagtgaatgagtaattttgatgccaaacaatttttttttttttagaaggcatctcgttctgtcacccaggctggagtgcaacctccatctctcaggttccagtgattctcctgcctcagccttcccagtagctgggactacaggcgtgtgccaccacactaggctattttttttgtatttttagtagagatggggttttgccacgttggctgggctggtctcaaactcctgacctcaggtgatccacctaccttggcttcccaaagtgcagggattacaggcgtgagccaccgtgcctggccctgatgccaaacaattaataataaaagtagattttcactttaaaaagggagctttatatataaattcatttttcctaaaatatttgatatggaagatgaaattaggaaaaaaactggtaaaattctaagtgctttattataaatctcttatcctgagtaagctcaagtgacagtccaagtatgtttctaattaattaggccaatatttctttttttttttttgttaggaaacttttacttcttaacttctatcaagtatatttcttttttttaattattattattctttaggttctagggtacatgtgcacaacgtgcaggtttgttacatatgtatacatgtgccatgttggtgtgctgcacccattaactcatcatttacattaggtatttctcctaatgctatcccttccccctccccccaccccacatcaggtaccggtgtgtgatgttccccaccctgtgtccaagtgttctcattgttcaattccctcctataagtgagaacatgcggtggtgtttggttttctgtccttgctatagtttgctcagaatgatggtttccagcttcatccatgtccctacaaaggacatgaactcatcctttttatggctgcatagtattccatggtgtatatgtgccatattttcttaatccagactatcattgatggacattggggttggttccaagtctttgctattgtgaataatgtaattaggccagtatttcttaactggaacaaagattgagtaacattatccacgcttatctcaaaataaaaccttttaacctcaaagaatataaagaggttttcaggtcggagatagtcctgagaaagtgatacctaggctggaagagaagaccaaaaagtcttacaaaattgtaaacttacaatttttttataaatgtggtaaattttgataaatgcttgcatgagaaaacatattgctgtgttttagaatttgtgagggacatcagtaagcaagagattctgactttgctctggaatgagagagtggaactcctagtgtctttcctcataatctgctgaacgactgaggttttcatcctaggaccatatgatgacctgctactccggggctgcctgtgctttataatgatgctatacagacactgttgtcctatgggatccgagctccagttgaagaatgtttcatatacttctattcttggtgcagagaattgtctctactatagaaagtgacttaacactcccaaactgcatcgtctgagaatgggcatttctcatgtactcaaaggttaatacatggaaatcaatctcaggccttaaaaaaggaaaacccagcgggcgcggtggctcacgcctgtaatcccaccactttgggaggccgaggtgggaggatcacttgaggtcaggagttcaagaccaacctggccaatatggtgaaaccctgtctctactaaaaacacaaaaaatatttgtcaggcctgtggcaggcatctgtcatcccagctactcgggaggctgaggcaagagaatcacttgaacccgggaggcagaagttgcagtgagttgagatggcgccactgcactccagcctgggtgacagggcaagactctatctcaaaaaaaaaaaaaaaaaaagggaaacccaaaatgcagcagaaaaccaatttaagtccgtgcttctatttctttgtgatttggttttcgtgggagtttgggcttgaggggatgtaggcgttcctctggggtgtgatagcacatttgtgccatgataaggccagtccctgtcccagtggagtcatttctcaagtcctgcgtcatggggaaagtgaaggtctcccctgcccactctactcccactcaagtggctgcagaagagggggatattaggcaccataggacaggaagagccacagactcagtgacctctatgcaatgaggaggttggtcagagcgcatttgtcagagaaccagctgtgaaatgtatggaggcggaggggaagcactttccaaggttaaccaatccaacccttagactgccagctgtcctcgcttagaaccacggctgatgcagctgtggttgaagcatggacatctagttatcgtaggaagagagatggagaattcttcccttgcagtcaatttgtaagctgatgcaagaaaaccaccattgtagccacaaaatcaaaatcaaagaggaaagtgaaagaataggacccaggcaggctgtgcaaccaagcatttcctttagctcctcttgaccaaaggcatcccaatttcccagtgaagatgaattagaaattacatgagaaccatacggtagattatttctggattggtgtataaatatatacagcactgacgccaattgtttgaaaaactgcatgtgttatatcagttgggtgagtttagctcttctcaatggatcttttagttattggaataaaattccaacttatagcaacttcttcagtataaattaggcagactttcagcaagtcctgtgaatacccagttcctggcacacagtgggcctcattacaagttgactgaagaacctcagttctgggttctgtgacaattctgggcccagagttgtcacagggcaaatgagataagcagcattgcttggagaatgttgcctcaatcattcgtgttcctttctggctgtgatgtatattacactctccatagtaattgtctgtctccataaacccatttaaaaatacacatgttaacataataggattgctaattatacttggatgttcttgttcctttttctgactctataataaagcctcaaaatgctagctaggtcatggccacctaaaagaaattacattttcttgaccgccttccagcagatacggccaagtgactacactttggtcaatggatataaacaaaagaaatgtgtaaggctccaggatctatcttttaggggtaggggtaggcccttctcctgcctcttttctcatccctacctgcaataacttaaatatgatggcttaagctagaagaaccattttggaccataaagtaaaaatcctctgttgaaaatagagggaacaggcctggtgcagtgactcgcacctctaatcccagcactttgggaggctgaggcaggagaattgcttgagcccaggagtttgagatcagcttgggcaacatagggagacctggtctctacaaaaaatacaaaaattagctgggcatggtggtgtgtgcctgtggtcccagctattcaggaggctaaggcaggagaatcacttgagcccagaaggcagaggttgtagtgatctgagattgcaacatggcacttcagcctgggtgtctcaaaaacagaaaacaaaaaacaaaaacagaaaaggaaaaagaaagaaaatagaatagcatgataaaaggagtgtaggttgagtgagagagaggatttggtggaacagaggttataaagtaaaataaaagcaaaggtattatggataaaatccatgttcagacatcatcattctgattgcttataggcagaattggtagaattcaggtaggccagatgccatgaaattttgtattgacaaagagaccagcagtctagactgcaaaagcctttgaccattcaatacctaaggtgaccacctttcccagacagccagcaggaaggctgtgaaagctgtgaaggctttaagaaaggcgcgcacttgggagtattatgagctgggaaacatttcctaaagggagagaaaacccacaggcggcagtgaatgaagaagttttgcccagtgagcaggtgagagtccagctgactctcatgattttgttttctctttctgcccagtgggatctgataatatttagagataaggaactattatgtgattctcagtttttaatttttctgagtgggagaaatgtttgtttatataaagtttgtcctgttcttattggacttgtaggtgtgttgtattaggtaacttacatttctattttgtagggcccctgaaaatgagtagttacactcagacctgataaagagcatggaatagtacctgaaaatcctggacttagaaccagatgcatgaatttggtggaactctgggattctttatctgggagtacaggggttgagtacatttcaaacatggaaataagggtacactgagatacatgggtggccaaaggggagacatagctgtctttcaatatctattatcccttaggttctgtgaacctgaaagaaccaaggactataaagagcagtggtgtacaactcccaggaattgtctttaatgggagatcatgtggctttatcttgtctcttttttgcatcctgctgcctggaacacagatgtgatggctggtgttgatcctgctgtgtgctgagggagatggaacatcaaggtagaaagcactggaccttctaagcatggtggagccattatgccagccctggatcatttacattcagatgagtgtgcaaatttttattcccttaaagccacttagattttgaattttcagtcactctcagctgaacccaatccttactgctcttggaagacatttacactgtgggaaccttaggatgcacttttttttcttttctttctttctttcttttttttttttttattgagatggagtctcactctgtcactcaggctggagtacagtggtgtgatcttggctcaccgcaacctctgcctcctgggttcaagcgattctcctgcctcagcctcctgagtagctgttattacaggtgtgcaccccacacccggctaatttttgtatttttagtagagaccgggttttggtttctccatgttggccaggctggtcttgaactcctgacctcaggtgatctgcccgcctcagcctcccaaagtgctgggaaggacacacttttataagcaggcatctttggagatcttggtgggagagatggctcaaggagaggaaaagagggaggtattagtacaactgggtttttataggcccagcactagcttcataaaagttatttgcaaaaaaagtacgggtctgtgtttttgtctgatgaggtggcccattgctctatggtaagtggaatacataattgctcacctttgcttttggaaattaaatgtatttagaattgaaaccagcttatggttttcttctcttactcatgttcatagaagatgatcctatgagtaacactttttttggagactcaatcattgtttccagtctcttgcattctctagtaggtggtttatcttcagtaacttttgcaaattgcatcaacatacttgcctcctacaaatttaaactcaataaaccaaggagaacaagggtgtgatggtgggaatttcttttgaaagagtcaagcccatgctgtgcaggctgcacccatctttactgcattcccataaaaggcttgggggaaggaagttaaaccataaccccgttaggtaataggctatgcattaatgcaatttatctggaatgaggaaggaagagattcagctttgaaagaatttcctcaacagcatattcctctttgctgcaaagctgtttcacttcaggtgagacccaggaagcagcctgggaattccttgaccatcgataccaaaggctttctggaggtgtgcagtctctagggatttgagggtgtcgcccaacagtcagtgtgattttacaaccagttctctctaggctaatttctaacgcagggaatgagaatggtcatgctgaaaagtggtagagagatttgactcgcctgattgcaaggaccaggtctaccacccagtaattgtgtggactagaaggtggtggagtgagtagtgacctctgaagagtgtgacctctcaagccaactgcttgagctccaattctgcctctgctactttgtgactgtgtagccctgggaaagttattttacctctctgtgcctcagtcatttcctctataaaataggtctctatatggacctaccttataaggttgctgtgagtggcacatgcaaatgtgtttataaagtatatcccatggtatgtggcgtaatttattttttgaggggagatagtgaggtgctagttacacactgggatgaggtagatccttgtttggatctcagccttgctgctccctgggtaggtggctttggatcgctgctcaccttgccaggtccctctggctcctccttggctgaacatgctttgactacactgacctttctgtttctccagccctcctcatggcctgtatataatatttcttccagttggaatgctctctattaaccattatgattctcatgaaggctgtttctgttctccaggctgggttcaggtctctgtgatatcactcaaggagttttgtacctaccctcctgagcacttagaattatgctagagtattcatttgtataattacagttgactcttaggcagctccagggatgggggcaccaactctccttgcagtcaaaaatccacagataacttttgattccccaaaaacttaactagtaataacccgctgttgaccagaaaccttagtgataacataaacagccaattaacacccattttgtatgttatatgtattatatactgtattcttatgataaagtaagttaagaaaagaaaatgttatttaaaaaatcataaagaagagaaaatatatttgctattcagtaagtggaagtggatcatcttaaagggcttcatccgtgatgtcctcacattgagtaaagaggaggaggaggaggagaggaggaggaggaggaggagaggaggaggaggaggggctggtcttgatgtctcaggggcggcagaggtggaagaggtggaggaggtgggagggaggcaggagaggcaggcacactgggggtaactattattaatgaaaatccttgtgcaagtggatctgcacagttcaacccatgttattcaagggtcaactgtattcctatctgtactcaggactgtgagttccaagcatggttaacaagtcctgtgtctttcaacccaatgtctatactggtgcctggctcagaggggacaaaacaaatacatgaatgctgaattaatacatacatacataaaatgaatgtgagaagaatttccttttctgatggttttataaactatgagctacttgaaggcagattacatttcatctatagttatatccccaaaagcctatagttagcaagaaattaatgtatttgtcatgtggatgagtaaactgtaccatagtgattcgaatttaattcactaacatataacattctgatttgaaaataaataaaattactttagaaaagcttatgttggatataacctaatttatacaaatattctttttttatttgtcaattgagaaagtatttagttactcgtgatggaaactttctctttttttatattagagatgttatctcactgcagactataagttaagacttgtagcacagccattctgatagattttgttctcagaattcatttatcctctaaaaagtattgaggatcccaggaagctttcatttgtgtgggttacatttactatatcaaagaatatatttactatatcaaagaaatattttaaaaacatttaaaatatttatttgtttaaagcaataataaactcaatacaaattaaataaattaactaattttaataaaattaattatattttcctatggatcttttgcccatgcatgattttgccaggtaatgcatcaatcatttggaaaatattaattcattgagtgaatatttgacctttcaaatgttggctatacaatattttgaaaaatcacatttgttactatcactgatctcattagaaaagtctttgaggcattgggaagctgtctggtttacacaagcaggtacaagtttaccaaaattcaaagttttgcttgaaggttcaaatgttattttcagcaacgaataatgtttgttattttccttgaagtgacaggctctctatttagtctcaagaaaaagtttgtcaaatagccaaggctgaataacaatagtttgcagtcagttgtcttttagggaaaaaatgcttccatgcaaaaataggctatttcagcctacaactcatagcaagtgtgtaagtattagagtatactgtaagtgcttcatgctcacaattactacaaatgtcaacatggtaaagaaggcaaataacgtcttaatattatttaaaaaatagttttgatcttatgctcctgcaaaaggatcttagagaagcccaggggtccacagaccacactttgagagccgctgctctagggtattctaacattaaattacaagactcattgaccccacacagatgaaatttacaacaatatggcttttaattaatcaacttttcctgcaacttctaccctatcaggaacaaataagccaagagcaaaatgttacaaaagattgtgataactgagtttaagatcaatgtacgagaactgtcttatcaatatcacccataattattagtgtaaagaagtcacaaaatgtcttttaaaaaattctaccctgaaaatatttcagaatgtaagaagtggcaataattataataagtggctacaaactccactttgaatgcattttcagcagcttttttcctaaacatttatcacgtattattttattttaacatgtctctgtgatatgggtacataggacttgatatcatctctgttttagagatgaggaaactgagaaaaaaatgactagattcacccaccattgcctaggaaactggcggtggaactgggtagctctcctgatgaatatttcaatgccttttctttttcaccctgtggaatcacttggccagccagactcagcaaacacgtttcatgtcaataaaaagagaatattccaatgagcccagtgactttggaaatccctaaagtggagtgttccaagtaactgaaaacctggggggaaagagtgacctctgtgagcttttccagcttccacttctgctgatgggtggaagttcataggaaggaagacattctcttagcctgggctatgtcccttgctaaatttaggattccagattatagaaacaccctcagatgaccaagccctccacccctgatgttctgggagctctcattctgaaagaacaaatgcatttttccttcccctttggaaccagatagtaaacttggccaaggagcctgccagccactggcctggtgtgagtgagtggggtgattatttggagatttaggcagaaagtgttgaagggaaagaggctgtgggtggggagggacaatcagctaaagagatgggaagtatcatcctcttgtcttgttaaagcagaaaactgtttaaaatgtcatgcccatatttactagctgtttataggtaacagaatctcctctgtgtgtcgtcagtgagtaattgccataacaatgttgtatttacatgacccatattttaccccacaaaggcagagaagatccttaaagaaattgcatatacctctatggtgtgtagagaagaacagctgctttcctccctcttcctactcatatggctcttgaaaatgctctcagcctcttcctttaataatttccatcatgtcctcctctgccctttagattgtagcacacgatttaaatggaaggtgcagggaagtgtttctgagtggtttgcctgactcaagccacctggtgggcaaggggcagcatctgagaggcttccttccacagcccgcctcttagggcccaagagataacaagaccacatgcagctcagatgcaatcacttcttaacatagctcagaggcaatcacttcttaacatttgagaaaacttgcctgggtgaaaaaccattttcccagacagcaatttgcctaggagttgcaatgacattgtctttctgtgccaagaatatcaatcagttcaaaagtccctctcctgagagccactggcaagatcctgggcgtgcacacacaagcagtatctgtaaccctgctgcttggggttagtgtaaatgtcaaggctgtgtattagaattaattgttctgccactatctgctcttcctttctcttttcctccttttgtataatcgtttgcctttttttttctccctcgatcttgttgtttctagctttattgttctcagtttagacttgtgagcctttgggatgaaaagtgatgtaaactcttggagaggaaagaaccctggatcataggccagaaggcctaagtcccagtaacactctcatccgttagtccagtcacatcactaacctatctccacctcagttttccatctcaaactggagataataatctgtatattgattgctttttgagacccttgtaagaatcaattgttattatgtagcactattgaacatatgtacttactttcattacagtacctagaacaccagctagtgcatagaaggtgcccaataaacatttgttgaattgatgactatatttggaaatattttgtaaactatgaaatcactacacacatgtttttatgacatttttcatagatgatgctactagcctgcagtgctctaggccttgggttgtgtcttgtcatgtcggctgtgtaaacaggctggctcctgccatgccacaggtggctggggatctctttccaggccacagcatctacttggtttctgtgattccaattcctgtgtggatctgacccactaactagaagtttgcgctaagtgaaccttggtgtgtaaatgttggtgctggagggtgaatgtgggtaccttgaagccctggtcttataaatgagtaacatgtttctgtataacatggaatgatacagatgatggcattgagaatcctgctggtctcggaaatttcaagggagttctagaccagatcaaacaagagacaatggaagataccagtatttggcaaaggaaagacatttagaatttagaagtattctaaaggatgatttctttttttttttctttctttctttttttttttttttttttttggagacagtctcactccttcacccaggctggagtgcagtggcgtgatctcagctcactgcaaactctgtctcctgggttcaagcgattctcatgcctcaatctcctaagtagctgggattacagacatgcgccaccacgccgggctaatttttgtatttttagtagagatgggatttcaccatgttggccacgctggtctcaatctcctgacctcaggtgatccacctgcttttgcctcccaaagtgctgggattacaggtgtcagcaaccacacgcagctctaatggatgatttctatttacatccttcttatagactgagacagatccccctaaaattcatgtgttgggaccctagcctctgtatttggagacagagtctgtgaggagatgttaaaggataaataaagtcataaggttagggtcttaatccaatgaggctggtgtccttataagaagaaaaggagatactagatctctctttctccctccactcttgtcttctgctccacaaaggaaatatcaatggatattcctcacaagtctaaactgagaacagtaaagctagaaacaacaagatcgagggaaagaaaaggcaaaagattatacaaaaggaggaaaagagataggaaaagcatagtggcagaacaattaattctaatacacagccttgacatggagcaagaaggaggctgtctgcaagccatgaagagagagctcttaccaggaactgaatcggccaccttggtctgaaacttctagcttctagcttccagaactgtgtgaaaataaatgtctgtgccttaagctacccagcctatggtatttattatggcaggccaagctgactaatacaaccctcacctcctctgtctcatttctgggtagctatcctgaaattcatgattctgacattctcctatatgcaaagaacattgaagtctatgagcagactgaatgtatatgtctaaatatccttgtgaactaagcatgatggaagagaggagacaagaatagccacactccactgaatgacaagtcaaagattgcaatgagcaactcttttacctggcttgcctgagataattttgatacattacactaagcttcaggagagtgaaagggtgtaggttatgtcaattagcttgtggactttttgcaaaccatgtgattaaggctggtgacattctgcatcaggttttaacaaaacaaggtttctaaagttagtttggatacattgatttttcttacaccaccactgttgctagttgggtatattaagtgatattatagcatacgtgcaaagggaaatgattatttgggacagtgaaaaaaactgtatattcaatgtgatgatagaacaagtgtgaaggctgagtttggcaattttgggtttgatcctcaacctctaatttttgcaagtttggagccatgatcttctaattatttagcacaaatgttgaagaagttagacacaaatgagtcaaagttaatctatcacattcatttttaaactttagtgtttctatgaatcatctgaagagcttgttaagagccagtgagttttcgggtgagcctcagattttgtatttctaacaagtttcagctcatatcaatgctgctggtccatgaactgtacttggagtagcaaggatctagaatactgtgtgtcagatggaaagatgagacatttagtttttctccatgaataaaatacgtgagattcaactgttgctttgttttgcaatactatgggattcctctctaaaaattgttcaaaatctcatgctttgaagaactacattatatggattgtaaaatgacttaagcaggaagtacttgacatgaatatgtgcttaataaatattgattggttgatttctgaaaagtagatcttcagagatgtaaggcaaatgtcttccagtaatataaaatgatacatccctacttaggggatctaggatgctatgcattgagacaagacaaagtttatggtgtcagagccagagagaaagcaagggtgatctgagcccagcaacacagctccattgtaaatagctgctcacctggggcaaacaagtgctataggatatgataaggaaggttcatttcatcattttatggctgtgtacatcctggagtgaaggaaaggcattaacgagtttggtaagagctggcaaagccagagtccttatttgcaaggcatggtaagcctatgggtgtgttatcatttacttcattgtgatttacgacagtcctactgctgattatacctctctgaccccctcagggttggttggaagttgtttagatcctacctttttctctccagacaccagagactgccagaaccacgctcacctgtgagtgaaacaagcaaattccctggcatcaaggggaagttaaaacttctatttgaaacaactttttgaacacttttggcatatgtggaaatgtacatatacacacacgcatatgtatgtggattatgtaaggggttataataaagtaaaattaagattcaagagcatttttgactttaagaaaaatattcctgacccatgcactcttatgtaacctgatccctcagaagcatacaagataaatcaaatttacagagttacaaaaaagtgttcactgactgctggaaaatatatgatgtcatatttgtggttaatggcctaagttagattaaaaagatgcacttttttgtattttgatgatttgtcctttgttttttctgttttagttagacttgaccatggaattgatactctgtcaataaattctttatctttcactgttgccactggtaagaatagctaaaggttttgcaggataattgccttgaagaaggtatcttcagaatccatttatttcaattatttgccaatattattcaaaatttcttgtcttcttatcactatcaatgagtagaaattttcaggattgctaagagtttgttatgctttgtgggagaaattctgtgtaagattgagttttgaaaaaattccatgttgcataatctggggaaatgcctgttcatgcctatatctgagcaaactttgtaacttgtttataaaggaaaaaagatttgaaatttaaaatttggcaattaggagcacattaactctccttgtctacaatttttatcgcaccataaaaatacacttttaagaaaaaaaatagcattccttttaaaggtttctttcaaaagatatccctttaggagatgggagttaaataacaaaatcaattactctctttgttgggtaagtcaaggactccctaagcccaaattttgagtgcctcttggcatctttgtatataacaactggaggttatccacacattaattgcctctaatactattcaaatgcataatcaaatccaacaagtcatttctgcttaactgatgcaacacagatgatgccatgcttcctgagactgaaaggtttgctaaggaaattgcctctaaaattatggcaagtataaaataaaggactattagaggagataccaccttagctgctgtacctagggatactgataaaggatatatcacaacactcattcataactgaaaggatattaaaatccaaataaagcaaaagaccaaagataacttaagccataccccaaaaagctaattaactaataagttattttactgtttaacagaatgaagagattaatggtagacaactctttagctggaatattggaaagacttacacaatgaaagcaataagtgacatagatcttgctaaaaatcaataaagcaagtaaagccagttggcacactatttaacgtgaatcaatgtgaatgatatctcatcaaacttgaggtgtcattcaagaaggaatacttgctatttctgtctcctaaatttccaagaagacattacaagtgtctaaaatgagcacactcttaggtgaccctgatagaagtgcattttatatttacttgtgaaacgtgccccaaaagttaaagaaaccaataactcatataaattcttgggtttgcaggatgtcagattaaaaacaagaaacagcttcctgaaacactaaaactccctccacttgtaagacaacaaaactggctgaaatcttttcgaaccgatacggccagctggagtttgcacagaacaagcttgctgatgtcacagcctggatttccactgcacgttttatattaactcctccccaaatttgcacatgggacctataaggaagcacaaagggatgactgtgcgtgcctgagggtctttcagacctctcctttcctcccaccaattacctgctaatctcagaatccaccctcttaaaccttttctagtaaaagtattgccttaaaaccagcacagagagatggatctgagcttgactcatatctccttgtaagtcaacttgcaataaatagcttttctttctcaaaaacttggtgttatattattggattctagaacattgggcagcaagttccttttgcttgatagcacttttgtataaatgtgtatgtgtaaggatgtaattaacagcatccttttaggtttctgcatttatattaggccaaatcgatataccttttagatattgctggacttggattcaatttgctttgttctccatttcatttactaaaatctgggtctaatgtaagagttcttggtatattacataaacataaatcattgctgctgaagttctaataggactctgttgctacttgcagaaaccagctttgaggtagtcaaggaaagaggataaagaaaagaacaaaactgaggttttaatttcatacatgatttgtgtctttgacagtgcttttgtttcatacaaaacacaaacgaaataaaaatttaggttccatttctgtcccttgccatgttaactgtccaatataaaaagaattgagcattatgaagtagggttctgcagcttgctagataataaagatattgaaagaagggttcaatttgggttggatgccaatagtggtgaagcatcctgcatgttcacgttggctctccagaactttcaggacttgataattctgcaggaacgcaaggtgggggcctgacttctcttaagccacaccaatgccagttaaatttgggattgacagaaaagtagagaaagaatagttaagtttgcatgaacttcatttatgtcttttttttcatgtgtgtattcatgcagtgttcactccctactcactataagccacgtgctctgcaagatactagatatacaaagatgaataaatttgtccttgaggaacaagtaattataaagttagaagataaagataaaaatatccaacctgtattgaaaaagaagaaaaagaagaagaggaagaggagaggtagaggaaatcacctatgaagaaactgaaatccaactaacctcagaggagttctttaaaactctacatgccaaagacaatgaagcaactaacagaattataggggaaaagtttgtgaccaaactataatctatgtttattctttgtggcgaggaccataaaatataaccccacacactcaaaggaaaaaatgcttgaacacattctatagcctgccaagattttcataaaatgaagagcttaaattgagttagaaacagactcccaatgagcaatgaaattagcaatttgaaagatgcatgcaactgtcaaaaaagggagagagaagatacataatgaaaaaataatcactttttcagtaatatttatcagaccccctcctatatgctagacagcattctagtcaatgagattacaacagtaaattaaacagacaaaaattaagcagaatgcaaagtctgcctttataaagcctatattctacaggagaatcagaaaataaacaagatacaaaaaatgcatagtatgaaatcacataatacctcaagtcttaatagcaaaaactagcatatggatataaaaggatagaaaaagtgagtgaaaaagggataaatattttagcttatgtagaagaggaagaaaagactttaatttccaggaatttacacacacatgtgcacacacaaaatgtagggtagaaaaattattgcttagacaaaatagaagtcaaagggaacagcattaaataggacaaaaagaataactcatactactactactaataataaaagtcaacacattcaaagtccatcttcttattcttcatctgactcagtttctgctcttgcctctctaagtgagggtattgtcctactgcccccaaagccaggccaaaaacctgggcatcattctttactcctctgtccctgcgcacacccaatctaccgcccgtgacgttttgtttttctacctcctaaatacatctcaatccatctccttccctttgtgctttctgacattttgctcatttagaacaccatcatctcttacctggccttctacctcaaccctctaactggttcccttgcctcctgcttgttttcatcaaatctactatccatggggagctccaaactatctttcagtctacccatctgattataacattattctgtgtaaaacacctttggcatcccattgtccagaactccttaatgtcaccctctgtagtttaactgttgattatctctccaggctggtatctcatttgttgctttttatatccagccacgtgagcatttgcaattcccaaatactgggtcagccgctttctcaccttgagggtgtttccctctgcctggtacccgctcttcttccgtctccctttgcctggccaattcctattgagtttagacaccacttctccttgtaaacctttcctcactctccactctgccaaatctggtcgagttcctattctgagtgttccaatagaaccttgaacttcccctatcatagtacctttccacttgattataacagcctgttgatatgtctggagtccacaccagattgtaaaccccataaagctgtggaccatatccccaggacaaagatctatccagaaggggttttaatattaaatgagaaatattatttaaaaagaagtaagaatgcaacctaagaatttggaaaagagaaaaataagccaaaagaaatcatcagaaggatttgaccaagattaaaagaaaataaatcagaaagtagaaaaattgtatttctttgaaagaaaacatgcacataataaagtgcataaatctgtagcattttatatataatcaagagaagttgtatgcactcaattgaaaaatttatgagaaatttatgagaaaggacaacagatatcaaggatattacaaattatgcttatgatttagaataatttttaaaatggcaaatttcctatctaaacataaatgatcaaaattgtcttaataattaagaaatctgaaatctccagtaactcagaaagaatttgaaaaagtactcaataattacttcaaaaaatggatactaacctgggcaactttacagctttcacattttcaagaagcaggtaatttctttcagataaattgttccgtgggataaaacaagatgaaagaaattatcaaattcattttatgaagatgaaatgattctgtttcaaaaagccaaataaataacaaacacaaaactcttgaattgaatagtaatcttatttttgaatacaactgcagaaatccttaaaagaattacaatttcaaaacaatagcaccttcataaattatccaccacatccaagtagggttattcctggaattcaagaatagctcaatcagagagaaataggttagcatactacatcaatggatctaaaaagaaaaaaattataaagctcataaagtcatttcaatgccatttggaataacaattcttagaaaacttaaacacaaatggcatttattcaaattgtcagtgatatctgatggctaaagagtagaggtactctctctcattaaaagcaggaacaagaccaggaagtctaatatcaccatgactatttataagaatgctctgaacatcagtttgtgcttgtatttgaccaaagtctgccaatgtgtttttgctttgattttggctgtggaagtctgaagagacaaatgtctgcttcaactggctttctagcgtccctgagatagttcctagtcaaggatcatgtgactatttaggttaccatcacatgtaatcattaatgattgggagttccaatattaactgacagaccaatagtctaagccctataacatcttcatttttataggtcaaaacaattgaatatcagctatttcttatgacttagtctaaaacaaatattgagagagagtgtgtttgctaaataactttaaagaggacagtaaatctctctaaagatatgttggaatgtctcacaaataagccaggttgttacaaggtacacaataaagagggatctgcttctagtttcaacttttcttattcatcctggataaggtgcttaggaaccactgtaacccactgtaactcctctaaaaagctggcacaggaaacaagacggcttggccagtgcagtagctcacgcttgtaatcccagcattttgagagcccaaggcaggaggatcattttagctcaggagtttgagaccagcttaggcaacaaagcgagagtcccatctctacagaaaaataaaataaaataaatagccagacatggtggtgcacgtctacagtcccagctacttgggaggctgaggcgggaggattgcttgagcccaggaggccgaggctacagtgagctatgattgcaccactgcgctccagcgtgggcaacagagtgagatcttgtcaaacaaaacaaaaccgcaaaacaaacaaaagaccccccgccccctaaaacccaaaaaagaccattaggcacctgaactcaaaatgtttaaaatgtttttcctcacctgcccacctctaccctccacagtgagccatcaacctaataacaagagagagcaacaaggcctggggtccctttgctggagcacagtcccaaggtgcatccgtctgtgacccaggtaagcatgtgtcttcccaagaggccaaaagcatggaaaccaaactgcagactttctccctatgagctcccgcatgcaccaagtgatgctggctgaggttttcttgggtctgtgacacaacaatatccattttgaaccatccaaggtttataaaaaacattaataacaatataatagaaggatagaattagattgtatagtagtagtaatctttgtagaattgtttgcatactagtactcattgtcaaataaaataatcattaagaaccatatttaatttgcagttaatgaactttacttaagctgtgactatttcaggagtaatggtcctgcttggaaattcttcacattctgtacagacgtattaaaggcagaatagcacattatcacctcaaattttcatgaggaggtaaaactaaaatcatgtacaacaatctcaatgacatatcgatataatgaagagtatgaaaaagagtaagagaaaaatgaggaaccaaaaacacatttgatgttgctaatctaatttcaaaatagcagacttaacaaacttactctaagaaaataatcctatatatctgagtctccaaggccagatatatttaaaagcctgttgaaatgatttatgcaaactgacaattgaagcagttgaagatgaaggagtattcagattttcacagtctgtgatgcctaggtacaaatctcttctttgtatgcattttactagaaaaatttcctaaattccattctgctgggcacaaaaagttattgacaaactaaatgtgccctttctgcatcttgtgtatcagttgttgggacaagcaggacccaccagaagtcctttaaatttaataggattcatggtgagtttatgctgaatttgctagatttctcttttacatgcttaatgagggattttggggatacaagctaacaaaataatgagtctggctgtggaaatatctgtagaatggaaaatcagtaaagacaattacaatcaacaatgagtacacatgatgaaggcctcaagtgatttaggaattgtaaatatacactattttaatcatagtttccagttgtgtttggaaaataattatgacacagtgtattttaacagaattacctatcttaagaaagattacaggactcttcatttcttactctttgattgaagacaaactacacggagtataagaggttttggaaccggtttgtcatgcttataaaagataaggaagcccctctcacatgtaaaaaggctgctcgaacaaaataaagcctcaatattgtatgctgcagataatagtcccagtcgtcccagtctagcagggcgtattacccaccaactattttttcagagaaagtagtttgttctttgcaaactgctcaagtaataatcaaactgaggagtgcaaaataattctgacatatttgcacatgaggagtgcaaataattctgacacgattactactattaaaatactcctatcaccctgctttggaagaaatgctgcaattagtatacagacagtgtatactaattagtataattgcaagcggaggtgcagagggaaaatcagaccttattcttttatttgaaaagctggctgggtgtggaggctcatgcctgtaatcccagcaccgagaggccaaagccggaagatcatctgaggccaggagttcgagaccagactgggcaacatagtgagacctccatctttaaaaaaaaaaaaattagctggtgtggcggtacatgcctataatcccagctactcaggagactgagcagggaggatggcttgagcctgggaggtcaaggctgccagtgagctgtgattgcaccaccacattccagcctgggtttcggagtgagatcatgtctcaaaaaaaaaaaaaaaaaaaaaagccaattacattcagtcgcaacatttctagatcaaaggtttgaggacatattttcatcaaaggatttatttaaatgtgaaatatactagacatcaaagtataagtgaaggtgaaaatccaaagcgtgattctgaaagtattcggaaagcatctcctttcaacgtgtgtgaaccttacattggtgaggctgtatcagtggaactgcatcatcaccattgaccaaatcttcaaggctagcagtttaaaacaaagtgtctttcccttctttggtactgctagagagaaaggaaagcccactgacaacatctctatctctatcagcagtgcctatcttgatagagctagacaagacagctccctaacaccatgatgaaactgagcattcattcaacatcatctgtgtgcagcatggtacccacaggagcaatctagtgactaacagggccatggaatttcctgtcctcagccataacatcaaatttaaaaaattaaactactaaaaatgttaatgacatttataatgatcactataattcataatataattatgataaattttgggttaattatatattcctgtcatttattttatgttgacaatacatggtgttggctaaatattaattcttaacatttggcctttctgtatttctttaaattgtatggtcactgtccttttacttaatagtatcctgaaaatactggctaatgcattaaatcatgaaatagaaatgaagtataattccttaaggagaggacataaaattaatactatttacagatgacacaaatataatgctaggaaacctataagcatcaactggaatagttttagaattaatgagcattcattagcaacctaaaatgttagcagcatagagatttaaagtaagatttaaataattaataatattaaataataaaaaataagatttaaataattagaaagattattataaaaagaaagaacttttactagtgaaaaaaattggcactgtaaagatgtaaatacttcttaattaatttgttggtttaacaaactcctggtcagatttccagcagaatttattttagagcttaaatatcaaaatatcatctagagaaataagtgggtaaaagtggctaatttttaaaaatcaaagtgatggaaagaaaattaatctttcagatagacactatattacaaagtaacattaagtaaaatggtacgattttactgaaagaaaaggaaagatagctctcaaacaaaatagatagaaatatattacaaacttatggagaagggaaggaatttgttgtaaataccttaaggataattagttctttagggagaaacaatcaagaacctcctctctttcatctaccattagttgagttaatatttaataagagcttctagtagtgcctgacacaaatatgctttatataggtatttattaaataaagaaaacaaataagataccaagataaatctaaaaactttaaagtaaaataattaaccctatagaaataaatactggtgacttgagaatttctctacaaaaaaaggtaaaaattgatttatttagtacatttgttccatgccggaaacaaaatagaaagcaaacaaacatctgggtaaaatatttcttagcaaacatggtagacaaatatataaaaaactcttaaaaaacaataagaaaattagtaagagttcttgttaagtaaagaacacagacaagtgacaaaaatatgacattctgatagatttatgataataatacttattcaaggtcataccatttttaaaaattggtgaagacttcaaataatctccatgttttagaatactgttaaaattttaaaatcaaaagtaaggagctcatggttaaaaggatctcataatgaaaatcaagcttcttgccttctcctccctactctgagttcaattctctgaaagcaagcacttaactttgttgcttcttatatttaccttcatgtctgtgagtggtaaacataaactgcttttcctgttttatcagttttataccttagatattgatttctgcagtaacaaatgaaatttcagctctcagtgcataccttcctctttttcccattctacccatgtaattatatcactttcaaatcactgatgtcatataaccacataaatattgttccctgcgaaactcactaatgttttacgattatatgccttttactgtcttttgcccccctgtccctctggagtttctgattgcctttatttttttatagcactaattgcctttatcatattgtttttttcaaactcttgattatatcaggtgtgttagcaaattatctcctggacttagaccactgttccataaacctagatctttttgcaccaatcttggctacttgctctgcaaagatttgttgaaatgctgtatgaaaagagggagtggtgagacactcaaacagtgctggtagagtataaattgttacaaccaccctagaaagcaagttgtcactatatgacaaaagctataaacattcaaagactttgaccctacttttcacttctagaaattgcttctaagaaaataatcagaaatgtcattcagatttgtgtgcaaagatgataactgtaatgatgtttatgtgaaaaagtggaagtaacttgagtgcccagttatttgtgagtcgttagtagattgtggagtactctagattcattaaaattcattggtagagcacttttgaaatctgcttcagggaaacacctcgaagagaaaagtagctatcttcaagcgactaaaactgaagtataataagagaaccatgtgattcaagcaagtccacaacagagccaacagagtctgctttcaaaggaatctgcaaattgaaggatacaaacaggtacgctccgaaatctcaagggtggaattaattgctgtagtgtgcctgtgcacatgggcaaccctatgtagcatgcatgttccagatgggtaacaggtgcttgattttttaatgtaatgatggttgtgctgctcccaagcaatgagaaagagggcagctgtggatgaatgggcgtagggaataggtaaggatgtgaaactgacaaggaagggaagcggtgttattaaataaaggctgaatattcacacaggggatacagaacacttcccctaatgctaaggtgctgaggaaaggctgtgtgggagatgagtagatgcacgttgagaattatgaggggaattacgtcatcaggggaaatccaggtttcacctgggatgaggcggcggcgcatgtatttaaagaacagattgcaaatttctgagctccttggcagcagagcagggtttccagaagacatatggttacagttaaaggaagggtcaaaaggggaaggatgtgtcacagaaaggtcaggctgctctcaatggaatggagacttatagacatggagagaggggtcgccagaattagtcccttagataactagggaggagaaattctgggagttgaggggtacagagcaaaggcatcaacagaacaaatgggacagaactggcagccccagcatttcaaatgaaacttcggaatgaactagggaagatacctccacaaactattgtttcaactatctcaggtacacctaattgccataggcctatcagcatttttgaggttgacttcatgcacaaatatttttgggaaaacctgatggctaagtaatttgaataaacgccttgaagggaatgaagtggtagggaagatggatttggaattatctgttccttttttgtaaaagcatagatgagagaggctttggggtacaccaacaaaagacagaaaatgcagtattagctcaagaaacctgggccaggttgtttgccttgagggagcaagaggcttgtgtgggagccttccgggactgtgtgaaatcttggctgagttttgagtcaaggtcgaccagatgcaggtctggcttcccagagagtagatctcactcccttgctgaagcacaaatctgctgggagtggcgctgactttggactcgaggaagagtgagagtaaattagtaaacaaactaaaataggaaataaaaatgatttatggtttgtaagtagacacataaaaattttcaaggccgggtacggcagctcatgcctgtaatcccagcactttgggaggccaaggcgggtggatcaccagagattaggaggtcgaaaccagcctgaccaacatagtgaaaccctgtctctactaaaaatataaaaattagccgggcgtggttgtgggcacctgtaatcccagttacttgggaggctgaggcaagagaatcgcttaaacctgggagggggaggttgcagtgagccgagattgcgccattgcactccagtctgggtgataaaagcaaaaccctgtttaaaaaaaaaaaaaaaaaaaattcaattaaccacttgacagtgttttactctcccagttctccaaatgtttatgtttaacctagggcacaattcaaagtgggtggtgttgtggaagctgagtgtttacatgtgctttcttatttctgggtaattataaggagtcaatgtgcacattttctttagagcaggacacataactgtgaagacagctctatcatcttaaaaatatttgcattttaaatattttattggcccaaattttgagaaatggaacacacagataaaagaaaatgtattgaatattctgacagacaaagaagattcagggaatctcaaatacttttaagccagcttgtgatctcattccagatttctaggtagctgcacttgagtttccaaatttcacctgctaaatgtacaattttgggtaaatatagggaacactgtccagtcttcaaaagcagtagaagcaacttcgttgaggagttagcataaaaagaatctgttacaaaaagtgggtgtacaaatgcaaatttttgatggagaataaaaattcacccaagatctggtttttagcttggctttcacacagggtactaggcattcacagaccagcctcatttttcattcctttgacataaactagccccaccagtttgggtttgtagagagacttctatcttcctaatactcatttattgacttccatcatcagagttctcacggaaaaacctttcaccaccataaatcaacttggatgttattgatacttggcctggagttacacaaacacaatgagatcgtggaagagagggttaaatggattattctggacatctttcatgaaaattgaggctcctcacatttctctcctgtgttttctttttctgagtggcttcttaatttcagtatcattttctttatcctttctggttccttgttaaaaaaaagaaaggaatgtgtttcctggaaactgtctttgtcaactaacctttgcattttaagtgatacttctgttgtcttgatttctaaagatcacgaaggtctagctttcctaaaacttgtttgagttcatgaattgttattttgaacctcactgaaatgtttaaaattatttccattgagaaaaaaatttcattctaccaatacaactatttttatctgtaatactcaaggagttttgctgagtgaagaatgaagagtaattcctaatataaaattaataggcatgtaagctttcataatagtacgaatattctcatttttcctatgggtaggtctttgctcatcaagatcccaatggctctagtaagagaagaaatccaggcacctgaagcagtacagatggtagcaagggttgtgactaggagcttggagtctaagagagagcaactctggcttgaatctttagctacaataactgtatagacatgaaataaagttcttaacctctctgattttcagtgtttataggtgactaaagtaggtatggaaaagtgggtttcagataatggttctgaaggacagaataatcatttttccaaaaataataggagcagatatataagagaatattatttgaattttttgtttaaatctagtaagtgggttggaagggaaagaattgaagagagagtgctctagaccaggggtgtccaatcttttggcttccttgagccacataggaagaggaagaattgttttgggccacacataaaacacactaacactaacgatagctgaggagctaaaaaaaattgcaaaaaaactcaatgttttaagaaagtctatgaatttgtgttgggccacattcaaagccatcctggaccacatgctgcccatgggccatgggttgaacaagtttgctctagacagaaggcgtggcatgaagatattctcagagccagaagatggggagaactagagaagatgaactgggcagtaggaggagatgaagaaagcaaaagatgactaagacagtcctctctgcccacaaattcataatattaataatggatcttttttactagaatttgatatcacaggtcacttgatttcttaattggggatttcttttatgagccatatcctccactttgctgttccctctctatttatttgaagagacaagtccaacacataaaagagaataaaggtctacaccatttagcctgatgttaagtgcaaaaggattgcagagtcagagatgaatatgggatggagtatggctgcaggctccaaggatgaaatcagggtttgaagaagtttccgaaatgccagcagcaagtggaggtcttgttgttagctttgacaggttgtagacagctttgtactatacctgtccaatgccaagcgatatgtgcctgatatgtaataatttctgaatttgttaagggaatgaaaataagaatctacctgctatgggctgaactgtgtcctcttcctcctcaaattcatttgttgaagggttaactcccagcacctcagaatgccactacatttgagatagggtgtttaaagaagtaattaaggtaaaatggggttatcagggtgtggtctaacccagtgactagtgtcctcataataagaggagtttatgacacagacagaaagacaccagacatgtgcatgcacagagggttgcccctgtgaagatagagggagttggtggccttctacaagctaaggaaggagacttcagaaaaataaccctgctggcatcttgatcctgggcttctatgctctagaattgtaagaaagtacactctgttgttgaagacacccagtctgtaattatttgttacggcagccctaataaactcacacacgatccattttgttttactttcccttcaaataacaatgatgtcttagttatatgtgttgtttagtcccatggtagtaactcattttttaccagtcctaaatgacttatttagggagagaggagaaaataaatttggagaaatgcagaacatacatgaatggggaacagaaacaaaagtatacatgaaaaagtctacagttgcgatggtgcaaggctaccctggttaagttgagggaacccttcccttatttcccaatgactcagagactggtcactgcttgacaaagtttctccctagagaagtcactagtattagaagaggacacagaaaccagggccaccctcttaagtcattgtttcagagataagccagtgtgggggaagatgtgatgaggcatagaagctggaatttgagccttggttaggtaactgaggagaccataggaattgcttgggtggggaaaaattaattacgtgattttgtctcacactttgatacatgcaaaataagtttagaacaaacatcaaggaagaaagaggagctaataatgattgagtcataccttttaaggaatatgtgtcaaagatagccttgaaagagaaaagaagtggagaagatactccagaaactcaaaatggaatgtcaggtgtgggcaaaaagagtttgagataacagggaaagggtttagagcagagcaggagggaagaacacagtgggaccggtgctattccaggtgctaatcaatgccctggaactgctcttactgatctaatggggatccaggcaggaaagacacagtccttcttctccaaaatctcccagcccagtgatgagcactaagtaaattgtgacgactctacagacttgcaagtgctaaggtgggaacccatggtgtgcactgggagcacagaggagggacctcttgcctggtgctaagggaagggggtcagaggagacctccccaggaaaggtgacaactaagcagagtcctgattgagagtgggggctaggccagcaaagagatgtgtggaaatgcatttcaggcctgcagaaagtttgtgagatgggagagcatgactcattcgtcaaaccacaggaaggtattatggctggagtagaatttttctgaaatgcggtcttttctcagtggtagggaagaagggcaaagggagagatgtatagtgtctctctcagaacttaaaaattcttttttgttaatgcaattaatatgtgtgtaccatgaatcacctggatgatttgcatatattcaggagtgattatagtaaagaagaatttaggacaacaccaaatccagcaatatattgttaaaatgttgttttgaattaatgcataattgtacatgcaggtatacctcattttattgtgctctcctttattgcccttcgcagatattgtattttttataagttgaaggtttgtagcaaccttgcattgagcaagtctattggcaccatttttccaacagaagtgctcacttcttatctctgtgtcacattttggtaattcttccagcatttcaaactttataattattattattctgttatggtgacctgtgatcagtgatctttgatgttactattataattgctatggtttaaaggtccctgtagcccatggatcaaggagtaattttgactttcaagccttataatttaagaaactcaagttagcacatgaattataagaaagcaaaatgccagattgttgatatggagaaagttttagtagtctgggtagaagaacaaaccaggcacagcatttccttaagccaaaatctaatccagagcaaggccctaactattcgattctatgaaggctgagagaggtgaggaagctgcagaagacaagtttgacactagcagaggttggttcatgaggtttaaggaaagaagctatctccataacataaaagtgcaaggtgaagcagcaagcgttaatgtagaagctacagcaaattatccagagatctacttaagatcattggggaaggtggttgcactaaacaacagatcttcactgtagataaaacagccttctattggaagaagatgctatctaggactttcatactagagagaagtcaatgcctggcttcaaagcttcaaaggacaggttgacactcttgtcagagacaaatgcagctggtaactttatgttgaagccaatgcctatttaccattccaaaaatcctagggcctttaagaattatgctaaaagctgctcttcttgtgccacataaatggaacaacaaagcctggatgacagtacatctatttacagcattatttactgaatattttaagctcaccgtttagacctattttttttaaatggctttcaaaatactactactcattgacaatgtacctggttacccaagagttctgatgaggatgtacaaagagattaatgttttcctacctgctaacacaacatccattctgtagcccatggatcaaggagtaattttgactttcaagccttataatttaagaaactcattttataatgccatagcttccatagacagcgattcctctgatggatctgggcaaagtgaatggaaagccttctggaaaggattaaccattctagatgcagaataatcccatttaactactttttcttttgttgcatatgcttttgaggtctgaaaaatccttgcccagacaaatatcacaaaatgttttccctatgttttcttctagtagatttatctttttaggtcttacatttaagtatttaatccattttgagttaatatttgtaatgatgagagatagaggtctagttttacttttctgtatgtggatatccagttttcctggcaccatttgttgaagagattgtcctttcctcaatatgtgttcttggcacctttgtcaaaaattagttggctgtaaatgtgtggatttatttctgtatcctctatcctgttccattggtctatgtgtctatttttatgccagtaccatgctgttttggttactatagacttgtagtacattttgaagtcaggtagtgtgatgcctccagctttattctttttgctcaaaattgctttggctatttggagccttttctggtttcatatgaattttaggatcaactttttctattcctgtgaaatataattttgtgaaaaatatcattggtattttgatagggattgtattgaatctatagatgactttgagtagtatggacattttaacaatattaattcttccaatccacgaacatgagaccgctttccatttatttgtgtcctcttcaattcctatcatcaatattttacaggttttattgtaaagagttttcacttccctggttaaatttattgctgggtatcttaatttttgtagctagcataaatgagattgcttcctttctttcttttttagggagttctttatagttttatagaaatgctactgatttttgcatgttgaattttgcatcctgaaacttaaatgaattagtttatcagttctaaaagttttttggatagagtctttaggtttttctatatgcaagttcatatcatttgcagacagacacagtttgacttcctctttttcaatttgaatgacctttctttctcttgcctaattgtacttgccaggacttccattactgtttttaatgaaagtgccgaaagtgggcatctttgtcgttttccagatcttagagaagagctttaaacttttcctcctttagcatgatttaactgtgggtttgtcatatatggcctttattgtgttgaggtctgttatttctatttgttgagagtttttattatgatgagatgttgaatttcatcaaatgctttttctgcatcaattgaaatgattatatggtttttgtccttgattctataatgtttattgatttaggtatgctgaaccatccttgagtccatgaaataaatctcacataatcatgataagtgatctttttaatgtgctgttgaattcagcttgctagtattttgttaacttttttttgcatccatgttcatcagggagattggcctgtagtttctttttttggttatgttcttctctggtgttagtatctgggtaatgctggctttataaaattaatttggaagaattacctccttttcaattttttggagtaatttgaaaagaattgatgttcatttaaaaaaaatgtttggtataatacagcagtgaagccacttggtccggggcttttctttgatgggagatgttttattattgatttaatcttgttatttattattgttctgctctgttttctgtttcttcataattcaattttggtagcatatgtacctagaaacttaaccatttcttctagatttagcttttcataaaagtcacttatgatcctttgtatttctaggatcagttctaatgtctcttttttcactgctgactttatgtatttggcttttatctttatttcatagttagtctggctaaagattggttgattttgtttatcttttcagaagaacaattattggttatgttgatctttttaatttttttctctattttgtttattgttgctctgatccttattatttattcccttctaccaattttggctttggtttgttcttgtttttctagtttcttgaggtacaatgttaggtttttatctgagatttttctattttgttcatatagatgttaattgctatatacttccctcttagaattgtttttgctgtatcccataggttttggtatattgtatttccattttcatttgtattaaattatttttaaatttcctttttaatttctttattgacccattggtctttctggagcatgtttaatttctatatatttgtatgttttccaaaatttctcctgttactgatttctaattttcttccactgtggtctgaaaagttacttgacataattttgatttaaaaattttttttgacacttggcttgtggcctaacatatgatctatcctgagaaggtttcatgtgctgttgcgaagaatgtgtattctgtagcggttgggtggaatgttccgtaatgactgttaggtacatttggtttagagtgcagtttaaatatgatgtttctttgttgattttctgtctggatgatctgtccattgctgaaagcagggtgttgaagtcctctactatcattgtattgaggtctctccctccctttagatctaataatatttgctttacgcatttgggtgctcaactgctagtttcacatatatttacaattgtcatatgtactactcctgctctcttttggtttctctttgcatggaatatgagcttccatcctttcactttcagtttatgtatgttcttacaggttaagtaagtctctcacaggcaacacatacttgttttttttttaaactctatttagccactttatatactttaattatgaaatttaatccatttacattcaagattattattgatatgtaaagacttactcctgctattttattaattgttttccggttgttttgtttatcttctgtctctttcttcctctcttatggctacatttatgtttgggtgattttctgtagtaataaactttgatttctttctctttctcatttgtgtatctgcagtaatatttttctttgcagttattctggggcttacataaaatattgtatagttataatagactatttttagctgataataataaactttggttgcatacaaatattctagacttttactatcccttctaaaatttttaattttgctgcttaatttacatctttttatattgtgtgttccctaacaacttattgtagctagacttatcatcgaccatgttgactttttaacttcacagtagagatatgaaagattatgtaccaacattatggtaatgaagtattttgaatgtgataataaatttacctctaccagtgagattagactttcatatgttttatgatagtaattattgtccttttgcttccaattggaatacttccttaaacattttttgtaaggctggtctagcggtgatgaattccctcagcttttgcttgtctgagaaaaaatgtatttctccttcatttctgaagatagctttcctgagcataatattatacatgacattgtaatgacactttgaatgtattatctcattttcttctggcttgtaaggtttcttttgagaaatctgctaatagtctaatagaaattcttgtgtatgtgacttgatatttttctcaaaaagcccccaaatccaaaaacttttttttaagaatgtcataaatgactaagctagaaagagagaagaaggtgaagttggagaaatgcaggacaatacatagttggagaacagaaacaaaagtctacataaaaaatacagttgagatggtgtaaggctaccataatgtaaggttaagggaatcctctctttctttttcagtgactcagagactggtcactgcttgataaaattttttttctagataattcactagtattagaagagaacacagaaaccagggctacctttttaactcattgtttcagatatagggtgaggagtggtgtgatgaggcagagaagctggaatttgagctttggttaggaaaataaagagaccataggaagagctggagtgaggaaaaattaattatgcgacttcttctcacacttacatacatgcaaaataagtttacataataattattgagtcatactcttttttttttttttttttttttgagacagagtttctctcttgtcgcccaggctggagtgcaaaggtgtgatcttggttcactgcaacctgcacctccttcattcaagcaattctcctacctcagcctccggagtagctgggattgcaggcatgcgccactatgcccagctaattttttttttttttgtatttttagttgagatggggtttcaccatgttggccaagctggccttgaactcctgacctccggtgatccacccgcctcggcctcccaaaatgctaggattataggcatgggccactgcgcccagctgagtcatactctttaaggaatatgtgtgtcaaagatagccttgaaagagaaaagaagtggagaagatactccaaaaactcaaaatggaatgtcaggtgtggacagaaagagtttgggataacagggaaagtgtttagagcagagcaggagggaagaacacatgggactggtgctattccaggtgttaatcaatgtcctggaactgctgtcactgatctaatggggatctaggcaggaaagacacagtccctcttctccaaaatctcccagcccagtgatgagcactaagtaaattgtgacgactctacagacttgcaggtgctaaggtgggaacccatggtgtgcactgggagcacagaggagggacctcttgcccggtgctaagggaaagggggtcagaggaggcctccccaggaaaggtgacatccaatccaagtcctgatagagagtgggggctaggccagcaaagagatgtgtggaaatgcatttcaggcccgtggaaagtttgtgagatgggagagcatgactcattcatggaaccgcaggtaggtattatggctaggataggataatttttctgaaatgtggtcttttctcagtgatagggaggaagggcaaagggagagatgcacagtgtctctctcagaacttaaaaattttaataaggcaatgaatgtatttgtatatatactctaaatcatttggatgatttacatatattcaagagctataacagtaaagaaaatttaagacaataccaaattcagcaatttttttttccttttaaaaggatatatttaaacactgatgagagatgcactagaattgtaggcaggacttttttttttttttttttttttcagttatggtttacctgcaaagttcctagtcctaacagtagttcaggaatccacagatgtctgaagtgtgagctagcaccccaattcatctgaccataggaaaggctttagtggactcatacaattcaagtgagactctggcactgtctttgagtttcagtaagtgcctttcctttcagagatactggagaattagaactgtgtagaaagtcttccagcgtacaatatagaagcacaaatagagccattgaccttaatgggacatttgaggaggtcatacaataaaacagcagtctctgttaagaaataacactaatgggcctgggggtgctaaatagagggtggattattttcttacccatatattgtacttggcttccacgtcatctggaagaaatttcatctgttatcttcacttggtcttcattcttattttaaaaaaccttctcttcagagcagagcaggtatctggtgaattatcactgtgactaaacaaattgtcctttgcccagtgatttttaaatatgtacactacccaccatggaatttactaggccagctaaaacttgagtaggagatataactaattctaatggattttattttgatgtagataagtactacaatttagaaaagcataaggcaattagcaacttaagaccttgctaataaaatgctatttcactttctggcagagagtgcaatagacagagatcaattctaagagtaaattggttgaatattagtgcagagccctgtgatgccatcaaaacatattaggcatatttactatatcatggaaagtcctcagtaatagcacatacctccctgatggaaaaagtggccatttatttttaaaacttttatttatctcagctcaaatttttagatatgtttctcccccaattagaatatgagaatagggactgtattgtttacaactaactgtattgccagcatctagaacactacctggtctgtaataggctctccataaatattttcagaataaattagaatcatctacacacctactttgacatacactgtctggtgctttcagatctgtctcactcaatttttgtcagagctagtcagacgtcccattggtccaatgtggaagctgagatcagagctaactgtccgaggacaaatagctagcaagtgtcagatgtgggattcagcccaagccttctaacttcactctcaagaatgttttcttgtctaccttggatttaaggaacaattctggttctttgctgagaggactgggtactggctgctagaaagctttttgaattatttcttccttctccttccttccttccttccttccttccttccttgctccttccttccctccctccctccctccctcccttccttccttccttcttttcccccaaagacccctgatatgaactagataggttactagtggatttccttccagatttcaatgtttactgatataccaagacattatgcaacctcaaaacacttctaacccaagagcacttctaatggtacccacgggggtccctcaggactctggctcataaatgggctgtcaggcctcagagccctcagttggccgcttggggctcttccctcatagacatgcttccttgtgtcacatggacagacagaaagtactgtcttgatgagtcatttttggctctgaagctacttgtcactcaggaggcaggagagggatgtgatagaatctttgcctgaattcttgaagggagaggcttggcctggttacacatatttcttttgttacaaaatcctcattaaaatagacaccttttacttgacccagcagatctgtagttgtttattttctctctattgtaaccaaaatttaatttttccccaacctttggacttgaccatgaagatgataaccatttttagatatttatataatatttgtaatagatttcatgtaatgtttatattatatatattttataaatgtaacgtttctcactttcttcttaacaagatctggggcaaacaatttcaggttaaagaaatggtatttttaaaagttgtaggggacattttatgggtagaggccttgcacccgtgtctcccttcttaattagctggctagattgcagactcactctttgagcgagaggacttggtttgtgtgtttgggggcaccgtgtgtgtgtgtgtgtgtgtgtgtgtggatgcatgcatgcccatttgtatttgtacataagttaaaaaataagaatcgtgccgggcatggtggctcacgcctgtaatcccagcactttgggaggccaaggtgggtggatcacaaggtcaggagatcgagaccatcctggctgacatggtgaaaccctgtctctactaaaaatacaaaatatcagctgggcgtggtggtgggtgcctgtagtcccagctactcgggaggctgaggcaggagaatggcgtgagcccgggaggcagaggttgcagtgagccgaggtcgcgccactgcactccagcctgggtgacagagcaagactccatctcaaaaaaaaaaaaaaaaaaaaaaaaaagaatcctaacacacaaggagttttattatttacccaaagatgttggtagtttcagggaaatttctaggaaggggacttccatccagatggcattttccagtagcagaaagagaaaggaacgatgtgactgttagtgcaagcactgtgctgggaactccacgtatgctatgaattcaatctgtacttaaaaataagttttactgagtaccttctaaaagtaagttactgtgcaaaagctgggaaaacaaagatgaaaaaagtacattcctttacgccaaggaatttacaatctgaaaggtgaggcaggtgaacaactggtgattttaattgggtgtaataagtgttatgcaagacgtttgtaccgagttatggtgcaggaggacttaggagaggcacccttattttgtcttagccagacaaggcttcataaatatgtacgttggaggggtttagggaagacttccccagggaggagacattcaccttgagtcttcaaacgccagtccagaagcatagggacagcttcactacaggcagagggaaaaacatgccaagctcccagaggcagagctggacaagttcaaggctcctgctcaaacaccaatgcctgggaggcagggccattttgaagagttcagattcattttgatggctgtggttaatgggtcaaggatttaaagtgggggagaaatagaattagttttgcattgataaatataaatctgttaacattttgaatatagacatatgattatttttgtttgctttttttggacatttgcttcttcagaatgataagaaaggaggaacagggactttgctgtaaacagacattcacaggatcacctgattattatgctggttgttggtctaattctcttcccatgaagcatttttttggaaaatctagtgtgttatgaaattaactgacttaagttaactgtcttgcaatatgatgaatggattaaggagaaaatgacactagaggctggaaaccaagcatgaggttgtttgagcaatccaatgagtggaaatgctactaggagatagaattgataagattgggaaggaagggttgaagagacacaagcgtcaaggatgtcttgagaatttctggcttgggcaactaggaggacagtgataaaacaatagtgaacatttattgagcaccaactacgcaccaggtactgttctatgtcgcctcacatatctgagctcatttcatacttaacaataatgcgtgatgtaagtactactcttttcacattttgcacatgaggaaatggaaacagaaaggataagtagcttgcccaaggttacacaattagtaagaaatagatttgggagtcgtcagttcataaaggtcttgcagacgaaaacaatggatagaattactcaggaggtatctgttggatgaggaaagggccaagggcgaatctctgggaaattccagtgtttcaggaatcggcagaggaagagtcgtatattacaatatgtcaggagtcagcctgtgaaagaggactgagttggaccagtacaagaggttggagaccctggaagacaatggcgtgatagacatcaagggcctccagggcgggactaccgcctgcttattctggataaggaaatggaacctggggaatggtagacctgagatttgctggaagccctgagatttgctggaagccttgtgttcttcccatggctccagaacccctttgggattaaggcatcatttgtaggacaagcctgtggtttttatttttggactcgataatctggattgatgaagaatgcaggagtgggacttggttatgaattaaagatcatattatctccttcttaaaatgctggttgtaaatttaatttcttctctaagaaatcgtttactggaacatttattgagtaagaaaattacttgctttgaattatagctcttcttttctctttagtaagaattagctaaaaagtatctaggaagagaagcagaaaaccccaattatcccccaaaaattcattctcatagtgaaccttcagaaagtctattggaaacccttattatttacaataactgttaagataagcttattgaatattcttcccagattaaaattcaccaaacctcatcagttctcagagatctctgccttcctcaagccactagatagccagtaatttcctttccttttattgaactgtatcagcttcagaagacgcgatataatcctattggtttctgtcctcacaataacactgctgggacccatcctgagggtccttgagggtctttaacctctaatgtctggccactctttcttcttagtctgagccatatatatcattctcatatatgccacacataccatacatgtcgcattcattcaaatcctacatttaaaaatctcttgcaaaagccagcttgggccaggcatggtggctcatacctgtaatcccagcactttcggaggccgaggcgggcagatcacttgaggtcaggagttcgagacctgaccagcctggtcaacatggtgaaaccccgtctgaactaaaaatacaaaaattagctgggcgtggtggtgcgcacctgtaattccagctacttgggaggctgagtcatgagaattgcttgaaccctggaggcggaggctgcagtgagccgagatcatgccactgcactccagcctgggtgacagagcgagattccgtaaaaaaaacaaaacaaacaaacaaaaaaagcctatctcaaatacttgagtcctttatgaagtttagcacttggcttattttttgaaagaatactaccttctttcctgaatttatattttcatctcccctcctccaactttctctggcagtgctttttgcagctggactcccaatttagtcatcataaaatcactgtagtcataaaacctgcttggatagccctgagcataacttgtcaatttttatttatttctttgtttacttgtttggcagtctcttccactaccttaaagatctaaggagataggaattgtgtatttttttatttactagtgtaaatcctatttgattgtaagtccagtgttctttttcccacaatatacaggtagcagaggcagagaggggaggtatgtccctgacacaaggaccctggaaaagagatgggaaaattaggaaatttacccactcctaaggctaaccagaaaactccacgctagatagctggtgtgagggctgggtctgtagggaagatgagctgggagccagaagatgaagtgttgagatggatgataaaggaaagtctcagccaagacaggagctggagctagctctgaaacacatgggaagctggcaggatgcggtcagaggtcagagagcgatcagggttggtttaggataaattttgctaagtcagggtgtctgaggacagatctttttttattaaccattttgcatgctcttgtcccgcaaggacctgtgcctccttgcacattcattttgcagaaagatgccactaggaaaatttgaactgaataggaaaaaaactatctggtgaaatgaaaatgatcagaaccttaaatgaaactgtggcttatcattcctttcaacagtccaaaaaggaaaatccagcatgaaggcaaacagatactagagattaccaaaagtagattacaaaatgtgcagtaaaaattaagtggaaaccttcagagattgttgaaagggaaagtgttcaagaagaatgggaagctaaaaattgaaaccaaaagtataactataagcaatcaaatcaaaagcaattttgatgaagaagaaaaaagataactagagatctgtataactacagggagaagttttattagagttgctttttaaaaaaaaatacataaataatgtaaggaggggaatataatcaagttcagatgtaaacatggctttaatttatctgtgaaaaactagctctgtgacaaaataagagaaaggaaaagctagtttctagagtgagtggaagatattgcaaaaattcttaagctgtgggaaatgggtaaacaataaaaggatctttcaaagctatattctcaatgaagacgagcagcaggagcctgcttttttccagtattgatgtaactttttaataatataaaaacataaggcctggaaagaaaatttcttcctgtatattctataataactcagacttattagtagcatattcttttgttaataactttataccccacaataagcttttatatgcactattttatttaataatatttaactactttctcctaacacattttcataaattagaaaatcaacagtcttccctctctaccttcttaagacactttagtgtgctgctttatctctacacaatcttcagtgctttgccttatatctacaactcaaaagaaaatttctgtaataaatgtagtccctggctgggtgcagtgattcatgcctgtaatcccagcatttttggagcctgagacggacggatcgcctgaggtcaggagttggagaccagcctggccaacatgacaaaaccctgtctccactgaaagtacaaattagccagacatggtgtcctgcgcctgtaatcccagctactcgggaggctaatgcaggagaatcacttgaatctgggaggcagaggttgcagtgagccaagaacatgccactgcactccagcctgggcaacagagcaagactccatctcaaaaaacaaacaaacaaacaaacaaaaccaaatgtagtccctaatgaagaaacaatgataggaaattaaagtaaaaatgccttttttctcagatcaattttttctttttaagatacttgcaaattacattttctccactgcctacttcatagctcaagtctggcaatttcagctggaattaggaagagctcgtgtgctcagcctcaaaaccatgtctacattccgactgcctgattcttcctcttacagacctctctgtacaacgtctgttgtgtttcagctttttgactcttcaaattagcatctcttccataaattggattttgatattgtttcaccccagtcaacattcagtaatagaacttatgtgaaacagttcttcctctgtcctttgcacattttctttcatgttctgcatttgatgatactttgctctttccttttcttctgaatccttgatttgaaaaaaactttgtgtagctctctgtatatcttactaactaaacttttccaattgatatgactgtctccatcatttcacctacagcctcaatgaaacactttcctctaggcatcttgagataagtggcacatgatattttctcattctataatggggaaaggcagagagaggctaagtgccttgactatctgagtgaggagttgatctccaaggagctgtttaatgagtagacattagtttctgttgagttggatgatctatatttggaccatgtcattattaatagtttcactttcctgaaaattaggtgttctcataatttggagttctggctaaattaaagattgtcattttccttagttatgttctatttttctgatatgttatatttgctcccacctctacattagtggatggtggcttttccaaaggttgttgctgttttcagattctgtgttccctagaagggatcgaagcccttgaaacatcactgagaatttattacttattgattatttctttcattttctatctagggttttgaattgatatcatatcactttcaatgtaaagaaataaagtgagtatagattagattaaaaaaaatgaaaagcataattagaattaggatgtcctagatgttttctggtcatcttttgagagcagaaaataatcaatatgtagatctacacatataggaaaaaatgctaaccagtcaaagtggagaggtgggcatttgcttgagtttctatgcatgttagaaacagttgctctttgctccattttgttggatccaagtcataacaaagtggtagaatgttccttgaaaggggaggagtatgggagagaattgggaaaaatgaagccactggtacagagacctggtcattagtccaaaaagatagtttcttccagataatgtacaaaaatagaatggatgtggtgggagaaagaacagccagaaagaaacaaggctgtgcaaatgctttatctagaaaatgttagccctccctggctaatcttgaaactgtttggaaaaaatcgtttaaacaagctactttccagttcatgagatcctttccactttgtagcaagattatgtgctttacatacacattagcagctttgagattgtctaaatcatgcttcattgcttttctcacactccaggaagaagggaggttgagaatggggccagggctccaggcctgatcctttctcctcttcctcctcctccttcttcctctcctcctcttccttcttcttctttaaaagtaagtcttcagtactgcttcccttaaagtcttgcaataaccttatcttgagctataagcacacacaaatgaagtatattccttcatttgatcccatttttctttttcccacatttctctaaaataaaccaacataataaagtcataatttggagttctggctaaattaaagattgacacacaatgtcattactcttcattctttgcttttgcaagtcacgtgtgagaaagaaacatatttctatggccttttgactgcccttgataaaatctgtacttagggaaatagcaatatcaataaatgctaaataatattaacacaatgtatgcaggggtttttaagcttcacagaaaaaaaaatttaagacatgaatttccaggaaaattattcttgtaactatcccaaatggtttttctttccatcaaaagaacaaatggaaaaaagtgcaaaagccaaatcaaacaaaatccttagtgtgaaatatattttggagcatgtggaattaaatttgcattttcaaataaaaacaaatgcccgtttaaaaagtcatctaacttttgtcctacgaagacaaccagtaaaatatactgtttcccttttcccagcatactcatgtttgggaaaacatgattatctgaagagaaatttacttttactaaattagagtttgaggggggagtgtggaaggaataattacagattatccttttattcaatctcttctcttggaaagaagagaagaaatgttgaaagccagagaacttttctaaaacgctgtagggaattttttttttctgtaaatatccaagtccaaaaggagatttgcaaaatgtggagttggagatttgaactcatacaatatttcctaatacaaataaaaatctgaaccttttaatggcgacacagtccacaccatgaatggtcaaaatcactggagctaatgtacaggaaggggttcctcagggaactgtggcaaattttagcatcttcctttccttttagggactggacactcatccttaggacttgagaatcatatgtgaaaattatatagacttcccacagtctactgacacattacagagatactggaaatattaaccacacatgttgtctacatactctctttctcatcatcataagccacctgtgttctcactgacactgcaaatccagtaaccaaaacatcctgttaaaattctctggcccttcttatccatgtgctgtcctttgccacagctcctaaatcctcttacctttgaaaatctttcctgctaccctttagaacctccattcagcctctttctcaagttgctttgtacatagtgtcttcatagcagccttgtaggaacctcagcttcctcaccccaatcattgttttcttttctccatttgtattgcttttcctccgggggtatctgttaatcctccatcagtctattgacttatgaatataaaatccatgctacctttacattggaacacataaggtacctagaattccttttgacacctagtggtggctcgataaacacggatgcacatcacgttagagaaaaggagttttctgagtccagatttcttacatgaaaacaaatttcaaatgaataaatttctcactgttattttaggatagctctctgccaaagaccttttattatacaaaagttgagaatgattgaagttattttgcttaattttttgccactaaatttgatgtgccaaagaaacttctaaaaaacaaaaaagaaatgcataatattcccagctactcaagaggctgaggtgggaggattgcttaagcccaggaggtccaggttgcagtgagccgtgattgcactccagcctgggtgacagagtgagaatttgtctaaaaaggaaggaaggaaggaaggaaagaaggaaggaaggaaagaaggaaggaaggaaggaaaagcaagcaagcaagcaagcaggaaagaaagaaagaaagagaaagaaaggaaagaaagagagaaagaaaggaaagaaagaaaaagaaagggagaaagaaaaagagagaaagaaagaaagaaagagagaaagaaaggaaaggaagaaagaaagaaggaaaggaaggaaggaaggaaggaaggaaggaagaaagaaagaaagaaagaaagaaagaaagaaagaaagaaagaaagaaaccatgaaggtagctgccgtatttctatgaaaccttccctccagaattagttccttttagcatctcattcagacacatggaaagcttgtttaaaatgacttcacaaattagacttaatgaagtgacttgactcaactcccctcaccctcaagtggtctttccctgcatcagagtccctttagtttcggatgtcactcatgacatagaatgaacatttaaaagatgaggaggcagccactggctatcacatttttctgagatgtgtgaaatatgattctaatattaataataataattaacaaacaataatatcctaatataattattaatcatgaattattaatacataatcatatcattattttctcttagtctaatttataggttcagatgtgtgcatgcatgtgtgtcagtggcactcagagaggagatttaaatagagagtgggaagggagataaaagataactgctgcagcactggagcaagaagccaaggggaggtatccgttaaaaactttttgggatgtgaaccttttgtttttctagattctggatgccggaacatctgttttttgagatgcatctgtaggagcatttttgtgtggaatatttgaaagttatcttgaaagtaaaggtcagagacaggaattgaaacagggcttaattcttaaacaagaaggtataagatattgcagttgatggaactgtggtgagcatgttccccaagggtggtgagcatgtctgtgtgggcatagaagatacatgtacgaacatttttattttattttatttagatggagtttcactcttgttgcccaggctggagtgcagtggcgcaatctcggctcaccacaacctctgcctcccgggttcaatgattctcctgcctcagcctcttgagtagctgggattacaggcatgcgccaccatgcctggctaattttgaatttttagtagacatgggatttctccatgttggtcagggtggtctcgaactcctgacctcaggtgatccacttgcctcggcctcgggattacaagtgtgagccaccatgcccagccaaacatttttatttttaatagcttaatgaatgtataatgattaaatgtttagcatgtattaaactatgatttcatgtagtttttttaaccataccaaccatgatttcatatggattatttaagcataccaaccataatttcatgtacattattgcttaagatagtaatgatataaaattttcttaaaattattcatgtaaaagaaacaagtcagttaaaaggaaaatattaagtaaaaatgtacaggtggcacccaaatatggtaaaaattgtgaagcagtttaatgaataactgaactttgggaaatgctagattagagaattggaaatactcttttaaataaacacttgctatctatgattatcctatggatgagtgtccaagaaatagagatgcatatgttgtatgtttttaaaatgtacaggatgaatttacattgaccatcatatttcctgtgctaaatcaaccattattttttggactatagggagaagctgtgtttcttcatagctcctggagatgtgtgatgtgtcacaggcctaataactatattacacattttttcacaaaagtgctacatagagtagaaatgtataatagttaccaacccaagactaaatcaggcctagtggtgtggatagcttcactttgcctatgataaaatacttcctcattctttgtgtgattttcaagcaacttaacgactgttagctttgctgagctaaatgcaacatctcataccaaatttattggcttggcaaagttacaaattttattacgcaaaagttgagaaaggaaagctggagaatgctaaaaacagtacaatttgctactgtgtagtatctgtattgggggctcagcatgttttatttatagatatctattaatacagagatacagaaagaaatacataaaaaatagttttatcaaatactttccagcattcaagtgtagcctcaaaagcaagaataggccaggagtggtggctcacgcctgtaatcacagcactgtgggaggccaaggtaagaggattgcttgaggccaggatttcaagaccagcctaggcaacatagtgagatccctatctctacgaaaaaattttaaaacttagctgggcatggtggcttgagcctgttgtcccagctactcaggaggctgaagtaggagtgtcacttgagcccaggaggttgaggctgcagtgagctataactgcaccactgcactccagccttggagacagagtgagaccctgtccccaaaaaaattaaaattgagaaaaaaaaaaaaggcaagaacagccacagcaaactttctattggggaaaaaaaaaaatcctcctctttacatctctcccttccttcccttccctttctgagagtgactgtggccaaaaggagcattttccccctgcagtcctctgaggggtggggtggggctatgaagctatccttcatattcactcctttgtccagctcttttcacctctagttcttctccccgcatctctgtctagcagtgccttaagtggaggaggggtgggggcatcaagcttgtaaaactggtttgttggggttctccttctcccctcatttcttgattcttgggaaaatgtcttgctgggaggctgcctggcagtgccctagctgccttctgtgggcttgaatggggcttccctctgcccctacaggaggaaaagggagctgctgccagagggagaaatggagagatggacagagaaggcaggtgccacccctcgcccctgacacacaaagaaaaagacacggaaattctctctctctcttctcttctcctatctctctctctctccctctctctctctctctctctctctcacacacacacacacacacacacacacacacacacacacacaggcgcgcgcgcgcgcgcgcaggcacacgtcttgcaaattcaggattcaaagagacaggggcaccattatatttggcacggtggggcccttccaggtctgaaatcctgcattcttccttactatttactttccccgagctcgagaagggccaggtgtgggcggatggctggccacgttttgtgtttccaattcatattcacgggatgacacagacggggcgtggtgagtgctgttggaggcgcttgggcagtttcattttgccccacttctccacctgaaggctgggcgttgctggaacctgcaggggcagcctcagcaaggtggggtggcgtggagtggggtgggagaagggactccagctgaagtagaacccaggctggacctgagaatattggggagggcatgggcggtggtttccgggtaggggccttgagaacatgttggtcctgactgttgtcagtgtttggtcaaagttgccaaaaggttaaaaaaaaaaaagtagggggagtccctgccaagacatatttcccaggccacctttcttccgcgggagtgttgggggggaggcgctgcttggaacctgtgaatgtgacatcagctctcctctcctctcccaaggtcggctttggagagggaggtcagggcacccttgcctggcacaggcggcagcgctggcttccggctcagtgccgcctgtcctccgggagctgtggcctccctgggccccggggctaggctgaggtaagcgcacagcggaggccaggggccccggcagaggccctggggatagggtggaggcatctctgggtgtgggtgtgggtgtgggtgtgggagggagagttcttgcctctctctctcccatctccaactcttgcttcagtggctcttttagaggatgcatgtcattatggacctgtcgctgccactgtccctgttcccccagctgtgacttcgagggaggtctggggatctgagtctgtccaaacccacggctttgctgttgggataaaaactgtccttttgattttagaaggaggagggaaaaaaggtttcccagcatgtgtgttgtgccagtcttggaaattcatccgtgcttgaattccaccctccatccccagaaaaactggagtaaaacaaaaagaggagatggacaaagtgtgtatttgatggcatcccctgggaagagactctaaatttatcccataggtcttactgggccactgtgagcgctttggtggagaacaaacaaaaattctgggtgctcagttgtctaacctgaaaaatgggactagcggaaaaagccaatgtgttccatgcaccttttgctttctttattaaggcatgatgtcacctgtacagtaactgccctgtgtgtacttcagggggggatttcaaggttagatagacaggaaattgttttgaaaatgtaaacacattattaaatgtgaagtattatctgattccttgttcgaatggcatttccttctcagcaccaccttccttgcatattcacttaaccttgtacaagaacacctttttgccctaaatgaagacacccccccaaaaaaaagagtcccagaaaatatgtccctgcttgtgcggggaataaatagaatattctgaggtgcattcctccttcctatgttaggcaacattccttgaccctcctcggcccccaagccaggttgcgtttttttctgccatttagaagggttttcctttttgtcctagtaaaacatcagcccctgtagctcttcatctccccctggtgttcttctcccgccatgtcttaagattggtggcaccgaccaatcttaagatttaagttctgtgtgaaaaacacctttgcttttcaatcagtttatcagcctcctccgcaggggaagtgtggacacacaaaagaacttatcggggcttctcatcagtgatagggaaaagactgggcatgtgcctaaacgagctctgatgttatttttaagctccctttcttgccaatccctcacggatctttctccgatagatgcaaagaacttcagcaaaaaagacccgcaggaaggggcttgaagagaaaagtacgttgatctgccaaaatagtctgacccccagtagtggggcagtgacgagggagagcattcccttgtttgactgagactagaatcggagagacataaaaggaaaatgaagcgagcaacaattaaaaaaaattccccgcacacaacaatacaatctatttaaactgtggctcatacttttcataccaatggtatgactttttttctggagtcccctcttctgattcttgaactccggggctggcagcttgcaaaggggaagcggactccagcactgcacgggcaggtttagcaaaggtctctaatgggtattttctttttcttagccctgcccccgaattgtcagacggcgggcgtctgcctctgaagttagcagtgatttcctttcgggcctggccttatctccggctgcacgttgcctgttggtgactaataacacaataacattgtctggggctggaataaagtcggagctgtttacccccactctaataggggttcaatataaaaagccggcagagagctgtccaagtcagacgcgcctctgcatctgcgccaggcgaacgggtcctgcgcctcctgcagtcccagctctccaccgccgcgtgcgcctgcagacgctccgctcgctgccttctctcctggcaggcgctgccttttctccccgttaaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagctccagaaacaggtaggcacgctcgttgacttgtaagtctcggaattacaagttagtgtgttcttatccaccttcatgcttttcttgcttctatttttccccgttctttttatgactgcagcttagagagcaagtgtctgagaattattgctgaaagctactttaagtcttctagtgtaaaatgtaaaattcctctattgaatacaattaggtgcaattgactataacatgacattaaaataacttatcgttttattattattattccattatgtgtttccttggcttttaaaaaatgagaagagtatggacatatacaatttagtcaaatgtatgtttgtaatatatgtgtttatacaggtacacaggccatataggaacttaaatcttatttaaacactattttaatagtgtgttaacgtgtaaaatatttaagcattccagcttgaagccaaggaattgtatccagtcgttcaagcaatgtatgttcagtaaaatcacctgcagagcaaaagtctgttgactaactaccgcctccccccccccccgccaccaccccccgcaggcggtttctgggtgaagcagatgttttctttaaaatttgtcatcattgactttaggtttcttttggcaggtttttggcacccaaaacagtgtgagctctcttttcagctttattcacctgtgctgggaggggagctaggataattcttggctgccgaaggatttaggcagtgcgtgtgcatctgcccgggtcccccccgtttttagggtcagtgcactttttttgtcttttcgtgaccctgactaaagagaaaggatgtcaagggaatgaaaatcctggaatgtgtctgatcatttgaaatgtacaaaattgggcagataagctgcatggctaaattgttaggaggaagaggcaaggcagtagtggagaagggggaggcagtggatcccacacaagcctgatgcccagggattcggaattcaaaatccccccagcctaccttcagtcccctgacctgcttctcagccccaccttaggtcactggtttctatggagttaccctcctgaattgaatattgaatagttaatttctctctccaatcattttccccacctaattttgaaagatatacatcatctggggtaccctgtgccctacacagcatgtgaagtggatgggtaccccctaaagagagggtcatcctgaatggggaagtggccccaaagctaggaataactgtgatttcttgtctttagtcatgtgccaatgttaagtaagcttcagtggatagtgctgtcctaccaagttccttgtagaagccagccggattttcaacaggcagcattccacagcatttccctgagcctgcttcaagaggggtgggggaagtcccttttcaggtgtttatctcctctgcatttgtgtaatctccctgaaggtggataagccaagggcatgagggggaggcaaaaggtgaactcatgttaaggagggaaaaaaataaagagcccttttttctgtgtttcttgctgatggcaggctgtgtgcttcatctgcttttatctgctctgctagctctgactctactgtgatccagcatgtctctcggcgtttgaggagacatcccccactgacctgctctttctctccccagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagtaagtctctagagggcattgtaaccctagtcattcattagcgctggctccactggagcccagttttagagtttcttttctagggactctgaaggtagtccttctaacaccatccaagtgcctcagtggggacagtttccctctattcctgaaaataacgacagcttcgttcttagcaaccaaggggagggtcttctgaggccccgtagctcaggctactcatgatgggacaagcaggaggccactgcacgtttcaaatgaggaactttcagtgagagggcctcagggggacactctcacagtggcatctgatggggtttcgggaataattgccgaggtcagatgtgggttagtgcaacctgtgcttctcatgggagggtggagactgagaggcagaagtgatgatatagagggttagaatcacttaattttacttacagaaaaacctaggctcaaagtgttgaagccatttgtgcaggagtgagtttgtagcagagctagaactggagcccggatttcctttgctgctatattttccctttagaaatgcccatttcagaactgaaatagaaatactgtccataggcttctctttcacctacagagaagaaaagcagatttcctccttctgccctggacactagttcatcatctgtcggaagcagtcataaacaagcacacatttactatgcatacaatgtaccgttatgacaaaggaggaccaaaatccaaacaatatcaaaccacaccaaaaaccacaaggagcctaataattactaaggtgatacttccaaagggaggactttatttcttagatgagaatgaaaatggacacattggaaattattggagagccctctggctatgagtccttccacaaccatatggtaccaccgactggcaggagaaatgtgtgaacatgtgcctcctcctcccccaaccactggggtcggtggggtgacggtggcacttttagcagtatcctccgtggtttgagttgaaaataagttttaaaaatcctgtgagtcatggttttgcattgaaacctcttcccactgtgtacccacaaatagttaactaaatagaccattagaaaaggaagaaaatataaagcagatgccaagcagagatgtcctaatttttgacaaaaaagcaatgttgcttgtgtcaagaagaaactgaactttgtgaagagttgaaatggaattccactgaattagaaaaacttgttttctcctgcctggatacatacagtcagggccattgatgcacaggtgttcctggctgttgttacactttaccctctgaaatgatgctcccaagtgctatgtgatgagctccttgtgtgcccagtggaataggtgtgtccatgtgtcattttaaagactattaattacactaatatagtttctttctctctttggataataggcacgttgttccgtatggacttggaagccctaggtccaagagagccttggagaatttacttcccacaaaggcaacagaccgtgaaaatagatgccaatgtgctagccaaaaagacaagaagtgctggaatttttgccaagcaggaaaagaactcaggtgagcagaaacacctttgcttttcaatcagtttaacagcctcctgaactccttcctatcatggtactgccttcctgttttagagagactaacagagacattgaaagtcagggtaaagctgaatataacattgctgaaatgtttttccttgtgtattttaacagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtaagagggaaggaagaaaaattaggtaagaggttcacaagaacaactagccccagtcagtgatgccagcagcctgttcctccagcccttcttacccgggcaggtgaaagacttagaaaacagtagcagaggagatctatgcatcctatagattaaaaggagcaaaagaatccctcttaaatatttccatgaagctctggaatgcaaaccgatgtcctctgtacttttagcacataccatttcatctacaggtagatttcccaaccaaaatatatccagagatgcctttgtcattgggttatatacagcctttgcctctctgagtcaatgtatttaccactttccctgagaaatcgaaaatcattttggggagcggacatttagaaaaagaatcaaagtgtcatggataatcaaattcttcaataagttgcagttattcagatggccaaaggaaaaataaagtcattagatagggttggtagaatttagaacatgctgtttttcaggtttatggtcttttttttttttttttttttaaatagggaaatgtgtttggtgcagagccaatgtcattccaaaaagctctctcttttcctggtcagtcatgtgctgggacagagaagggatctggattaggcaacatcatagagttgctctgagctgctctttggtgataacccttccaaatcctaaactttttggaattcacaagctcaaaggaggaaacctactctctgatctaccacatgttctgcatttttctatcatggtctatggaaacttctcttagaaatccagtggcaagaagttctatgattaaagtgttctgagctcaggccaggcagtcatgaactacttctgagttatttactactgatttgtggggcagcctcagctatcggtttcttcacacctgcttatgagagtatccatatttatggtcgcaggccagtaatgctccccacgagatcagtttctgaactaacctggaattttttatgggtttttattatgccaactattaaatcaacattacagttcttccctctgtatttctcctgtaaaacattaggcctgcaaaaaaaaaaaatctttttaaaaataattgccataaagtatttgctctgggcctactgtatgcttcttttctttttctctcttttcaactaagtcaccgtcaatttattaagatggccataactattcaaaacctatgctgagttcctcaaggcagggtcacatagtgatgaaggttgggatggggctacggaagaaaccagaacaactctagtttatttaaaacctgtatttactgcccacttccccttagacttgaccatatgacccctcgctcccattctaagcataggggcaggctttatttttacaatggtaatagatatcacttgaggttttatcaaagagttgcggcgggtggtgaaagttcacaaccagattcaggttttgtttgtgccagattctaattttacatgtttcttttgccaaagggtgatttttttaaaataacatttgttttctcttatcttgctttattaggtcggagaccatgagaaacagcgtcaaatcatcttttcatgatcccaagctgaaaggcaagccctccagagagcgttatgtgacccacaaccgagcacattggtgacagaccttcggggcctgtctgaagccatagcctccacggagagccctgtggccgactctgcactctccaccctggctgggatcagagcaggagcatcctctgctggttcctgactggcaaaggaccagcgtcctcgttcaaaacattccaagaaaggttaaggagttcccccaaccatcttcactggcttccatcagtggtaactgctttggtctcttctttcatctggggatgacaatggacctctcagcagaaacacacagtcacattcgaattcgggtggcatcctccggagagagagagaggaaggagattccacacaggggtggagtttctgacgaaggtcctaagggagtgtttgtgtctgactcaggcgcctggcacatttcagggagaaactccaaagtccacacaaagattttctaaggaatgcacaaattgaaaacacactcaaaagacaaacatgcaagtaaagaaaaaaaaaagaaagacttttgtttaaatttgtaaaatgcaaaactgaatgaaactgttactaccataaatcaggatatgtttcatgaatatgagtctacctcacctatattgcactctggcagaagtatttcccacatttaattattgcctccccaaactcttcccacccctgctgccccttcctccatcccccatactaaatcctagcctcgtagaagtctggtctaatgtgtcagcagtagatataatattttcatggtaatctactagctctgatccataagaaaaaaaagatcattaaatcaggagattccctgtccttgatttttggagacacaatggtatagggttgtttatgaaatatattgaaaagtaagtgtttgttacgctttaaagcagtaaaattattttcctttatataaccggctaatgaaagaggttggattgaattttgatgtacttatttttttatagatatttatattcaaacaatttattccttatatttaccatgttaaatatctgtttgggcaggccatattggtctatgtatttttaaaatatgtatttctaaatgaaattgagaacatgctttgttttgcctgtcaaggtaatgactttagaaaataaatatttttttccttactgtactgatttggaatcattactgaaatttgtaaggagtgggccaacgtgattaagtaccataaaggcaaataaatggttaaagacggtttcatagaaaagtgacaattagaaggatattacggtctaagctaattatataaagaattttatctgtatcttaaatgttgattttatactgcattgaggtaaaaacacaaaacaaaaaagcagctttaacacctctgtcttctcttgggtagcagcctcctgcttctccttcacctgaaaaattctccagggacttcatccattaacttggctcaggctattaggcaggattcaacagtttaagctgatggtgtggtgagagatgctttatccatattaatggactgaaggaagtaatggcaagacaaccccccaaaacatacctaattatacaaagttatataccaaagttgcttttagaaaatggcctgctcagagcaagtagaggtttccaatggctttttattttctcacattaaggatgttgtttcttaaggaacattgagtaccattgcttcttcgtgatagcctaggactggccgtgtgcccatggaggtagagacaccaggtactgattctaggtcctctgccacaaagcaccacttcctctccactttgccttggctggccttgtcagctcactggagagcacagtattgcaattgcagtattgcaaatggtcactactaactgaattctctaagagcttgattagccctcgagaatcttccttgcccttctctaatagtgtctgaaggaattcctggcatttaacaaatattagcatgtagtgatcactgtcgtcctaacagtgacacatcagaaggatttcaaataacagtcttcaggcatgcgtaatcaatgtcctgtgcagagtctccgtcctcattgatcctcatttttctctttaaggcacagtccaatgtctttggggaattgtttataaagcttactttatccataaactgtttctcagtgcgtgactctgaagaaaattttgaagttttgcccatgttgacaaggtgcttggtctgaacttggccagtatttaatcttgagcaaacgattcaatttccttctatcgtgagttttctcatctatgaaacaagggagttgaggggagtttctttcatacctctgagaaagagtttgagattacataaagaagttgaagtggcatgaaaaaaaataaagatctgagcttagaagacatggatctaatacatttaagaggaagtcagaatcagagaagccactgaacaaaacagtccaaaggacagcatagtaagtcagattgatgagttttggttgggtttttcatcagtcaaacccttgagcccccctttcccatgcttcctgcttcagtatccagtaggaaaaatgaaagggatgatgtagacactctagggcatgaggatttgcagtaaataagttgggagactcacagaaaattaatatttttcaaacatgaagacgaaacattcaattatattacagtccacatcagcttgaagggtaaactgatgggatggtctgtcacatttcttgctctgtttccagtaaaagcatggtttctggaaacccacttaggacagctttctctctttacactgatagcccaggcaagctttgatctcagaactccagaaaccagagaactctaggtggaatgtggtaacttttgccagggcagagggaacacctactaataggtacttcatttgcaccaccagagattggcatcttttttgatggatccactggctttgatactgcctgtactcccccaaaacacagcttgggtattggactaatctagagctccctcaggagaactcttgctgacattaagaaagagcaacattttgtctttccaggtgaaaatccaaggcccaaaaagggaggtgactcacctaagatcacagaaggagctgtagcatctctggagcctgaacacttaagttaagcacgactatttcagccagagggcccctggaggtctgcagaaacagatgcccaggcaaaccttgctagaaggctctgggtactaaagccaaaatcagtctttctgaaagctttgcctcccagccttgtagacagtgctagcagacccatcccctcaagaggaaagagaagaaatgcaggggaagaaaaaaggactataaggaggctatctcatggcatcgttaagtaaatgtctataggataaaatagaatataaccaagaatttttaattattatttttagactttactcaccaaagaatctatctgagtgaagggggaaatctctcggtcagttctctttttttttttttttttttttttttttgagatggagttttgctcttgttgcctaggcaggagtgcaatggcgctatctcagctcaccacaacctctgcctcctgggttcaagcgattctcctgcctcagcctcctcagtagctgagattacaggcatgtgccaccatgcccggctaattttgtatttttagtagagacggggcttctccgtgttggtcaggctggtctcgaactcctgacctcaggtgatccgcccaccttggcctcccaaagtcctgtgattacaggcgtgagccactgtgcctggcccggtcagttctcttttactctacaatgcaaggcagtggtctgcttttttttttccccctctctgtttctaaggtcttgaaaatgcctgttttcacatttaagttctcaatgtgtaaaacactgctttcaagcttatgttcttaatagagtcccttctgcattggctttatctcacctttatactcacagttcctggggactttgggtctggagctcagaatttatctactgcaagtgaatgttacacatttcacagtaacctctcatctttgtagataatttggtttcctgtagcttttcacatcctttctctcattcgacccctaccctcacacaacaatcctttcgggaagctggaaccatatcatgtatccatcagccaaccaaggaaagagacatatcgagagggtgagtgactcatccagagtccaacatcaggcaaatagcaagaccagaacccgggacccctgaccctttcttcttaaactcctccagagccaaggggatacactggacctgcaacttggactaacgtagattatttccagcttgtgagtgaactgtgagtggaatgaacttgttcttattctcttaggaagatcaaagctccgtatgtaccctgagccctagttttgaacgtctctgtcgtatgattatttgtttactgcatcattcttgttatgacaatgcatggttggcagaaaacttgctggcatttacaaagcttgcctcaaatctggtgccatgaagccatgacaacttcattgacaactactggccacagagccaatacatcggaggaaatagagtgatgaatgtgatggcagcggccatcaaatttcctcaaatcttgggcagtaatcagagagagagctagggctggtgccactaggtagtaaaaagagaagggcttcggccttggagacaatgaatgatggaaatgttgggtcccagggatgacaggctgcccgtagctcagaaaggcagcacatcgggagtccggcatgcagttttgcccgtttagagttttctgggtttctgcatcctattgttatttttcacattccgagtcattccagtccccttctgtgtaagttgctgtggaagtatccagggctaataatcacaatattaagcggccattgacatggcatgggcactgaccaattagaaaaagctgggacagagctgttgggcaggctcctgatagactctggctgggctaggtcttcaacctcctgatgttggcctgtaggggaccttgtgatctgggacaggggtgggcagccagggtggaatttccaagcttcttgttgctagggctatttactaaccaggatgaaatcatttcactgttttacagactagaatagtctcactggtatgtgctcctgtgtacgtcagatttaaaagtatcgaaaccattctctgcttgataaattattctctttacagcttcactgagtgagatgacattatcttcatgcctgcggaggtttggcaccaaatctttctgctcatgcacctttggtgtcctgctgcacagtcatctctggcaatgcccctctctctgcatgttcccctccctcgtgctctggtgccctatgggagaagtaaaaagaccttaagggtctccttgtctccttggagattttactttaaattttttttttattttatgtgttttctatttcaatagctttaagtggtacaagtggtttttagttacatggatgaattgtgtagtggtgaagtctaggattttagtgcatcccttactcaagtagcgtgcactgtacccagtaggtagtttttcagccctcaacccctcctatcctccccaacttctgggtctccaatgtccattataccattctgtatgcctttgtgtacccatagcttagctcctacttataagcgggaacatgttgcatttgattttcaattgctgagttacttcactaagaataatgacctccagttccaaccaagttgctgcaaaagacatcatttagctcttttttatggctgaagagtattccatggtatatatatattacattttcttggccttctcatcagttgatgagcacttaggttgattccatacctttgcaattgtgaattgtgctgtgataaacatactcacgcaggtgtgtttttgatgcattgacttcttttcctttatgtaaattccaagtagtgggattggcggatagaatggtagatctactttttagttctttgagaactctccatactgttttccacagagggtgtatgaatttacatccttcttggagatttttagcagggcaattttgtggttgccaaagtcaagatctgccttagaccaggtcttcctagggccctagctctgcaataggaaggagcaagagggacgagctgcctgtaagattaaatcttgccctcttagattaatttgggactaggaaagggtggccccataatttactatttaaactgggacatatttgacagcacttagtgacattcttaacaattctcacggagctacacattggactgttgtggacaaacttggacatatggtcatcttagatcaaaataagccttgaatcagttcagaatttcagaaccgcctttgcttcttggatgtgcttggcactgtgtaagcacacgcaatgtctgtatgcctttacctgcagaccaggctgcagcagagcacctcccccttttctggtgcctacagcctcaagttcagagcttagggcataatatatcattcaataaatgcttattcaatggatgaataaatacagaggtgagttccactgttcagtaagagcggggtgaaagtgatactcttctagccagaatgcgactagacaacagaagatttataagacccatttccagggctctctttgcagagctgccatagctgaaatcttcttttattgaggctccacgctagtggtaaattatttgacacaatggggtattctttctacttctttctactccaacccatcctgcatttcaccataatcataataacaataatcttctccaaatgctttaattataatttcaataaatacagagctagaaggaaattaaaaataattttggctattaccctcatctcaaaaaccttccactgccactctgcctatacaataaaaatatgctatctactgagacaggcaccagactaggaagaaaggttgggaaatgttacaatttcagtattgagcatattaagttttaaatttttcatcatagtgtgaaagttgtcagagtcaaatgaagtcacctgtgttaggaaccctgacaaatggagccaggaaaggccataaagggagaattctcacacacaaatgcctgataaccaaagctatcacagaatactttgcaaaaaccacaatcttgcacaaaggccatcacaaccttacacaaaaaatacttcagtgaagttatctgcccagcaactgcctgtctagcctcggactggtgccacccttgttattgataactgtagccaagaataatgatctcaaaacaattacgttatcctcctgatttttcctttaacaatctttgttttgctttacctcctcgaatacgcacatagtttactatggcatgtgtattttcattgcaatgtccatccctgaataaaaataattttcttttcatttattaatgtatttgttttttgaaactgggtctccctctgtcacccaggctggagtgcaatggcacaatcacagctcactgtagccttgacctcccaggctcaagtgaccctcccacctcagcctcctaagtagctgggaccacaggtgtacaccaccatgctcagctaattaaaaaacatttttgtagagatagggtcttcctatgttgcccaggctggttttgaactcctgggctcaagcaatcttctcgccttggcctcccaaagtgctgggattactggagctgttgtgcccagctcaatttcttttagggagtctctctgtctgttatctaggttgacagtggttatcttcaaatatatccaacagttgagagaatagtataactctaaagccaatatacctagcatatagtttatatagttgttgagatatgctgggtgttgctttatctctacctgcatcctgccctccttccttgaattactttgaagccaatcccagctatcattatcatcttacctataaatatttcagtgtagttgtaataaacttgatatgctttgctcgatcaggtaaaatgtgaaatagtcaactagatatgtggacctagagcttacagtggatgaagaactcacaataacaaacgcctcacggaactcacaataacgaaagagacaatgcatgttgatagcttgtgtttatgtggtgcttactaatatcccagacagtcttttgagaactttgcatgcatcagctcattttattcttactaaaacactatgaggtatttatcctatgttgcagatgaggaaacttgaagacagaaattcagtagtaacttatgcaaggtcaggcagctagtgaagaggtaagaaccaggtaacctcattcactatgcttttttgattcagaagaatccaaagtgataaaggttacattatgggataatcaagtcctatgatggtatcaaagtgacagtgatcaactctgcctggggacagggaagggaagtggtggtttcagggaaggtttcatgtaaagaaacagtgaaaaatcagccttgaattcctaagaaggataccatttgagaaatttaaagctgaatcaaaggcgtgaagaagtctcaataaaatgctttgtcgatagatgacttgataaaaagaacataacctaacatgctcatgtgactgactcataagaaaagtaagcaggtatctagatcattaaaagatgatttatcttgcaataatccaatgatattggaaatcttaaagcaacacaacaacactaggacagaaatctctaattttcaagtggcattgattgaactgaaattttgaggtatgactaaaaatagcaagaaagcaattctaacagattttcactaaattttcagaagttaactacatttaacattataatacaatgaaataattccactgaaaatcactgtacttactgaatgtaagcttattttatagttcaaatgccctatcaacctcctactctttaatgcatggtcacaaatgacctctcaccatgtcaagaattccctcttgatgtatggataggaataacccacctgtcaacttaggtgtcttccttgtgtttgaacaatttgctaaggtaccattaccaatcgaccctgctctgacattggtcaccggtgtggttctttccatataacctcatcgttttgccctgatcagcggccacaggcaggcagtaaccgttggtttggtgaaagacaaaatgccctagacaattaaggagatgatcttattcaggctactacaataaggaaaatgttcattaatgaagactgtctcaaggaaaggaaggaaacctggggtttcatagagacagtaaacaagggagcaactgaatcttttggtagtcatgaggatggagatggtcttttcttaaccatgaagaaagggtggaaaggaagaggctcgagcagggtgtttccttgcagttagccatttctcaggacacaaagaatgaggagatattttgagggacacaggactcagataaatttcttcattgtcagtccctggtttttttcaagatgaatatcattcatcaaggaaacactcaatgacaatcataaatctcctgagtggggtgaagtaaggtctcagaaatcatttctcaaagagtcttagtgttattcggacaaagacagttgaagcatctgtagaaagataatgtcaaggtcagttgtgttgggattctggggactaggcaccgtaaaagaggattgcttaaaaagaaaaagagaaatataagtataaaaggtttactaaagagccagaaattgaataatgagggtaaccagttcagtggattccaagaggtcagtggaggaaaatctttcagttctgtaacactgctctttgagtctcttagagctactgaataggacatcagtgctttcagtggtgctgtccacagggcatgtctgactctagcattccacaggcttcttagtggcccaggcagtgcctttccagggagaaactgggcccatgaatggtctgctacagtggtgagtcctttgaagtatatatagcaaattttagcttataatgcttcttcagatcctagggaaaaggacccagatacaagacaaccttgagtccttacagggatccgggtagtcagattttagttctcggtgacactgagtcaggcagcagtgagaaaaattgaaaaaactgttggaaaccagaaagattgaagatctgaaaaggattgataatttgggaagtttacaggatccagtccaacttacagataggtacaaaaactaacagctagggagaaagtcaattacaattccaccagacaagacagtttgcctattaatcattactcacattaaaaaaaaacccaaacagctcaaaaacaatgaacaaggttagaatgagataacctgggagactgtgctatctgatgtatagtttttcattaaaatgcaaaatttatttgtgtagtcactgactcttgatcaagaataatctcaaagaaagattattcttgccattacaagccatctcattagatctagaatggttattcagattggtacagcaagaatggtaatttaccatattggccttttaagtttgctttttggaaattttcataaggaggctcagattggactgttaaaaaaaatctctctagacaggaagccaagccaataacttgctaccagatttcacctgtcaagtacctataaatctgggcaaatttattttttcttcaagtcccccaaatatcctaggattcctaggcatgccaggaaaggaccttctttacctctttactcacctaaaaggaagaggatctgtaaatcaggtatcaggcctgttttcccgggaaggctttgtaagcatcatttccgtaaagccaaccttagcttcttaaaagcatctggtcatatctgattaaatgagcatcattctcaactatgacattccaagcactgccttagttgtataacctgtattttcaattttgtcatggtaacaagaaggacagattcttattgaacctatgcaaatgattatgttgccatgaaaataaaaatactcaataagagtttccaaattctggaggagtcaggcagagagaatcaaataccactatcaaatgtttcatttcgtaagtgatatcttattctctgagtttataaactgagtttcagtttataaaagcaaaatctaaattgctatgtattatgtattatagaccacttaagaggaaaaagaaagggcttccctatatatccagaaaacagaatatcagaatgatcatattccaaacaaaaagcacaaacagtcctcgtcagctcattcagtgctatgtagttaatttgtattccactcagtcttgagtgaagagtctcatgaacccctttgtttcttggctagagttctaggaatcctaactcagtccactggtatgttctcaatattgtttaagtgacaccatcagaagccagtacccaaaagtatctggcaaaagtgaaggacaatctggcatcatccttttctacagggctttgagaagataccacgccgaggacaagcactatggcctctagctgatttgcaaagactgagatgcatcagagtaaaacaaagactatctgtagatgacaaaagacataaagtggcactgtggttaacttcttactgataattttcaaaagtgaaagatctggtgacaggtcattagaacaataatgcaactgacaaggaaatctggctgttcgtgtggagtacaaaacaagatcaggaagtcgatccaaaaggttttagacaagaagataatttcccctgttttcaaagagtggctattacatttgatttataaaattgggttgtattagtccattctcaggtagtgctataaagaactacctgagactgggtaatttataaagaagtttaattgactctccacaggtttaacagaaagcatggcaaggaggactcaggaaacttacaatcatggtggaaagcgaatgggaagcaggtactttcttcacaatggtggcaggagagagagagagccaagggggaggtgcttttaaaacatcagatcttgtgagaactcactcactatcatgagaatagcaagggggacgtctgcctccactgagccaaactgtatcacgggtaaacattatttttacagagaaaaaaaatcttgataaataactgtaataatcctgacataatataccatgaatatatcaagcatatagttagaatataccacgaatatatcaacaatatattctgaatgagtctggagtacatcctaaatatctatatgttaataaaactccatagggaggtgatatgtatccccatttgaaaatcactggcctagaagattctaggttcaaatcaaagaagcaaagttgtgaccaagttaatatttttaaaaataactgaaattatgactgatagcactgtactattgtataacattaggcacagcagaactaggaccgtgacaaatagaaacatatgaagagtgatggtatggacaattctttagggatttctacagcacacactttctgaaatattaagaacacacattttagctagagaaagctaagcaaatctgatttgctaactcatcagttgtaacatatcaaatacacctaattatttccagcatctctctttttcatctttgtagataaaagtaaaaatcttgttacttttccaggccccctgcccttgaaaaattacagaaatagttttaggtgcaaaagaaatcattaagattgtgtttggggaagacaaacaccaaaagttgttaggcgatttgaacacttggtcaggtaagactatgggttactgagaaacaatacttggctacgtatttgactaaaacaacagtaaaaaatttaaaaagtaaatataagaggtaacatgattttaaagaagcttagctctttcctaagaaactctgttttcttaaataatcaaggacattgttgtggttcgaatgtttgtgtccccccaaaaattcatatgttgaaagctaattgccaatgtgatggtatttgcaaatggagcctttgggaggtgattaggtcatgaaggcagaccctcatgaatgggattagtgcccttagaaaagaggcccaagacagcttccctgagccttctaccatgggaggacacagcgaaaaggcaccacctatgagccaggaagcggccctcaccaccagacattgaatctcctagtgccttgatcttggacttcccagcctctagaactgtgggaaataaatttctgttgtttataagccacctagtttatagtattttgttatagtatcccaaacagactaagacatgataaagtcagcataaaccaggttattctagtaagacacagacactttgttttctaggcagattgcacagaagataaagaataaccatttacaatcttttatcaaaagcaaaccaataatccaagaaaaaaattcatctaacagaaagaaaacaaaattctagttttctaaagctctttttttttttttttttttaaagacagagcctcactctgttgcccaggctggagtgcagtgctgtgatctcagctcactgcaacctccaccttccagtttcaagtgattctcatgcctcagcctcccgagtagctgggactataggcgcacaccactgcgcctggctaatttttgtagttttagtagagacagggtttcaccatgttggccaggctggtcgtcaaacgcctgatgtcaggtgatccacctgccttagcctctcagagtgctgggattacaggcatgagcccctgtgcccagcctctaaagctgtttttgaaaattttatatgtatacccattaaactttacctaactttggccatgacaaagaaaattccttttctgagaagctcctatacttttctgtgttcatgtgtatccctactagtgtaacttctcaaaatgccagaaattcattaatagatatatacagatttttaccacataaaataagaggcaaattatataaacttaaaatgatgtttagcaattaacgttcagtattctgcttttctcagaaatgatctaggcagctactggatatccattcattaactcaatttattattggcccaaggttttaagtttcctaaagactttgcaaattatcttcaagctgacacattgtataacataattactgctgaaataaaatttgtcaaaataagtcttcaaattacttaaacacaaattcgcattttttcatcattttaaacatttagtagaagtgatgctagcttcctcaattagtaaatctgtataagtttaaaaaatatttacaaatataacaaaatctgtgcttatgcaatatttaacactgataaatcagagaaaacttagctgtatttattaaatcaaaattattaagctagactcatttgccaaaaaagtacctaaattacataaaattagatttctaaattatttctgagttaatatctataacaataccttttttaatccccacactgaaagtattagaggttcaattttcttaatttctttctcttcataaagggatattcttaataaaggaatatttagtcaatataaatgcttatttatctctaagcaaatcagactagagattctttaagacatttcctaatctagcttattgatattatcaggagatagaaaaatattatacactcacacgatgagaagtaaaggactttctaaatacagacatactcacagagaaagcttataatttcatttctaaaatttcagtcagatgtcaaatataaaccaacaaatacaaaacccgccagtccagatattaacttcctggttggcataatattcttaatagatttgaactaaaaatagacagacagagaggagattctagctaaattctgtcacctttcactcaacagagacaattacctctataaaccatcagttgcttaaagagatcatcaaatgttcagaccatgaaaataaaagttgtcgctagaaattgagtaagtgctcaggaaaagtaacaatcaaatctactgtgctactgatgaacaaaagtcacatgagttggactcacctttgggtccccgaatctgtagtccggaacacagagggctccagctgcctagatacccctgtggaataacagagctctgaagtgaggcccaagctattcaagttattgcacccaacagagggagtccttgtaaggagaatggggtaagaaagggagcctggtcagcacaaatgtatcaggaaagtggctcaagtgcagtaaaatgactaagagtatggtcattgtattcagatatacctggattcaaatcatggctccactgcttataagccccttgggaagctattttaccttcccaagtctccatctattcatctataatatggagataataatacctaccacacagtgtgattgtagattaccattccatatgaaactctggagaccggccctttattcatcacacaaacccttcaccatatgaccctaacttacttttcctgtttttgtttgtttgtttgtttgtttgtttgagacagggtctctctctgttgcccaggctggactgcagtggcatgaacacagctcactacaggctcgacctcttgggctcaagcgatcctcccacctcagcctcctgagtagctgggactacaggtgcacaccaccatgcccagctaatttttttattttttgtagagatgcgtctcactttgttacccaggctggtctcaaactcctggcctcaaataatccttctgcctcgatctcccaaagtgctgggattacaggtgtgagccaccatgcccagccctcctgttttatcttcaccctacattggtactatcgtaggggaacatttctcaaattttgtgtgcaacagagttaacctagaaaaattgataaaatgaagattcccaggtcacaaaccccagaaattaagacctagtaaatgatagaacctagaaatctttgttttagcaagaacccaggtggttcagacataggtgttttttagattatgtttaattttaaaaatataccacgtgagcagccgggcgcagtggcgcatgcctgtaatcccagcattttgggaggccgaggtgggcagatcacgaggtcaggagatagagatcattctggccaacatggtgaaaccccgtctctactaaaaatacaaaaattagctgggcgtggtggcgcacacctgtagtctcagctactcaggaggctgaggcaggagaatcgcttgaacccgggaggtggaggttgcagagctgagatcacgctactgttctccagcctggtgccagaatgagactccatctcaaatgtatatatacacacgtatatatacatataagtgtatatatatgtatatatgtgtatatatacatatattacatatataacataacacatatattacctctataacatacatattacatatatattacatagatgttacatatattatatatacacgtatacatatatctatatatacacgtatatatagatatatataaaatgttagctattcctcaccctggctgtgaactttcgtttctctccacccaggcttttgaaatgccactttctcatagaagaatggcttttcttcaaggttctgctggtgagctgttttttaaatgtgttctcttctgtgaggctttcctggacttctcaaggcaaaaaggctttgtggtctctcctctggtatttgttaacaagtctgattccccacttgacagagagctcttgtaggggaggcacggcatcttttttttatctctgtgttctcagcatccagagaaggcacgctttgaatgtctgttaattgagatgacaatcccaaccccatattgcaaggagaatgattggaatctctcccatacttggctttaaagtcatctacaagcctgaactgagagtcttagagactttcaggccagaaggtactaagaggtttgtcaagtcacagactgaagaaaatatttgcaatacatatatctgataaaagactgggcatctctctggaatatcagtttctaagaaatggtgctaacacatccttataataaatcagaaaaacggtgcctggtaactcaggtttccacatgcaactcctttcattactggaaataaaaattgttttactttgaccaaagtagatgatgtcttccagtataaaaaaaaaaaaaacactcctccccagcatcaacctacctctgttgtttttgtacatgaatatgttattagcagcaatgacagacggctaacatctgagtccagccacttgacaatcattcgaagtcagcaggaacagacgtgctgaaataaaaaaagacgagatgaagtgctctacctagggaacaccgccttgcaatatatacgtgtcactgacctctacttaggaatgtgagtgcaaattgtcctctggctatgggcaacaacataagccacgaaatgaaagagctccaaataaattaaaatgataaatttagcgatttagtgtgaacataaaaagctttacaacttcacgaattaaaaaaacagtttcaatgttagaagtcaagagagcagctccctttagggggtggggtggtagtgggcagaggagagcctgggggacttctgtcttctgtttcttggtctgggtgctgagaacataggcgtgttcactttggaggaatgctccaagctgcacatcaagatatatgctctttcccgtatgtgttatgtttcaaaaaagcttaccccttcctcctctcaaaaagcattttttccctttcatttttttatttcttcttcttcttctctctctctctttttttttaactagatacacgctgaacaaacaaaatatgcaaagaataatagtttgaaggggtgccagcatgctagcgtgcctctggaagcgtacatgccttggtctggactgggcatcagatgtgcattttcacttgatgtaattcatttccttgttccatgacccagctttcctctttggggtaaaggcccgtccagcatttttcttggacaaggtctccacctttgctgcatccagggagactccttcagtgccacggtcctgcctcaggttagtttccttgtacgtgttgcctggctttgtcaggtcttaatgccatcctgaagctggtaaaatgagccaagtgacttcatctctcatggcacaccttgaggacattacttgggcacaagccaattcactgctcacagcccgagagacatttttttcccatgagctagctcctctcctcctctctcacaaataacttcctctatgggaaaggacacatcagagaacaaggagcattgtaacgggtgagaaatatgcttgacatggggaaggagatgccgtcctcactccctccttccccaccctcccacactcatctgtcaactctaatttctcactaatgcttgggtttcactctaagctccctttgcttgctcgtgcccaccaacagaaatatccaggaaaagaaagagcatcaggaatgactggcctgtgtgcaagcaattccttgttgttgctgactgccttctgcagcttggcgaatttgactcttggaatgcacggacagaagtagcatttttctctccaccagaagtctgacactgaccatgtcaatgagcgtcaattggcagtacgtgtgtgttggttgaccctttacatcaagaccaagctaagcatttgctttcctttggtcaattccactggtccttccttttggtcaatcccatcgctctctgacccagggccagaggaacatagcatttctcctaacttggacaatgttgcaacaccaaaaaggcttgtactgaagtacggaggaggtagatgtgaagagtcatccttttgtcagattgcaaaggtgttgcagcccattagtttcaggatgtttttattttgttcccttccagtaaatgttttcagagattttgcctcgggtttttaaagatgttttataagttatgggtctctttaaaatttttaatcatcttaatgaggtataatttatatacaataaaatgcctagacagtgatcccatttgatggattttacaattatatatacccatccaaaccaagatagaaaacacctccttcacccagaaaacccctcatttccttttccagtcagttccctcacacacactcactcctgccctacccccagccccactagagcctaggagaagtcactgttctgatttctttcaccataagttagctttgcttctataaaatgtagcacctcagtatatattcttttaggtcttgctaattttgctcaacataatgtttttgagatttgttcatgttattgcctttgtcagcagtttgttctactttattgctgagtaatattccattgtacaaatatatgacaatacatttatcaattctcctgtatgagtacaatggaattgtttctatttggagctgttactaagagtgctgtgagcactcttgtaaaagtcttttttatgttctttttttccatttctcttgcataaatatctgagagtagaattgcggggccataagtaggtttatggttaactttatatgaaagttccaaaatttttccaaactggttataccattcttatattcttaccagcagtgcctgggatatcgaattgcttcatgcttggtattgtcaatatttttattttacttagtccaacatttggtattatcacagtttatttacttcagctatgatgatttgtgtgaaatgccatttcactgtggcattagttggcatttccctgatgatctacggtgttgagcttactggccattcgttcttcctatgtgataggcacgttcatgtattttgcctgtttttacaaattgacttttttgtgtttttattgttggtttttaagagttctctatacatcctggatatgagtcctttattagacatatgtattgcataggtattttccagtttgtagcttgagaatttattttttaatgatggcttctgatgaacagaaattattaatatttatgaagtctgacatcattttttttaaattactcctttttatgtcctgcttaagacatctttgtcttcccaaatgttgtgctatattctcaaggttcctcttagagactttaggtttttagtctttatgtttttgtctatgatccatatcaaattaatttttgtgcacggtataaggcggggggtggtcaagactcattttttcccccaatatccaggtgttctagcactatttgtctaaaagtctttcatttccccattggaatgacttggtgcttttgttaagaagccattagtatatgtgtgtgtgtggctatttctgggttctctattttgttccattgatctatttctggactcctttcatttccatagatcactatcacaatatctatagctgtagagtatgttttcagattgattagtgtaagatctctaactttgttcttttttatcaagatcgtttggctgttttgtgccctctgggtttctgtatttattttagaaacaacttggcacttcctttaaaaaatgatgcttgaattttgatcgaattgggttgaatttatcctcgatttaggaaggatggatatcttaacaacattcagtcttacaaaaacagtatgcctttttgccgggcacagtggctcacgccagtaatcccaacactttgggaggttgaggtggacagaacacaacaggccaggagttcgagaccagcctggtcaacatggtgaaaccccatctctactaaaaatacaaaaattagccagatgtgatggtgcatgcctgtaatcccagctacttgggaggctgaggcacaagaatcacttgaaccaggggggtggaggttgcagtgagctgagattgtgccactggactccagcctgggcgacagagcaagaccctgtctcaaaataaataaataagtaaacagataaaactagactaaataaaagaaaccagtgtggctttttattaagttaggttttcagtttcttttaacaatgttttgtagttttcaatgtagagatcttgcatatttcattatatttattcctagccatttaattgtgtggtcatattttaaaatttttatttgccagtttttattgttagtgcagaatatatactgacttttaccctaaagctttgctgcactcttttattacttttaaaaatatttgtgtagattccatggagtttgctatgtcacatgttctttgaataacaatgattttttcctttagaatgtttgtgctttttattgcattgacttactctataaaattaccatatcttctagaatattgctgaatagaagtgttgagtgaatgttcttgccttgctcctaatcctagtggaaaaggatttaatgttactagtttaaagaataatctgtaatttgattgagaaaatttcctctatttctagtttgctgatagtttttatcgtgaatcagtgttgaatttcatcaagttcattttctgcatctattgagatgatttttttctcctttattctcttaatgtggcaatttacactgatttattttcacacattgaaccaatcttacatttctgaatatatcccacttggtcatgacttattttttcattatagctttttgtgtgttgccagatttgatttgccaatatttccttaaggcatatataggtgtgtgtgtgtgtatatatatacacacacatatatatatatatacatatatacatatatatacacatatatacatatatacatatatacacatatatatacatacatacctatatatacatatgtgtgtatatatatacatatgtgtgtgtgtatatatatatatatatttataagtttaaaagttatttcgaaaatataggctgagtatcccttacccaaaatgtttgggacccaaggtggtttggaatttggactttttttggattttgaatagttagataaatataataagatatcttggggatgggacccacgtctaaacacaaaatgcatttatattttatatataccttacatacatagcctgaaggtaattttacataatattttaaataattttgtgcatgaaacaaattttgattgtgttttgactatgacctgtcacatgaggtcaggtgtggaattttctacttgtggtgtcctgttggcactcaaaatgtttcaggttttggatcattctggatccagaatggatccaggtataggttggattccacctataccaacgtttttgccatcttagggtctggttttattgacttttttttcttttttgcttgtttgtcttcttgcttttttgtgtttgtttttaaatttcagattgaacgccagacattaggtatttttttaaaaaacagtagagactgagacaccacttatttaccctcagaaaagggcatgcctcatcttctgtccagttattcgtataggagtttggcagatgaagtctgtcgttgaggtgggcctgaactttgttgtggctttagttatggtaatacaacccaaatttgaaatgaaactacactctgatgccttttatttagtataagcactgggtgccagagagcatccctcaggtctcccactatgactacagacttcaggagaccctatgcccctgcacctcaggagagatctctcccagcattcctgccccttggccagtggccaactgctatttcctggcacttggtgtaaagcaggggcgtgggagggtttctctgagttcccctgatccacgattagactaaggcagcccttctgcacctgcagctcctcagctcctctgcccctcccccagatgtagacaagggcacctcatgcttagcacacagttctgaatgctggacccatttccttggctttcctgttccatcctcagtcttagacaggcccagtgccactatgctcagggaggtctctctcagacctcctgacccttcctggtgtcaggctgctctgcctaataggcagtgtaagttctggagcatagcaagattacttttggttttcctgctttgtcttcactcttaggttgatcatgttagcctgcatttccctggatttctaacccttttccatatgtggactgtcactttctgccacttagtgtaaggcctggagggtgagaaagttttctctcttcttctgccactcctggtgctgcccatattctcattcggtgtggacctggcacttcttttgtgaagcagtagttaaggagactttagaactcaccatggccaaggaaaggcccaagggtttgtcagtgagacagatcagattccaattgatgggcaaccaatcaatgaaacagacacaccctcacagttggaaatggaggatgaagatacaattgatgtgctccagcagcacgcaggaggtgtctactgaaaggggcacctgtctctttcctccagaatgctgttcttacagaccagtattaaattctcagtcagaaaactgcaatttggtttcaccacatcttgactaatagactaatacagtacagttttctctatcctttcctttcccttttcctcattcctttataaaatcacataaagtaactaatgtatttgcacaagaatatcacatttttaaactttcttattatattcagtggccagtggtatgttttaattgacatcgagtggagacaggatgggggaaaacactgattctgtgaaaataccccgtttctccactaccggcatgctcattcagctcttatctttatattccagtaagttattttgctctcactgttttaacaacaaccaccacaacagaaaaaacataaaaatctttgcataccttgttcaattgaataattttaatgatttcgtatctattattgtaaaaccaaggacaattttataactttttttgtacgtagctgttacatgtaaggcaatctgtttttaagtagggttaaattactcttaaaaaaaaaaaagaattctagacagttttctcttcaagtcaagcatgttgttgtttaagcttcttgtttaaaaataaattaaagttttctcgattcctctgctctctttctagtgtaggcagctggcatactgtgttacagtctgttcaccactggtttcaaatcttttttttttttttgagatgcagtttcactcttgtcacccaggctggagtgcaatggcgtgatcttggctcactgaaacctctgcctcctgggttcaaatgattctcctgcctcagtctcccaagtagttggtattacaggcacccgccaccatgccgggctaatatttgtatttttagtagagacgtggtttcaccatgttggccaggctggtcttgaactcctgacctcaggtgatctgcccacagacaggatcaggacttttcattcagctgggcttgagatctaaatatcaatgagatcccagagatctctttatggtttaaaactcggctttctgaaccttggtgtatctctctctctgtgtttgaccatctggcagctggatttttatattagatttttctcttcagtcactcctctagttttctggctcttgtgaggtctttctccttccttttgtcctgtccctagacattagcacttactgcttgcccttggtgaaagcctagtgttccttgaaaagactcctctcttggttcttctgcccttgttccagccttcagcaggccactgctggtgaggacttggagtatctcagggtacatttcctagttctcctgccctgctttcagtgaagttcctgagtgctttattggggttttatgagctcttctgccctgtctccagcctttggctgcctctgccttgcacttgttgaaggcgccatgtatctcatggcagatatctttcaggtcttctgctctgctctcagccttttgtgtactatctctgtgtactcagtaaaggcccataaaaaaacaaggagagggggtgcaaactcaatttgtgactagagccccttgagactccagtccatcatttcaaccaaccaacgggcatcaaaagttcatcaaggacaggcatggtggctggtgcttgtaatcccggcactttgggaggctgaggtgggtgtatcagttgagctcaggggtttgagactagcttgggcaacacggcaaaaccctgcctctacaaaacaaacaaataaacattggtctaatatggtggcacaggccagtagtcctagctactcaggaggttgaggtgggaggatcacttgagcccaggaggttgagccctgatcatgccactgtattctagcctgggtgacagagtgagaccctgtctcaaaaaaaaaaaaatgttcattaacagttcagctgaattctcactccatccacagcagattcctcctttctccaccttcattcgaggttcacagtagctacggttttctctctgctgaaattcatttcatttagattgtcttgcatcctcagttctttggtaggtttatttatttatttatttatttatttttgaggcagggtcttgctctgtcatccaggctggagtacagtggcacaatcacagctgcctgcagcctcagcctcctgggctcaagcaatcatcccaccttagcccctggaatagcttcacagctgggaccacagacatgtgacaccacgcccagctattttttttttttttttgagagatggtggtcttgctatgttgctctggctagtcttgaactcatgggctcaagcaatcctcctaccttggtctcccaaagtgctgggattatagacatgagccaccatgccttggcctctggtgagtttctttaaactatcattatatagcttgtctagatgtatcttgctgtaaaggtggaagtaatggtttcttgctactttttatatcctaataagaagcaagtgtctaaaagtggtcttaaaaattattaggtagctgaaattacacactattaattaagtacacacacacacacacacacacacacacacactccatttcctaatttctctttatttttttttcctactcagtcatccacagcactttctattgtccacctgtagttctcaattgctaatgatttttctttccaggggacagttggcaacatctggagacattttttattgttacagctggggagatggtgctattggagtccagtggtgtagagatcagggatgctactaaacatcgtatggtaagtaggaccgctctcaacaacaaaaaattatctggtccaaaatatcaatggttctgaggttaagaaactctgttctaagggaatatgaacaatcgcctgtagcagatacagtcattgtttttccaccaataaattctggtggctaccaagcaaatcaaaaagcaatagaagagaaaggaataatgtgtgtattgtctttctgatttcaggaaggcagtgtgtggatc

SEQ ID NO: 2 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 3 - DNA, 31 bp, Homo sapiens
ttaaagactattaatcacactaatatagttt

SEQ ID NO: 4 - DNA, 31 bp, Homo sapiens
caagctgaaaggcaatccctccagagagcgt